(19) United States
(12) Patent Application Publication
(10) Pub. No.: US 2021/0017110 A1
(43) Pub. Date: Jan. 21, 2021

(54) POLYCYCLIC AROMATIC COMPOUNDS AND METHODS FOR MAKING AND USING THE SAME

(71) Applicant: Board of Regents of the Nevada System Higher Education, on behalf of the University of Nevada, Reno, Reno, NV (US)

(72) Inventors: Wesley Chalifoux, Reno, NV (US); Wenlong Yang, Qingdao (CN); Khagendra Hamal, Vermillion, SD (US); Paban Sitaula, Lexington, KY (US)

(73) Assignee: Board of Regents of the Nevada System Higher Education, on behalf of the University of Nevada, Reno, Reno, NV (US)

(21) Appl. No.: 16/977,793

(22) PCT Filed: Mar. 4, 2019
(86) PCT No.: PCT/US2019/020609

§ 371 Date: Sep. 2, 2020

Related U.S. Application Data
(60) Provisional application No. 62/638,815, filed on Mar. 5, 2018.

(51) Int. Cl. C07C 43/205; C08F 132/08; C07C 323/21; C07D 333/10; C07D 333/76; C07D 213/06; C07B 37/10

(52) U.S. Cl. CPC C07C 43/205; C08F 132/08; C07C 323/21; C07B 37/10; C07D 333/76; C07D 213/06; C07D 333/10

(57) ABSTRACT

Disclosed herein are embodiments of polycyclic aromatic compounds and methods of making and using the same. Various different types of polycyclic ring systems are disclosed, including, but not limited to, polymeric aromatic compounds (e.g., nanographene compounds), pentacene-like compounds, chiral aromatic compounds, asymmetric arene compounds formed from naphthalene-, anthracene-, phenanthrene-, and pyrene-based starting compounds, and dimerized aromatic compounds. Also disclosed herein are novel benzannulation-based methods for making the disclosed polycyclic aromatic compounds.

### CROSS REFERENCE TO RELATED APPLICATION

This application claims the benefit of and priority to the earlier filing date of U.S. Provisional Patent Application No. 62/638,815, filed on Mar. 5, 2018; the entirety of this prior application is incorporated herein by reference.

### ACKNOWLEDGMENT OF GOVERNMENT SUPPORT

This invention was made with government support under Contract No. CHE-1555218, awarded by the National Science Foundation. The government has certain rights in the invention.

### FIELD

Disclosed herein are embodiments of aromatic compounds and polycyclic aromatic compounds as well as embodiments of making and using the same.

### BACKGROUND

Polycyclic aromatic hydrocarbons (PAHs), such as, acenes and pyrenes, have recently attracted considerable amount of attention in the fabrication, for instance, of electronic devices, such as, organic transistors, light-emitting devices or organic photovoltaics. However, methods of making these compounds limits the ability to functionalize such compounds and stunts derivatization. As such, the ability to manipulate the photophysical properties of such PAHs often is limited to changing their degree of π-extension, shape, and/or width. There exists a need in the art for novel methods of making new compounds that exhibit enhanced and tunable properties (e.g., electronic properties and/or positions suitable for functionalization) thereby expanding the types of devices that can implement such compounds.

### SUMMARY

Disclosed herein are embodiments of polycyclic aromatic compounds having one or more fused aromatic rings and/or assemblies and polymers of such compounds. In particular disclosed embodiments, the compounds include, but are not limited to, acene-containing compounds, such as, pentacene-containing compounds, hexacene-containing compounds, heptacene-containing compounds, octacene-containing compounds, or the like. In particular disclosed embodiments, the polycyclic aromatic compounds also include nanographene compounds, chiral aromatic compounds, asymmetric arene compounds formed from naphthalene-anthracene-, phenanthrene-, and pyrene-based starting compounds, dimerized aromatic compounds, or the like.

Also disclosed herein are embodiments of methods used to make the polycyclic aromatic compounds described herein. In particular embodiments, compounds comprising a first aromatic group functionalized with (i) one or more alkyne moieties; and (ii) a second aromatic group are reacted in presence of a catalyst to form an intramolecular bond between the one or more alkyne moieties of the first aromatic group and a carbon atom of the second aromatic group. In some embodiments, the further comprises pre-forming the compound by using a transition metal to couple a starting material comprising the first aromatic group and further comprising a boronic acid or a boronic ester with a starting material comprising the second aromatic group and further comprising a halogen atom. In some additional embodiments, the compound further comprises a third aromatic group functionalized with one or more alkyne moieties. In some embodiments, the catalyst is effective to promote forming an intramolecular bond between the one or more alkyne moieties of the third aromatic group and a carbon atom of the second aromatic group.

The polycyclic aromatic compounds and assemblies described herein can be used in a variety of applications, such as chemical and biological applications. Solely by way of example, the polycyclic aromatic compounds can be used in electronic devices, such as organic transistor, a light-emitting device, and/or organic photovoltaic devices.

The foregoing and other object and features of the present disclosure will become more apparent from the following detailed description, which proceeds with reference to the accompanying figures.

### BRIEF DESCRIPTION OF THE DRAWINGS

FIGS. 1A-ID provide the ¹H-NMR spectra and ¹³C-NMR spectra of compound embodiments 204a (FIGS. 1A and 1B, respectively) and 204c (FIGS. 1C and 1D, respectively).

FIG. 1E is an X-ray image of Compounds 204a & its corresponding endoperoxide product (204a-O₂) disclosed herein.

FIG. 1F is an X-ray image of the core structure of Compound 204a (phenyl and tert-butyl substituents omitted for clarity).

FIG. 1G depicts a normalized UV-vis absorption spectrum of a representative compound disclosed herein.

FIG. 1H depicts an excitation spectrum of Compound 204a disclosed herein.

FIG. 1I depicts an emission spectrum of Compound 204c disclosed herein.

FIG. 1J is an excitation spectrum of the ¹O₂ generated by Compound 204a disclosed herein.

FIG. 1K is an excitation spectrum of the ¹O₂ generated by Compound 204c disclosed herein.

FIG. 1L is an excitation spectrum (left) and emission spectrum (right) of Compound 204a as a thin film disclosed herein.

FIG. 1M is an emission intensity of the peak at 560 nm for the film of Compound 204a as a function of time.

FIG. 1N is a near infra-red emission spectrum of Compound 204a in the absence of light excitation.

FIG. 1O is a comparison of a ¹H-NMR spectrum of Compound 204a with the ¹H-NMR spectrum of Compound 204a in O₂-saturated CDCl₃ solution under dark for 24 h.

FIG. 1P is a ¹H-NMR spectrum depicting photodegradation of Compound 204a disclosed herein in O₂-saturated CDCl₃ solution under ambient light.

FIG. 1Q is a graphic plot of absorbance as a function of time for Compound 204a disclosed herein.

FIG. 1R is a graphic plot of absorbance as a function of time for Compound 204c disclosed herein.

FIG. 1S is a graphic plot of emission area as a function of absorbance for Ru(bpy)₃Cl₂.6H₂O.

FIG. 1T is a graphic plot of emission area of a representative compound disclosed herein, which further provides quantum yield.

FIG. 2 illustrates an embodiment of a method for making polycyclic aromatic compounds disclosed herein.

FIG. 3A depicts X-ray images of representative compounds disclosed herein.

FIG. 3B depicts X-ray images of how representative compounds disclosed herein can interact with one another.

FIG. 3C depicts an X-ray image of Compound 400h′ disclosed herein.

FIG. 4A depicts a normalized UV-vis absorption spectrum (solid lines) and emission spectrum (dotted lines) of representative compounds disclosed herein.

FIG. 4B depicts a normalized UV-vis absorption spectrum (solid lines) and emission spectrum (dotted lines) of representative compounds disclosed herein.

FIGS. 5A-5X provide the ¹H-NMR spectra and ¹³C-NMR spectra of compound embodiments 400′ (FIGS. 5A and 5B, respectively), 402a (FIGS. 5C and 5D, respectively), 402b (FIGS. 5E and 5F, respectively), 402c (FIGS. 5G and 5H, respectively), 402d (FIGS. 51 and 5J, respectively), 402e (FIGS. 5K and 5L, respectively), 402f (FIGS. 5M and 5N, respectively), 402i (FIGS. 50 and 5P, respectively), 402j (FIGS. 5Q and 5R, respectively), 414 (FIGS. 5S and 5T, respectively), 436 (FIGS. 5U and 5V, respectively), and 440 (FIGS. 5W and 5X, respectively).

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently.

Additionally, the description sometimes uses terms like “produce” or “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

To facilitate review of the various embodiments of the disclosure, the following explanations of specific terms are provided. Certain functional group terms include a symbol “-” which is used to show how the defined functional group attaches to, or within, the donor compound to which it is bound. Also, a dashed bond (i.e., “---”) as used in certain formulas described herein indicates an optional bond (that is, a bond that may or may not be present). A person of ordinary skill in the art would recognize that the definitions provided below and the donor compounds and formulas included herein are not intended to include impermissible substitution patterns (e.g., methyl substituted with 5 different groups, and the like).

Such impermissible substitution patterns are easily recognized by a person of ordinary skill in the art. In formulas and donor compounds disclosed herein, a hydrogen atom is present and completes any formal valency requirements (but may not necessarily be illustrated) wherever a functional group or other atom is not illustrated. For example, a phenyl ring that is drawn as

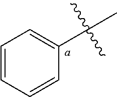 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Acyl Halide: —C(O)X, wherein X is a halogen, such as Br, F, I, or Cl.

Aldehyde: —C(O)H.

Aliphatic: A hydrocarbon group having at least one carbon atom to 50 carbon atoms (C₁-C₅₀), such as one to 25 carbon atoms (C₁-C₂₅), or one to ten carbon atoms (C₁-C₁₀), and which includes alkanes (or alkyl), alkenes (or alkenyl), alkynes (or alkynyl), including cyclic versions thereof, and further including straight- and branched-chain arrangements, and all stereo and position isomers as well.

Aliphatic-aryl: An aryl group that is or can be coupled to a compound disclosed herein, wherein the aryl group is or becomes coupled through an aliphatic group.

Aliphatic-heteroaryl: A heteroaryl group that is or can be coupled to a compound disclosed herein, wherein the heteroaryl group is or becomes coupled through an aliphatic group.

Alkenyl: An unsaturated monovalent hydrocarbon having at least two carbon atom to 50 carbon atoms (C₂-C₅₀), such as two to 25 carbon atoms (C₂-C₂₅), or two to ten carbon atoms (C₂-C₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, with exemplary embodiments including, but not limited to, methoxy, ethoxy, n-propoxy, isopropoxy, n-butoxy, t-butoxy, sec-butoxy, n-pentoxy.

Alkyl: A saturated monovalent hydrocarbon having at least one carbon atom to 50 carbon atoms (C₁-C₅₀), such as one to 25 carbon atoms (C₁-C₂₅), or one to ten carbon atoms (C₁-C₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkyl-aryl/Alkenyl-aryl/Alkynyl-aryl: An aryl group that is or can be coupled to a compound disclosed herein, wherein the aryl group is or becomes coupled through an alkyl, alkenyl, or alkynyl group, respectively.

Alkynyl: An unsaturated monovalent hydrocarbon having at least two carbon atom to 50 carbon atoms (C₂-C₅₀), such as two to 25 carbon atoms (C₂-C₂₅), or two to ten carbon atoms (C₂-C₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Ambient Temperature: A temperature ranging from 16° C. to 26° C., such as 19° C. to 25° C., or 20° C. to 25° C.

Amide: —C(O)NR^{a}R^{b} or —NHCOR^{a} wherein each of R^{a} and R^{b} independently is selected from hydrogen, aliphatic, heteroaliphatic, haloaliphatic, haloheteroaliphatic, aromatic, or any combination thereof.

Amine: —NR^{a}R^{b}, wherein each of R^{a} and R^{b} independently is selected from hydrogen, aliphatic, hetero-aliphatic, halo-aliphatic, halo-hetero-aliphatic, aromatic, or any combination thereof.

Annelation/Annulation: A chemical reaction in which one cyclic or ring system is added to another to form a polycyclic, or annulated compound. In some embodiments, annulation can comprise adding a ring system as illustrated below:

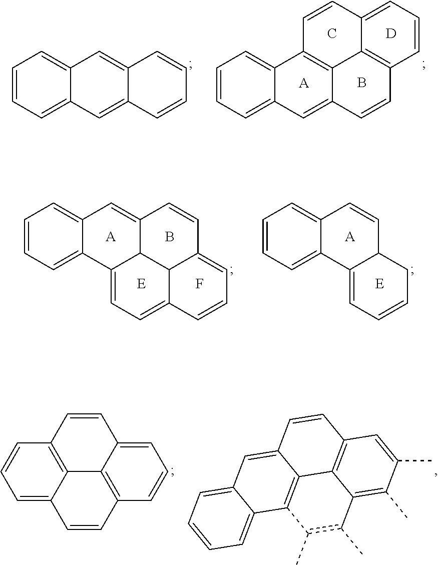

or the like.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

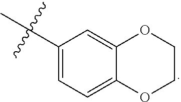 [SMILES: *c1ccc2c(c1)OCCO2]

However, in certain examples, context or express disclosure may indicate that the point of attachment is through a non-aromatic portion of the condensed ring system. For example,

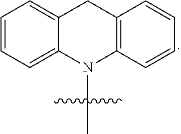 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, hetero-aliphatic, aromatic, other functional groups, or any combination thereof.

Benzannulated ring: An aromatic cyclic group comprising one or more rings that are fused to a benzene ring. In some embodiments, a benzannulated ring may be substituted with one or more groups other than hydrogen, such as aliphatic, hetero-aliphatic, aromatic, other functional groups, or any combination thereof. Examples of benzannulated rings, include, but are not limited to, benzopyrenes, such as benzo[a]pyrene

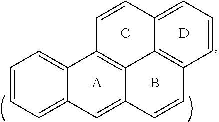

benzo[e]pyrene

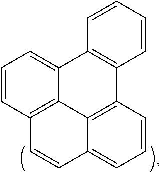

or the like; quinolines, such as quinoline

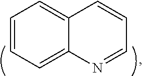

isoquinoline

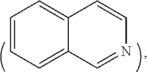

or the like, or any combination thereof.

Carboxyl: —C(O)OH or an anion thereof.

Disulfide: —SSR^{a}, wherein R^{a} is selected from hydrogen, aliphatic, heteroaliphatic, haloaliphatic, haloheteroaliphatic, aromatic, or any combination thereof.

Electron-Donating Group: A functional group capable of donating at least a portion of its electron density into the ring to which it is directly attached, such as by resonance.

Electron-Withdrawing Group: A functional group capable of accepting electron density from the ring to which it is directly attached, such as by inductive electron withdrawal.

Ester: —C(O)OR^{a} or —OC(O)R^{a}, wherein R^{a} is selected from aliphatic, hetero-aliphatic, halo-aliphatic, halo-hetero-aliphatic, aromatic, or any combination thereof.

Halo-aliphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a compound disclosed herein, wherein the aryl group is or becomes coupled through a haloaliphatic group.

Haloaliphatic-heteroaryl: A heteroaryl group that is or can be coupled to a compound disclosed herein, wherein the heteroaryl group is or becomes coupled through a haloaliphatic group.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroaliphatic-aryl: An aryl group that is or can be coupled to a compound disclosed herein, wherein the aryl group is or becomes coupled through a heteroaliphatic group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroalkyl-aryl/Heteroalkenyl-aryl/Heteroalkynyl-aryl: An aryl group that is or can be coupled to a compound disclosed herein, wherein the aryl group is or becomes coupled through a heteroalkyl, heteroalkenyl, or heteroalkynyl group, respectively.

Heteroalkyl-heteroaryl/Heteroalkenyl-heteroaryl/Heteroalkynyl-heteroaryl: A heteroaryl group that is or can be coupled to a compound disclosed herein, wherein the aryl group is or becomes coupled through a heteroalkyl, heteroalkenyl, or heteroalkynyl group, respectively.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, aromatic, other functional groups, or any combination thereof.

Heteroatom: An atom other than carbon, such as oxygen, nitrogen, sulfur, silicon, boron, selenium, or phosphorous. In particular disclosed embodiments, such as when valency constraints do not permit, a heteroatom does not include a halogen atom.

Ketone: —C(O)R^{a}, wherein R^{a} is selected from aliphatic, heteroaliphatic, aromatic, any combination thereof.

Polymer unit: A component of a compound disclosed herein comprising a repeating structural unit. In some embodiments, a polymer unit can comprise repeating alkylene oxide units (or a combination of different alkylene oxide units) and/or can comprise repeating units formed from an olefin-containing monomer.

Silyl Ether: A functional group comprising a silicon atom covalently bound to at least one alkoxy group.

Sulfonyl/Sulfonate: —SO₂R^{a}, wherein R^{a} is selected from hydrogen, aliphatic, heteroaliphatic, haloaliphatic, haloheteroaliphatic, aromatic, and any combination thereof.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated therein.

### II. Introduction

Described herein are embodiments of novel aromatic compounds and polycyclic aromatic compounds, as well as methods of making and using the same. Extended polycyclic aromatic hydrocarbons (PAHs), such as acenes, having linearly fused benzene rings, have attracted considerable attention. For instance, pentacene, which consists of five linearly-fused benzene rings, has been used in electronic devices, such as, organic transistors, light-emitting devices, organic photovoltaics or the like; however, functionalized acenes having more than five linearly-fused benzene rings are typically unstable and have poor solubility, making their applicability in the fabrication processing of such semiconductor devices difficult.

Prior to the present disclosure, modifying pentacene to add additional sextets through the central ring of pentacene has resulted in the loss of the acene characteristic of the resultant molecule, presumably due to electronic modulation. As such, conventional synthetic methodologies reported thus far are limited to making pentacene derivatives or pentacene-based compounds that are functionalized through their central ring. In contrast, the present disclosure provides compounds and methods of making such compounds that are able to provide stabilized pentacene derivatives or pentacene-like compounds with non-functionalized central rings having enhanced structural stability while maintaining the desired acene characteristic which, for instance, leads to improved solubility and material properties.

Furthermore, in another embodiment, extended polycyclic aromatic compounds, such as nanographenes, having irregular shapes as well as irregular edge topologies, can be synthesized using methods disclosed herein. For example, method embodiments disclosed herein allow Lewis-acid catalyzed regioselective intramolecular cascade benzannulation reactions of electron-rich 1,3-diynes moieties of such derivatives, thereby resulting in a series of polycyclic aromatic compounds having irregular shapes with enhanced solubility and/or enhanced chemical and/or electrochemical properties.

### III. Compound Embodiments

Disclosed herein are embodiments of novel polycyclic aromatic compounds. In some embodiments, the compounds can have a structure satisfying the following formulas and can be made according to method embodiments disclosed herein.

In one embodiment, the polycyclic aromatic compound can have a structure satisfying Formula 1.

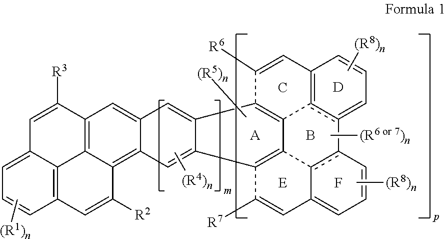

With reference to Formula 1, the following variable recitations can apply in any combination:

each R¹, R⁴, R⁵, and R⁸ if present (such as when the corresponding n variable is not 0), independently can be selected from halogen, aliphatic, haloaliphatic, haloheteroaliphatic, heteroaliphatic, aromatic, aliphatic-aromatic, heteroaliphatic-aromatic or any combination thereof;

each R², R³, R⁶, and R⁷ independently can be hydrogen, halogen, aliphatic, haloaliphatic, haloheteroaliphatic, heteroaliphatic, aromatic, aliphatic-aromatic, heteroaliphatic-aromatic or any combination thereof;

each n independently can be an integer selected from 0 to 4, such as 0, 1, 2, 3, or 4;

m can be an integer selected from 0 to 10, such as 0, 1, 2, 3, 4, 5, 6, 7, 8, 9, or 10;

p can be 0 or 1, and when p is 0, none of the rings A, B, C, D, or E are present; and

ring A, when present, can be phenyl such that none of the dashed bonds illustrated in Formula 1 are present, or ring A can join together with (i) rings B, C, and D to form a pyrene group; or ring A can join together (ii) rings B, E, and F to form a pyrene group; or (iii) ring A can join together with ring E for form a naphthyl group.

In some embodiments, each R¹, R⁴, R⁵, and R⁸, if present (such as when the corresponding n variable is not 0), independently can be an aliphatic or aromatic ring comprising one or more electron-donating groups, one or more electron-withdrawing groups, or any combination thereof. Exemplary electron-donating groups may include, but are not limited to, alkoxy, amide, amine, thioether, hydroxyl, thiol, acyloxy, aliphatic (e.g., alkyl, alkenyl, alkynyl), silyl, cycloaliphatic, aryl, or any combinations thereof. Exemplary electron-accepting groups may include, but are not limited to, aldehyde, ketone, ester, carboxylic acid, acyl, acyl halide, cyano, halogen, sulfonate, nitro, nitroso, quaternary amine, pyridinyl, or (pyridinyl wherein the nitrogen atom is functionalized with an aliphatic or aryl group), alkyl halide, or any combinations thereof. Each of R², R³, R⁶, and R⁷ also can be selected from such groups.

In an independent embodiment, R², R³, R⁶, and R⁷ are not hydrogen. In some embodiments, R⁶ can be the same as R² and/or R. In some embodiments, R⁷ can be the same as R² and/or R. In some embodiments, R²=R³. In some embodiments, R⁶=R⁷. In some embodiments, R⁶=R⁷R²=R³.

In particular embodiments, the polycyclic aromatic compounds disclosed in Formula 1 can have a structure satisfying any one of the Formulas 1A-1E below.

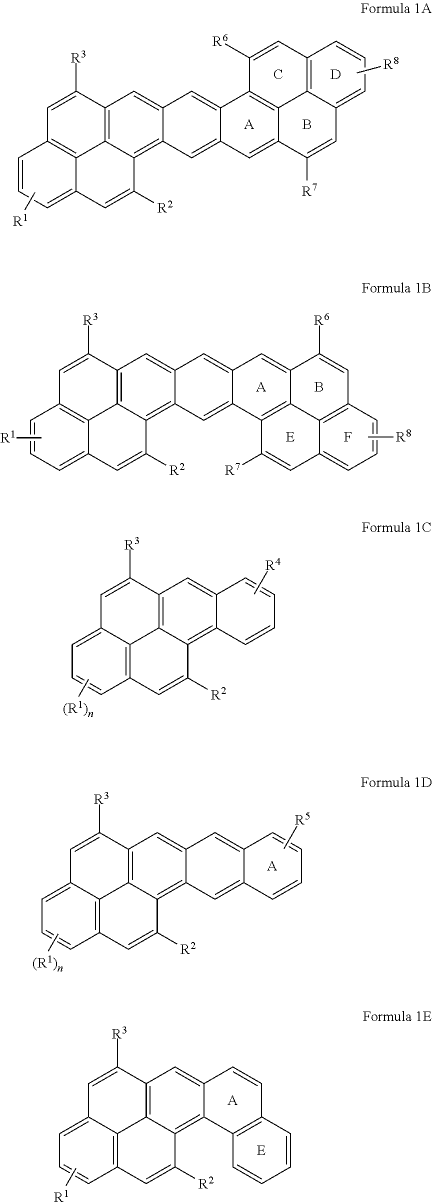

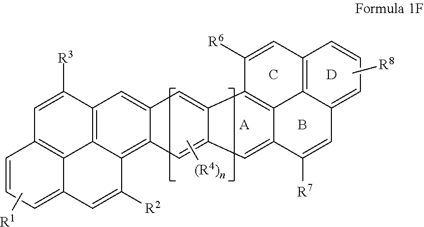

With reference to these formulas, R¹, R², R³, R⁴, R⁵, R⁶, R⁷, and R⁸ can be as recited above.

In particular disclosed embodiments of any of the above formulas, each of R¹, R², R³, R⁴, R⁵, R⁶, R⁷, and R⁸ independently be alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-aryl/heteroalkynyl-aryl, heteroalkyl-heteroaryl/heteroalkenyl-heteroaryl/heteroalkynyl-heteroaryl or any combination thereof. In some embodiments, the aryl and/or heteroaryl group can comprise one or more electron-donating groups, one or more electron-withdrawing groups, or any combination thereof. In such embodiments, the one or more electron-donating groups, one or more electron-withdrawing groups can be in any suitable position on the aryl and/or heteroaryl ring, such as meta, ortho, or para to the bond connecting the aryl and/or heteroaryl ring to the rest of the compound. The one or more electron-donating groups and the one or more electron-withdrawing groups can be as described above for Formula 1.

Exemplary compounds satisfying any one or more of Formulas 1, and 1A-1E are provided below.

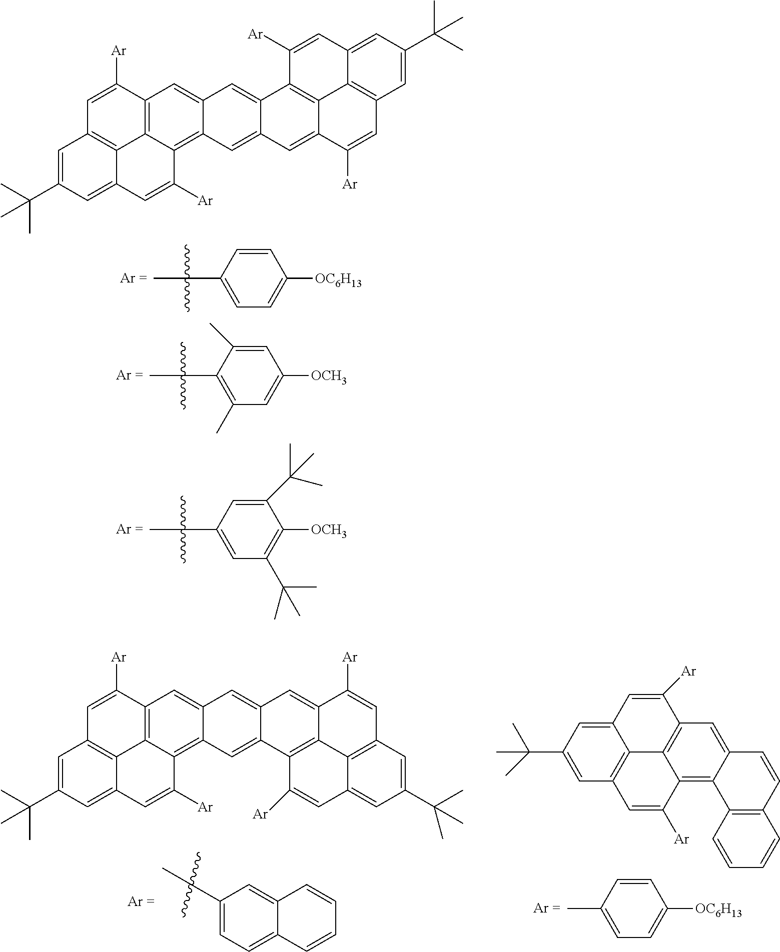

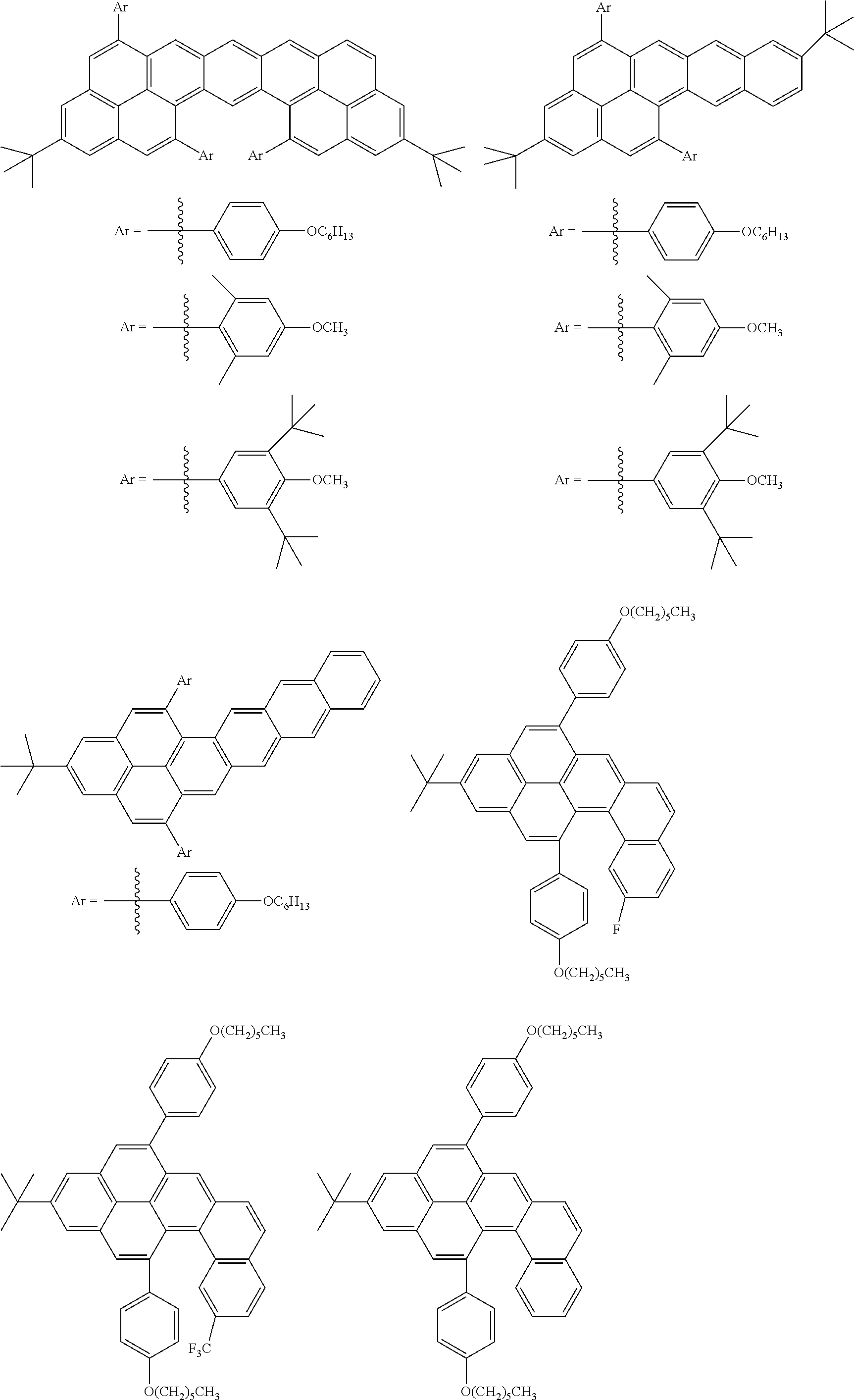

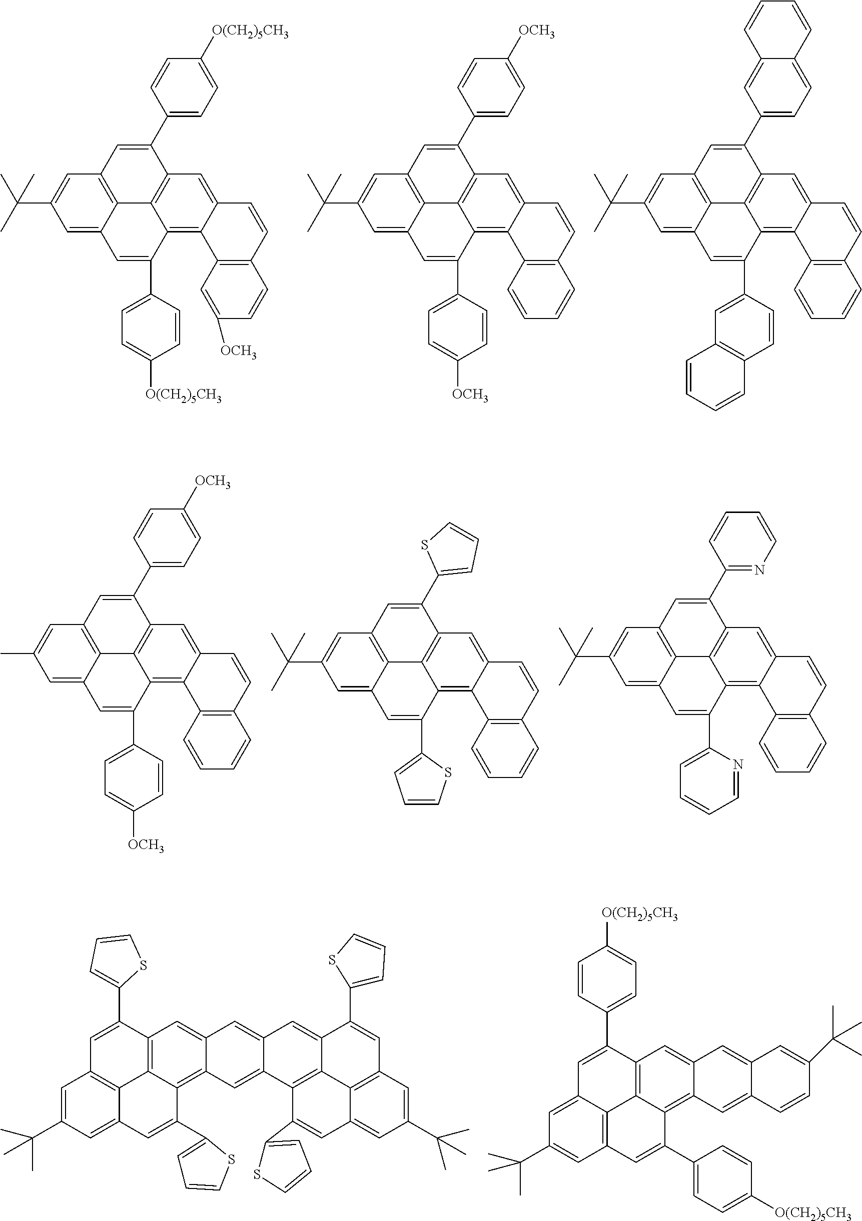

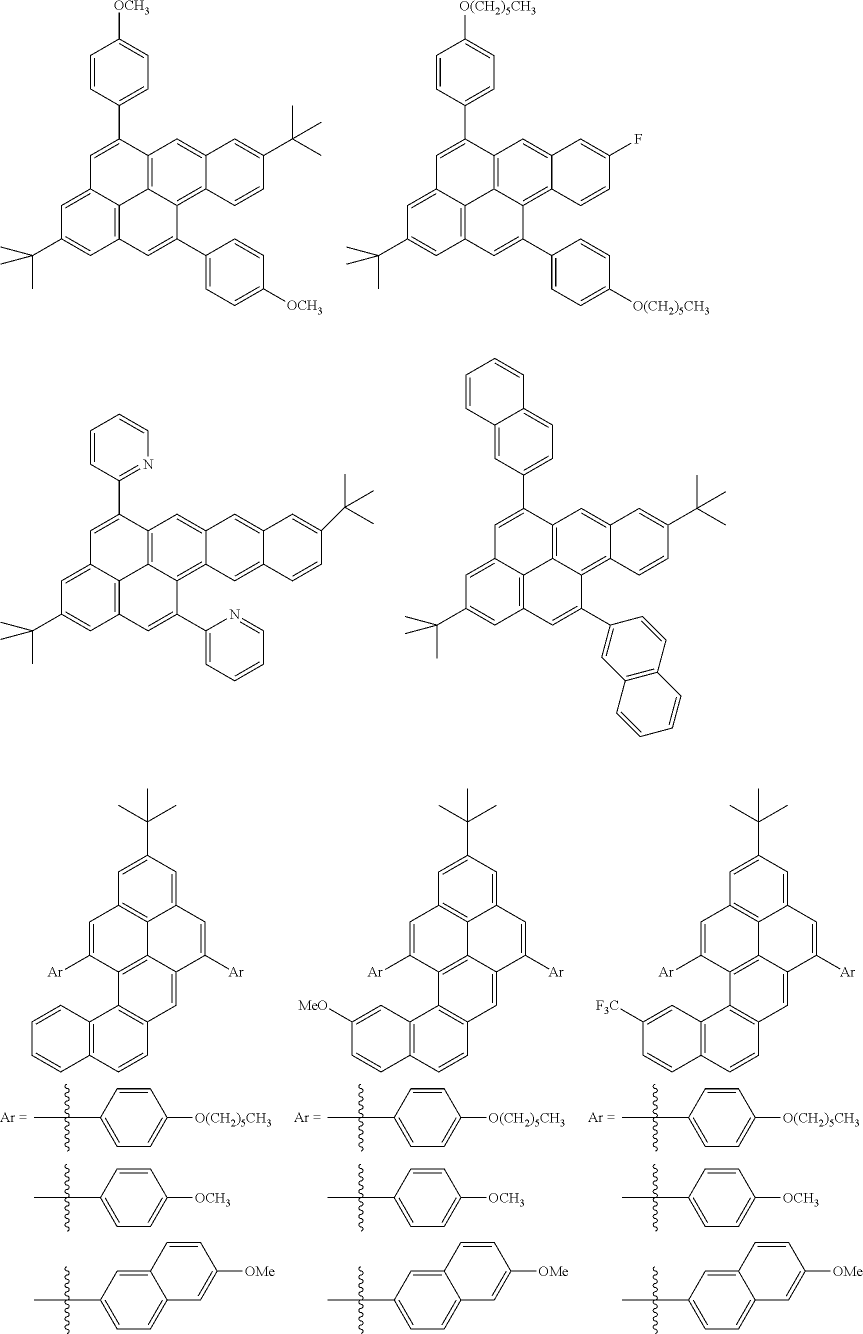

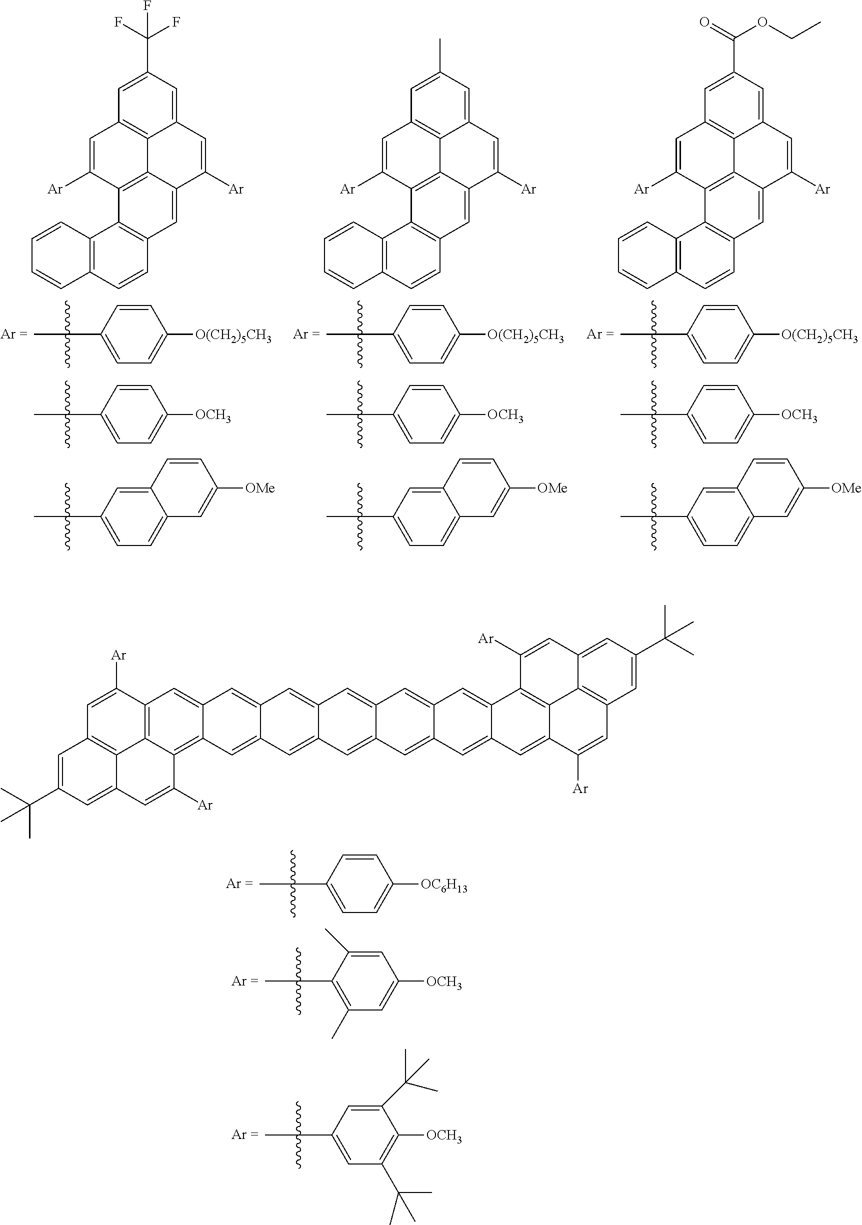

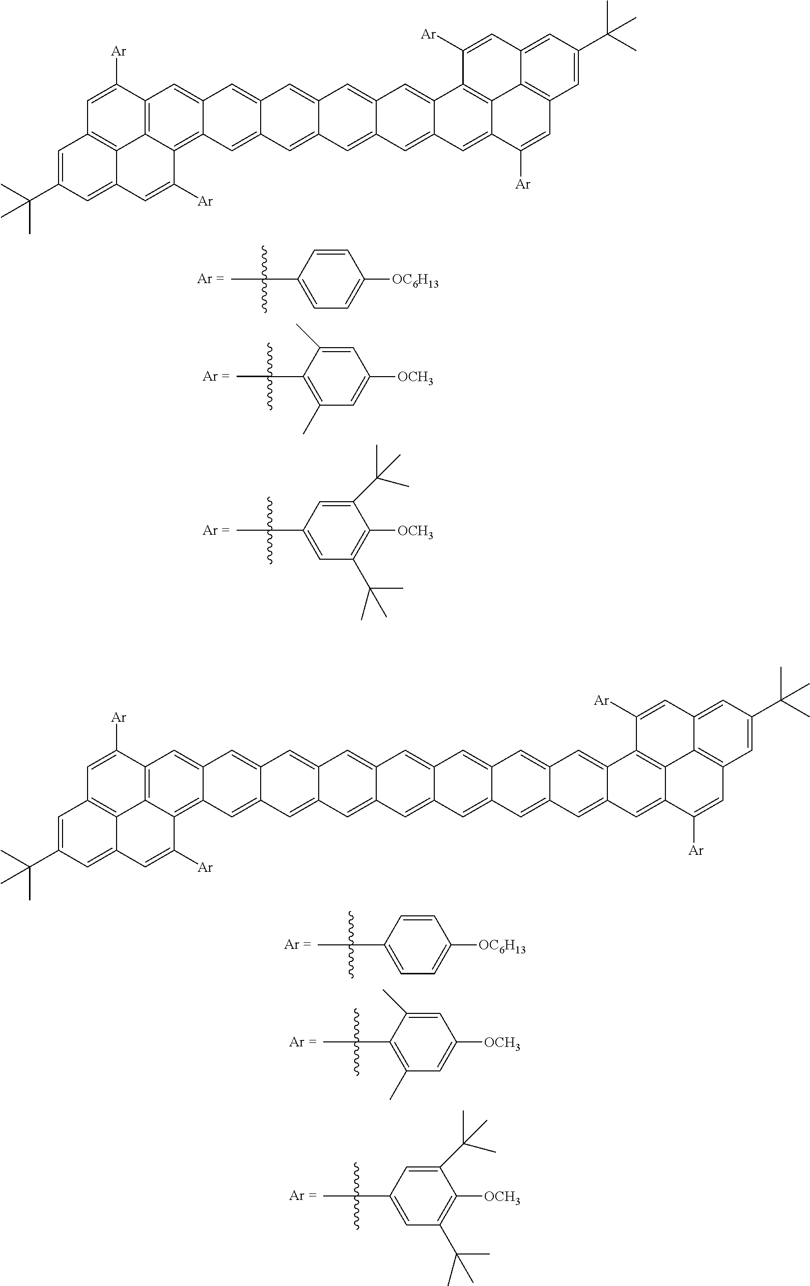

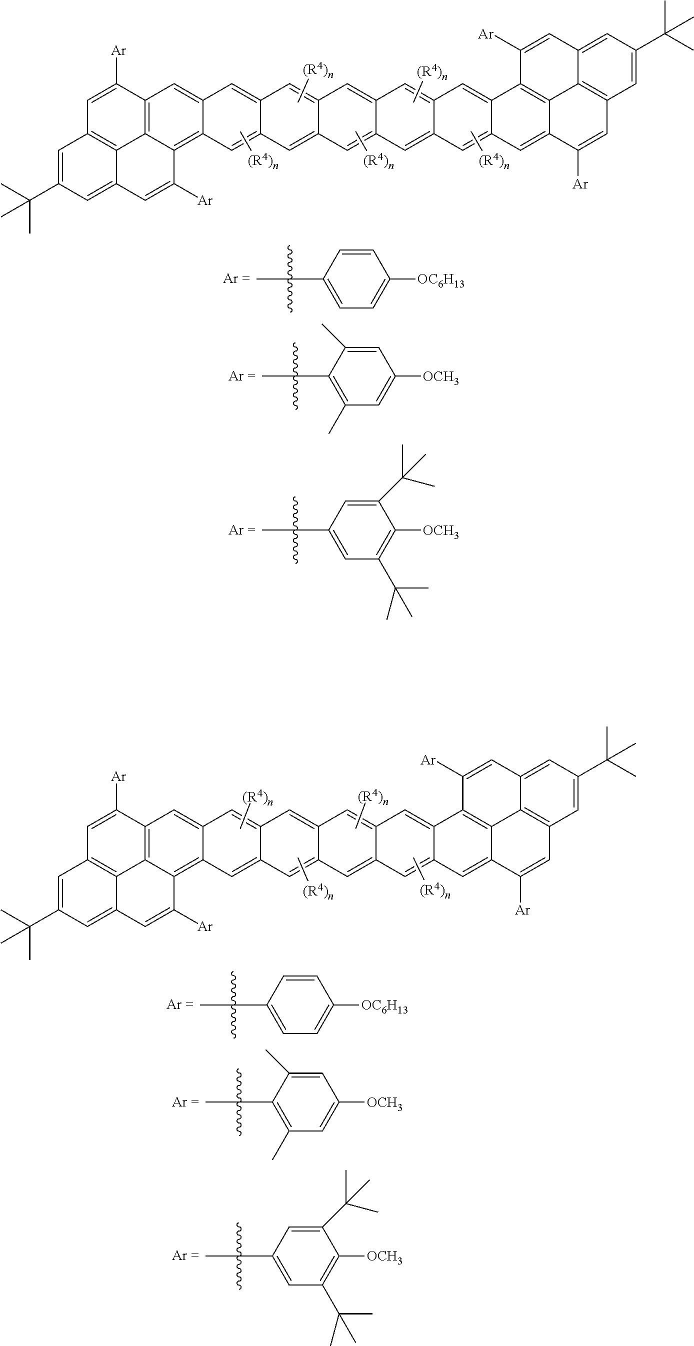

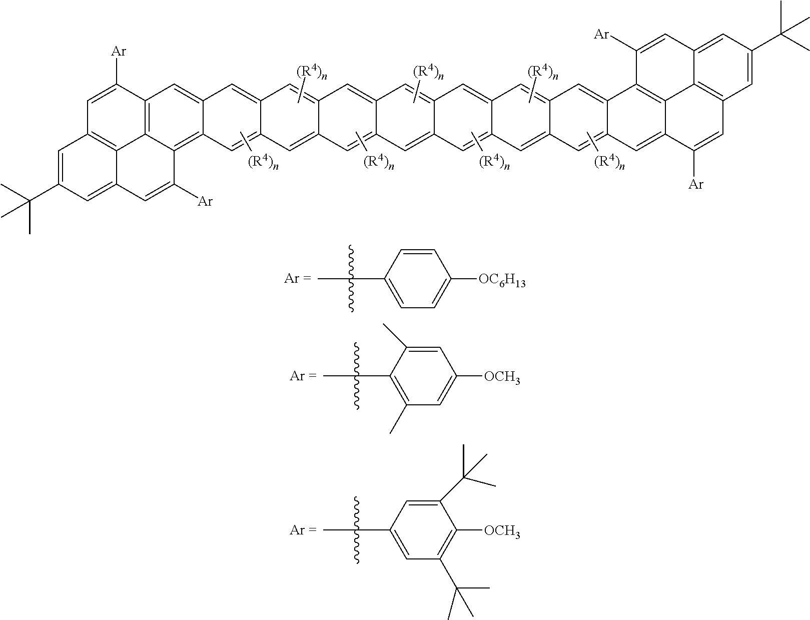

In additional embodiments, the polycyclic aromatic compound can have a structure satisfying Formula 2.

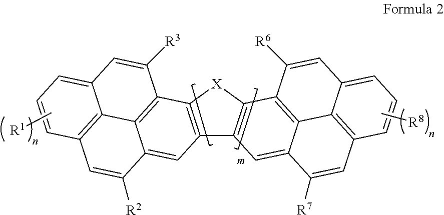

With reference to Formula 2, the following variable recitations can apply in any combination:

X can be selected from O, S, C=O, C=S, SO₂, SO, C(R′)₂, N(R′), or N⁺(R′)₂ wherein each R′ independently is hydrogen, aliphatic, haloaliphatic, haloheteroaliphatic, heteroaliphatic, aromatic or any combination thereof;

each R¹ and R⁸, if present (such as when the corresponding n variable is not 0), independently can be selected from halogen, aliphatic, haloaliphatic, haloheteroaliphatic, heteroaliphatic, aromatic, aliphatic-aromatic, heteroaliphatic-aromatic, or any combination thereof;

each R², R³, R⁶, and R⁷ independently can be hydrogen, halogen, aliphatic, haloaliphatic, haloheteroaliphatic, heteroaliphatic, aromatic, aliphatic-aromatic, heteroaliphatic-aromatic, or any combination thereof;

m can be 0 or 1; and

each n independently can be an integer selected from 0 to 3, such as 0, 1, 2, or 3.

In some embodiments, each R¹ and R⁸, if present (such as when the corresponding n variable is not 0), independently can be an aliphatic or aromatic ring comprising one or more electron-donating groups, one or more electron-withdrawing groups, or any combination thereof. Exemplary electron-donating groups may include, but are not limited to, alkoxy, amide, amine, thioether, hydroxyl, thiol, acyloxy, aliphatic (e.g., alkyl, alkenyl, alkynyl), silyl, cycloaliphatic, aryl or any combinations thereof.

Exemplary electron-accepting groups may include, but are not limited to, aldehyde, ketone, ester, carboxylic acid, acyl, halogen, acyl halide, cyano, sulfonate, nitro, nitroso, quaternary amine, pyridinyl, or (pyridinyl wherein the nitrogen atom is functionalized with an aliphatic or aryl group), alkyl halide or any combinations thereof. Each of R², R³, R⁶, and R⁷ individually can also be selected from such groups.

In an independent embodiment, at least one of R², R³, R⁶, and R⁷ is not hydrogen. In another independent embodiment, all of R², R³, R⁶, and R⁷ are other than hydrogen. In some embodiments, R⁶ can be the same as R² and/or R³. In some embodiments, R⁷ can be the same as R² and/or R³. In some embodiments, R²=R³. In some embodiments, R⁶=R⁷. In some embodiments, R⁶=R⁷=R²=R³. In some embodiments, m is 0. In some embodiments, m is 1.

In particular embodiments, the polycyclic aromatic compounds disclosed in Formula 2 can have a structure satisfying any one of the Formulas 2A-2F below. With reference to these formulas, the compound can be the M or P stereoisomer, or can comprise a mixture thereof.

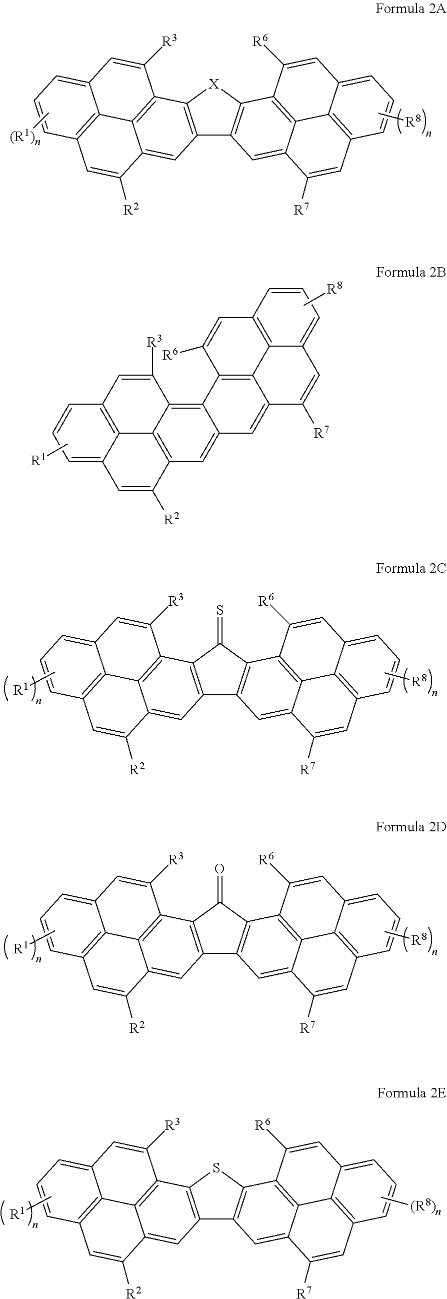

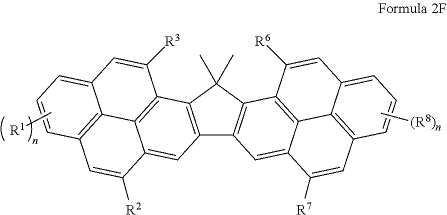

In any of the above embodiments, X can be selected from O, S, C=O, C=S, SO₂, SO, C(R′)₂, N(R′), or N⁺(R′)₂ wherein each R′ independently is hydrogen, alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-aryl/heteroalkynyl-aryl, heteroalkyl-heteroaryl/heteroalkenyl-heteroaryl/heteroalkynyl-heteroaryl or any combination thereof;

each of R¹ and R⁸, if present (such as when the corresponding n variable is not 0), independently be alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-aryl/heteroalkynyl-aryl, heteroalkyl-heteroaryl/heteroalkenyl-heteroaryl/heteroalkynyl-heteroaryl or any combination thereof. In some embodiments, the aryl and/or heteroaryl group can comprise one or more electron-donating groups, one or more electron-withdrawing groups, or any combination thereof. The one or more electron-donating groups and the one or more electron-withdrawing groups can be as described above for Formulas 1 and 1A-1E. Each of R², R³, R⁶, and R⁷ independently also can be selected from such groups. In an independent embodiment, R⁶=R⁷=R²=R³.

Exemplary compounds meeting Formulas 2, and 2A-2F are provided below:

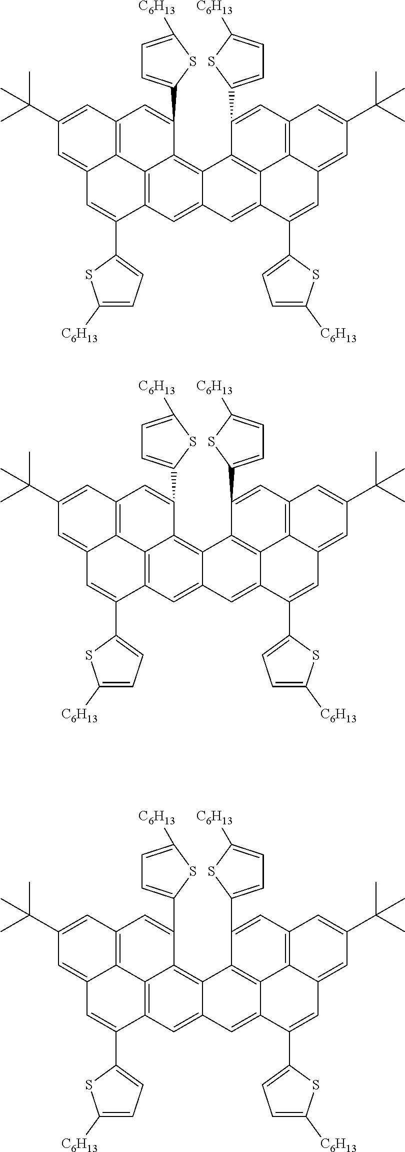

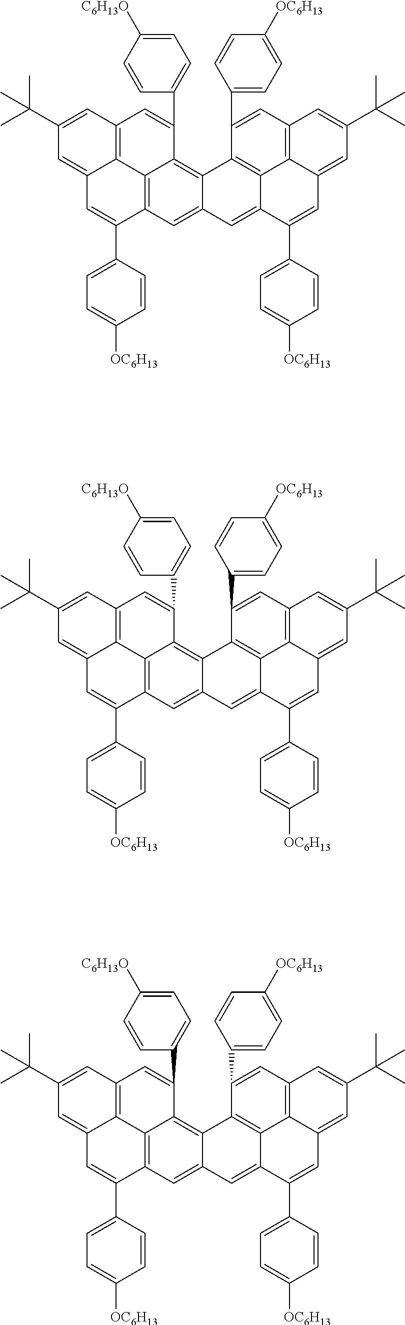

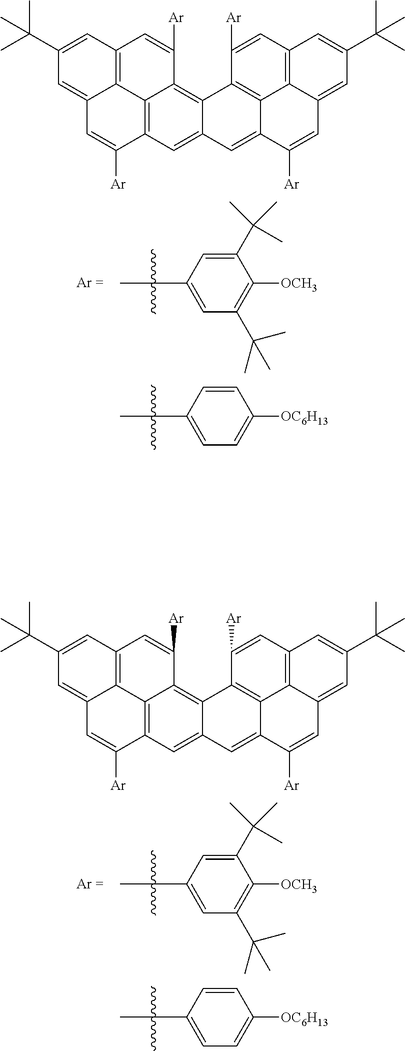

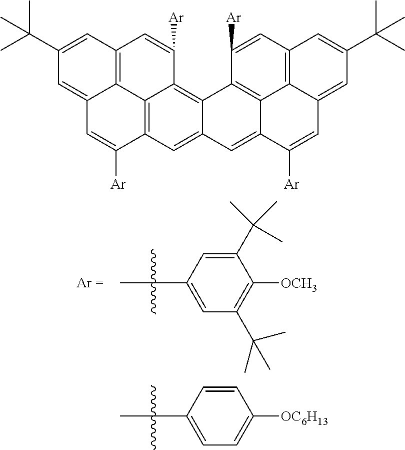

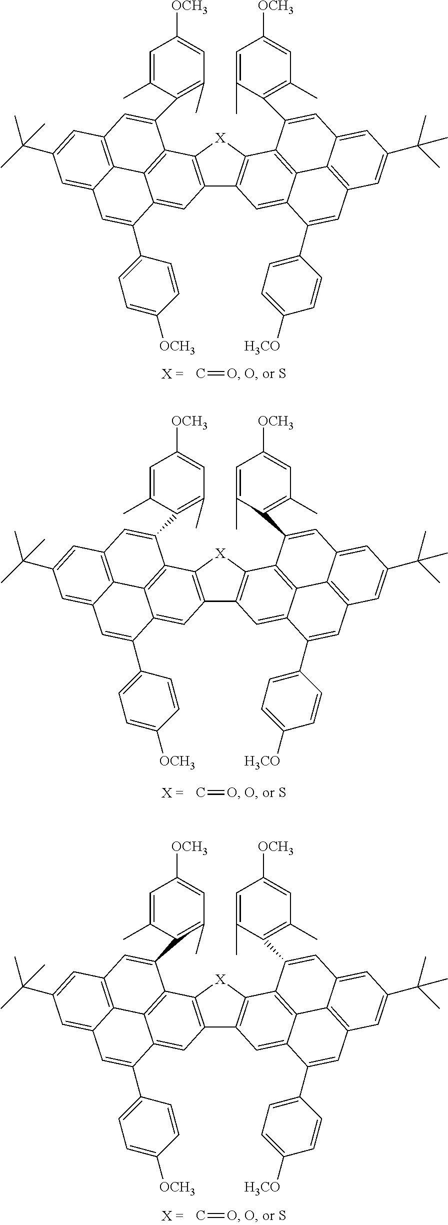

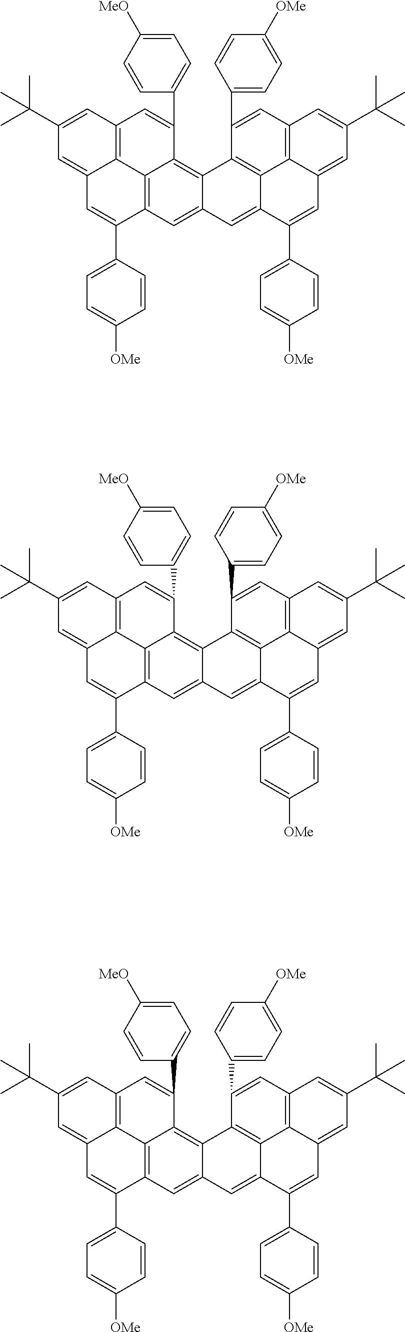

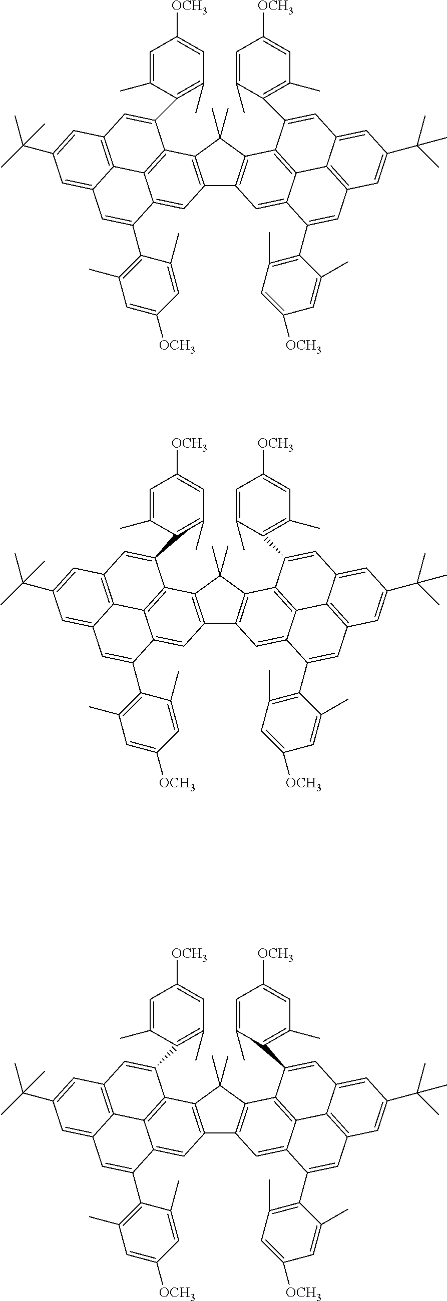

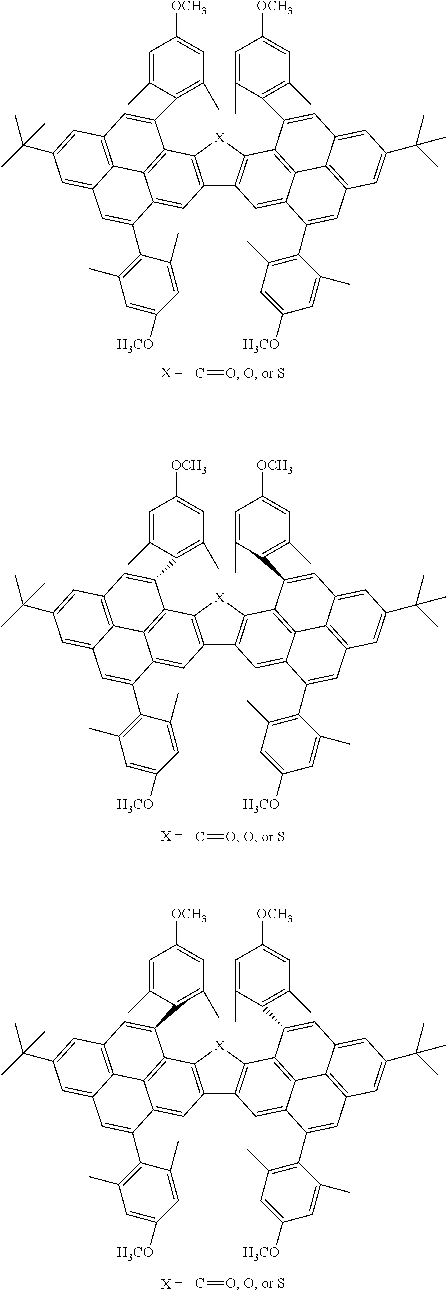

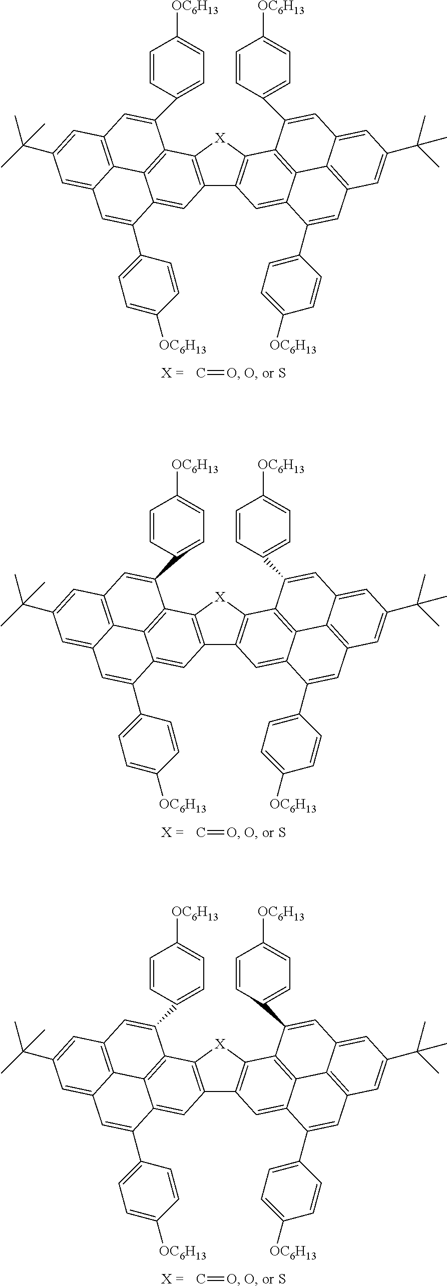

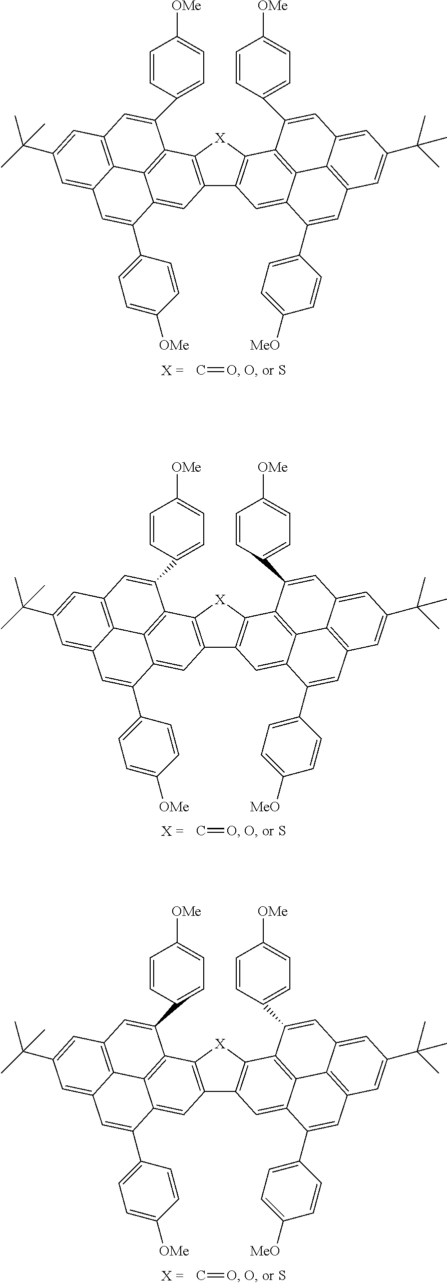

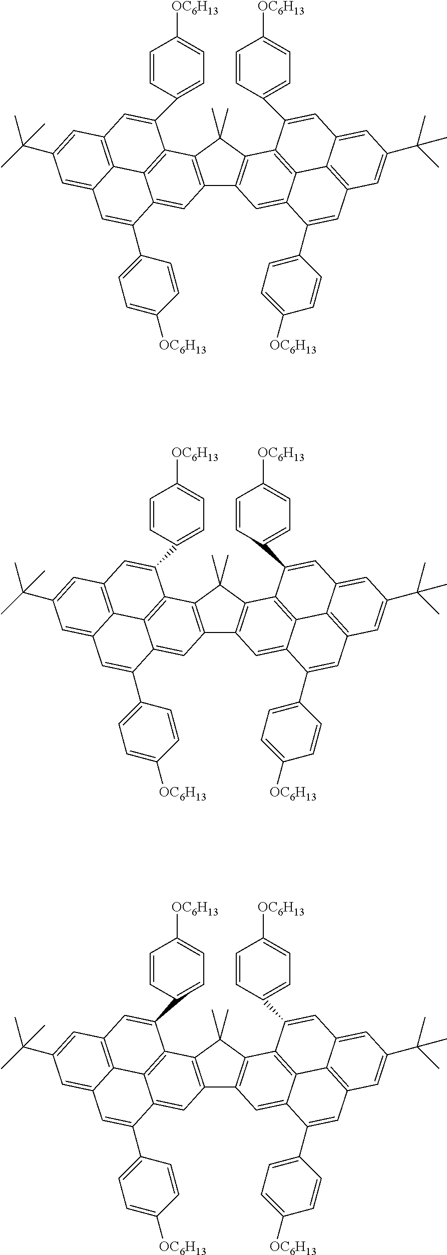

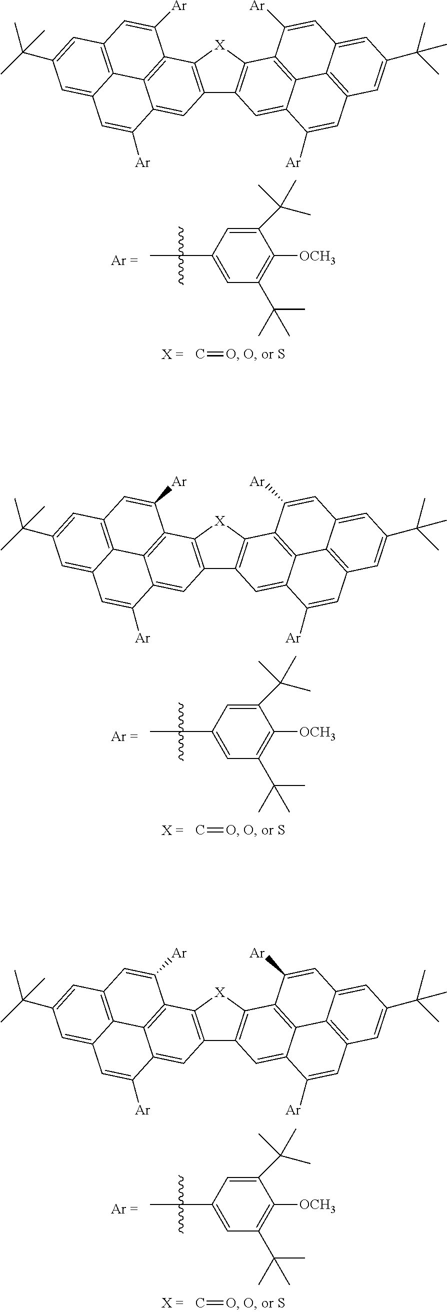

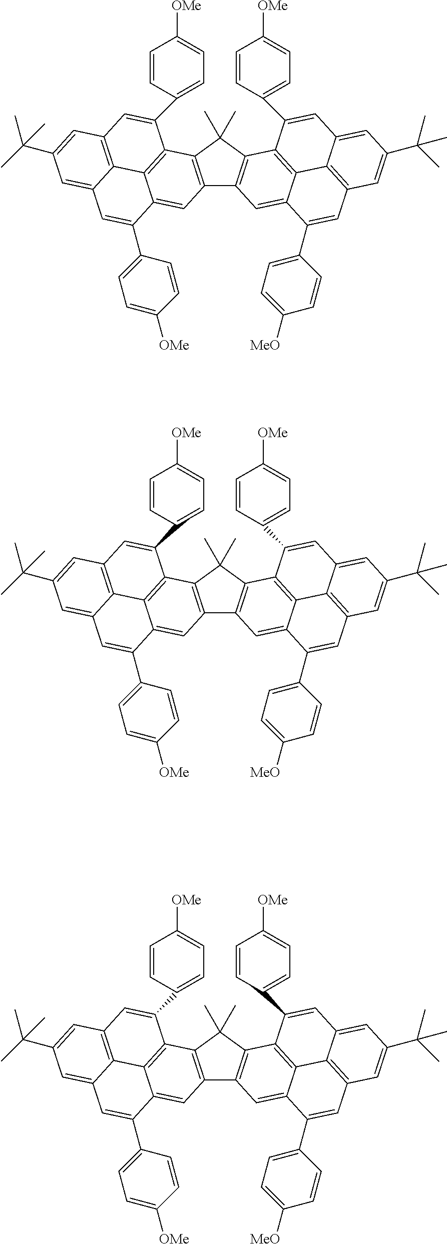

In yet another embodiment, the polycyclic aromatic compound can have a structure satisfying Formula 3.

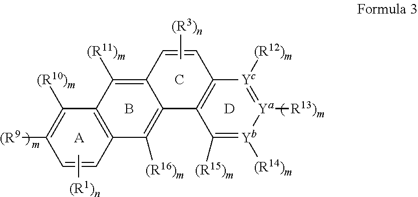

With reference to Formula 3, the following variable recitations can apply in any combination:

Y^{a} can be carbon, CH (when R¹³ is not present, as in when m is 0), or nitrogen;

Y^{b} can be carbon, CH (when R⁴ is not present, as in when m is 0), or nitrogen;

Y^{c} can be carbon, CH (when R¹² is not present, as in when m is 0), or nitrogen;

each of R¹, R³, R⁹, R¹⁰, R¹¹, R¹², R¹³, R¹⁴, R¹⁵, and R¹⁶ independently can be selected from halogen, aliphatic, haloaliphatic, haloheteroaliphatic, heteroaliphatic, aliphatic-aromatic, heteroaliphatic-aromatic, or aromatic; or any one or more of the following can apply,
R¹ and R⁹ can join together to form an aromatic ring, such as aryl (e.g., phenyl, naphthyl, pyrene) or heteroaryl (e.g., heteropyrene), or any combination thereof,
R⁹ and R¹⁰ can join together to form an aromatic ring, such as aryl (e.g., phenyl, naphthyl, pyrene) or heteroaryl (e.g., heteropyrene), or any combination thereof,
R¹⁰ and R¹¹ can join together to form an aromatic ring, such as aryl (e.g., phenyl, naphthyl, pyrene) or heteroaryl (e.g., heteropyrene), or any combination thereof,
R¹² and R¹³ can join together to form an aromatic ring, such as aryl (e.g., phenyl, naphthyl, pyrene) or heteroaryl (e.g., heteropyrene), or any combination thereof,
R¹², R¹³, and R¹⁴ can join together to form an aromatic group, such as a phenalene group having a structure

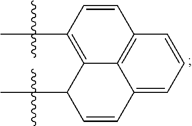 [SMILES: *c1ccc2cccc3c2c1C(*)C=C3]

or a benzoquinoline group having a structure

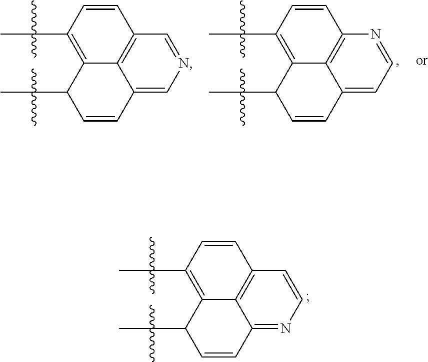 [SMILES: *c1ccc2cncc3c2c1C(*)C=C3 ; *c1ccc2nccc3c2c1C(*)C=C3 ; *c1ccc2ccnc3c2c1C(*)C=C3]

R¹³ and R¹⁴ can join together to form an aromatic ring, such as aryl (e.g., phenyl, naphthyl, pyrene) or heteroaryl (e.g., heteropyrene), or any combination thereof,
R¹⁵ and R¹⁶ can join together to form an aromatic ring, such as an aryl group (e.g., phenyl, naphthyl) or a heteroaryl ring (e.g., furan, thiophene);

each n independently can be an integer selected from 0, 1 or 2; and

each m independently can be 0 or 1.

In some embodiments, each R¹, R³, R⁹, R¹⁰, R¹², R¹³, R¹⁴, R¹⁵, and R¹⁶ if present (such as when the corresponding n or m variable is not 0), independently can be an aliphatic or aromatic ring comprising one or more electron-donating groups, one or more electron-withdrawing groups, an additional aromatic group, or any combination thereof. Exemplary electron-donating groups may include, but are not limited to, alkoxy, amide, amine, thioether, hydroxyl, thiol, acyloxy, aliphatic (e.g., alkyl, alkenyl, alkynyl), silyl, cycloaliphatic, aryl or any combinations thereof. Exemplary electron-accepting groups may include, but are not limited to, aldehyde, ketone, ester, carboxylic acid, acyl, acyl halide, halogen, cyano, sulfonate, nitro, nitroso, quaternary amine, pyridinyl, or (pyridinyl wherein the nitrogen atom is functionalized with an aliphatic or aryl group), alkyl halide or any combinations thereof. In embodiments where the aromatic group comprises additional aromatic group, the additional aromatic group can be a benzotetraphene group, or a naphthyl group. In some embodiments, the benzotetraphene group and/or the naphthyl group can comprise one or more groups selected from aliphatic, haloaliphatic, haloheteroaliphatic, heteroaliphatic, aromatic or any combination thereof.

In some embodiments, if any one or more of R¹ and R⁹; or R⁹ and R¹⁰; or R¹⁰ and R¹¹; or R¹² and R¹³; or R¹², R¹³ and R¹⁴; or R¹⁴ and R¹⁵, or R¹⁵ and R¹⁶ join together to form an aromatic group, the aromatic group can comprise one or more electron-donating groups, one or more electron-withdrawing groups, or any combination thereof. Exemplary electron-donating groups may include, but are not limited to, alkoxy, amide, amine, thioether, hydroxyl, thiol, acyloxy, aliphatic (e.g., alkyl, alkenyl, alkynyl), silyl, cycloaliphatic, aryl or any combinations thereof. Exemplary electron-accepting groups may include, but are not limited to, aldehyde, ketone, ester, carboxylic acid, acyl, acyl halide, halogen, cyano, sulfonate, nitro, nitroso, quaternary amine, pyridinyl, or (pyridinyl wherein the nitrogen atom is functionalized with an aliphatic or aryl group), alkyl halide or any combinations thereof. Representative placement of such groups can be as illustrated in any of Formulas 3A-3R.

In particular embodiments, the polycyclic aromatic compounds disclosed in Formula 3 can have a structure satisfying anyone of the Formulas 3A-3R below.

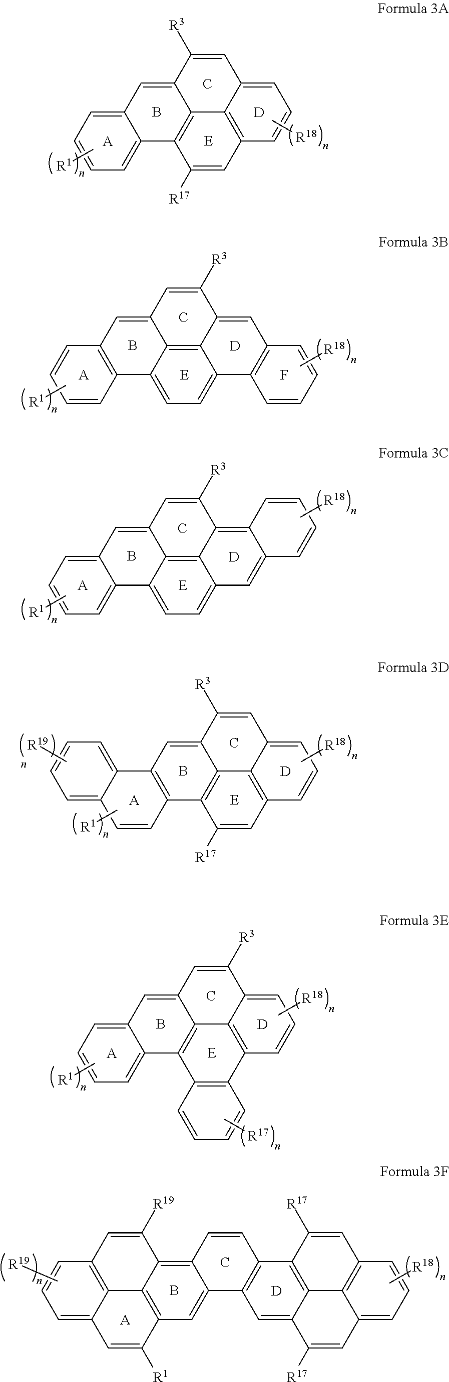

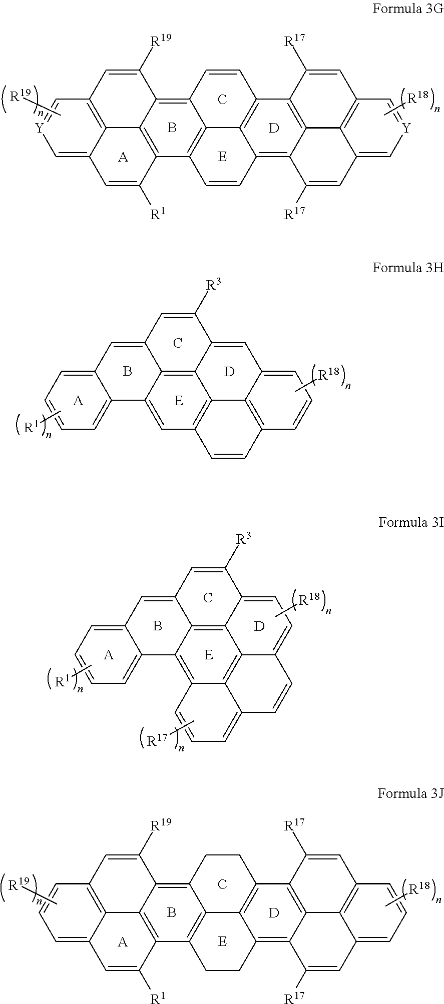

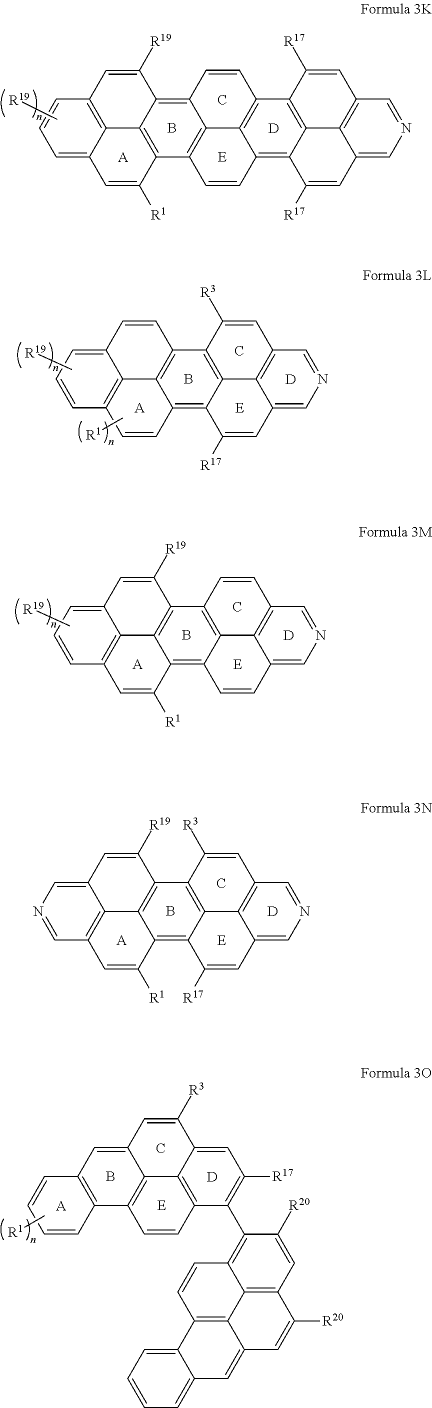

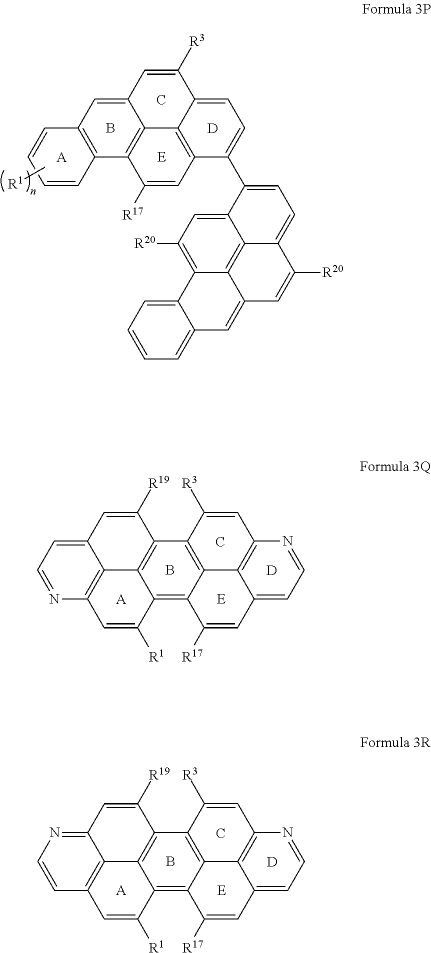

With reference to any of the formulas above, each of R¹⁷, R¹⁸, R¹⁹, and R²⁰ independently can be selected from aliphatic, haloaliphatic, haloheteroaliphatic, heteroaliphatic, aliphatic-aromatic, heteroaliphatic-aromatic, or aromatic. In some embodiments, each of R¹⁷, R¹⁸, R¹⁹, and R²⁰ independently can be selected from aromatic comprising one or more electron-donating groups, one or more electron-withdrawing groups, an additional aromatic group, or any combination thereof. In some embodiments, R¹⁷ and R²⁰ independently can be an alkoxy or hydroxyl group.

Exemplary compounds meeting Formulas 3 and 3A-3R are provided below:

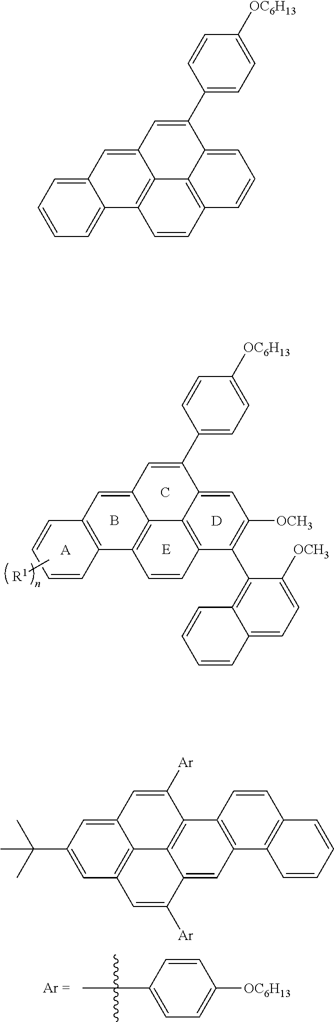

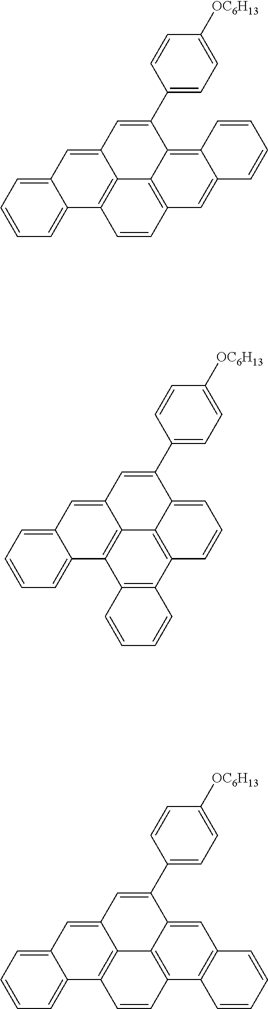

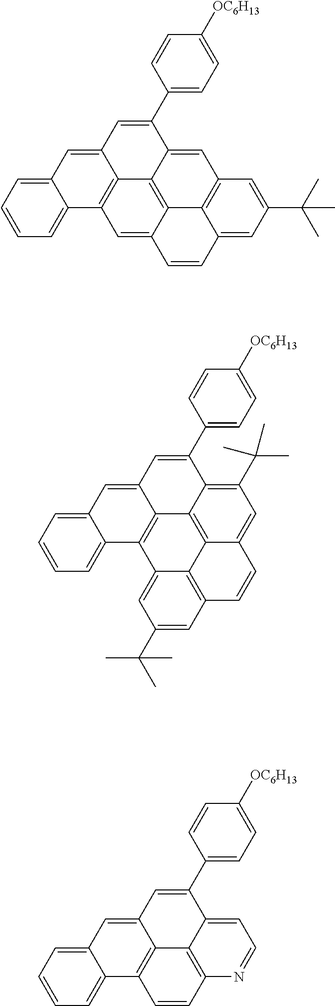

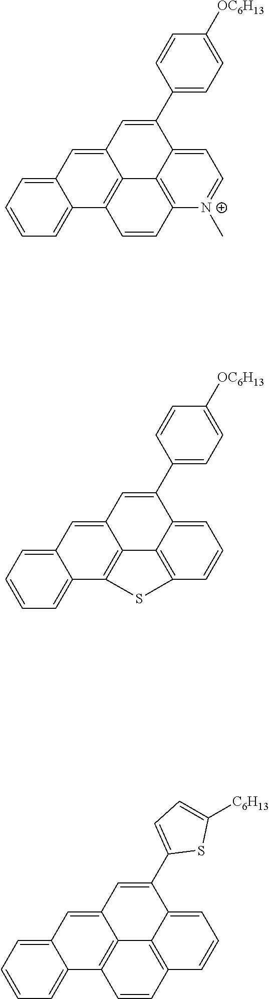

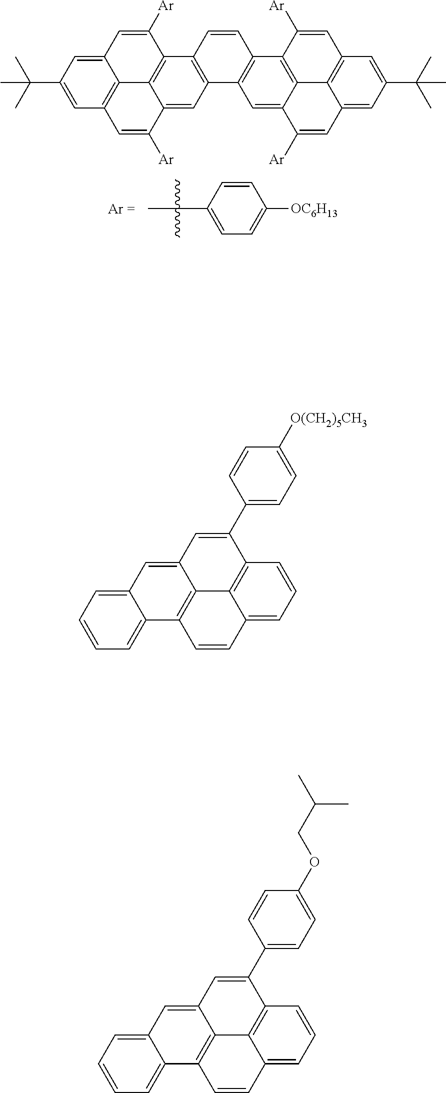

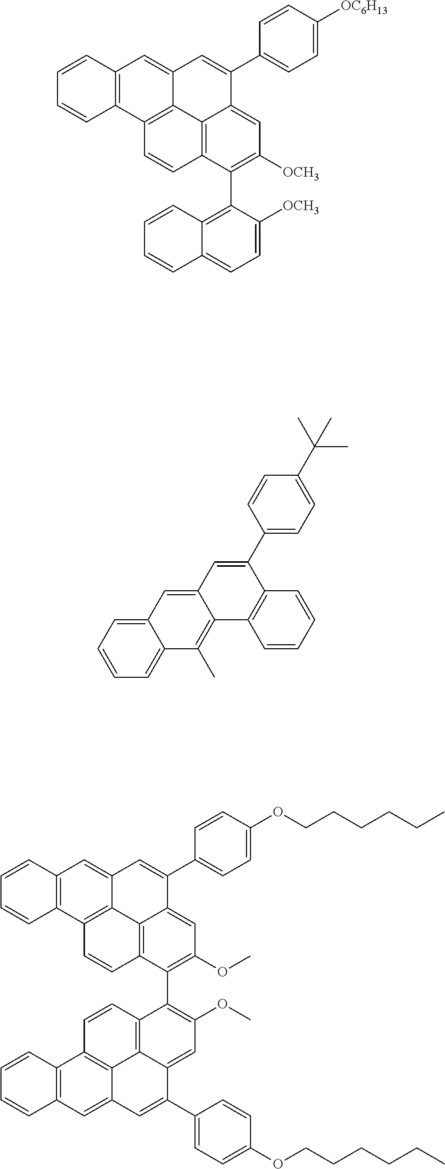

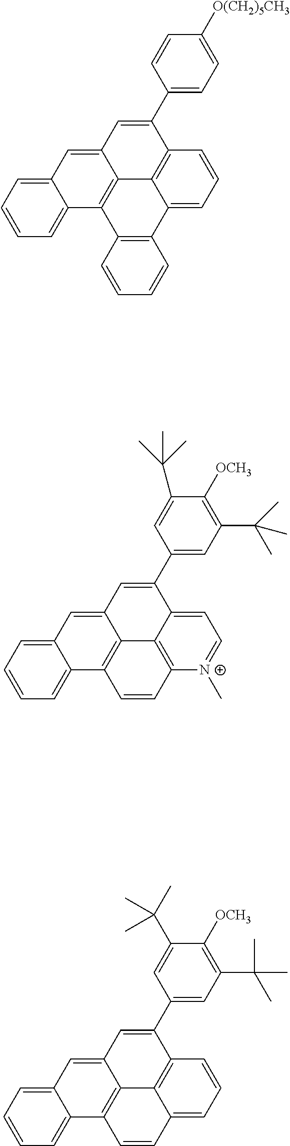

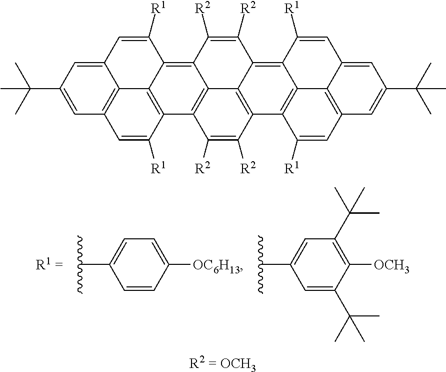

In yet another embodiment, the polycyclic aromatic compound can have a structure satisfying Formula 4 or 4′, or a hydrogenated version thereof (e.g., wherein one or more double bonds having an optional RS group as illustrated below is hydrogenated to a single bond).

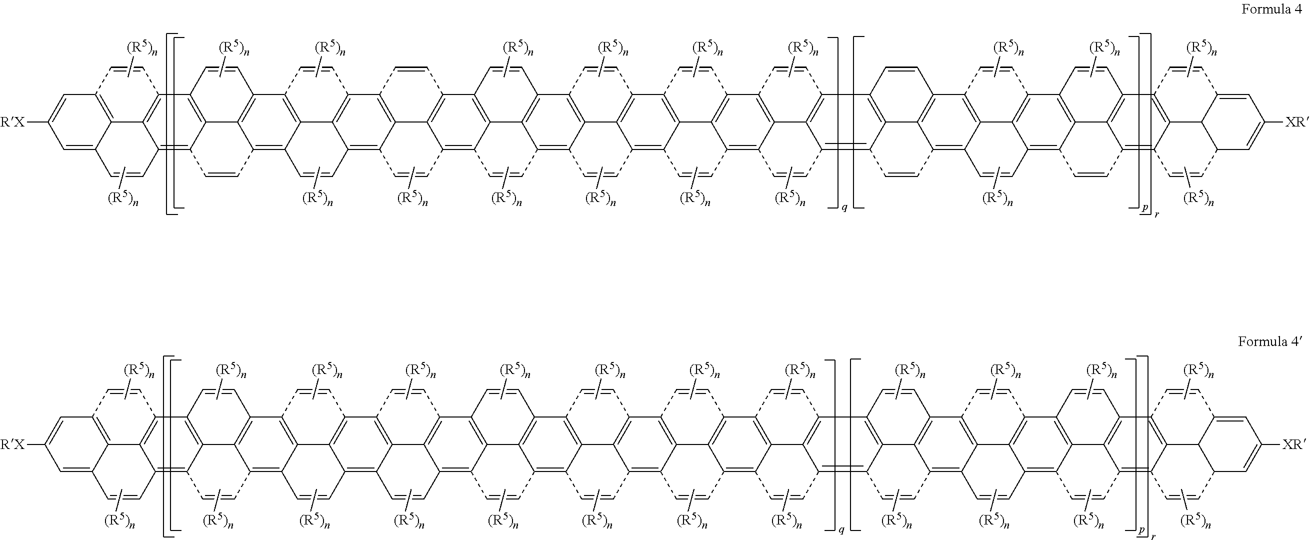

With reference to Formulas 4 and 4′, the following variable recitations can apply in any combination:

each X independently can be a heteroatom, such as O, S, or NR wherein R can be hydrogen, aliphatic, haloaliphatic, haloheteroaliphatic, heteroaliphatic, aromatic or any combination thereof, and in some embodiments when X is NR, the resulting NRR′ group can be converted to provide an azide group, a triazene group, or a diazonium group;

each R′ independently can be selected from hydrogen, aliphatic, or ketone (e.g., —C(O)R²⁰, wherein R²⁰ is hydrogen, aliphatic, haloaliphatic, haloheteroaliphatic, heteroaliphatic, aromatic or any combination thereof), or any combination thereof, and

each R⁵, if present (such as when the corresponding n variable is not 0), independently can be aliphatic, halogen, heteroaliphatic, aliphatic-aromatic, heteroaliphatic-aromatic, aromatic, or any combination thereof;

each n independently can be 0, 1, or 2;

each of p and q independently can be an integer selected from 0 to 1000;

r can be an integer selected from 1 to 1000, provided that when p is 0, r is the same integer as q.

In some embodiments, when one or more of R⁵ are aromatic, the aromatic group can comprise one or more electron-donating groups; one or more electron-withdrawing groups; a repeating polymer unit, wherein the polymer unit comprises a repeating alkylene oxide unit (e.g., —(OCH₂CH₂)—, or the like) or a repeating unit formed from a methyl methacrylate monomer; or any combination thereof.

In some embodiments, X and R′ together can be —SH, —SCH₃, or —SC(O)CH₃, —OH, —OCH₃, or —OC(O)CH₃, or —NH₂, —NHCH₃, or —NHC(O)CH₃.

In some embodiments, each R⁵, if present (such as when n is not 0), independently can be alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-aryl/heteroalkynyl-aryl, heteroalkyl-heteroaryl/heteroalkenyl-heteroaryl/heteroalkynyl-heteroaryl or any combination thereof. In some embodiments, the aryl and/or heteroaryl group can comprise one or more electron-donating groups, one or more electron-withdrawing groups, or any combination thereof. Exemplary electron-donating groups may include, but are not limited to, alkoxy, —O(CH₂)ₙCH₂OH, —O(CH₂)ₙCH═CH₂, or —O(CH₂)ₙCH₂OPG (wherein PG is a protecting group), amide, amine, thioether, hydroxyl, thiol, acyloxy, aliphatic (e.g., alkyl, alkenyl, alkynyl), silyl, cycloaliphatic, aryl or any combinations thereof. Exemplary electron-accepting groups may include, but are not limited to, aldehyde, ketone, ester, carboxylic acid, acyl, acyl halide, halogen, cyano, sulfonate, nitro, nitroso, quaternary amine, pyridinyl, or (pyridinyl wherein the nitrogen atom is functionalized with an aliphatic or aryl group), alkyl halide or any combinations thereof.

In particular disclosed embodiments, each R⁵ independently is an alkyl group, alkoxy group, an alkylene oxide group, or an aromatic group comprising an alkylene oxide group.

In some embodiments, q can be 1 and p can be 1 and r can be 1 to 1000. In yet additional embodiments, q can be 1 and p can be 0 and r is 1. In yet additional embodiments, q can be 0 and p can be 1 and r can be 1 to 1000. In some embodiments, any of the aryl rings illustrated in Formulas 4 or 4′ (or any of Formulas 4A-4H, below) can be replaced with a heteroaryl group, such as a pyridine ring.

In particular embodiments, the polycyclic aromatic compounds of Formulas 4 and/or 4′ can have a structure satisfying any one of the Formulas 4A-4H below. With reference to Formulas 4E-4H, each “A” independently can be CH or nitrogen (or an ionized form thereof, such as =N+Me).

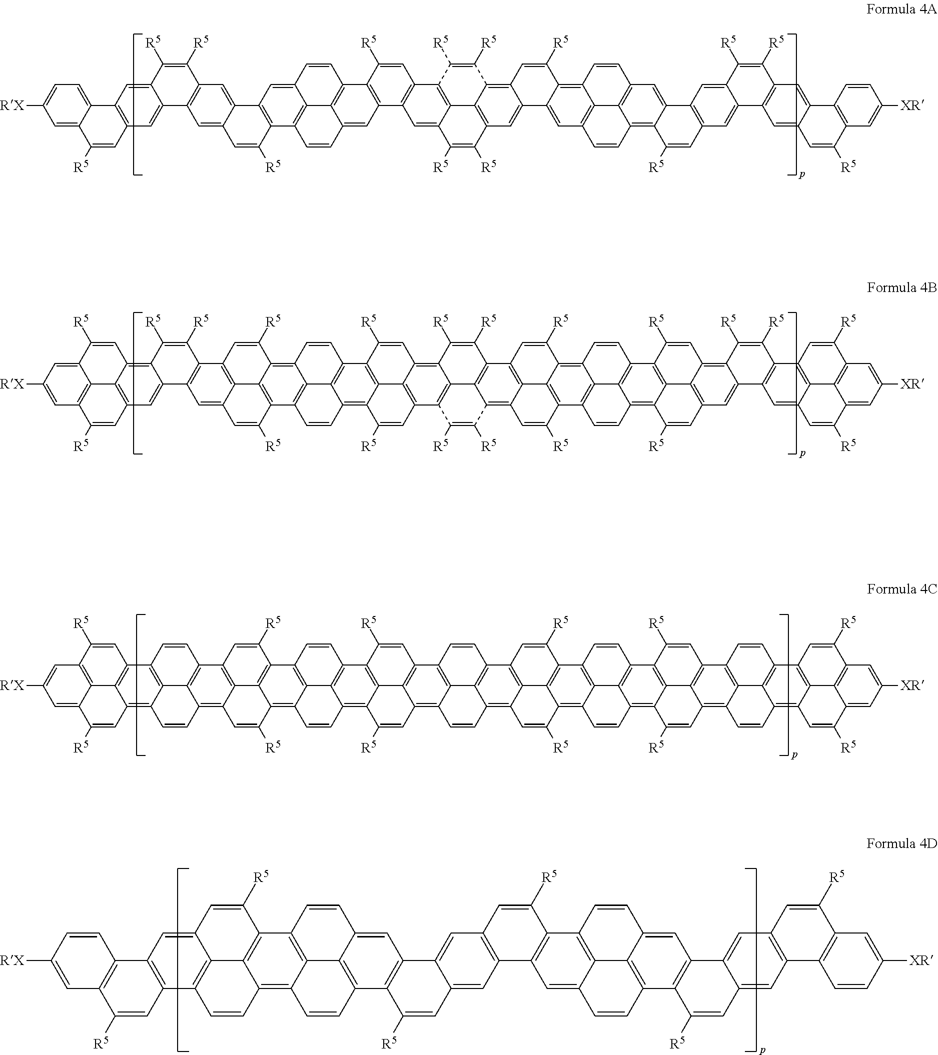

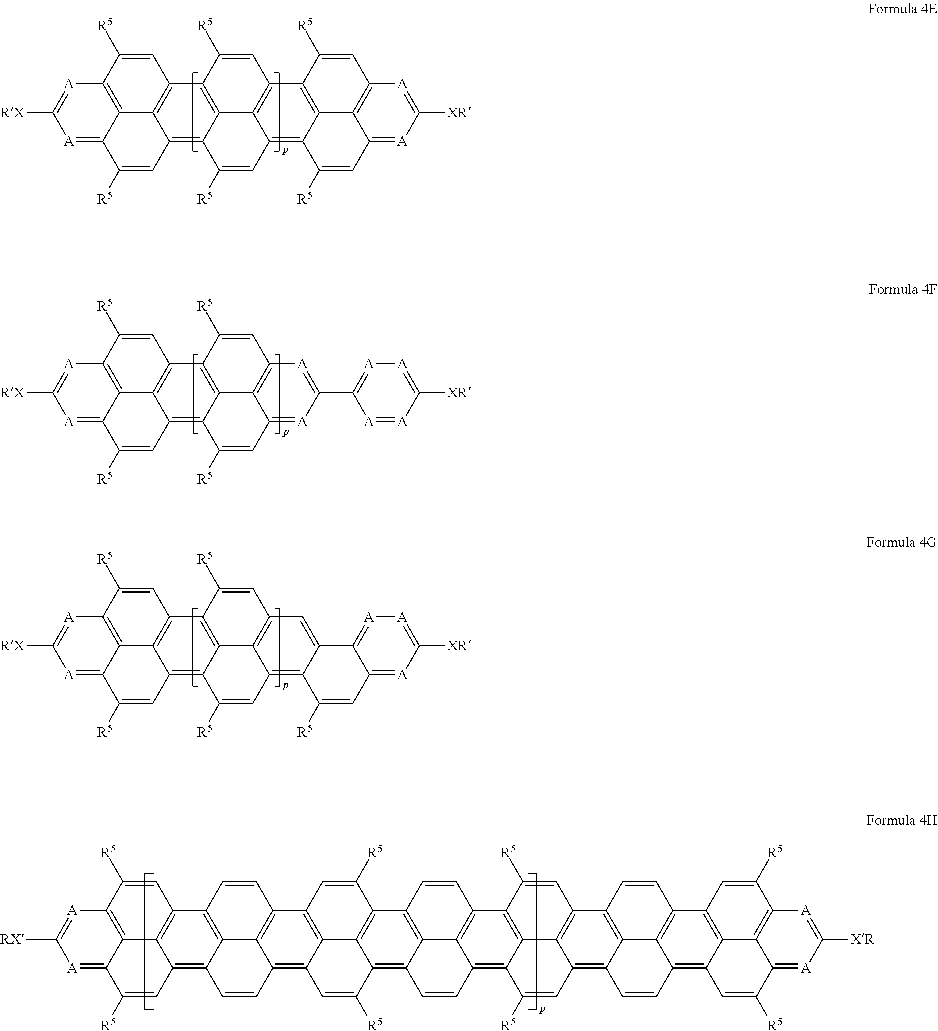

Exemplary compounds meeting Formulas 4, 4′, and 4A-4H are provided below:

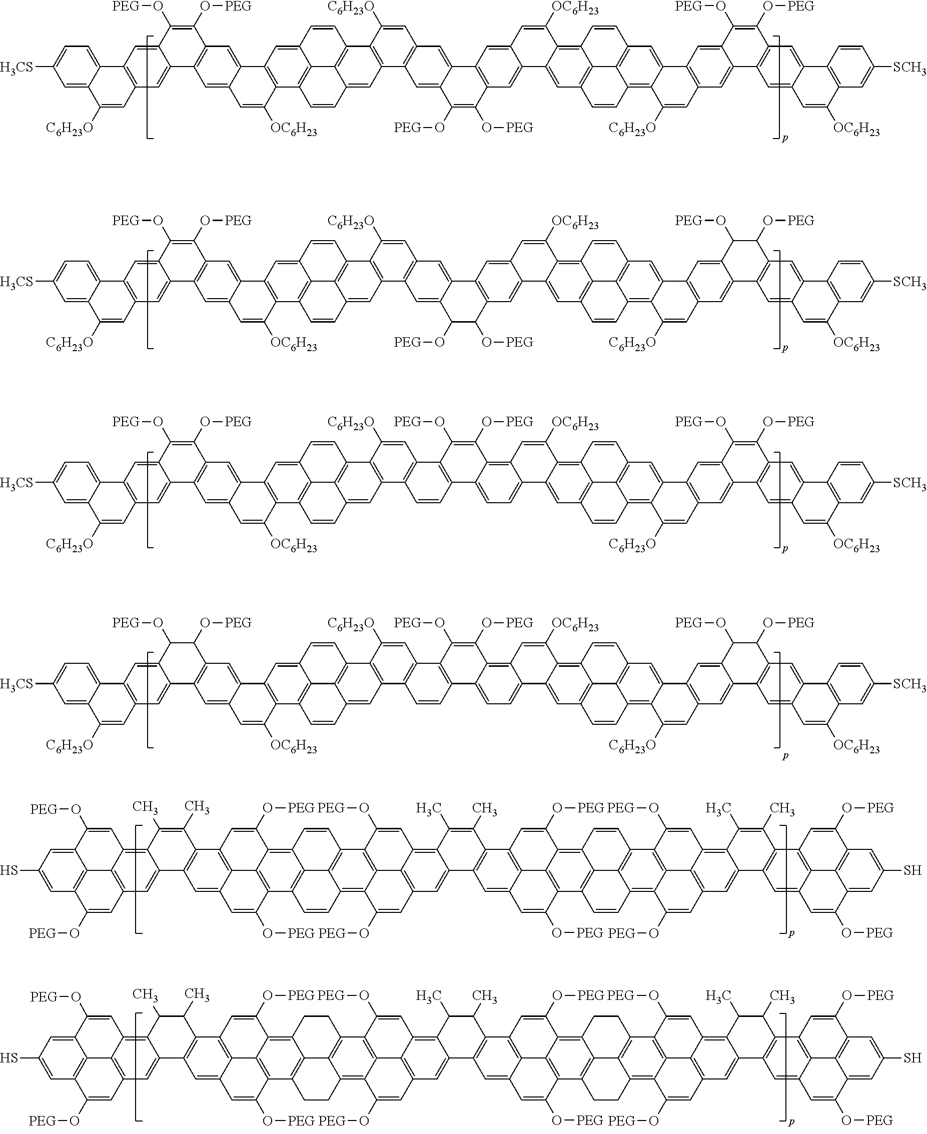

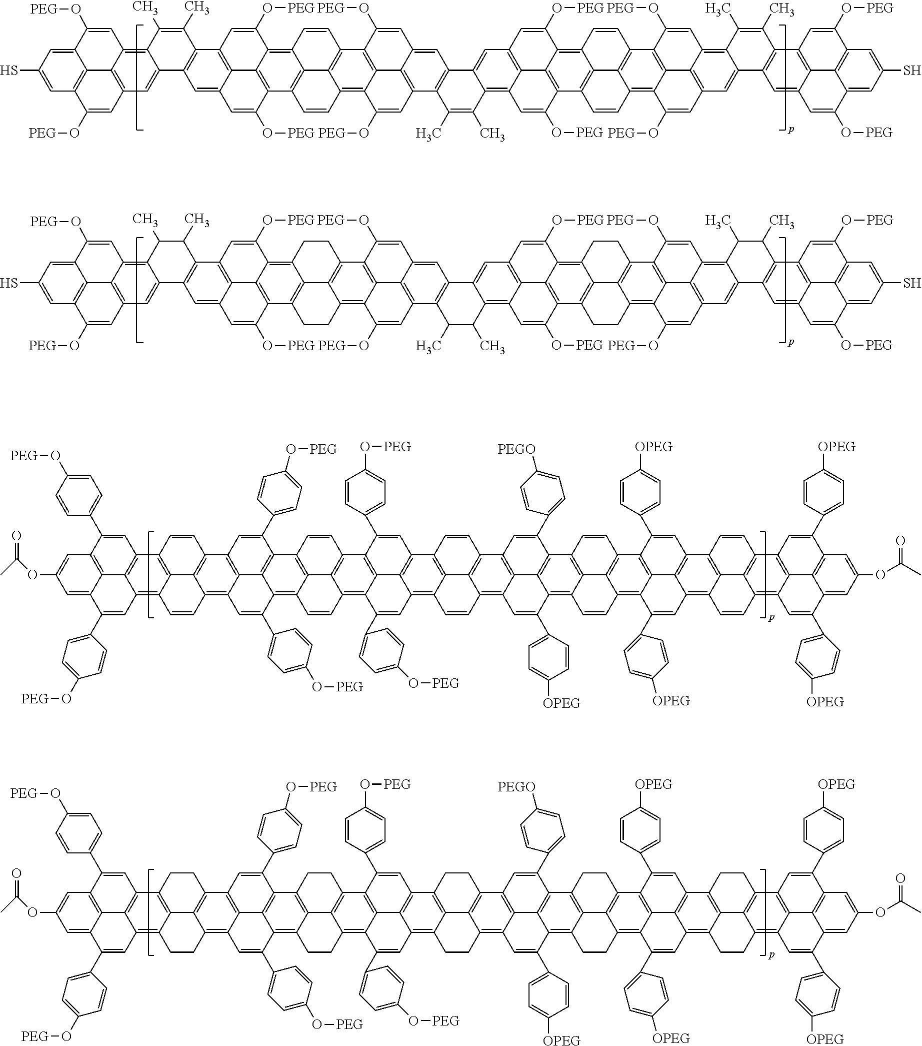

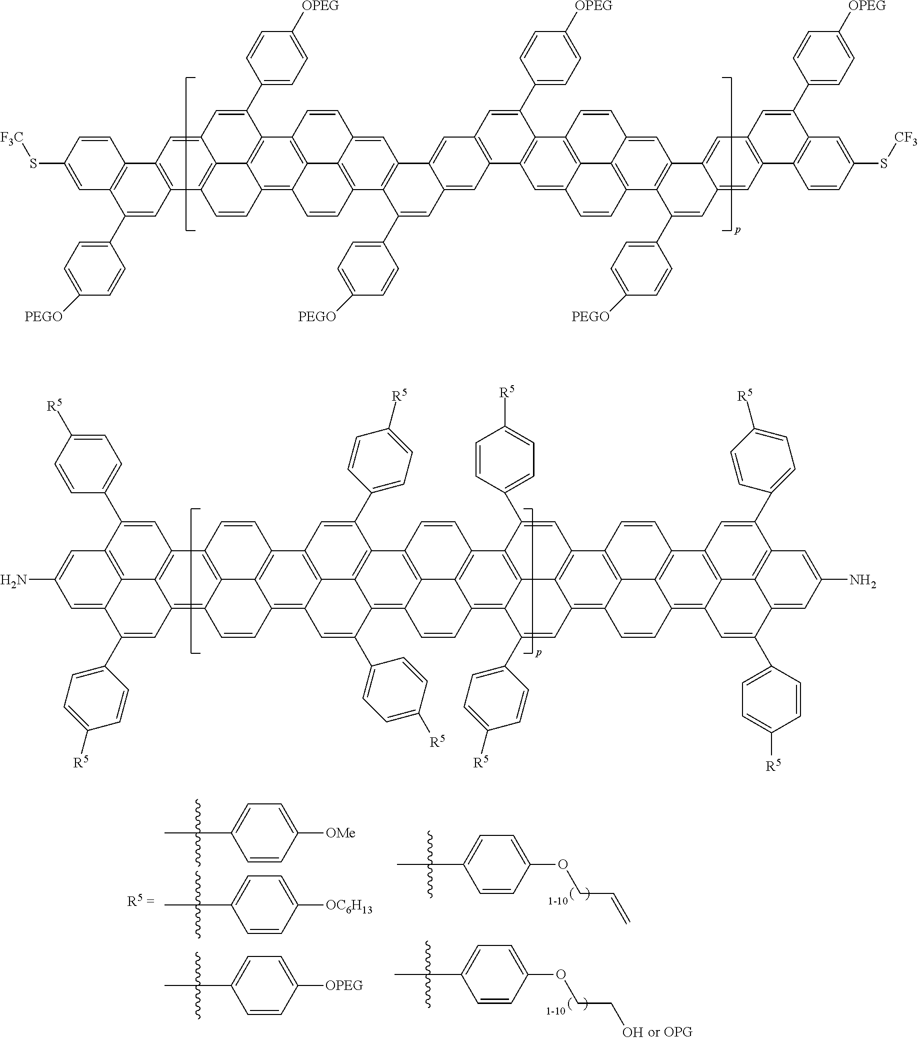

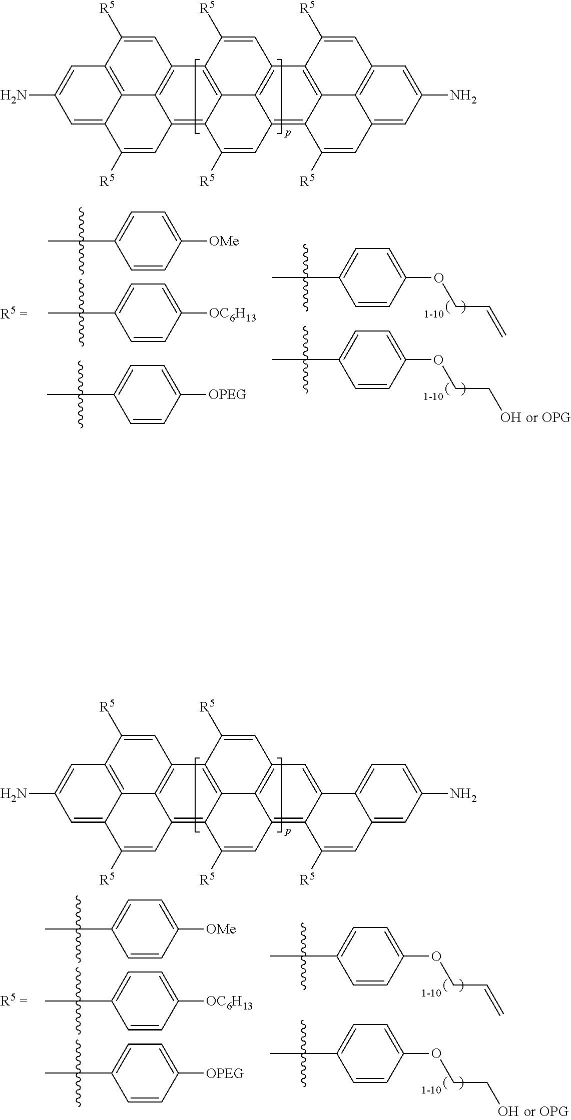

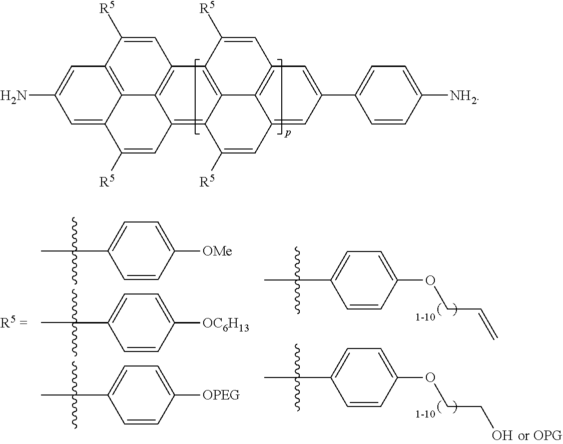

In yet another embodiment, the polycyclic aromatic compound can have a structure satisfying Formula 5.

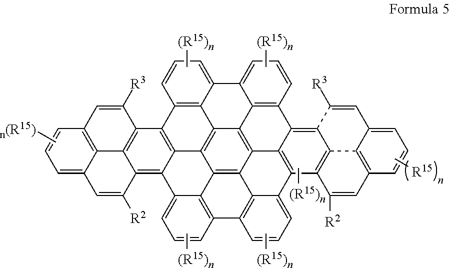

With reference to Formula 5, the following variable recitations can apply in any combination:

each of R² and R³ independently can be as recited above for any of the preceding formulas;

each R¹⁵ independently can be aliphatic, heteroaliphatic, aliphatic-aromatic, heteroaliphatic-aromatic, aromatic, or any combination thereof, or two or more R¹⁵ substituents can, together, provide an aromatic group, such as a pyrene group (together with the aromatic ring of the core structure to which each R¹⁵ is bound), a phenalene group, a perylene group, or the like;

each n independently can be an integer selected from 0 to 3, such as 0, 1, 2, or 3.

In embodiments where or two or more R substituents can, together, provide an aromatic group, the aromatic group can in turn be substituted with one or more additional R¹⁵ groups (e.g., see Formula 5C below). In particular embodiments, the polycyclic aromatic compounds disclosed in Formula 5 can have a structure satisfying any one of the Formulas 5A, 5B, or 5C below.

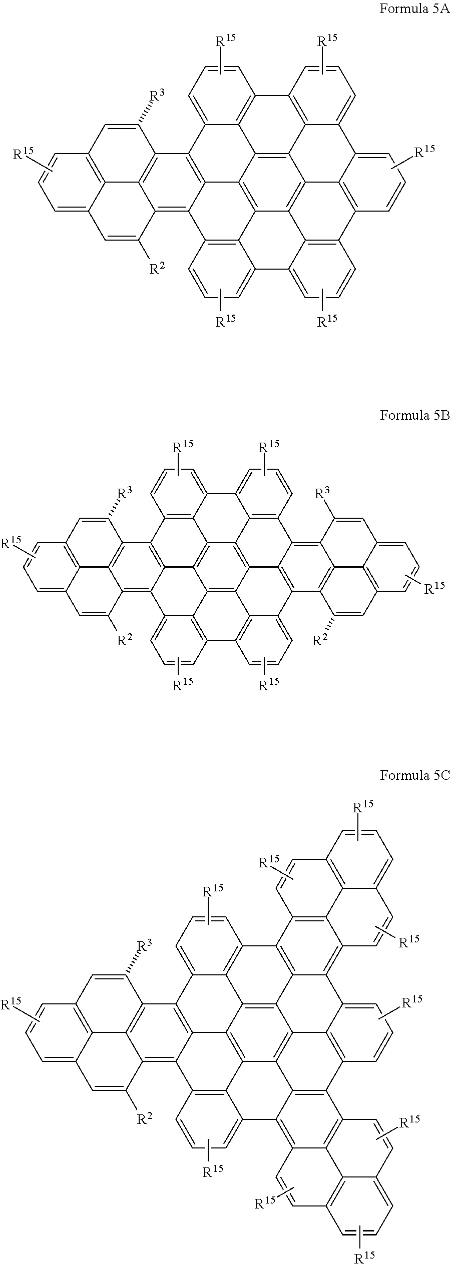

With reference to these formulas, each of R², R³, and R¹⁵ independently can be alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-arylheteroalkynyl-aryl, heteroalkyl-heteroaryl/heteroalkenyl-heteroaryl/heteroalkynyl-heteroaryl or any combination thereof. In some embodiments, the aryl and/or heteroaryl group can comprise one or more electron-donating groups, one or more electron-withdrawing groups, or any combination thereof. Exemplary electron-donating groups may include, but are not limited to, alkoxy, amide, amine, thioether, hydroxyl, thiol, acyloxy, aliphatic (e.g., alkyl, alkenyl, alkynyl), silyl, cycloaliphatic, aryl or any combinations thereof. Exemplary electron-accepting groups may include, but are not limited to, aldehyde, ketone, ester, carboxylic acid, acyl, acyl halide, cyano, halogen, sulfonate, nitro, nitroso, quaternary amine, pyridinyl, or (pyridinyl wherein the nitrogen atom is functionalized with an aliphatic or aryl group), alkyl halide or any combinations thereof. In an independent embodiment, one or more of R², R³, and R¹⁵ can be hydrogen.

Exemplary compounds meeting Formulas 5 and 5A-5B are provided below and also are disclosed in the Examples section:

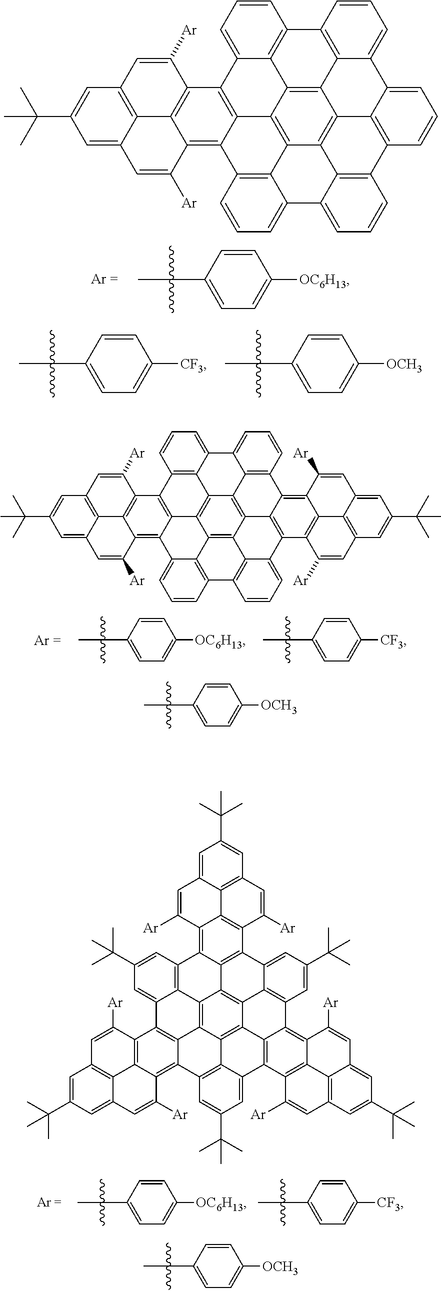

In an independent embodiment, polycyclic aromatic compounds satisfying any of the formulas above are not selected from the following compounds:

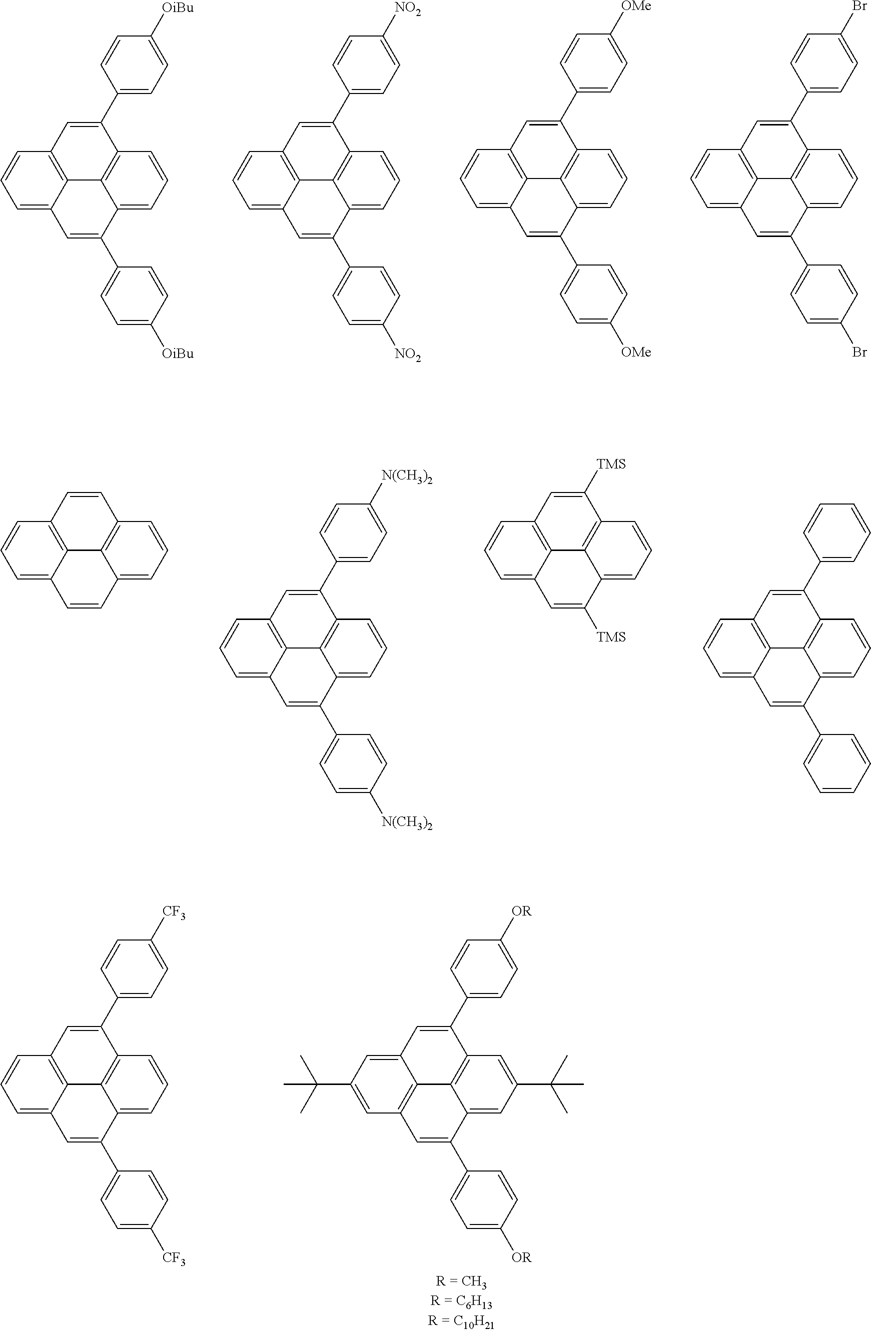

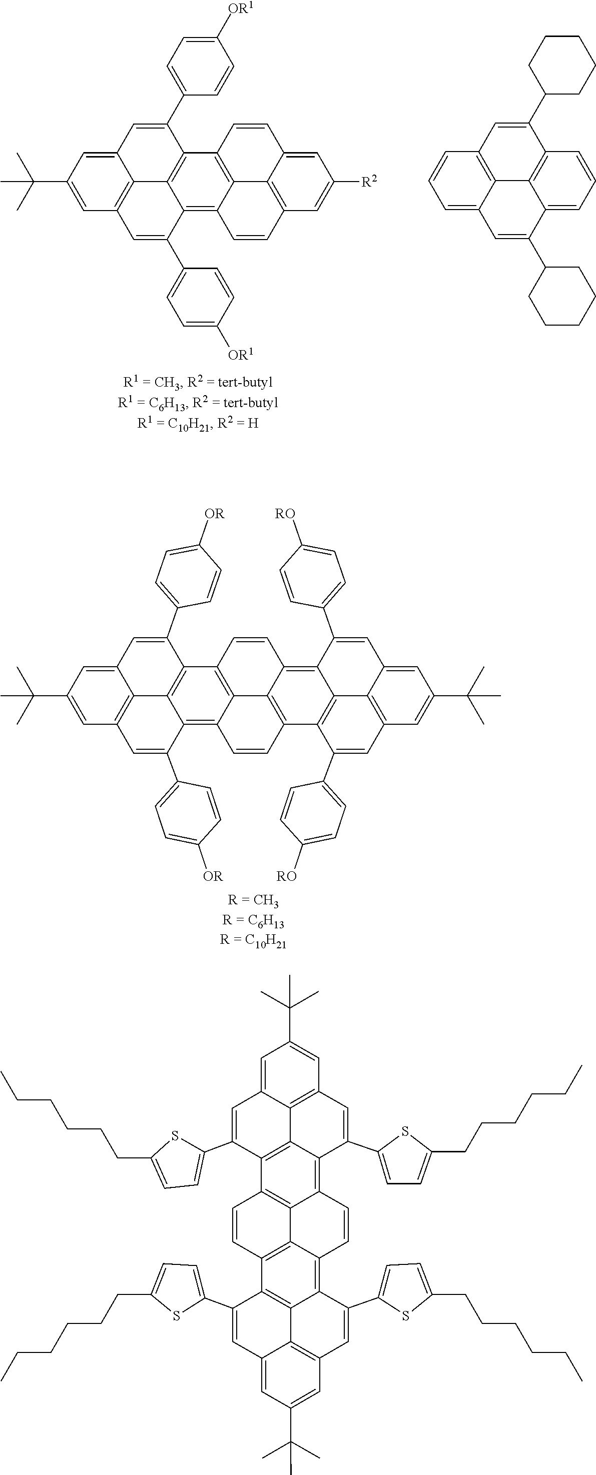

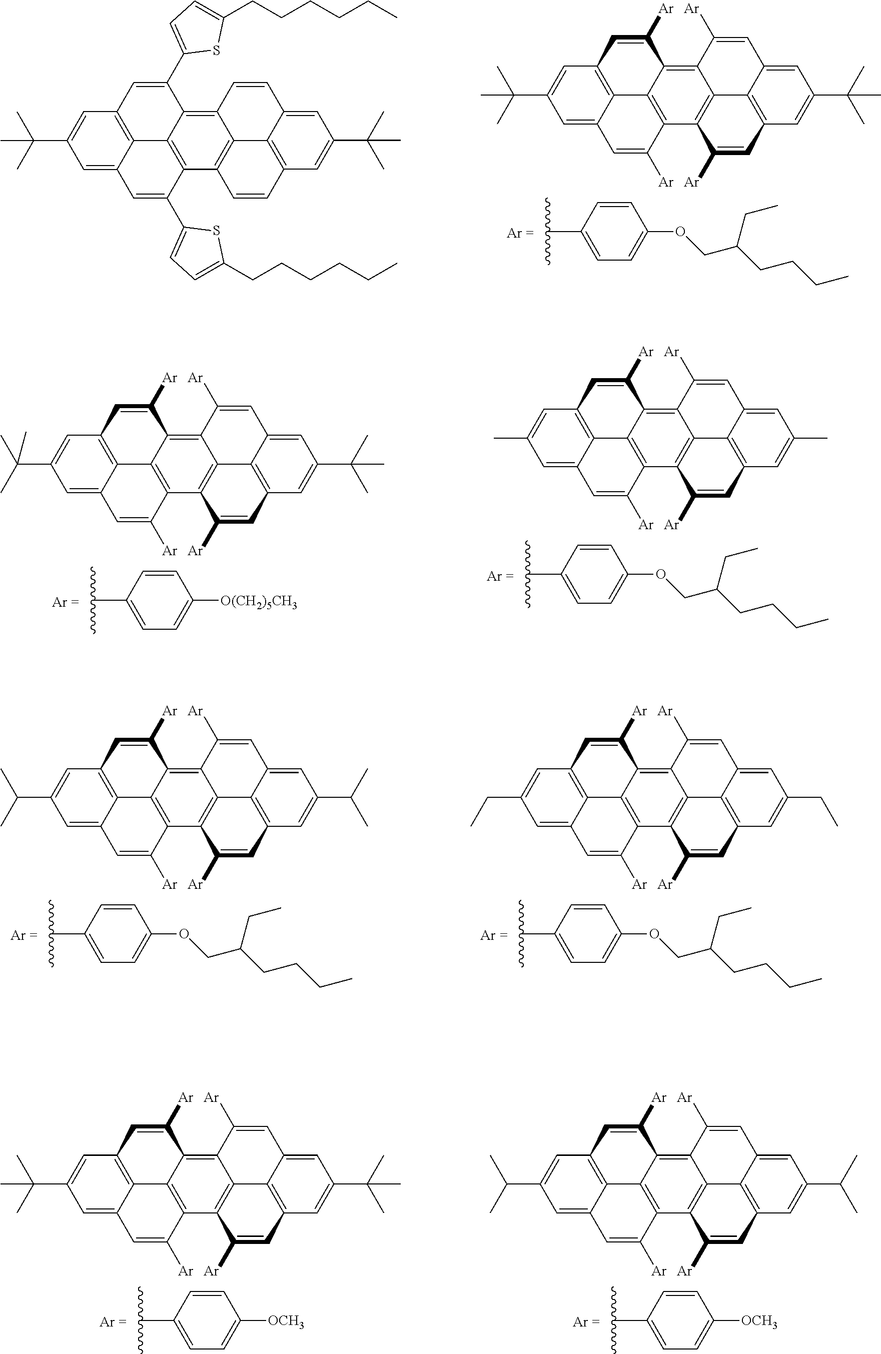

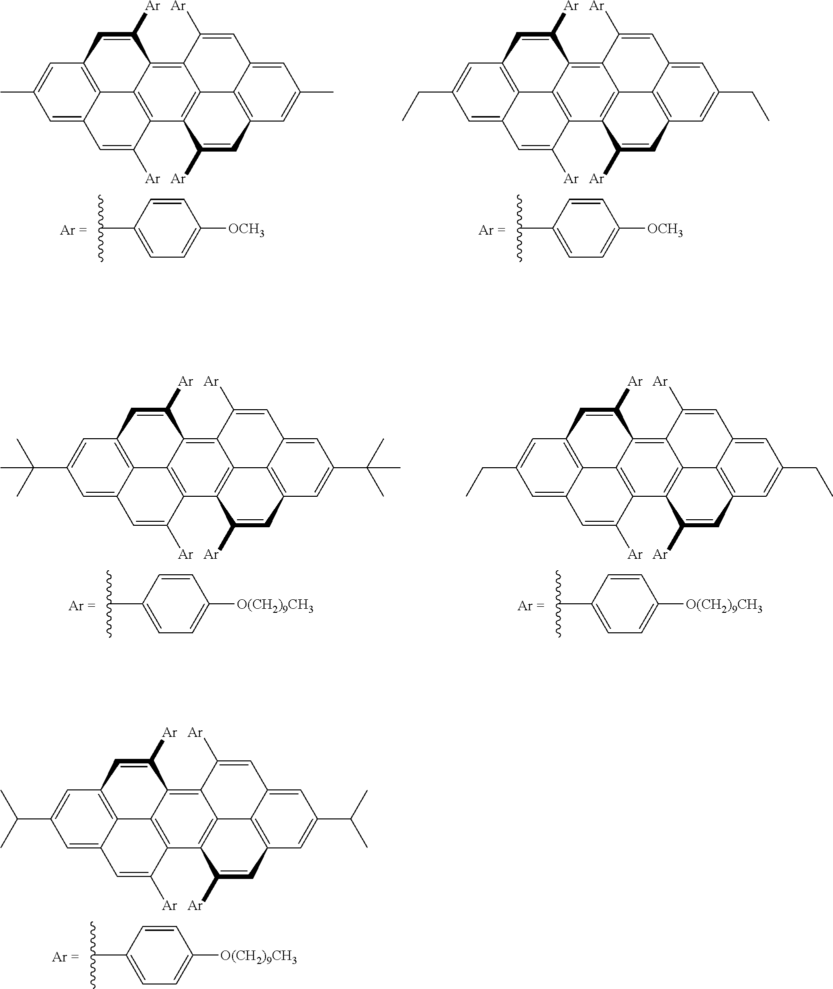

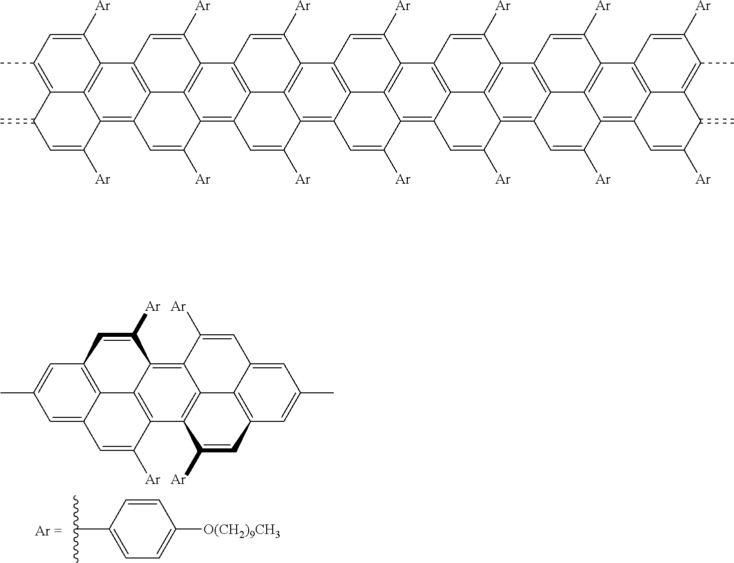

### IV. Methods of Making Compound Embodiments

Disclosed herein are method embodiments for making the polycyclic aromatic compounds disclosed herein. Certain method embodiments disclosed herein concern making of making benzene-based compounds, pyrene compounds, pyrene-based compounds, peropyrene compounds, peropyrene-based compounds, acene-based compounds, and coronene-based compounds satisfying the formulas described above.

A representative method for making pentacene-based compound embodiments is described below in Scheme 1. With reference to Scheme 1, a quadruple alkyne precursor, such as compounds 104 and 106, can be prepared and then subjected to cyclization conditions using a variety of using Brønsted acids, for instance, for the purpose of invoking a quadruple cyclization reaction, to produce the desired pentacene compounds 108. Exemplary acids may include, but are not limited to, HCO₂CF₃, HOSO₂CH₃, HOSO₂CF₃ and the like.

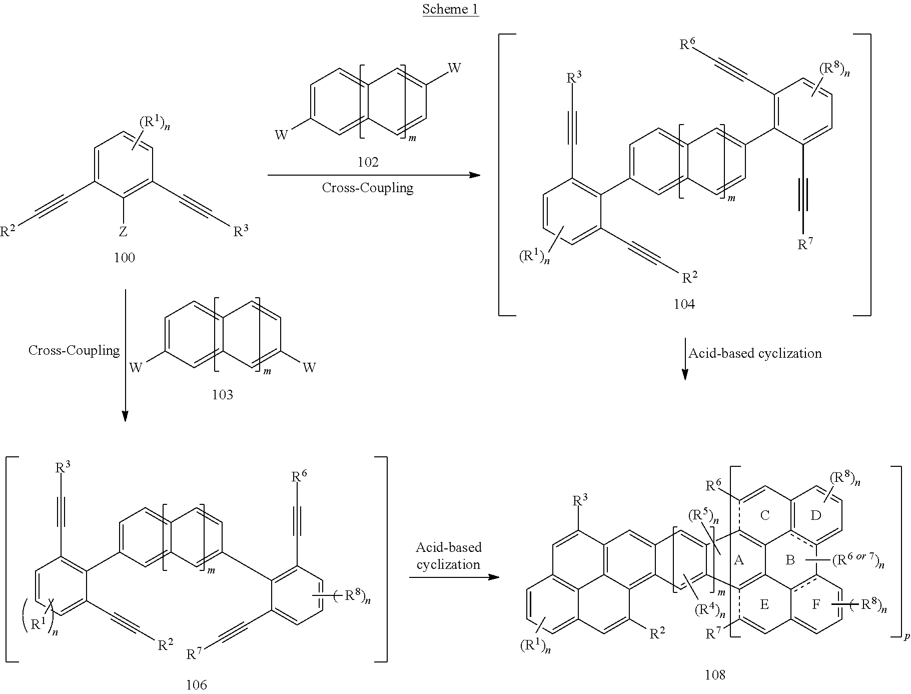

In some embodiments, each of the Y, R, R¹, R², R³ and R⁴ are recited herein; n can be an integer from 0 to 4; and Z is a boron-containing group, such as B(OH)₂, pinacol borane or a derivative thereof, and the like. In some embodiments, boronic ester or boronic acid formation can include exposing a corresponding halogen-containing precursor (e.g., halogen, such as, I, Br, F, and Cl) to a metal-containing compound (e.g., n-BuLi, s-BuLi, t-BuLi, and the like) in a solvent to facilitate halogen-metal exchange, followed by coupling of a boronic acid or a boronic ester (e.g., a boronic ester having a formula described above). In exemplary embodiments, a boronic ester, such as, a pinacol boronic ester, is used. In some embodiments, cross-coupling reactions can comprise using a palladium-based reagent to facilitate coupling of a boronic ester compound or a boronic acid compound with compound 102. In some embodiments, W can be any suitable coupling unit, such as, halogen, triflate, and the like. Suitable palladium-based reagents may include, but are not limited to, Pd(PPh₃)₄, Pd(OAc)₂, PdCl₂, Buchwald palladium reagents (e.g., XPhos Pd, SPhos PD, RuPhos PD, CPhos Pd, and the like), or Hartwig palladium reagents (e.g., Bis(tris(2-tolyl)phosphine)palladium Pd[(o-tol)₃P]₂, QPhos Pd, and the like). Further, in some embodiments, the one or more double bonds of any of the disclosed compounds can be hydrogenated via conventional hydrogenation reactions to tune their electronic and/or optical properties. An exemplary embodiment of the above-described method is provided below in Scheme 2.

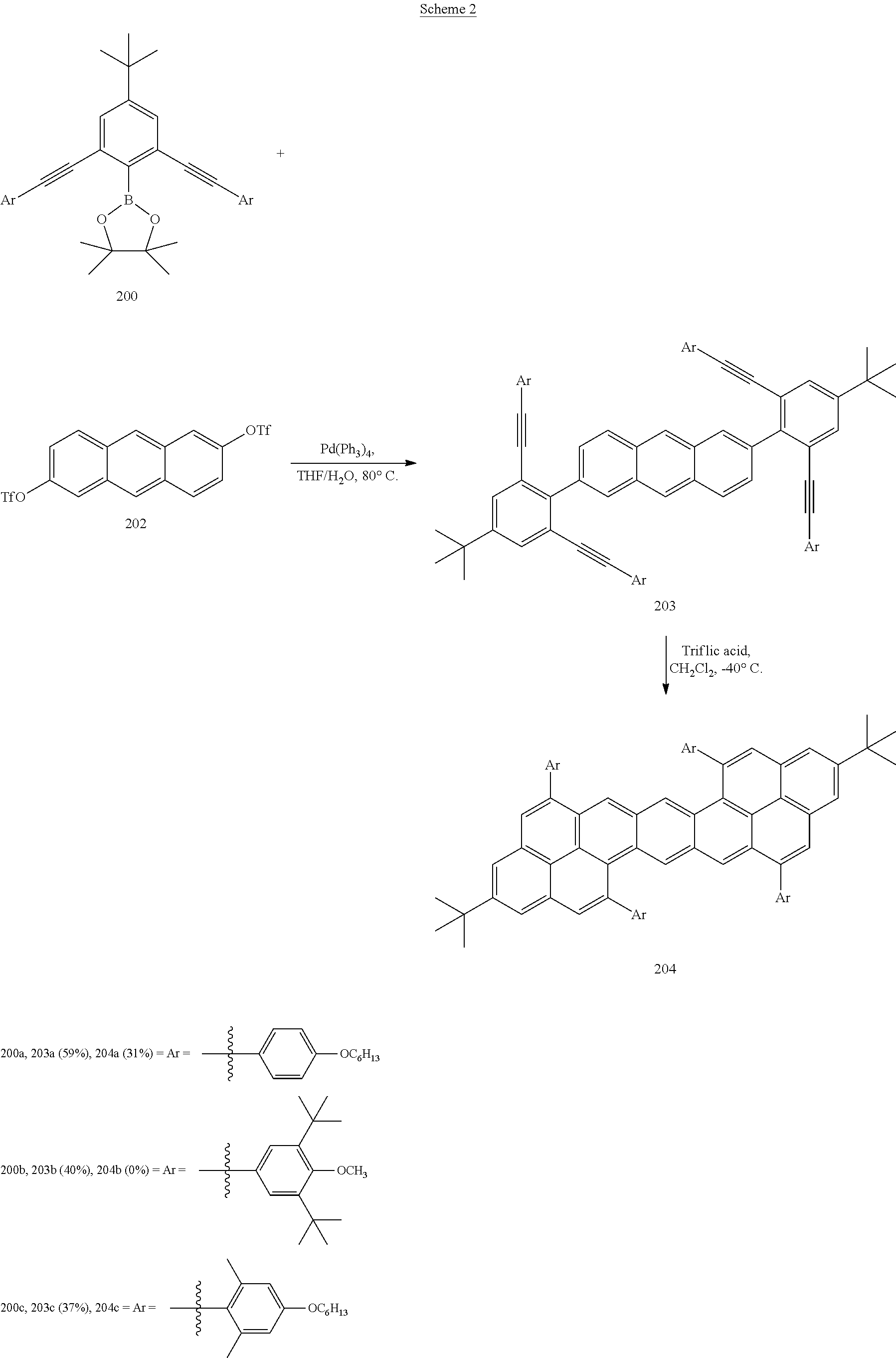

Additional method embodiments that can be used to make polycyclic aromatic compounds are illustrated below in Schemes 3-8. With reference to Schemes 3-8, each of the illustrated variables can be as recited for any of the formulas disclosed herein. As disclosed, W and Z can be as described above in connection with Scheme 1. With reference to Scheme 7, each R independently can be an electron-withdrawing group or an electron-donating group. The acid catalysis step can comprise using a Brønsted acid or a Lewis acid as disclosed herein.

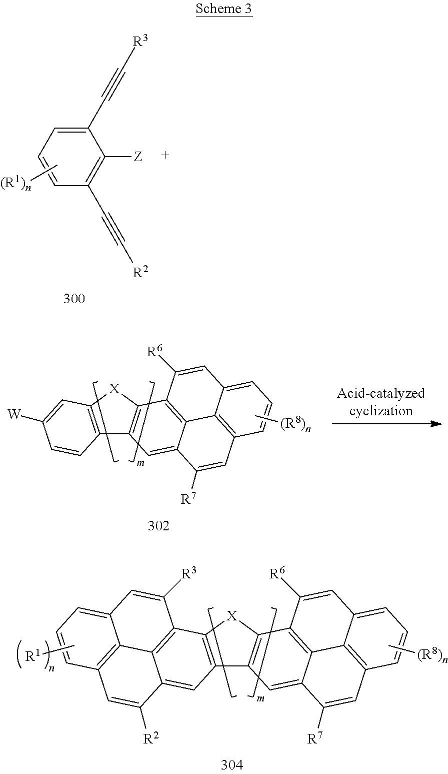

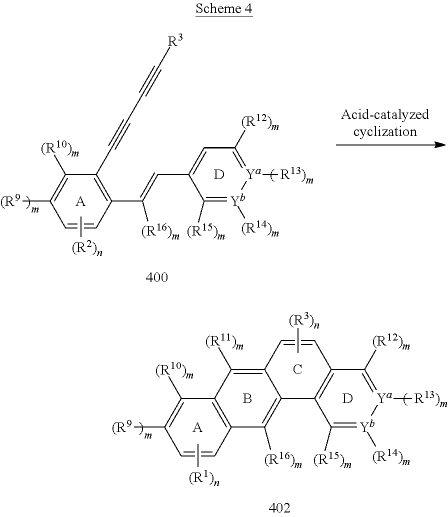

The acid-catalyzed cyclization methods used in the following schemes can be applied to any appropriate halogenated starting material (e.g., a pyrene group or other polycyclic aromatic starting material optionally comprising one or more R groups), a corresponding boronic acid coupling partner comprising one or more alkyne moieties that can optionally be functionalized with R⁵ groups, and suitable capping groups comprising at least one XR′ group to arrive at a coupled product having a backbone as illustrated in any one of Formulas 4, 4′, or 4A-4H.

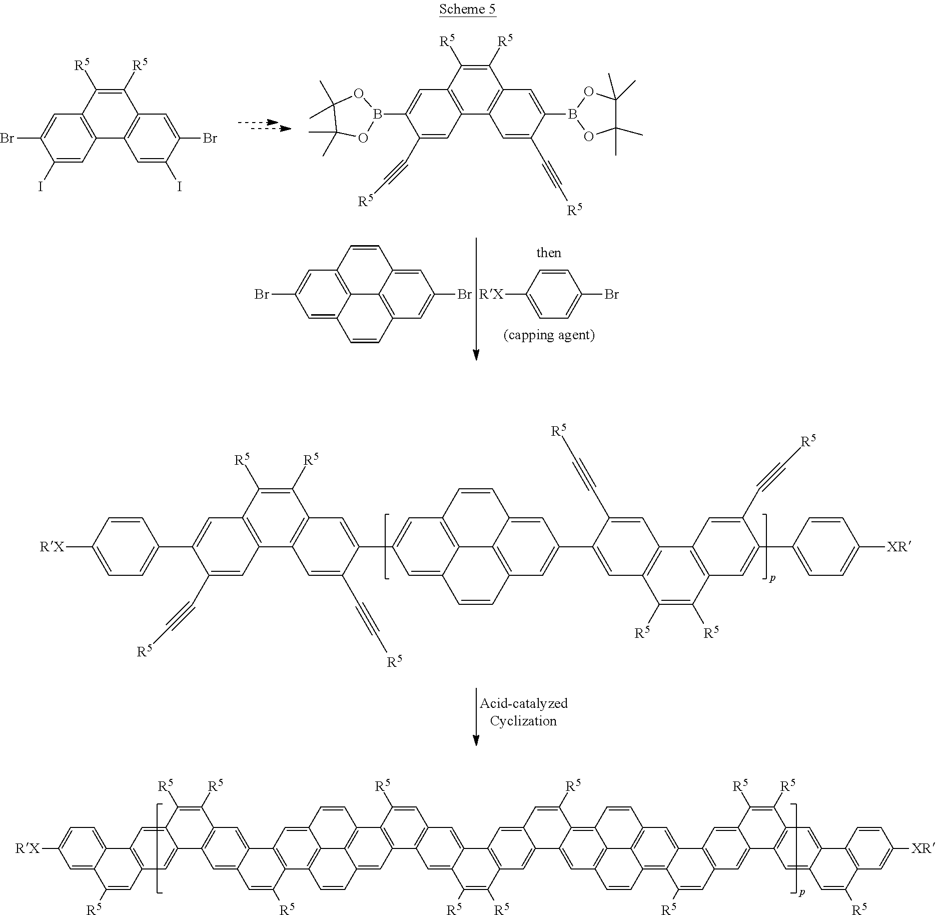

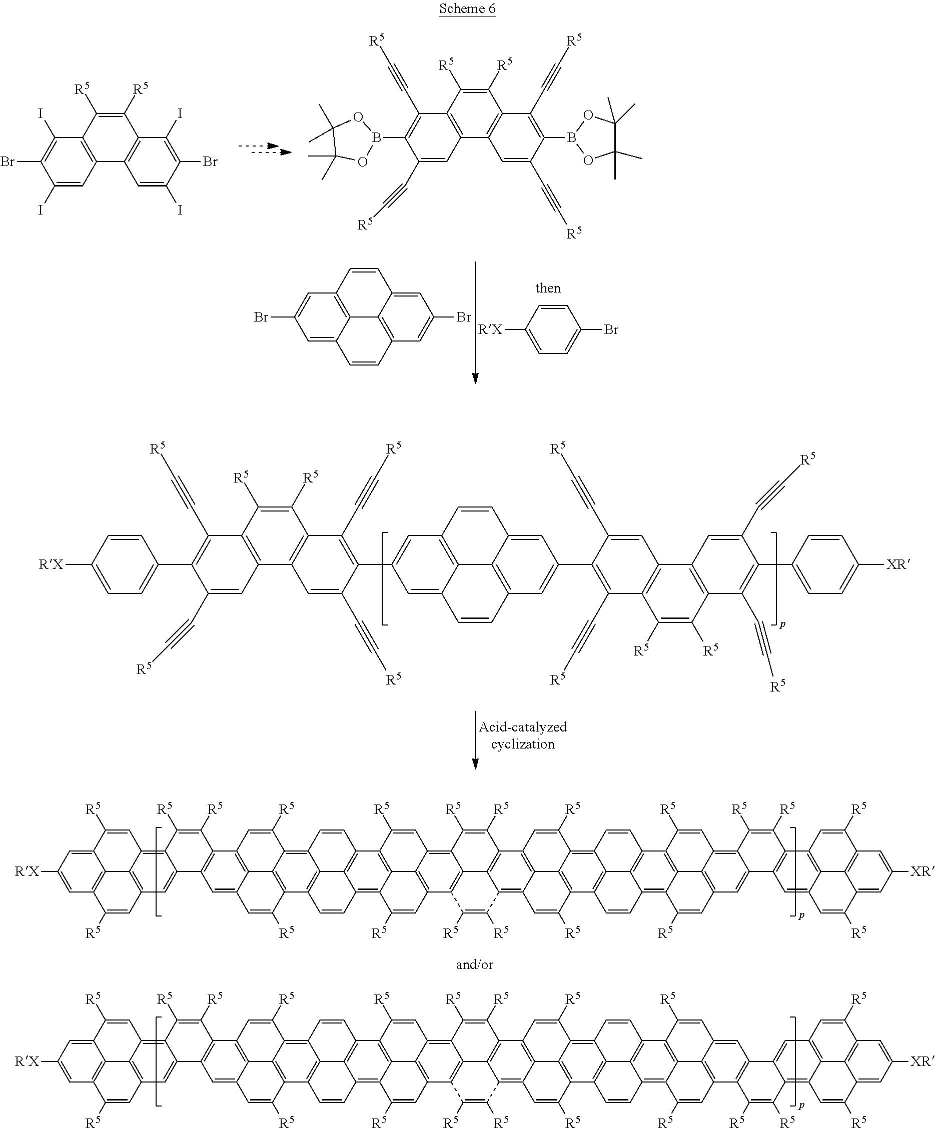

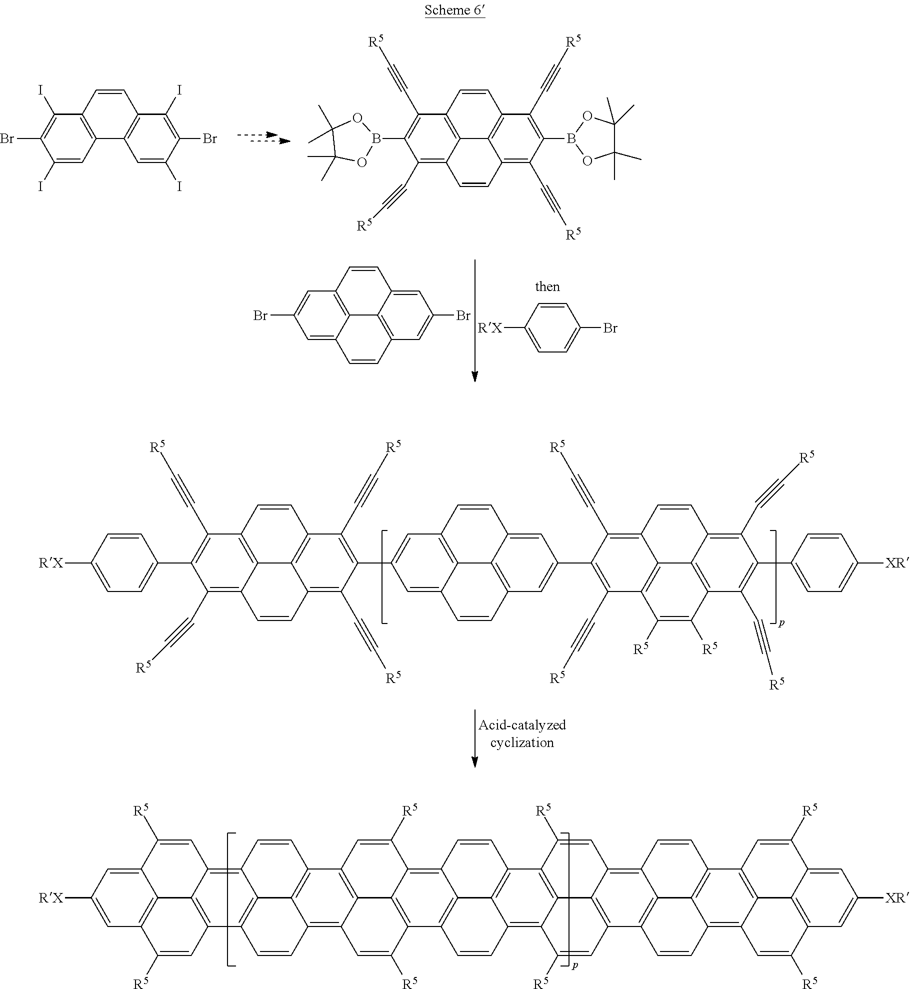

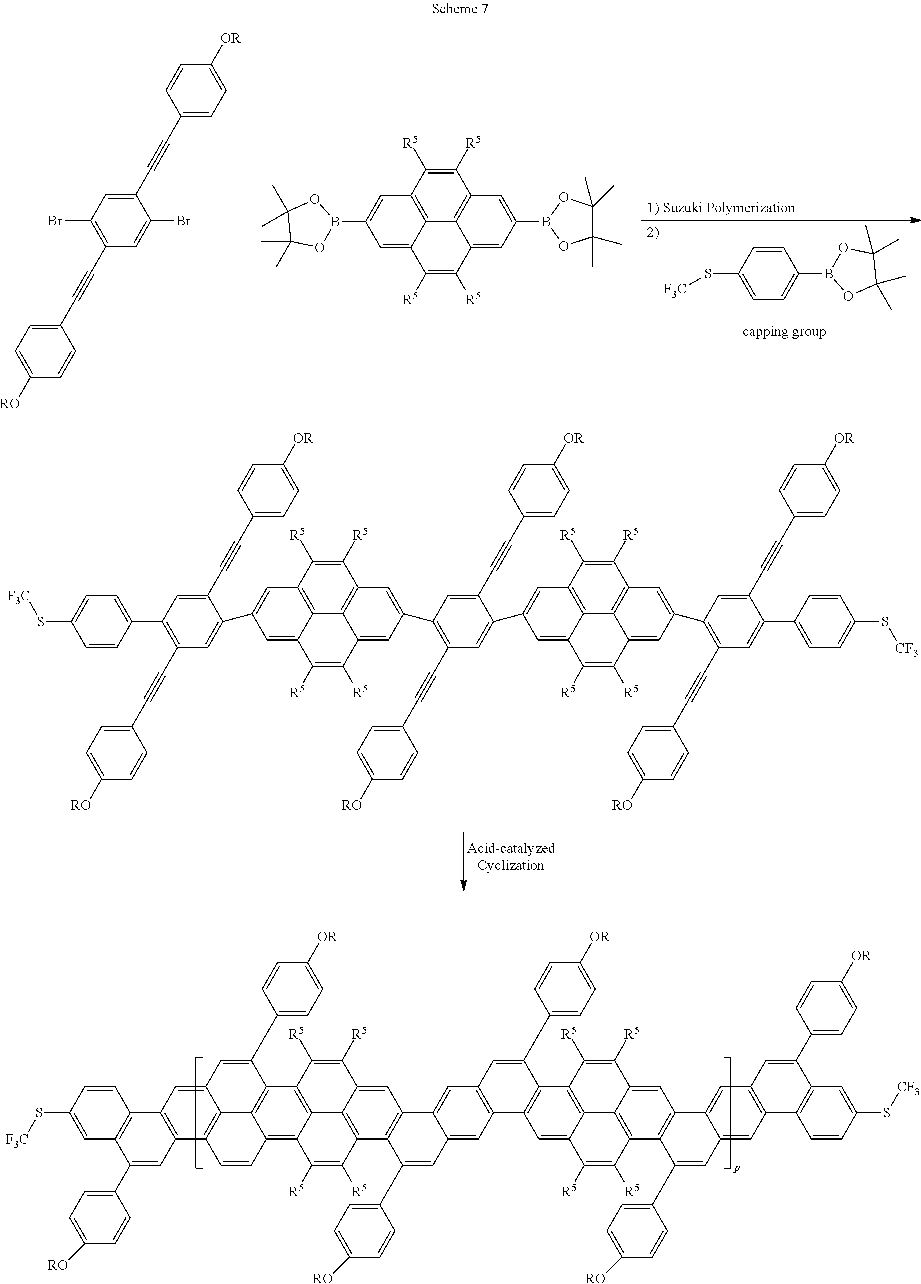

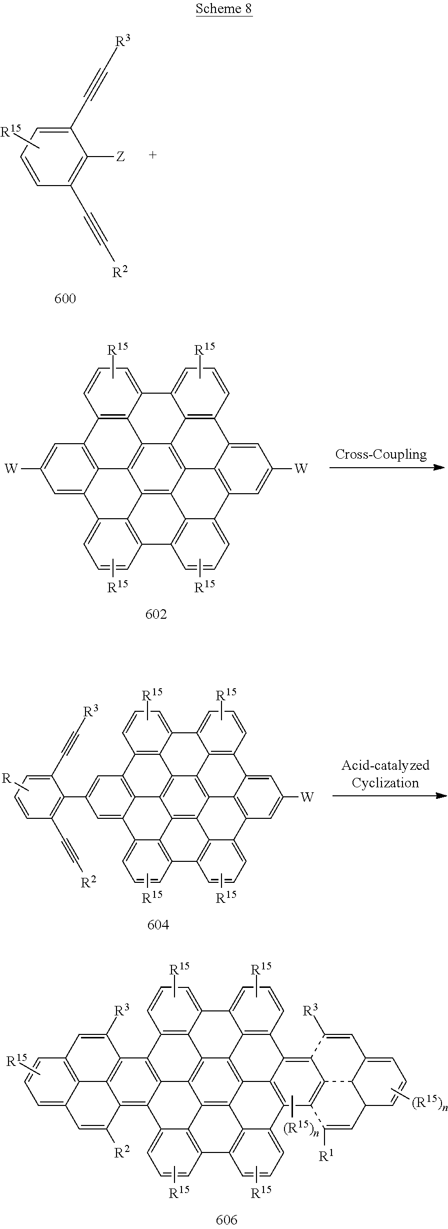

Exemplary embodiments of the above-described methods depicted in Schemes 3-8 are provided below in Schemes 9-11.

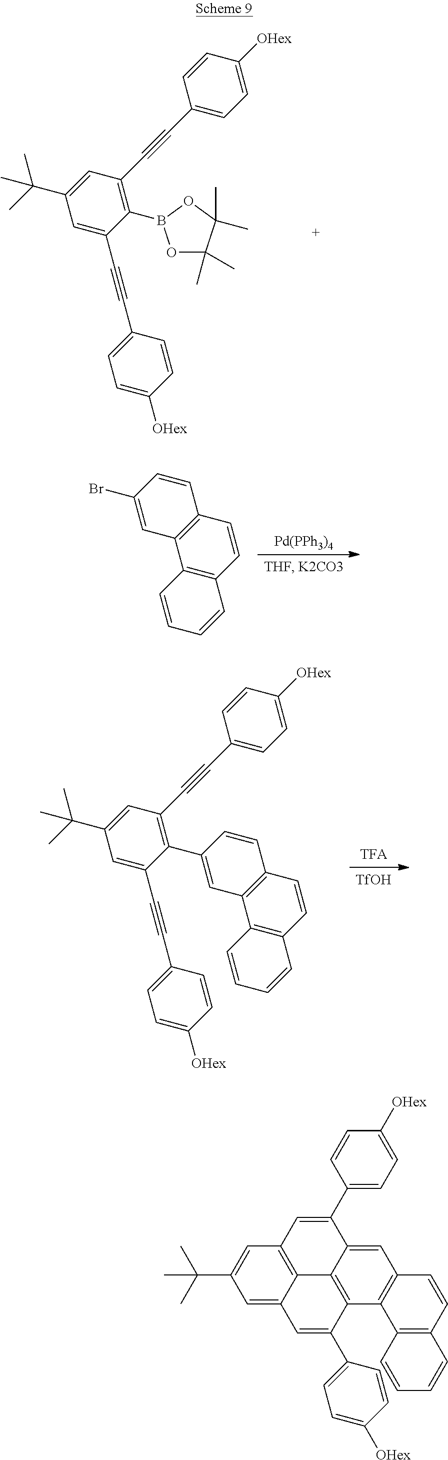

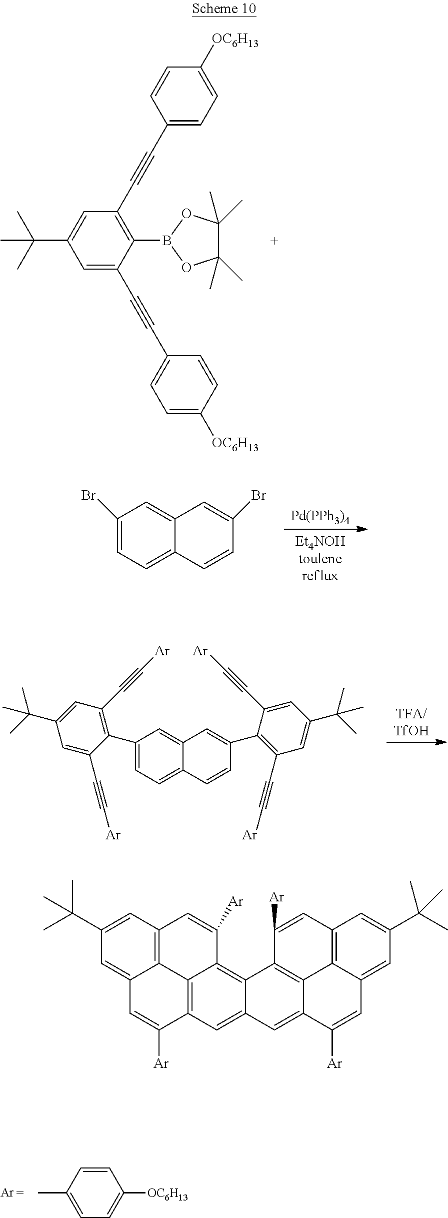

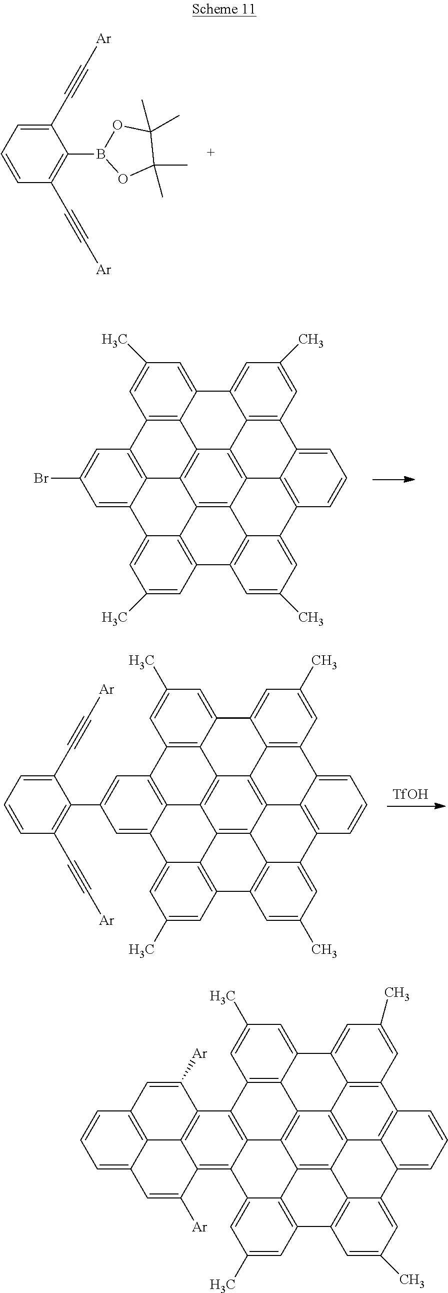

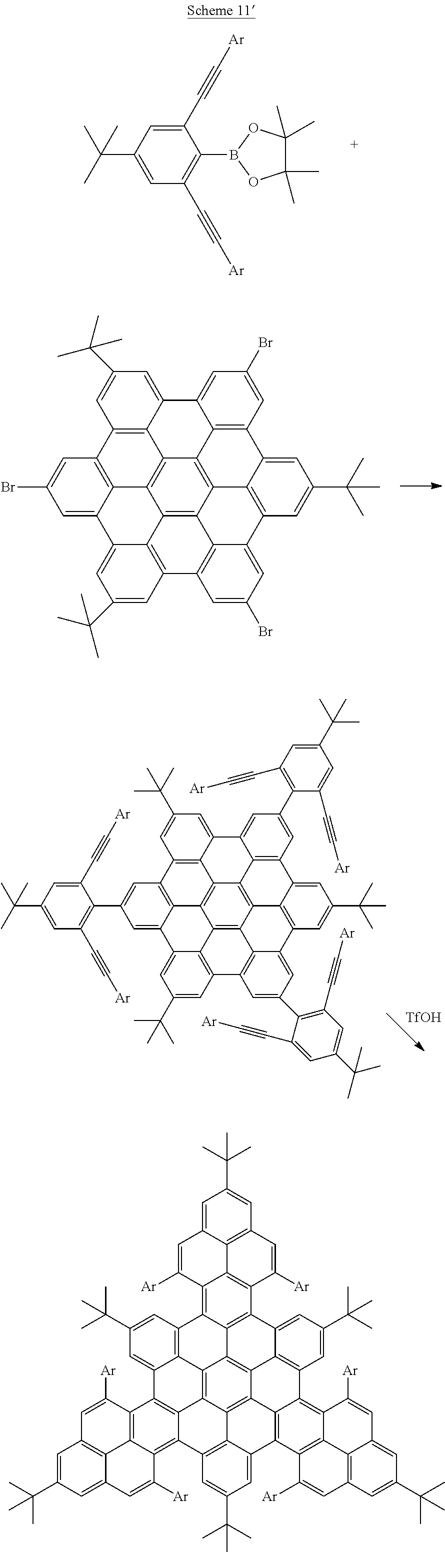

Additional representative method embodiments also are provided in the Examples of the present disclosure.

### V. Methods of Use

Disclosed herein are embodiments of methods of using the compound embodiments described herein. In some embodiments, the compounds can be used in electronic devices, such as in organic transistors, light-emitting devices, or organic photovoltaics. In some embodiments, the compounds act as sensitizing materials to generate singlet oxygen and thus can be used in photochemistry and phototherapy (e.g., cancer treatment). In additional embodiments, the compounds can be used as visible- to near IR-absorbing and emitting materials for biological applications as these wavelengths have deep biotissue penetration. Also, embodiments of chiral compounds disclosed herein are useful in chiroptic applications and as chiral sensors.

In some embodiments, compound embodiments having structures described herein, particularly compounds satisfying Formulas 4, 4′, and 4A-4H, are used in electronic devices by coupling the compounds to electrode components of a device. In some embodiments, the compounds can be coupled to an electrode component through a functional group of the compound, such as the —XR′ group (or groups) illustrated in Formulas 4, 4′, and 4A-4H, positioned at termini of the compounds. Compound embodiments of the present disclosure, such as compounds satisfying Formulas 4, 4′, and 4A-4H, can be used as semiconducting molecular wires that are covalently linked to electrodes or act as the semiconducting organic layer between two electrodes (source and drain or anode and cathode). The conductivity of the molecular wires can be modulated by an external field (e.g., a gate electrode) or other stimulus (e.g., adsorption of a small molecule) and any resulting change in conductivity can be detected. In additional embodiments, changes in conductivity can be detected when compound embodiments are used in sensing applications, wherein the conductivity changes can be specific for a particular molecule that is sensed (e.g., such as when a small molecule or other compound is adsorbed on or near a compound embodiment of the present disclosure). Compound embodiments of the present disclosure also can be covalently connected (or used to detect) to small molecules (e.g., chelators) or large molecules (e.g., biological molecules, such as enzymes, or proteins) that can perform a reaction or work or act as a sensor. The molecules can be used for applications such as gene sequencing (genomics), organic field-effect transistors, organic light-emitting diodes, organic memory devices, and the like, or electronic components used in such devices (e.g., semiconductor chips, electrodes, or the like).

### VI. Overview of Several Embodiments

Also disclosed herein are compound embodiments wherein the compound has a structure satisfying any one of Formula 4, 4′, or Formulas 4A-4H, as disclosed herein.

In any or all of the above such embodiments, X is a heteroatom, such as O, S, or N(R″)₂ wherein R″ is hydrogen, aliphatic, haloaliphatic, haloheteroaliphatic, heteroaliphatic, aromatic or any combination thereof, each R′ independently is selected from hydrogen, aliphatic, or —C(O)R²⁰, wherein R²⁰ can be hydrogen, aliphatic, haloaliphatic, haloheteroaliphatic, heteroaliphatic, aromatic or any combination thereof; and each R⁵, if present, independently is aliphatic, heteroaliphatic, aliphatic-aromatic, heteroaliphatic-aromatic, aromatic, or any combination thereof, each n independently is 0, 1, or 2; q is an integer selected from 0 to 1000; p is an integer selected from 0 to 1000; and r is an integer selected from 1 to 1000, provided that when p is 0, r is the same integer as q. In some embodiments, each A of Formulas 4E-4H independently is CH or nitrogen (or an ionized form thereof, such as =N+Me).

In any or all of the above such embodiments, X, together with R′ is —OH, —OCH₃, —OC(O)CH₃, —SH, —SCH₃, —SC(O)CH₃, —NH₂, —NHCH₃, or —NHC(O)CH₃.

In any or all of the above such embodiments, each R independently is an alkyl group, alkoxy group, an alkylene oxide group, or an aromatic group comprising an alkylene oxide group.

Also disclosed herein are compound embodiments wherein the compound has a structure satisfying any one of Formula 1 or Formulas 1A-1E, as disclosed herein.

In any or all of the above such embodiments, each individual R¹, R⁴, R⁵, and R, if present, independently is aliphatic, haloaliphatic, haloheteroaliphatic, heteroaliphatic, aromatic, aliphatic-aromatic, heteroaliphatic-aromatic, or any combination thereof; each R², R³, R⁶, and R⁷ independently is hydrogen, aliphatic, haloaliphatic, haloheteroaliphatic, heteroaliphatic, aromatic, aliphatic-aromatic, heteroaliphatic-aromatic, or any combination thereof; each n independently is an integer selected from 0 to 4; p is 0 or 1, and when p is 0, none of the rings A, B, C, D, or E are present; m is an integer selected from 0 to 10; and ring A, when present, is phenyl, or ring A joins together with (i) rings B, C, and D to form a pyrene group; or (ii) rings B, E, and F to form a pyrene group; or (iii) ring E to form a naphthyl group.

Also disclosed herein are compound embodiments wherein the compound has a structure satisfying any one of Formula 2 or Formulas 2A-2F, as disclosed herein.

In any or all of the above such embodiments, X is selected from O, S, C=O, C=S, SO₂, SO, C(R′)₂, N(R′), or N⁺(R′)₂ wherein each R′ independently is hydrogen, aliphatic, haloaliphatic, haloheteroaliphatic, heteroaliphatic, aromatic or any combination thereof; each R¹ and R⁸, if present, independently is selected from aliphatic, haloaliphatic, haloheteroaliphatic, heteroaliphatic, aromatic, aliphatic-aromatic, heteroaliphatic-aromatic, or any combination thereof; each R², R³, R⁶, and R⁷ independently is hydrogen, aliphatic, haloaliphatic, haloheteroaliphatic, heteroaliphatic, aromatic, aliphatic-aromatic, heteroaliphatic-aromatic, or any combination thereof; m is 0 or 1; and each n independently is an integer selected from 0 to 3.

In any or all of the above such embodiments, each of R¹, R² and R³ can independently be aryl; heteroaryl; aliphatic-aryl; aliphatic-heteroaryl; heteroaliphatic-aryl; heteroaliphatic-heteroaryl; or any combination thereof.

Also disclosed herein are compound embodiments wherein the compound has a structure satisfying any one of Formula 3 or Formulas 3A-3R, as disclosed herein.

In any or all of the above such embodiments, Y^{a} is carbon, CH (when R⁷ is not present, as in when m is 0), or nitrogen; Y is carbon, CH (when R⁸ is not present, as in when m is 0), or nitrogen; each of R¹, R³, R⁹, R¹⁰, R¹¹, R¹², R¹³, R¹⁴, R¹⁵, and R¹⁶ independently is selected from aliphatic, haloaliphatic, haloheteroaliphatic, heteroaliphatic, aliphatic-aromatic, heteroaliphatic-aromatic, or aromatic; or any one or more of (i)-(iv) apply, wherein (i) R¹ and R⁹ join together to form an aryl group or a heteroaryl group, or any combination thereof; (ii) R⁹ and R¹⁰ join together to form an aryl group or a heteroaryl group, or any combination thereof; (iii) R¹⁰ and R¹¹ join together to form an aryl group or a heteroaryl group, or any combination thereof; (iv) R¹² and R¹³ join together to form an aryl group or a heteroaryl group, or any combination thereof; (v) R¹², R¹³, and R¹⁴ can join together to form an aromatic group having a structure

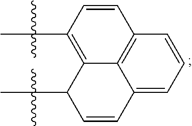 [SMILES: *c1ccc2cccc3c2c1C(*)C=C3]

or an aromatic group having a structure

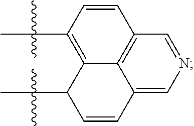 [SMILES: *c1ccc2cncc3c2c1C(*)C=C3]

(vi) R¹³ and R¹⁴ can join together to form an aryl group or a heteroaryl group, or any combination thereof, and/or (vii) R¹⁵ and R¹⁶ can join together to form an aryl group or a heteroaryl group, or any combination thereof, each n independently is an integer selected from 0, 1 or 2; and each m independently is 0 or 1.

In any or all of the above embodiments, when R⁹ and R¹⁰ can together be an aryl or heteroaryl, R⁸ can independently be an aryl, heteroaryl or any combination thereof.

Also disclosed herein are compound embodiments wherein the compound has a structure according to any one of Formulas 5, 5A, 5B, or 5C.

In any or all of the above such embodiments, each of R¹, R² and R³ independently can be selected from halogen, aliphatic, haloaliphatic, haloheteroaliphatic, heteroaliphatic, aromatic, aliphatic-aromatic, heteroaliphatic-aromatic or any combination thereof, each R²¹ independently is aliphatic, heteroaliphatic, aliphatic-aromatic, heteroaliphatic-aromatic, aromatic, or any combination thereof, and each n independently is an integer selected from 0 to 3.

Also disclosed herein are embodiments of a method for making the compound according to any or all of the above compound embodiments, comprising: exposing a compound comprising a first aromatic group functionalized with (i) one or more alkyne moieties and (ii) a second aromatic group to a catalyst effective to promote forming an intramolecular bond between the one or more alkyne moieties of the first aromatic group and a carbon atom of the second aromatic group.

In any or all of the above embodiments, the method further comprises pre-forming the compound by using a transition metal to couple a starting material comprising the first aromatic group and further comprising a boronic acid or a boronic ester with a starting material comprising the second aromatic group and further comprising a halogen atom.

In any or all of the above embodiments, the compound further comprises a third aromatic group functionalized with one or more alkyne moieties.

In any or all of the above embodiments, the catalyst is effective to promote forming an intramolecular bond between the one or more alkyne moieties of the third aromatic group and a carbon atom of the second aromatic group.

In any or all of the above embodiments, the catalyst is a Brønsted acid or a Lewis acid.

In any or all of the above embodiments, the catalyst is HCO₂CF₃, HOSO₂CH₃, HOSO₂CF₃, InCl₃, PtCl₂, AuCl₃, or AuC(PPh₃).

Also disclosed herein are embodiments of a device, comprising a compound according to any one or more of the above compound embodiments, wherein the device is an electronic device selected from an organic transistor, a light-emitting device, a sensor device, or an organic photovoltaic device; or a device for detecting biological compounds.

### VI. Examples

General Experimental Section-Chemicals and solvents were purchased from VWR, Oakwood Chemicals, and Sigma-Aldrich, and used directly without further purification unless otherwise stated. All reactions dealing with air- or moisture-sensitive compounds were carried out in a dry reaction vessel under nitrogen. Anhydrous toluene was obtained by passing the solvent (HPLC grade) through an activated alumina column on a PureSolv MD 5 solvent drying system.

¹H and ¹³C NMR spectra were recorded on Varian 400 MHz or Varian 500 MHz NMR Spectrometers. Spectra were recorded in deuterated chloroform (CDCl₃). Tetramethylsilane (TMS, set to 0 ppm) was used as internal standard for chemical shifts. Solvent peaks were referenced as 7.26 ppm for ¹H and 77.16 ppm for ¹³C NMR, respectively. Chemical shifts (δ) are reported in parts per million (ppm) from low to high frequency and referenced to the residual solvent resonance. Coupling constants (J) are reported in Hz. The multiplicity of ¹H signals are indicated as: s=singlet, d=doublet, t=triplet, dd=double doublet, m=multiplet, br=broad.

High resolution ESI mass spectrometry was recorded using an Agilent 6230 TOF MS, and trifluoro acetic acid (TFA) was added to samples to promote ionization.

MALDI-TOF mass spectra were recorded on a Bruker microflex MALDI-TOF spectrometer.

TLC information was recorded on Silica gel 60 F254 glass plates. Purification of reaction products was carried out by flash chromatography using Silica Gel 60 (230-400 mesh).

UV/vis spectrum of PDAPP was obtained on a Perkin-Elmer Lambda 750 UV/vis spectrophotometer.

Certain embodiments of starting materials used in methods disclosed herein can be made according to the following procedure: To the solution of suitable1,3-diiodobenzene derivative SM 1 (1.00 equiv.) and the terminal alkyne SM 2 (2.5 equiv.) in Et₃N (40 mL) and THF (80 mL), were added Pd(PPh₃)₂Cl₂ (10 mol %) and CuI (20 mol %). The resulting mixture was stirred under a N2 atmosphere at room temperature for 14 h. The ammonium salt was then removed by filtration. The solvent was removed under reduced pressure and the residue was purified by column chromatography to afford the product. To a solution of the product (1 eq.) in THF at −78° C. was added a solution of n-butyllithium in hexanes (2.5 M, 1.2 eq.). After stirring for 1 hour at −78° C., isopropoxyboronic acid pinacol ester (1.2 eq.) was added, the reaction removed from the cooling bath and allowed to warm. Upon reaching room temperature the reaction was quenched by the addition of H₂O, and then extracted with DCM. The extract was washed with water, dried with Na₂SO₄, filtered and concentrated in vacuo. The residue was purified by flash column chromatography. Representative starting materials are summarized below.

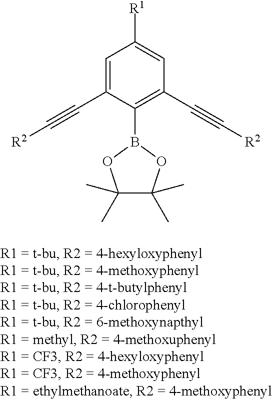

Synthesis of Compound 203:

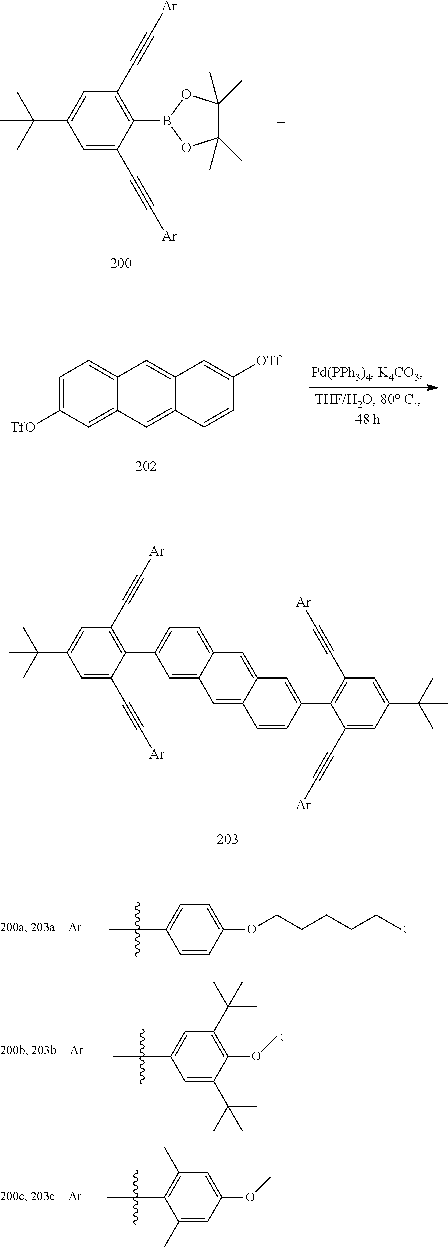

General Procedure for the Synthesis of Compound 203:

2,6-Diyl bis(trifluoromethanesulfonate)-anthracene 202 (118 mg, 0.250 mmol), 2,6-diynylphenyl borate 200 (0.600 mmol) and K₂CO₃ (138 mg, 1.00 mmol) were dissolved in THF (15 mL) and water (3 mL) solution. Pd(PPh₃)₄ (30.0 mg, 0.0250 mmol) was added to the solution before degassing the mixture via bubbling nitrogen for 30 min. The resulting mixture was stirred under a N₂ atmosphere at 80° C. for 48 h. After the reaction was complete, the mixture was diluted with DCM, washed with H₂O and dried over Na₂SO₄. The solvent was removed under reduced pressure and the residue was purified by column chromatography.

Compound 203a:

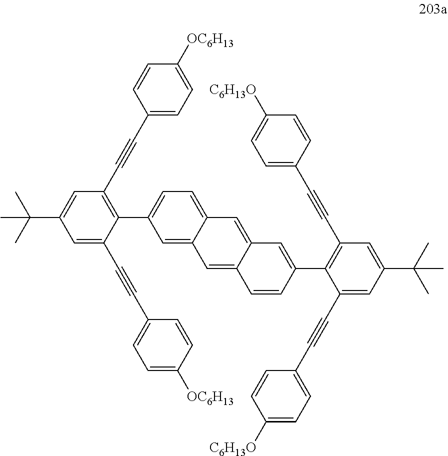

Purification by flash column chromatography (silica gel, hexane:DCM=4:1, v/v) yielded pure compound 203a as a light yellow solid (183 mg, yield 59%). R_{f}=0.3 (hexane/DCM 4:1); FTIR (neat) 2955, 2931, 2869, 2208, 1605, 1508, 1468, 1283, 1247, 1171, 1025, 829 cm⁻¹; ¹H NMR (400 MHz, CDCl₃) δ 8.64 (s, 2H), 8.52-8.40 (m, 2H), 8.24-8.12 (m, 2H), 7.88 (d, J=8.7 Hz, 2H), 7.73 (s, 4H), 7.25-7.02 (m, 8H), 6.77-6.55 (m, 8H), 3.80 (t, J=6.6 Hz, 8H), 1.69 (dq, J=14.7, 6.7 Hz, 8H), 1.59-1.42 (m, 18H), 1.46-1.17 (m, 24H), 1.02-0.78 ppm (m, 12H); ¹³C NMR (100 MHz, CDCl₃) δ 159.2, 150.3, 142.6, 136.2, 132.9, 131.7, 131.4, 130.0, 129.3, 128.9, 126.8, 126.4, 123.4, 115.1, 114.5, 92.8, 88.5, 68.0, 34.7, 31.7, 31.3, 29.2, 25.7, 22.7, 14.1 ppm; MS (MALDI-TOF): calcd for C₉H₉₈O₄ [M+H]⁺ 1243.754, found 1243.760.

Compound 203b:

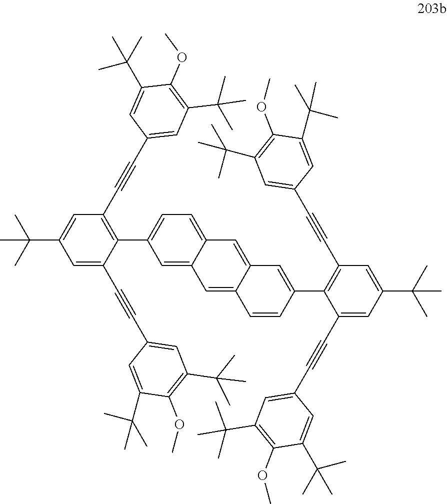 [SMILES: COc1c(C(C)(C)C)cc(C#Cc2cc(C(C)(C)C)cc(C#Cc3cc(C(C)(C)C)c(OC)c(C(C)(C)C)c3)c2-c2ccc3cc4cc(-c5c(C#Cc6cc(C(C)(C)C)c(OC)c(C(C)(C)C)c6)cc(C(C)(C)C)cc5C#Cc5cc(C(C)(C)C)c(OC)c(C(C)(C)C)c5)ccc4cc3c2)cc1C(C)(C)C]

Purification by flash column chromatography (silica gel, hexane:DCM=7:1, v/v) yielded pure compound 203b as a light yellow solid (141 mg, yield 40%). R_{f}=0.2 (hexane/DCM 7:1); FTIR (neat) 2961, 2869, 2208, 1590, 1446, 1418, 1394, 1363, 1255, 1221, 1115, 1009, 884 cm⁻¹; ¹H NMR (400 MHz, CDCl₃) δ 8.60 (s, 2H), 8.47-8.43 (m, 2H), 8.13-8.08 (m, 2H), 7.88 (d, J=8.7, 2H), 7.74 (s, 4H), 7.09 (s, 8H), 3.52 (s, 12H), 1.46 (s, 18H), 1.18 ppm (s, 72H); ¹³C NMR (100 MHz, CDCl₃) δ 160.1, 150.3, 143.9, 143.1, 136.0, 131.9, 131.4, 130.2, 130.0, 129.3, 128.9, 126.9, 126.4, 123.3, 117.5, 93.4, 88.2, 64.3, 35.6, 34.8, 31.8, 31.4 ppm; MS (MALDI-TOF): calcd for C₁₀₂H₁₂₂O₄ [M+H]⁺ 1411.942, found 1412.127.

Compound 203c:

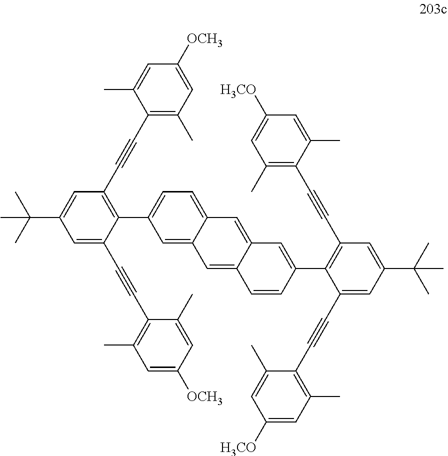 [SMILES: COc1cc(C)c(C#Cc2cc(C(C)(C)C)cc(C#Cc3c(C)cc(OC)cc3C)c2-c2ccc3cc4cc(-c5c(C#Cc6c(C)cc(OC)cc6C)cc(C(C)(C)C)cc5C#Cc5c(C)cc(OC)cc5C)ccc4cc3c2)c(C)c1]

Purification by flash column chromatography (silica gel, hexane:DCM=1:1, v/v) yielded pure compound 203c as alight yellow solid. R_{f}=0.2 (hexane/DCM 1:1); FTIR (neat) 2958, 2902, 2833, 2198, 1603, 1466, 1436, 1314, 1279, 1264, 1194, 1148, 1061, 837 cm⁻¹; ¹H NMR (500 MHz, CDCl₃) δ 8.40 (s, 2H), 8.15 (d, J=1.6 Hz, 2H), 8.01 (d, J=8.6 Hz, 2H), 7.64 (s, 4H), 7.56 (dd, J=8.6, 1.7 Hz, 2H), 6.38 (s, 8H), 3.65 (s, 12H), 1.91 (s, 24H), 1.46 ppm (s, 18H); ³C NMR (125 MHz, CDCl₃) δ 159.1, 150.4, 142.5, 142.2, 137.6, 132.0, 131.4, 128.9, 128.7, 128.1, 128.1, 126.1, 124.3, 115.5, 112.3, 96.2, 90.5, 55.1, 34.7, 31.4, 20.9 ppm; MS (MALDI-TOF): calcd for C₇₈H₇₄O₄ [M+H]⁺ 1076.454, found 1076.434.

Synthesis of Compound 204:

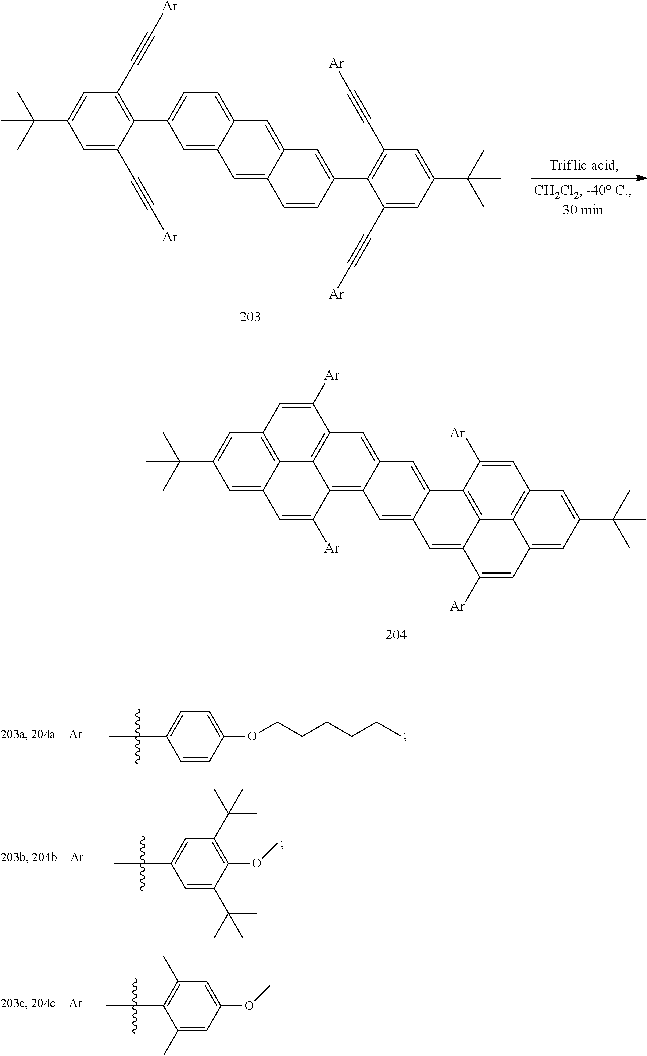

General Procedure for Synthesis of Compounds 204:

To a solution of triflic acid (60 mg, 0.400 mmol, 20 equiv.) in 10 mL of anhydrous CH₂Cl₂ at −40° C. was added dropwise anhydrous CH₂Cl₂ (30 mL) solution of 203 (0.0200 mmol, 1 equiv.) by syringe. After stirring for 30 min-40° C., the solution was quenched with saturated NaHCO₃ solution (5 mL), and then washed with H₂O (2×20 mL). The solvent was dried (Na₂SO₄) and removed under reduced pressure. The residue was purified by column chromatography give compound 204.

Compound 204a:

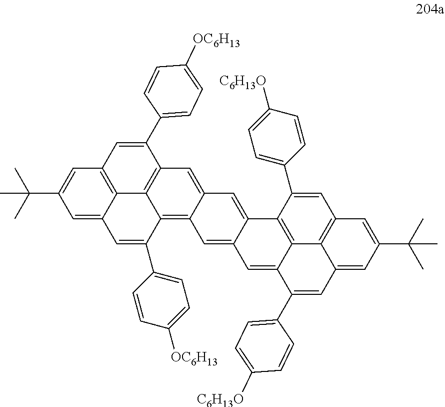

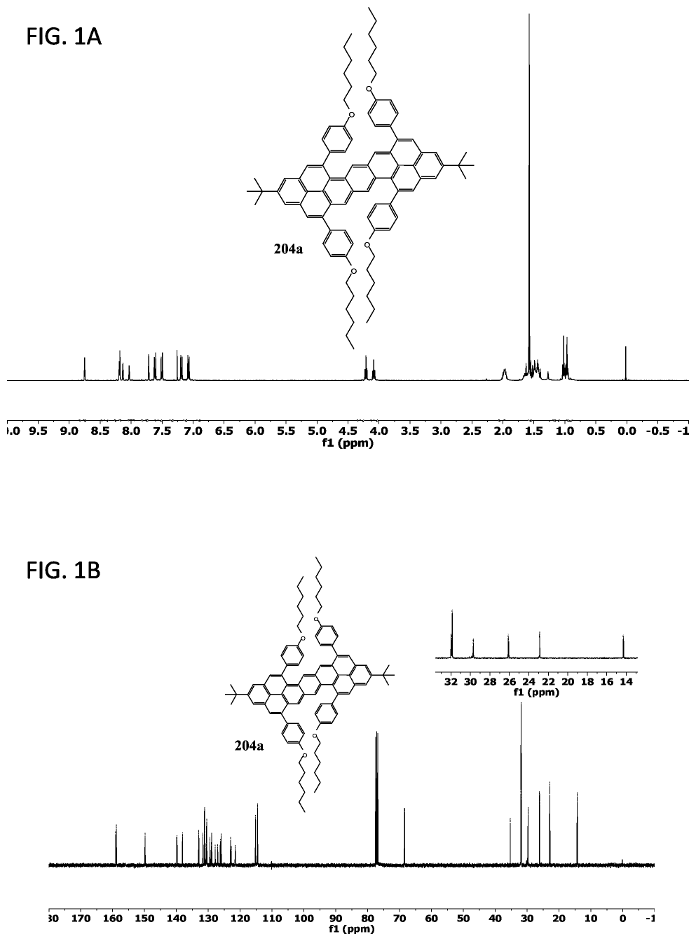

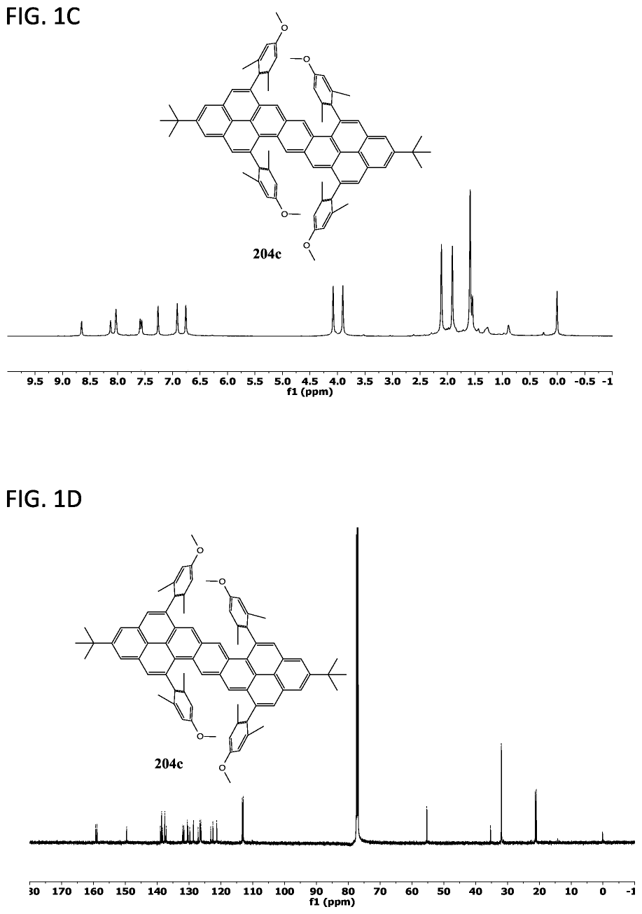

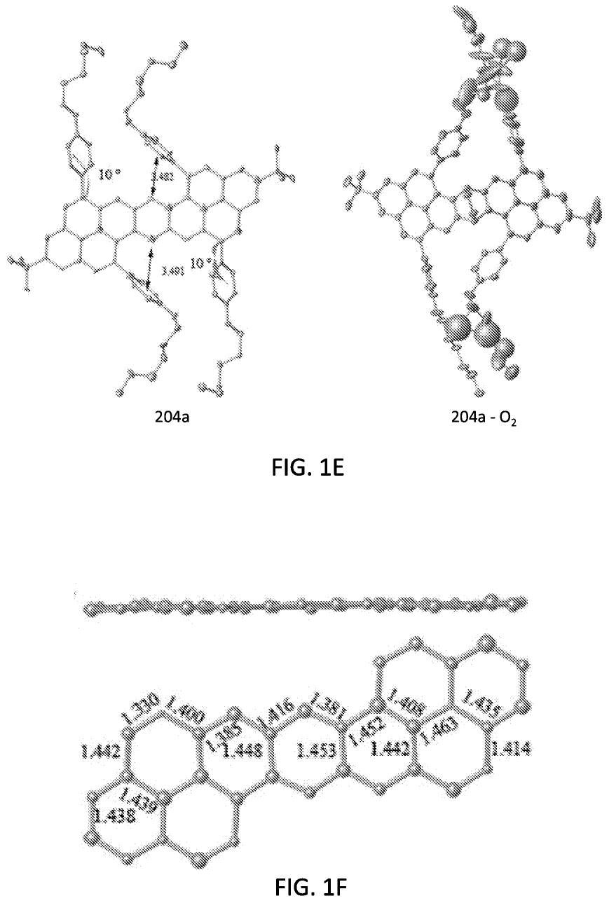

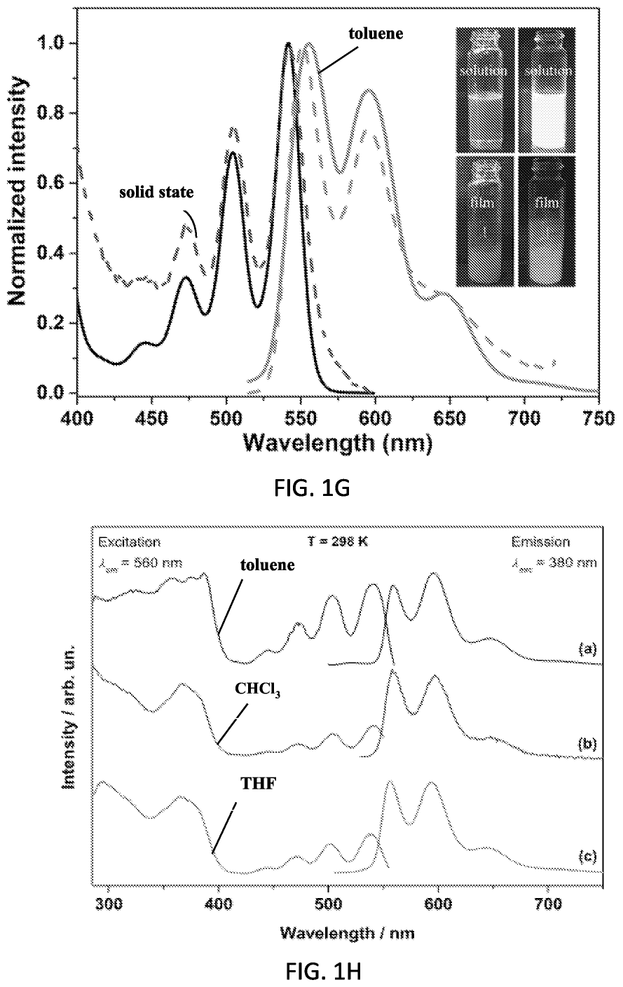

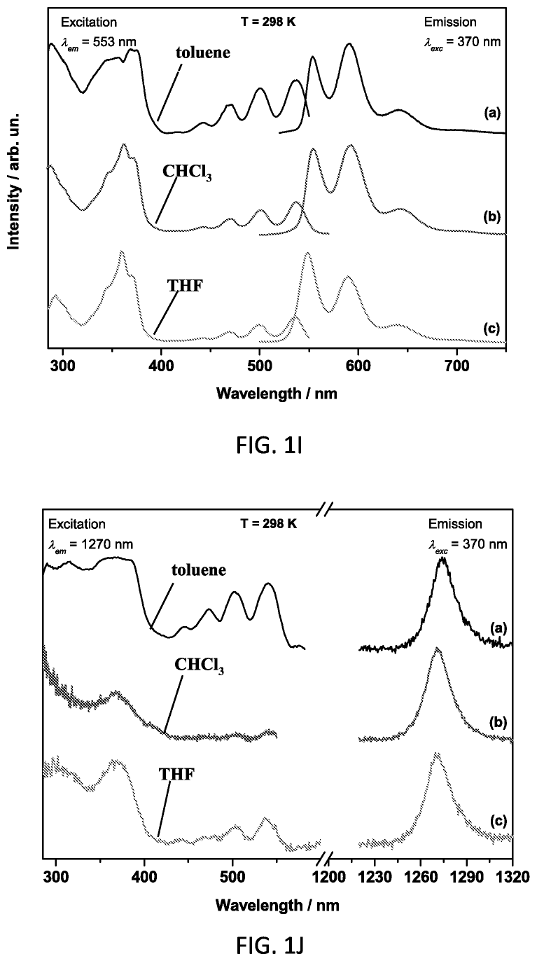

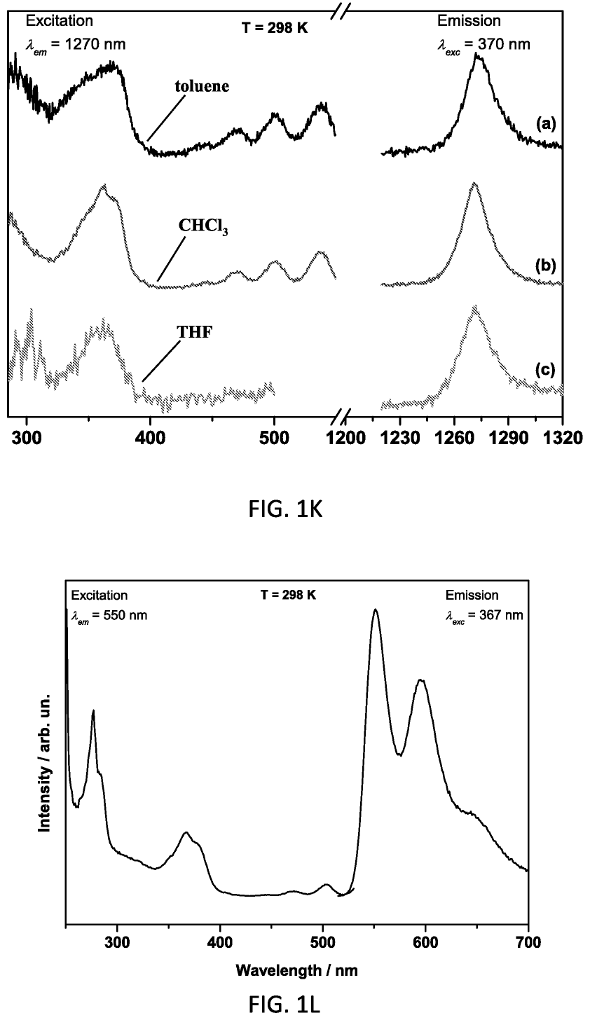

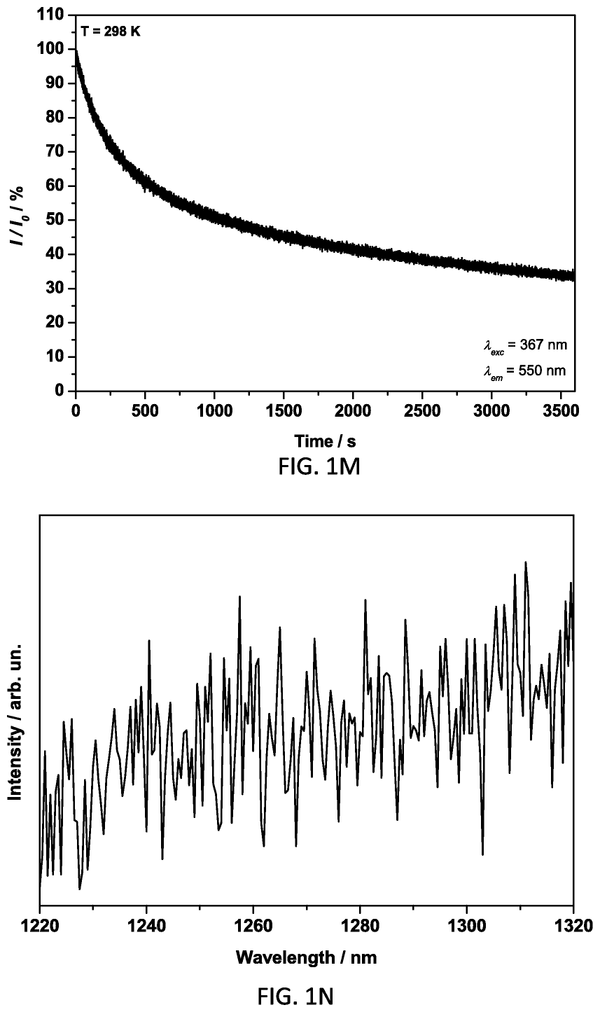

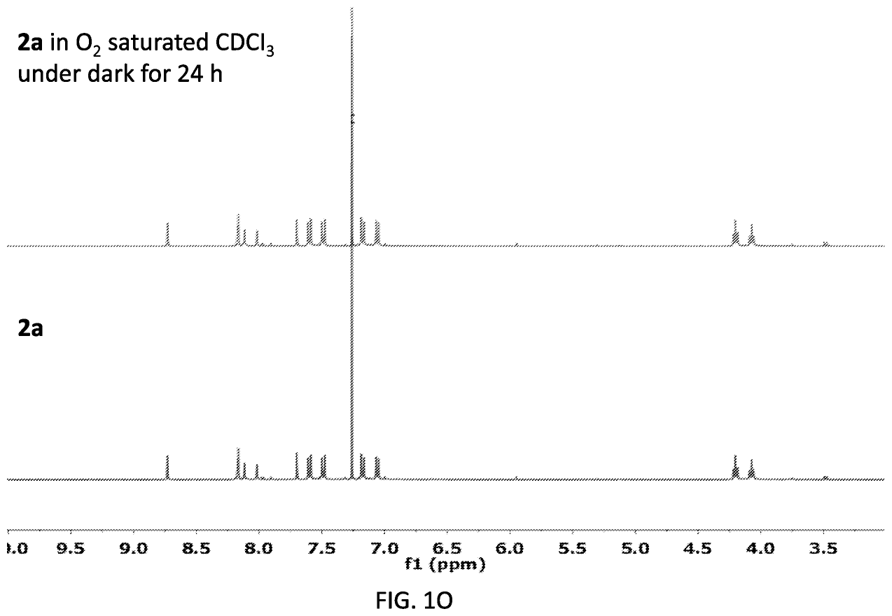

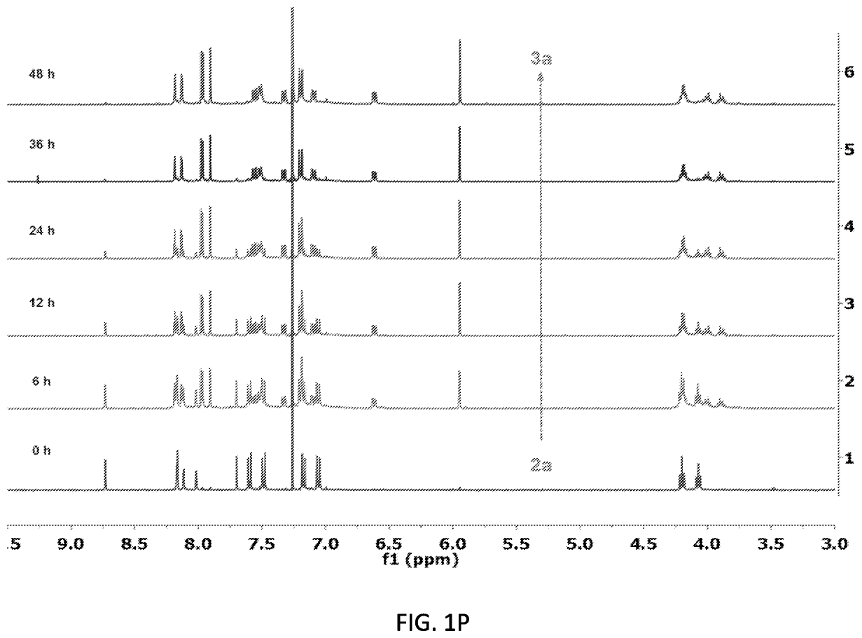

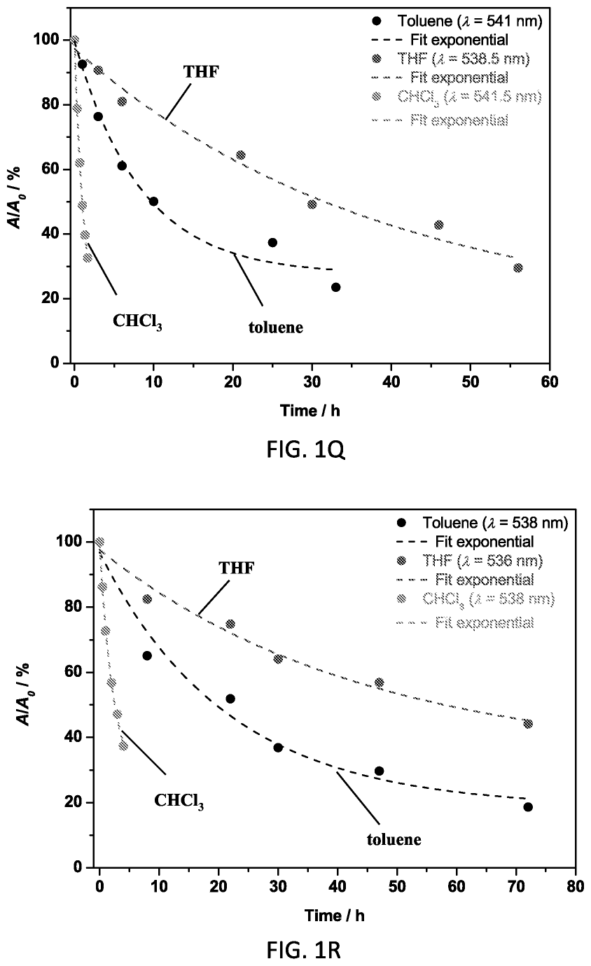

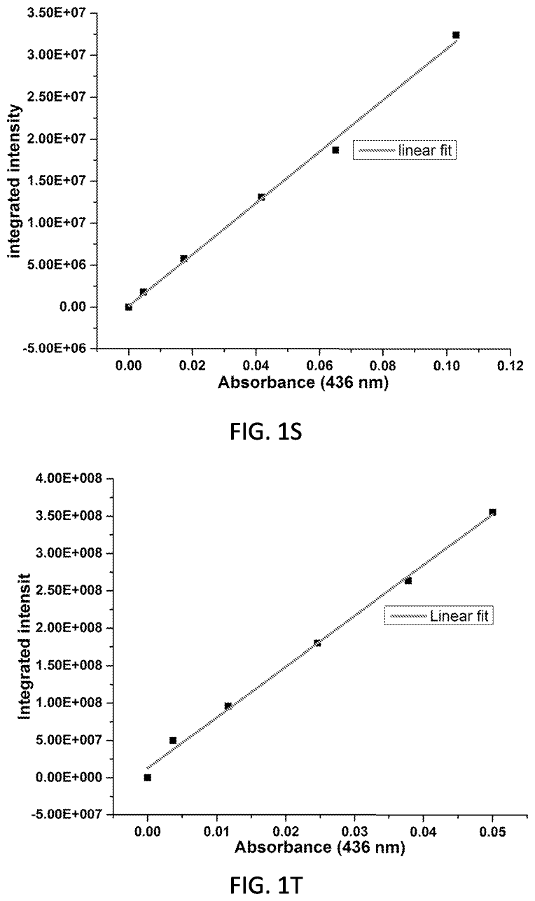

Purification by flash column chromatography (silica gel, hexane:DCM=4:1, v/v) yielded pure compound 204a as a purple solid. R_{f}=0.3 (hexane/DCM 3:1); FTIR (neat) 2954, 2923, 2856, 1725, 1604, 1579, 1463, 1315, 1266, 1154, 1067, 836 cm⁻¹. (FIG. 1A)¹H NMR (400 MHz, CDCl₃) δ 8.75 (s, 2H), 8.23-8.16 (m, 4H), 8.13 (d, J=1.8 Hz, 2H), 8.06-8.01 (m, 2H), 7.72 (d, J=0.7 Hz, 2H), 7.66-7.58 (m, 4H), 7.56-7.46 (m, 4H), 7.24-7.14 (m, 4H), 7.12-7.02 (m, 4H), 4.21 (t, J=6.6 Hz, 4H), 4.09 (t, J=6.6 Hz, 4H), 2.05-1.97 (m, 8H), 1.67-1.38 (m, 42H), 1.08-0.99 (m, 6H), 0.99-0.92 ppm (m, 6H); (FIG. 1B)¹³C NMR (100 MHz, CDCl₃) δ 159.0, 158.9, 149.8, 139.9, 139.8, 138.1, 133.0, 131.7, 131.2, 131.1, 130.7, 130.5, 129.5, 129.0, 128.9, 127.9, 127.0, 126.2, 126.0, 125.9, 123.0, 122.8, 121.5, 115.2, 114.6, 68.4, 35.3, 32.0, 31.93, 31.86, 29.73, 29.69, 26.12, 26.08, 22.91, 22.88, 14.31, 14.27 ppm.

Compound 204c:

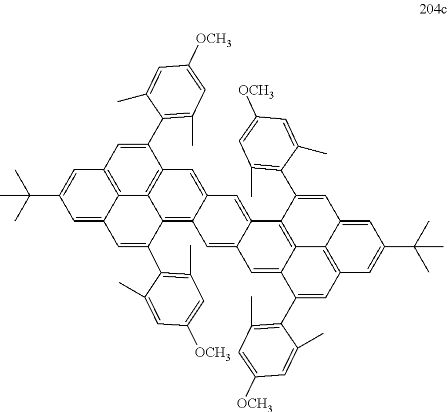 [SMILES: COc1cc(C)c(-c2cc3cc(C(C)(C)C)cc4cc(-c5c(C)cc(OC)cc5C)c5c6cc7cc8c(-c9c(C)cc(OC)cc9C)cc9cc(C(C)(C)C)cc%10cc(-c%11c(C)cc(OC)cc%11C)c(c7cc6cc2c5c34)c8c9%10)c(C)c1]

Purification by flash column chromatography (silica gel, hexane:DCM=1:2, v/v) yielded pure compound 204c as a purple solid. FTIR (neat) 2957, 2907, 2837, 1728, 1605, 1478, 1464, 1316, 1155, 1067, 899 cm⁻¹; (FIG. 1C)¹H NMR (500 MHz, CDCl₃) δ 8.65 (d, J=4.0 Hz, 2H), 8.13 (d, J=3.9 Hz, 2H), 8.02 (d, J=5.1 Hz, 4H), 7.57 (dd, J=15.1, 4.1 Hz, 4H), 6.91 (d, J=4.1 Hz, 4H), 6.76 (d, J=4.1 Hz, 4H), 4.08 (d, J=4.1 Hz, 6H), 3.90 (d, J=4.1 Hz, 6H), 2.11 (d, J=4.1 Hz, 12H), 1.91 (d, J=4.1 Hz, 12H), 1.59 ppm (d, J=4.2 Hz, 18H); (FIG. 1D)¹³C NMR (125 MHz, CDCl₃) δ 159.3, 158.9, 149.6, 139.0, 138.6, 138.3, 137.6, 137.1, 132.0, 131.9, 131.6, 130.5, 130.3, 129.7, 128.6, 127.1, 126.5, 126.3, 126.3, 126.2, 123.2, 122.5, 121.3, 113.2, 113.0, 55.3, 55.3, 35.3, 31.9, 21.2, 20.9 ppm; MS (MALDI-TOF): calcd for C₇₈H₇₄O₄ [M]⁺ 1076.454, found 1076.428.

X-Ray Crystallographic Analysis of Compound 204:

As disclosed herein, although the reaction of precursor 203b bearing more bulky tert-butyl substituents failed to yield the corresponding pentacene derivative 204b, presumably due to the strong repulsion of the tert-butyl groups of two aryl substituents on the same side, the remaining derivatives (e.g., compound 204a) were supported by the X-ray analysis, as illustrated in FIG. 1E.

As depicted in FIG. 1E, single crystals of compound 204a, suitable for X-ray crystallographic analysis, were obtained by solvent diffusion methods, such as, by slow diffusing methanol to the CDCl₃ solutions under ambient light. As depicted, the structure of compound 204a is flat with the aryl substituents twisted out of conjugation. The distance between the carbon atom on the central ring and the plane of the pointing aryl ring has been calculated to be around 3.49 Å. Due to the strong repulsion of two aryl substituents on the same side, one of the aryl substituents recurved with a torsion angle of 10°, which also supported why the synthesis of compound 204b failed. The bond length of the core has also been found to have similar bond alternation trends as that of a conventional silylethyne-functionalized pentacene derivative. Further, the shortest bond length of the outermost ring (1.331.35 Å) approach the values of nonaromatic conjugated double bonds (for instance, at 1.34A), and the longest bonds of the core structure was around 1.46A which is close to the C—C single bond. Notable least bond alternation was observed in the central ring, with the “vertical” bond length (1.453 Å) being longer than that in the silylethyne-functionalized pentacene (1.449 Å), which means that the central ring owns low aromaticity than that in the pentacene. All the analysis indicated that the central ring is potentially more reactive than those in the general pentacene derivatives. FIG. 1F is an X-ray crystallographic image of the core structure of compound 204 with the phenyl and tert-butyl substituents omitted for clarity.

Further, as depicted in FIG. 1G, the optical properties of the desired pentacene derivative were studied using a normalized UV-vis absorption and a normalized fluorescence emission spectra. In one example, the pentacene derivative exhibited a blue shift of 30 nm in its corresponding UV-vis absorption spectrum, thereby indicating an extended n-conjugation along the short axis of the molecule. Such a blue shift in its corresponding UV-vis absorption spectrum is similar to an UV-vis absorption spectra of a pentacene derivative that has been synthesized using a variety of conventional synthetic methods, and thus confirming that the pentacene derivative synthesized using the methods disclosed herein still maintains the desired acene character. Further, compared with the absorption in solution, compound 204a demonstrated a significant red shift of the absorption spectrum in thin films grown from their CHCl₃ solutions which, for instance, indicate strong electronic interaction between molecules in the thin films. As depicted in FIG. 1G, the solid lines represent the samples in the solution (i.e., toluene), while the dotted lines represent the thin film samples.

Further, as described in detail herein, FIGS. 1H-1N independently depict excitation and/or emission spectra of compounds 204a and 204c, respectively. The emission maximum of the compound 204a is in the yellow at ˜560 nm and the emission band shows vibrational fine structure, analogous to the excitation spectrum that is independent of the solvent used. The observed emission spectrum is the mirror image of the excitation spectrum, which indicates that the same states are involved in excitation and emission.

The characteristic phosphorescence emission band at ˜1272 nm, shown in FIGS. 1J & 1K, confirms the generation of singlet oxygen (¹O₂). The same profile of the excitation spectra, monitoring the emission at 560 (luminescence, FIGS. 1H & 1I) or at 1272 nm (¹O₂ emission, FIGS. 1J & 1K), indicates that the same states are involved in these two processes. The absence of the ¹O₂ emission band in the absence of light confirms that this process is photoactivated, as depicted in FIG. 1N. As summarized below in Table 1, the compounds' emission lifetimes are ˜6 ns, which correspond to an emissive rate of ˜0.154 ns⁻¹ and are independent of the lateral substituents in the core ring or the solvent used. All decay curves were observed to fit to a mono exponential indicating that emission occurs from one excited state. The quantum yields of ¹O₂ generation are 27±1, 70±2 and 4.9±1.6% in toluene, CHCl₃ and THF, respectively, and are independent of the lateral substituents.

TABLE 1
Emission lifetimes (τ), singlet oxygen emission quantum yield (Φ₁₀₂)
and half-life time (t_{1/2}) obtained for the compounds.

| Compound | Solvent | τ / ns | Φ₁₀₂ | t_{1/2} / min |
|---|---|---|---|---|
| 204a | Toluene | 6.5 ± 0.1 | 0.27 ± 0.01 | 583 |
| | CHCl₃ | 6.2 ± 0.1 | 0.70 ± 0.02 | 58 |
| | THF | 6.5 ± 0.1 | 0.046 ± 0.016 | 1894 |
| 204c | Toluene | 6.8 ± 0.1 | 0.29 ± 0.01 | 1176 |
| | CHCl₃ | 5.9 ± 0.3 | 0.74 ± 0.08 | 154 |
| | THF | 5.6 ± 0.1 | 0.017 ± 0.001 | 4612 |

Further, as one skilled in the art will understand, the utility of conventional pentacene derivatives or higher acenes was typically limited due to their photochemical decomposition, particularly in the presence of O₂ with light irradiation. In contrast, as illustrated below, photodegradation studies of the pentacene-based derivative with non-functionalized central ring disclosed herein were found to be fairly stable in THF. For example, the photostability of compound 204a was tested by tracking the scheme below. As depicted, this resulted in a corresponding endoperoxide product 204a-O₂.

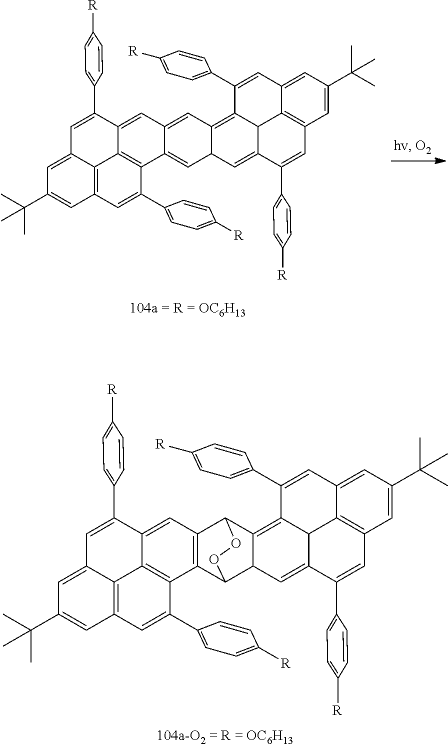

As depicted in FIGS. 1O & 1P, no obvious decomposition was observed after putting the sample CDCl₃ solution under dark for 24 hours in its corresponding ¹H NMR spectrum. However, the sample significantly decomposed even under ambient light and only trace amount of compound 204a residue after 36 hours was observed (See also FIGS. 10 & 1P).

Additionally, the photostability is a key prerequisite for potentially using the pentacene derivatives synthesized using the methods disclosed herein as organic semiconductors. While the lateral substituents do not affect the photophysical properties of the compounds, they do impact their photostability. Initially, photochemical stability tests were carried out to determine the potential utilities of the newly synthesized pentacene derivative under prolonged irradiation in the presence of O₂. For example, photostability was monitored by the decrease in the absorbance, under fluorescent white light exposure, in different solvents (FIGS. 1Q & 1R). Compound 204a, in THF, showed a decomposition half-life time (t_{1/2}) of 1894 min. This is in the same order of magnitude compared as the highest value reported for this type of polycyclic aromatic compounds. Modification of the lateral group improves the photostability of the compound by ˜2.5 fold which results in a record high t_{1/2} of 4612 min in THF which corresponds to the most photostable pentacene-based compound ever reported. The product of photobleaching has an emission maximum at 460 nm caused by a decrease in the electronic conjugation, which corroborates the formation of the endoperoxide in the central aromatic ring. As observed, compound 204a has been found to be photostable over a period of at least 1 h under continuous illumination at 380 nm in THF, while a dramatic photobleaching has been observed in toluene and CHCl₃, with the photobleaching being higher in CHCl₃, presumably due to a combination of higher concentration of dissolved oxygen and generation of chlorine radicals. As such, the newly synthesized pentacene derivative has been proved to be a good singlet oxygen sensitizer with a value of 702% quantum yields of ¹O₂ generation in CHCl₃.

Further, as depicted above in FIG. 1E, an X-ray crystallographic analysis of single crystals of the corresponding endoperoxide product 204a-O₂ was also observed in a corresponding CDCl₃ solution under ambient light. The structure of the peroxided product 204a-02 unambiguously confirmed the [4+2] cycloaddition reactions of compound 204a with singlet oxygen (¹O₂) occurring on the central ring. Quantum yield for compound 204 (see FIGS. 1S and 1T): As illustrated in FIGS. 1S & 1T, the quantum yield (QY) was calculated by preparing solutions of the sample at varying concentrations in toluene, and measuring the absorbance at 436 nm and the area under the emission peak at each different concentration. The absorbance was plotted against the area and compared to a standard of known quantum yield (Ru(bpy)₃²⁺). This data was used to obtain a value for the quantum yield.

The quantum yield of compounds in toluene was calculated using the following equation:

$${Φ}_{x} = \frac{{Grand}_{x}}{{Grand}_{std}} \times \frac{{n}_{x}^{2}}{{n}_{std}^{2}} \times {Φ}_{std}$$

Grad is the slope of the plot ‘Emission area vs Absorbance’, n is the refractive index of the solvent and Φ is the quantum yield for sample x and standard std.

Here Ru(bpy)₃Cl₂.6H₂O was used as reference, which has a quantum yield of 2.8% when dissolved in H₂O. H₂O had a refractive index of 1.3333, while the refractive index of toluene is 1.4968.

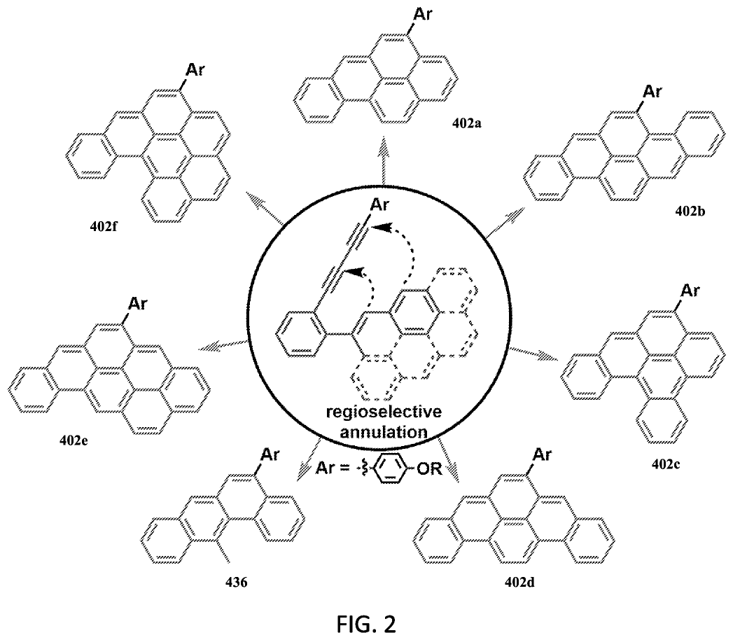

In some embodiment, as depicted in FIG. 2, and as described above in connection with Formulas 2-5 and Schemes 3-8, electron-rich 1,3-diynes 400 are subjected to a regioselective intramolecular cascade benzannulation reaction using a variety of Brønsted acids, in one or more synthetic methods disclosed herein to afford a series of polycyclic aromatic compounds having irregular shapes.

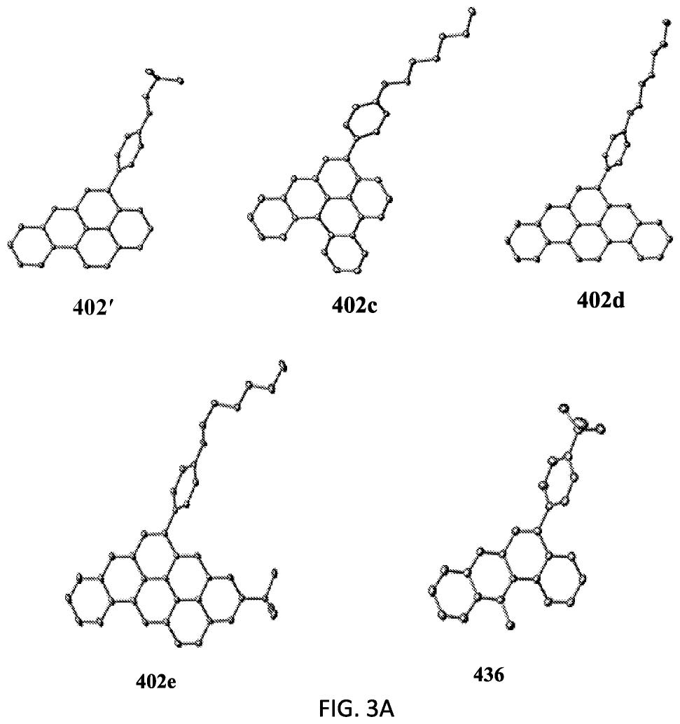

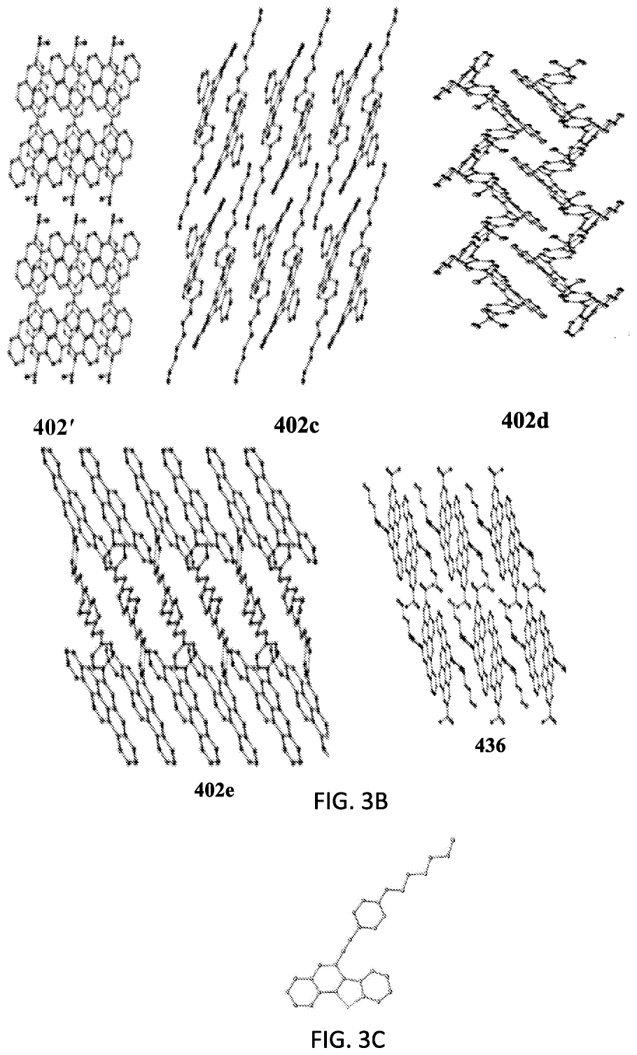

In particular disclosed embodiments, the electron-rich 1,3-diyne 400′ was used as a standard to optimize the cascade benzannulation reaction conditions, and the results were summarized in Table 2. As depicted in Table 2, conventional Lewis-acids catalysts InCl₃, PtCl₂, AuCl₃, and AuC(PPh₃) were initially utilized for the benzannulation reactions disclosed herein. Disadvantageously, the formation of desired benzopyrene derivative 402′ was not observed, as evident from the entries 1-4 in Table 2. However, 75% conversion of compound 400 to mono-cyclized intermediate 405 was achieved in the presence of InCl₃. In another example, silver salt additives combined with InCl₃ were tested, particularly, in an attempt to increase the electrophilicity at the metal centers by providing weakly coordinating counter ions. For instance, five different silver salts were tested, among which AgSbF₆, AgNTf₂, and AgOTf, combined with InCl₃ showed outstanding catalytic efficiency. The desired annulated product 402 was formed exclusively and isolated in 50%, 83%, and 71% yields, respectively, as evident from the entries 5, 8, and 9 of Table 2. In another example, a yield similar to that of InCl₃/AgOTf was observed, when In(OTf)₃ was used as a Lewis acid in the place of InCl₃/AgOTf, as evident from entry 10 of Table 2. Gratifyingly, excellent yield was obtained after decreasing the amount of AuCl₃/AgNTf₂ to 10 mol % (See entry 12, Table 2). The reaction was also tested in 1,2-dichloroethane (DCE), in which the precursor 400′ preferred to polymerize rather than cyclize, and the product 402′ was isolated in 25% yield (See entry 13, Table 2). No reaction occurred in the control experiment which was carried out with only AgNTf₂ (See entry 14, Table 2). In yet another example, Brønsted acids, such as, triflic acid (TfOH) were also investigated, in the place of Lewis Acids. For instance, upon stirring the precursor 400′ and 2 equivalents of TfOH in dichloromethane (DCM) at −40° C. for 30 mins, the target compound 402′ was isolated in 51% yield (See entry 14, Table 2). It should be noted that the first alkyne benzannulation is regioselective, and the regioselectivity was unambiguously confirmed by the crystal structure of compound 402′, as depicted in FIGS. 3A and 3B.

Thus, the optimized conditions for the regioselective cascade benzannulation reactions of 1,3-diynes were established as follows: heating compound 400 (0.01 M) in toluene at 100° C. in the presence of InCl₃ (10 mol %) and AgNTf₂ (10 mol %) under N₂ atmosphere.

TABLE 2
Optimization of the cascade benzannulation reaction conditions:

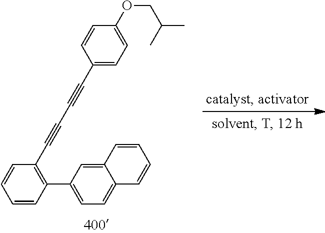 [SMILES: CC(C)COc1ccc(C#CC#Cc2ccccc2-c2ccc3ccccc3c2)cc1]

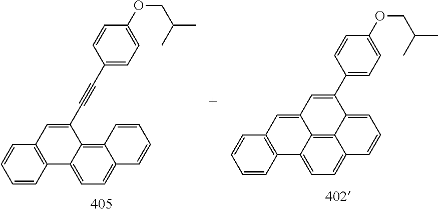 [SMILES: CC(C)COc1ccc(C#Cc2cc3ccccc3c3ccc4ccccc4c23)cc1 ; CC(C)COc1ccc(-c2cc3cc4ccccc4c4ccc5cccc2c5c34)cc1]

| | catalyst | activator | | T | ratio | yield |
|---|---|---|---|---|---|---|
| entry | (%) | (%) | solvent | (° C.) | (400′:405:402′) | 3aa (%) |
| 1 | InCl₃ (20) | — | toluene | 100 | 1:3:0 | 0 |
| 2 | PtCl₂ (20) | — | toluene | 100 | 8:1:0 | 0 |
| 3 | AuCl₃ (20) | — | toluene | 100 | 22:1:0 | 0 |
| 4 | AuCl(PPh₃) (20) | — | toluene | 100 | 1:0:0 | 0 |
| 5 | InCl₃ (20) | AgSbF₆ (60) | toluene | 100 | 0:0:1 | 50 |
| 6 | InCl₃ (20) | AgBF₄ (60) | toluene | 100 | 290:1:0 | 0 |
| 7 | InCl₃ (20) | AgCO₂CF₃ (60) | toluene | 100 | 3:6:1 | — |
| 8 | InCl₃ (20) | AgNTf₂ (60) | toluene | 100 | 0:0:1 | 83 |
| 9 | InCl₃ (20) | Ag(OTOf)₃ (60) | toluene | 100 | 0:0:1 | 71 |
| 10 | In(OTf)₃ (20) | — | toluene | 100 | 0:0:1 | 72 |
| 11 | InCl₃ (10) | AgNTf₂ (30) | toluene | 100 | 0:0:1 | 85 |
| 12 | InCl₃ (10) | AgNTf₂ (10) | toluene | 100 | 0:0:1 | 94 |
| 13 | InCl₃ (10) | AgNTf₂ (10) | DCE | 80 | 0:0:1 | 25 |
| 14 | — | AgNTf₂ (10) | toluene | 100 | 1:0:0 | 0 |
| 15 | TfOH (200) | — | DCM | −40 | 0:0:1 | 51 |

[a] Determined by ¹H NMR.
[b] Isolated yield.
[c] Not isolated.
[d] Reaction was quenched after 30 minutes.

In a further embodiment, the optimized benzannulation reaction conditions were employed with a variety of precursor materials to enhance the solubility and properties of the resulting polycyclic aromatic compounds, as depicted in Table 3. For instance, hexyl chains were incorporated to enhance the solubility of 1,3-diynes precursor 400 and their corresponding annulated products 402. All precursors 400a-400h were prepared via Suzuki cross-coupling reaction in good yields. The benzannulation reactions of the precursors 400 were carried out under the optimized reaction conditions, and the results were summarized in Table 3. Advantageously, naphthalene-based derivatives, anthracene-based derivatives, phenanthrene-based derivatives, and pyrene-based derivatives can be used as suitable precursor compounds in this reaction, affording the corresponding larger polycyclic aromatic compounds in moderate to high yields, as evident from the compounds 402a-402h of Table 3

TABLE 3
Substrate scope with respect to parent polycyclic aromatic compounds.

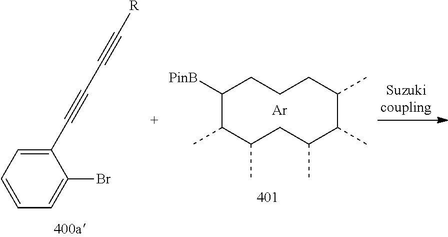

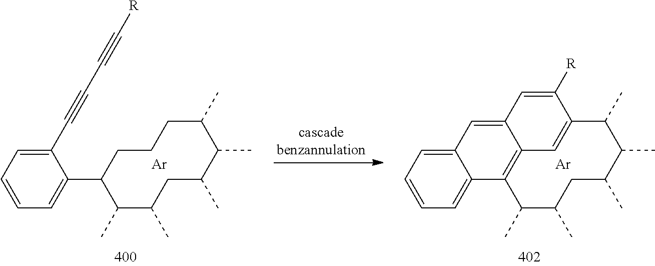

| 401 | 400 | 402 |
|---|---|---|
| 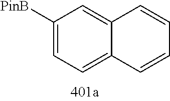 | 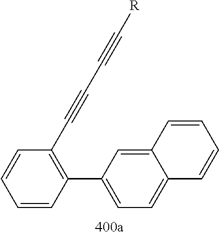 | 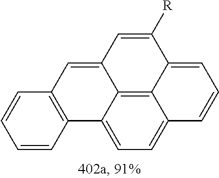 |
| 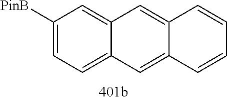 | 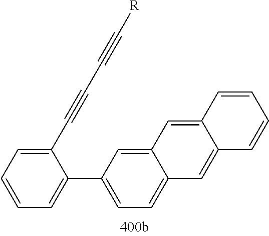 | 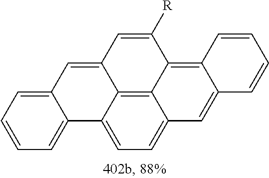 |
| 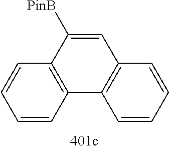 | 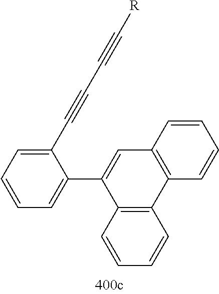 | 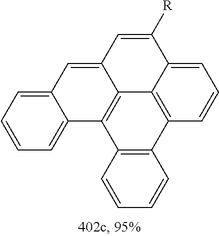 |
| 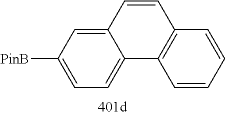 | 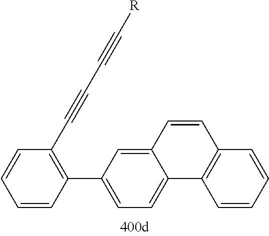 | 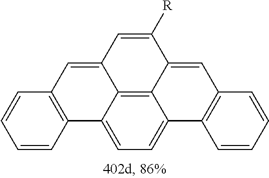 |
| 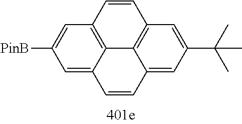 | 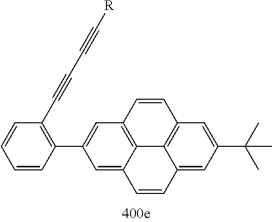 | 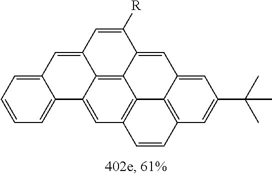 |
| 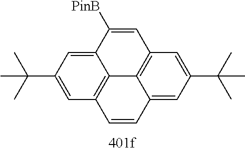 | 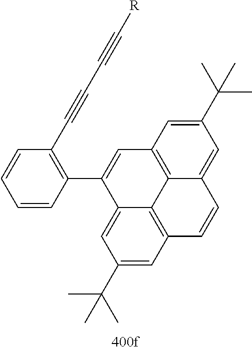 | 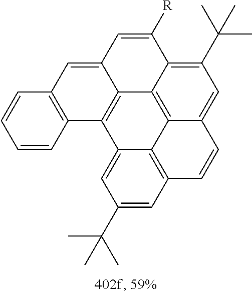 |
| 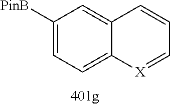 | 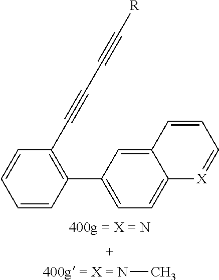 | 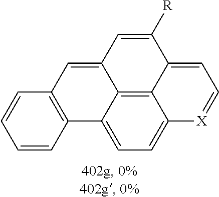 |
| 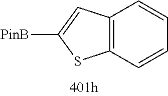 | 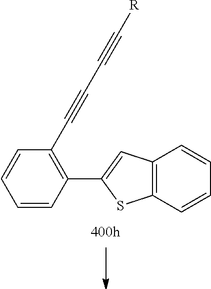 | 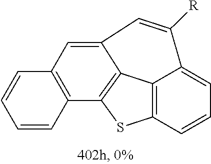 |
| | 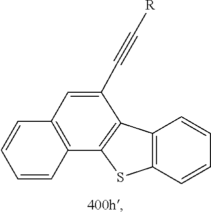 | |

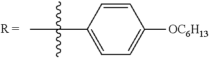

^{[a]:} [a] Reaction [symbol] toluene, InCl₃ (10 mol %), AgNTf₂ (10 mol %), 100° C., 12 h.
[b] Isolated yield.
[c] Yield of compound 400h [symbol]
indicates data missing or illegible when filed

For example, the benzo[a]pyrene derivative 402a was isolated in 91% yield by treating precursor 400a under optimized reaction conditions. Using anthracene instead of naphthalene, the dibenzo[b,def]chrysene core which has never been previously reported, was formed in one-step, and the dibenzo[b,def]chrysene derivative 402b was obtained in 88% yield. Interestingly, subsequent to benzannulation method disclosed herein, changing the position of the substituent group in their corresponding precursors afforded different polycyclic aromatic compound cores, such as, 402c and 402d, 402e and 402f, respectively. With proof that a cascade alkyne benzannulation can afford larger compounds with irregular shape, the benzannulation strategy disclosed herein was applied to a hetero-aromatic system, such as quinoline and benzothiophene. However, none of these hetero-aromatic systems yielded the desired fully annulated products. In one example, for the precursor 400g with quinoline motif, no reaction occurred and most part of the starting material was recovered. Upon converting quinoline to 1-methylquinolinium 400g′, anion exchange was observed instead of the desired benzannulation. Similarly, for the precursor 400h with benzothiophene motif, the desired product 402h could not be obtained either by one-pot or step-wise process. That was, presumably, because the distances for the second cyclization are much longer in the mono-annulated intermediate product 400h′(˜3.5 Å, in Table 3). FIG. 3C depicts a X-ray crystallographic image of compound 400h′.

To further demonstrate the synthetic utility of this newly developed method, three transformations of the precursors 400i-400k with 1,1′-Binaphthyl-2,2′diol (BINOL) motif were tested, as illustrated in Table 4. BINOL and various modified BINOL derivatives have been typically used as ligands for both stoichiometric and catalytic asymmetric reactions. As disclosed herein, this cascade alkyne benzannulation strategy was successfully applied to prepare new modified BINOL-like ligands (See compounds 402i-402j, Table 4). After treating the precursor 400i with the established reaction condition, the one side modified BINOL-like ligand 402i was achieved in 92% yield. Furthermore, the both sides modified BINOL derivatives 402j and 402k were both investigated. However, only the precursor 400j gave the corresponding BINOL-like ligand 402j in 63% yield, while no corresponding product 402k was observed, and a dramatic polymerization occurred in the reaction of precursor 400k. This may be due to the fact that the two 1,3-diyne substituents in the precursor 400k could react with each other due to the free rotation.

TABLE 4
Synthetic application of the cascade annulations of 1,3-diyne precursor:

| 401 | 400 | 402 |
|---|---|---|
| 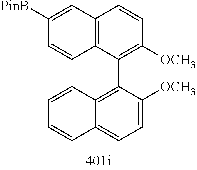 | 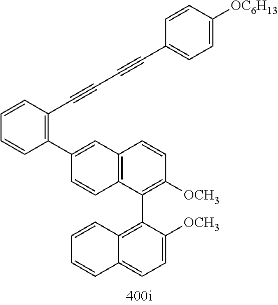 | 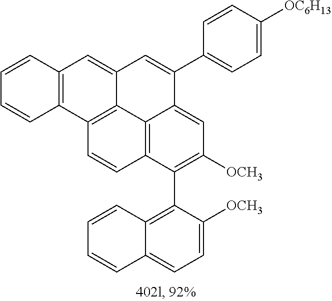 |
| 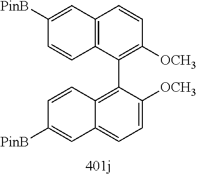 | 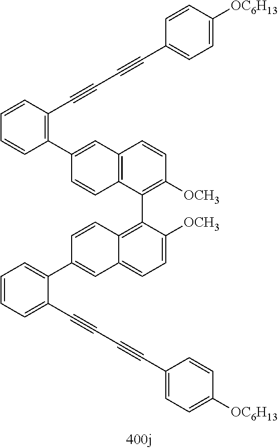 | 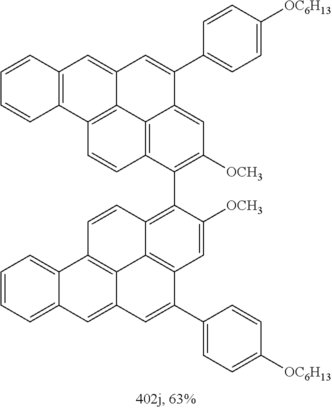 |
| 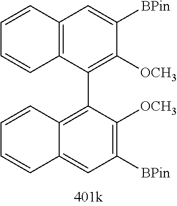 | 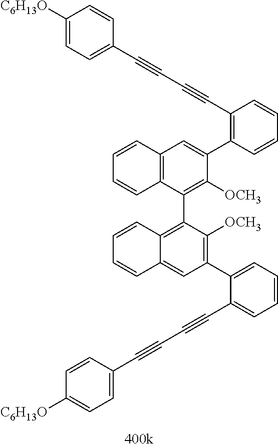 | 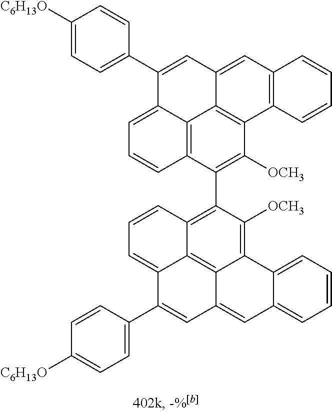 |

[a] Isolated yield.
^{[b]}The product 402k cannot be isolated from the reaction.

In yet another example, the method disclosed herein was used for the cascade benzannulation of diynylthiophene precursor, as illustrated in Scheme 12. Upon treating precursor 412 under optimized reaction conditions, the thiophene functionalized compound 414 was isolated in 82% yield. Combining all the above results, it is evident that electron-rich 1,3-diynes (for example, electron-neutral and electron-deficient 1,3-diynes, such as compounds 416 and 420, respectively) worked well for the cascade annulation reaction. However, only trace amount of mono-cyclized product was observed after treating the electron-deficient 1,3-diynes 416 under optimized reaction conditions, and most part of starting material was recovered even after 12 hours heating in mesitylene at 150° C. In the case of electron-neutral precursor 420, except trace amount of the desired product 422 which was hard to isolate from the mixture of starting material and mono-cyclized intermediate, a large amount of side-product was obtained. The side-product was mainly the addition product of molecular toluene to the precursor mixed with some polymer. In attempt to avoid this side reaction, mesitylene was used instead. No reaction occurred when the precursor 420 was treated in mesitylene at 100° C. for 12 hours, while one major product was isolated after keeping the reaction at 150° C. for 24 hours. The NMR results indicated that it was the addition product of solvent to the precursor, and the structure was unambiguously proved by the X-ray crystallography (See compound 424, Scheme 12). From the crystal structure, it is evident that the molecular mesitylene attacked the carbon which was closer to the larger polycyclic aromatic compounds part. The formation of compound 424 was not observed in nonaromatic solvents either, such as DCE. As understood, for electron-neutral diyne, the second cyclization step seems to be too slow to compete effectively with solvent trap or polymerization.

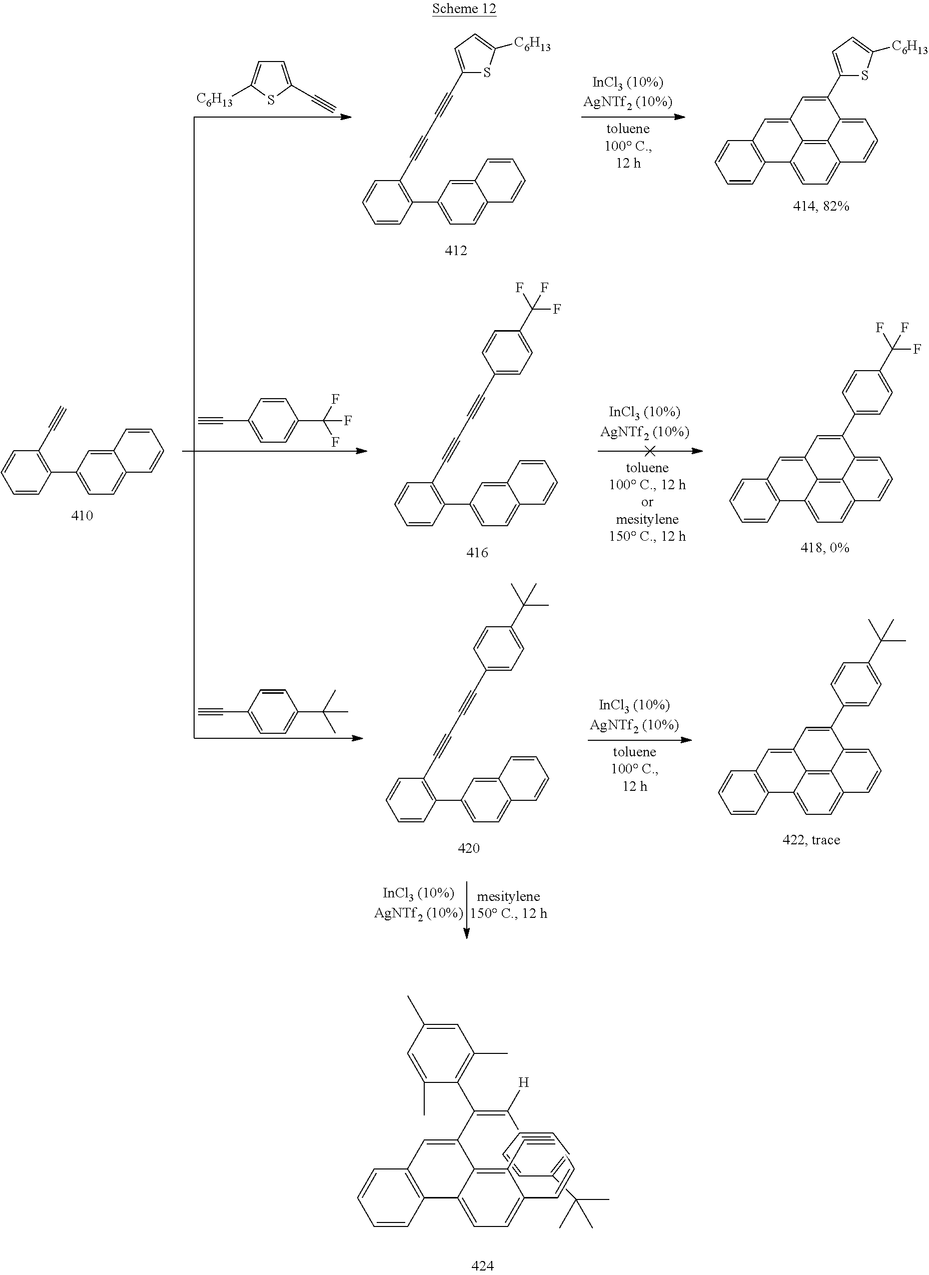

Further, during the preparation of compound 422, the second alkyne benzannulation reaction was hard to achieve. This may presumably due to the low efficiency of the second nucleophilic addition. As such, in order to prove the hypothesis and to further expand the substrate scope, stilbene derivatives as building blocks were investigated, and significant difference was observed, as illustrated in equations 1 and 2 of Scheme 13. For instance, upon treating compound 136 under the optimized reaction conditions, the formation of compound 138 was not observed. Additionally, no product (e.g., compound 142) was observed even when the precursor material 140 was substituted with a methyl group on the ortho position so as to enhance regioselectivity. However, the reaction was successful even for electron-neutral 1,3-diyne when the order of the cascade benzannulation reaction was reversed as in the case of compounds 144 and 148, respectively. The desired compounds 146 and 150 were isolated in 84% and 82% yield, respectively. Combining all the results, it may be deduced that the first alkyne benzannulation was high efficient, while the second benzannulation reaction was the rate determination step.

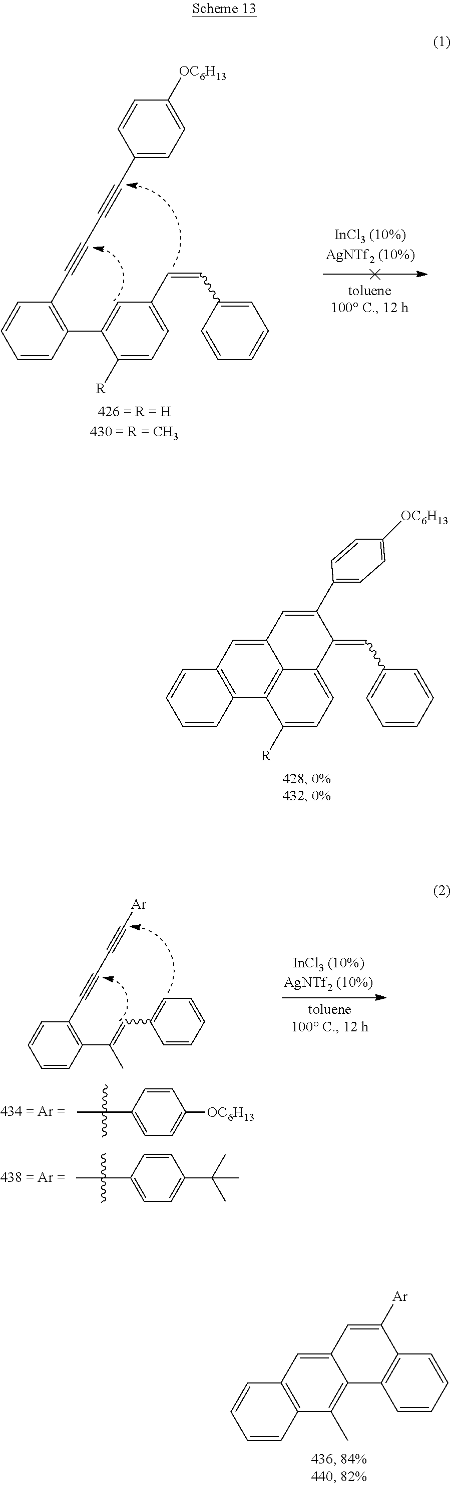

Further, as depicted in FIGS. 3A and 3B in one embodiment, single crystals of 402′, 402c, 402d, 402e, and 438, suitable for X-ray crystallographic analysis were obtained by solvent diffusion methods, which ambiguously proved the regioselectivity of the cascade benzannulation reaction. Their structures and corresponding crystal packing are shown in FIGS. 3A and 3B, respectively. As evident from FIG. 3B, the molecules formed dimers first and the dimers stacked side by side to form the layered structure in the crystal packing, and the formation of the dimer may be attributed to the steric hindrance of the substituent on one side.

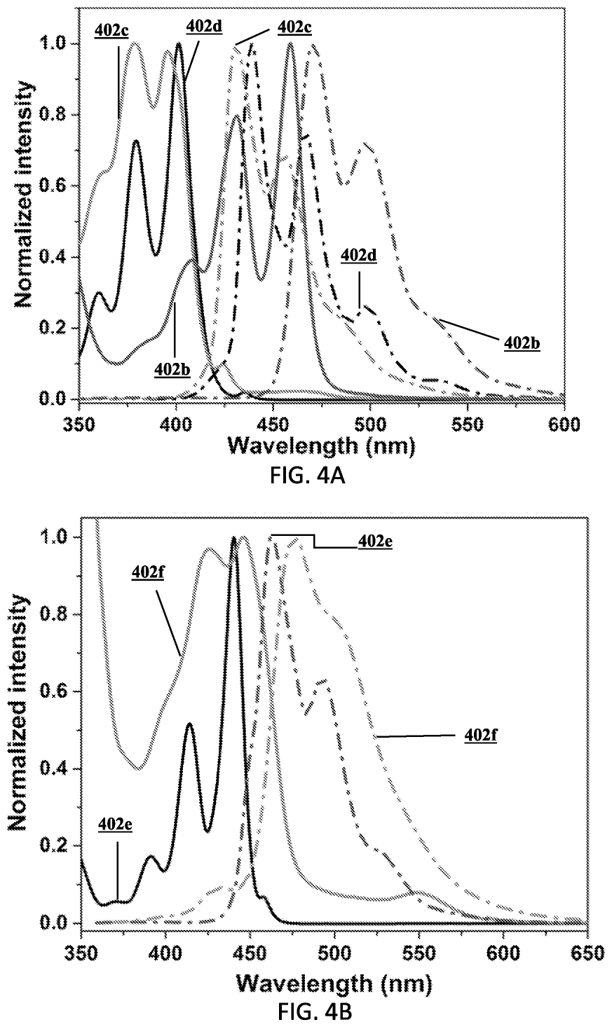

Still further, a normalized UV-vis absorption and a normalized fluorescence spectra of selected compounds 402b, 402c, 402d, 402e, and 402f, respectively, are shown in FIGS. 4A & 4B. Vibronically coupled bands with significant fine structures were observed in the UV-vis spectra of these compounds. Compounds 402b, 402c, and 402d, were three dibenzochrysene structural isomers that showed totally different absorption. Compared to compounds 402c and 402d (λₘₐₓ=396 and 401 nm for 402c and 402d, respectively), a dramatic bathochromic shift of −60 nm for compound 402b which exhibited a fine-structure absorption band at λₘₐₓ=459 nm. Compounds 402e and 402f could also be considered as isomers, which both owned seven fused benzene rings. Compound 402f showed relatively bathochromic shift absorption than 402e. As evident from the fluorescence spectra of FIG. 4B, compared to the planar isomer 402d and 402e, a weak absorption peak in the long wavelength region was observed in the absorption spectra of the non-planar isomer, such as 402f and 402c, respectively.

As such, the first cascade benzannulation reactions of 1,3-diynes catalyzed by a variety of Lewis acids, such as, InCl₃—AgNTf₂ was established, and a series of irregular polycyclic aromatic compounds were successfully synthesized through this new strategy. The electron donating group attached to the 1,3-diynes precursors were crucial for the second benzannulation reaction which provided resonance stabilization of the positive charge and favored 6-endo dig cyclization. The regioselectivity of this new strategy was unambiguously confirmed by the X-ray crystallography. Moreover, the double benzannulation of 1,3-diynes was also used as a facile and efficient route to construct BINOL-like ligands. As such, this cascade benzannulation of 1,3-diynes disclosed herein has immense potential for application in the precise synthesis of large polycyclic aromatic hydrocarbons with irregular shapes.

Synthesis of Compound 400′:

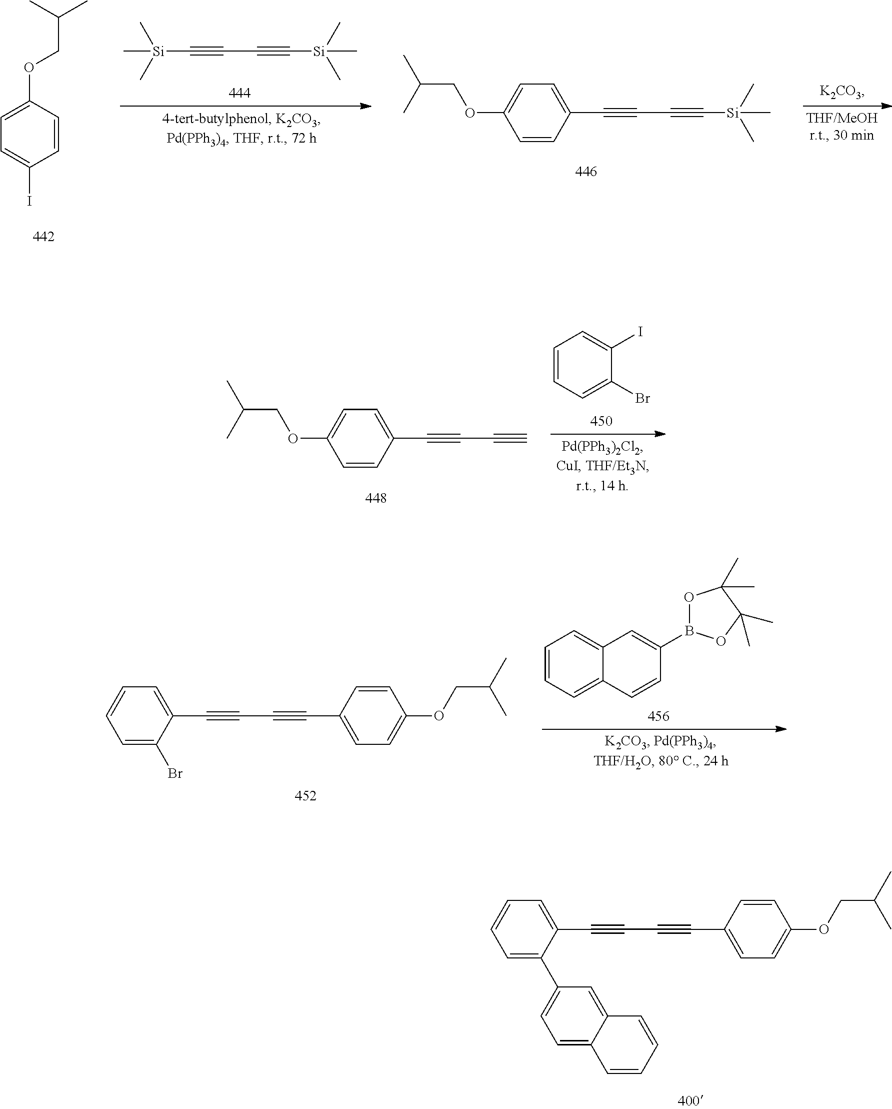

Compound 446:

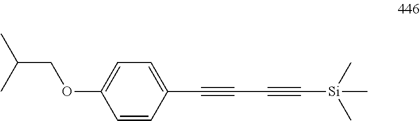 [SMILES: CC(C)COc1ccc(C#CC#C[Si](C)(C)C)cc1]

To the solution of compound 442 (3.81 g, 13.8 mmol), compound 444 (3.01 g, 15.5 mmol) and 4-tert-butylphenol (2.25 g, 15.0 mmol) in THF (50 mL) was added Pd(PPh₃)₄ (159 mg, 0.138 mmol), CuI (52.4 mg, 0.276 mmol) and K₂CO₃ (5.71 g, 41.4 mmol). The resulting mixture was stirred under a N₂ atmosphere at room temperature for 72 h. Then, the reaction mixture was extracted with CH₂Cl₂, washed with 2 M HCl_{aq} and brine, and dried over Na₂SO₄. After removal of the solvent, the residue was purified by silica gel column chromatography (eluent: hexane) to afford compound 446 as colorless oil (1.87 g, 50%). R_{f}=0.1 (hexane); FTIR (neat) 2958, 2873, 2201, 2101, 1602, 1508, 1469, 1416, 1295, 1246, 1169, 1107, 1026, 1002, 828, 759 cm⁻¹; ¹H NMR (400 MHz, CDCl₃) δ 7.44-7.37 (m, 2H), 6.85-6.78 (m, 2H), 3.71 (d, J=6.6 Hz, 2H), 2.14-2.03 (m, 1H), 1.02 (d, J=6.7 Hz, 6H), 0.23 ppm (s, 9H). ¹³C NMR (100 MHz, CDCl₃) δ 160.3, 134.4, 114.8, 113.1, 90.0, 88.3, 77.4, 74.6, 28.3, 19.3, −0.2 ppm. HRMS (ESI, positive) m/z calcd for C₁₇H₂₂OSi [M+H]+ 271.1513, found 271.1518.

Compound 448:

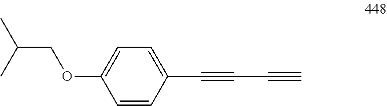

A mixture of compound 446 (1.62 g, 6.00 mmol), K₂CO₃ (0.966 g, 7.00 mmol), MeOH (15 mL) and THF (15 mL) was stirred at room temperature for 30 min. Then, the reaction mixture was extracted with CH₂Cl₂ and washed with brine. By evaporating the solvent to 10 mL and used directly for the next step without further purification.

Compound 452:

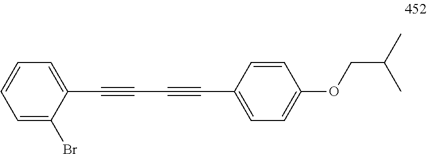 [SMILES: CC(C)COc1ccc(C#CC#Cc2ccccc2Br)cc1]

1-Bromo-2-iodobenzene 450 (1.70 g, 6.00 mmol), Et₃N (40 mL) and THF (80 mL) were added into the above solution of compound 448. After bubbling N₂ for 30 mins, Pd(PPh₃)₂Cl₂ (65.0 mg, 0.0926 mmol) and CuI (35.0 mg, 0.184 mmol) were added. The resulting mixture was stirred at room temperature for 14 h. The ammonium salt was then removed by filtration. The solvent was removed under reduced pressure and the residue was purified by column chromatography (eluent: hexane:CH₂Cl₂=10:1, v/v) to afford compound 452 as colorless oil (1.29, 61%). R_{f}=0.4 (hexane:CH₂Cl₂=6:1); FTIR (neat) 3069, 2958, 2923, 2871, 2212, 2145, 1601, 1508, 1467, 1288, 1246, 1169, 1026, 829, 752 cm⁻¹; ¹H NMR (400 MHz, CDCl₃) δ 7.59 (m, 1H), 7.54 (m, 1H), 7.50-7.44 (m, 2H), 7.27 (m, 1H), 7.23-7.16 (m, 1H), 6.88-6.81 (m, 2H), 3.73 (d, J=6.6 Hz, 2H), 2.08 (m, 1H), 1.03 (s, 3H), 1.01 ppm (s, 3H); ¹³C NMR (100 MHz, CDCl₃) δ 160.4, 134.5, 134.3, 132.6, 130.1, 127.2, 126.2, 124.6, 114.8, 113.3, 83.7, 79.2, 78.7, 74.6, 72.6, 28.3, 19.3 ppm; HRMS (ESI, positive) m/z calcd for C₁₀H₁₇BrO [M+H⁺ 353.5036, found 353.0560.

Compound 400′:

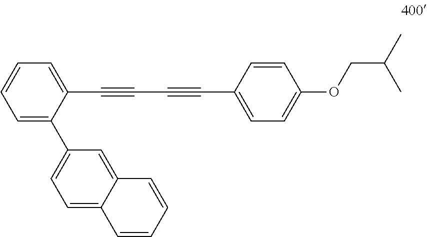 [SMILES: CC(C)COc1ccc(C#CC#Cc2ccccc2-c2ccc3ccccc3c2)cc1]

Compound 452 (353 mg, 1.00 mmol), compound 456 (254 mg, 1.00 mmol) and K₂CO₃ (276 mg, 2.00 mmol) were dissolved in THF (60 mL) and water (10 mL) solution. Pd(PPh₃)₄ (58.0 mg, 0.0502 mmol) was added to the solution before degassing the mixture via bubbling nitrogen for 30 min. The resulting mixture was stirred under a N₂ atmosphere at 80° C. for 24 h. After the reaction was complete, the mixture was diluted with CH₂Cl₂, washed with H₂O and dried over Na₂SO₄. The solvent was removed under reduced pressure and the residue was purified by column chromatography (eluent: hexane:CH₂Cl₂=6:1, v/v) to afford compound 400′ as yellow solid (264 mg, 66%). R_{f}=0.2 (hexane:CH₂Cl₂=6:1); FTIR (neat) 3053, 2957, 2870, 2538, 2210, 2142, 1600, 1508, 1466, 1285, 1245, 1169, 1025, 829, 756 cm⁻¹; ¹H NMR (400 MHz, CDCl₃) δ 8.10 (s, 1H), 8.00-7.90 (m, 3H), 7.86-7.81 (m, 1H), 7.73 (d, J=7.7 Hz, 1H), 7.58-7.52 (m, 3H), 7.48 (m, 1H), 7.44-7.34 (m, 3H), 6.83 (d, J=8.3 Hz, 2H), 3.72 (d, J=6.6 Hz, 2H), 2.09 (m, 1H), 1.05 (s, 3H), 1.03 ppm (s, 3H); ¹³C NMR (100 MHz, CDCl₃) δ 160.2, 145.0, 137.9, 134.6, 134.2, 133.5, 133.0, 130.1, 129.4, 128.6, 128.3, 127.9, 127.8, 127.6, 127.4, 126.4, 126.3, 120.9, 114.8, 113.6, 82.5, 80.8, 77.4, 74.6, 73.1, 28.4, 19.4 ppm; HRMS (ESI, positive) m/z calcd for C₃₀H₂₄O [M+H]⁺ 401.1900, found 401.1906.

Synthesis of Compound 400a:

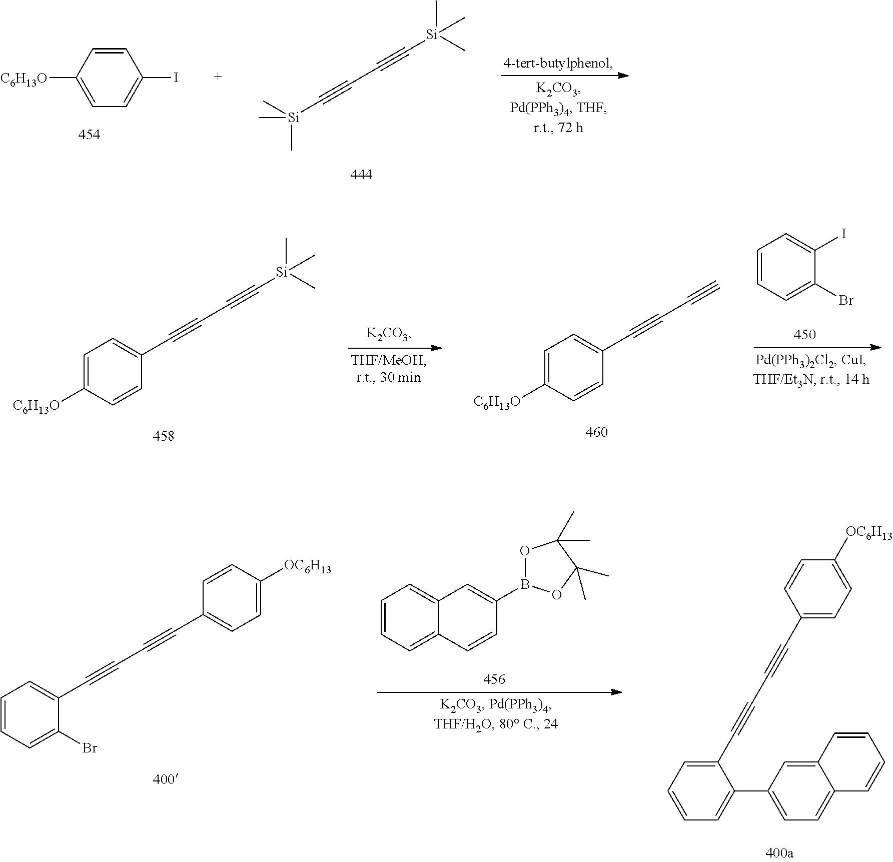

Compound 458:

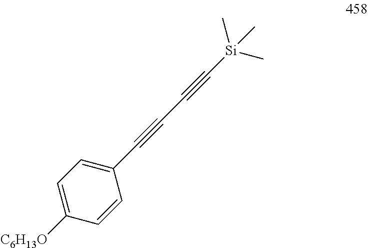

Compound 458 was prepared as described for compound 446, using compound 454 (6.08 g, 20.0 mmol) and compound 444 (7.76 g, 40.0 mmol) as starting materials. Purification by flash column chromatography (silica gel, hexane:CH₂Cl₂=10:1, v/v) yielded pure compound 458 as a yellow solid (4.54 g, 76%). R_{f}=0.1 (hexane:CH₂Cl₂=10:1); FTIR (neat) 2953, 2925, 2859, 2201, 2101, 2067, 1602, 1508, 1468, 1253, 1172, 843, 831, 761 cm⁻¹; ¹H NMR (400 MHz, CDCl₃) δ 7.47-7.35 (m, 2H), 6.85-6.76 (m, 2H), 3.95 (t, J=6.6 Hz, 2H), 1.82-1.71 (m, 2H), 1.45 (m, 2H), 1.33 (m, 4H), 0.90 (t, J=7.1 Hz, 3H), 0.23 ppm (s, 9H); ¹³C NMR (100 MHz, CDCl₃) δ 160.2, 134.4, 114.8, 113.1, 89.9, 88.3, 77.3, 73.1, 68.3, 31.7, 29.2, 25.8, 22.7, 14.2, −0.2 ppm; HRMS (ESI, positive) m/z calcd for C₁₉H₂₆OSi [M+H]⁺ 299.1826, found 299.1825.

Compound 460:

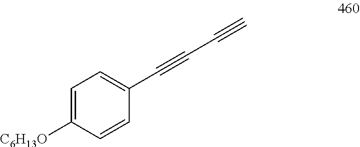

Compound 460 was prepared as described for compound 448, using compound 458 (4.51 g, 15.1 mmol) as a starting material. Compound 460 was used directly for the next step and without further purification.

Compound 400a′:

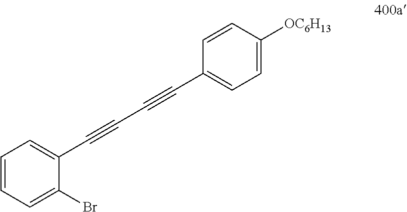

Compound 400a′ was prepared as described for compound 452, using the solution of compound 460 and 1-bromo-2-iodobenzene 450 (4.53 g, 16.0 mmol) as starting materials. Purification by flash column chromatography (silica gel, hexane:CH₂Cl₂=10:1, v/v) yielded pure compound 400a′ as a yellow solid (4.33 g, 71%). R_{f}=0.3 (hexane:CH₂Cl₂=7:1); FTIR (neat) 2947, 2858, 2545, 2208, 2141, 1600, 1508, 1465, 1290, 1249, 1170, 1026, 829, 751 cm⁻¹; ¹H NMR (400 MHz, CDCl₃) δ 7.61-7.56 (m, 1H), 7.57-7.52 (m, 1H), 7.51-7.44 (m, 2H), 7.30-7.26 (m, 1H), 7.22-7.17 (m, 1H), 6.89-6.80 (m, 2H), 3.97 (t, J=6.6 Hz, 2H), 1.78 (m, 2H), 1.49-1.42 (m, 2H), 1.38-1.31 (m, 4H), 0.91 ppm (t, J=7.1 Hz, 3H); ¹³C NMR (100 MHz, CDCl₃) δ 160.3, 134.5, 134.3, 132.7, 130.1, 127.2, 126.3, 124.6, 114.8, 113.3, 83.7, 79.2, 78.7, 72.7, 68.3, 31.7, 29.3, 25.8, 22.7, 14.2 ppm; HRMS (ESI, positive) m/z calcd for C₂₂H₂₁BrO [M+Na]⁺ 403.0668, found 403.0673.

Compound 400a:

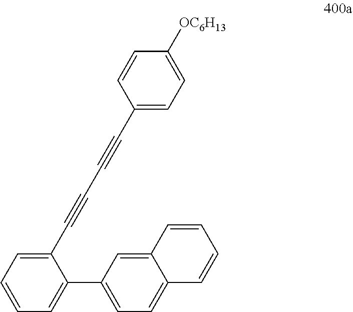

Compound 400a was prepared as described for compound 400′, using compound 400a′(381 mg, 1.00 mmol) and compound 456 (254 mg, 1.00 mmol) as starting materials. Purification by flash column chromatography (silica gel, hexane:CH₂Cl₂=10:1, v/v) yielded pure compound 400a as a yellow solid (330 mg, 77%). R_{f}=0.5 (hexane:CH₂Cl₂=6:1); FTIR (neat) 3054, 2929, 2857, 2212, 2144, 1602, 1509, 1466, 1288, 1250, 1171, 1024, 831, 758 cm⁻¹; ¹H NMR (400 MHz, CDCl₃) δ 8.06 (s, 1H), 8.00-7.86 (m, 3H), 7.82-7.76 (m, 1H), 7.73-7.66 (m, 1H), 7.57-7.48 (m, 3H), 7.49-7.43 (m, 1H), 7.42-7.31 (m, 3H), 6.86-6.74 (m, 2H), 3.94 (t, J=6.6 Hz, 2H), 1.83-1.70 (m, 2H), 1.50-1.26 (m, 6H), 0.97-0.85 (m, 3H). ¹³C NMR (100 MHz, CDCl₃) δ 160.0, 144.9, 137.8, 134.5, 134.2, 133.4, 132.9, 130.1, 129.3, 128.5, 128.2, 127.8, 127.8, 127.5, 127.3, 126.3, 126.3, 120.8, 114.7, 113.5, 82.4, 80.7, 77.2, 73.0, 68.2, 31.7, 29.2, 25.8, 22.7, 14.2 ppm; HRMS (ESI, positive) m/z calcd for C₃₂H₂₈O [M+Na]⁺ 451.2032, found 451.2052.

Synthesis of Compounds 400c-400h:

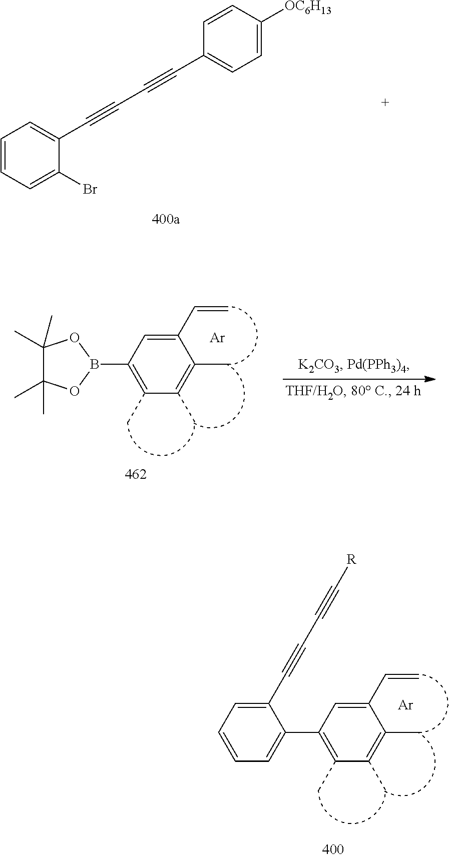

General procedure A: Compound 400a (190 mg, 0.500 mmol), Compound 462 (0.500 mmol) and K₂CO₃ (138 mg, 1.00 mmol) were dissolved in THF (30 mL) and water (5 mL) solution. Pd(PPh₃)₄ (29.0 mg, 0.0250 mmol) was added to the solution before degassing the mixture via bubbling nitrogen for 30 min. The resulting mixture was stirred under a N₂ atmosphere at 80° C. for 24 h. After the reaction was complete, the mixture was diluted with CH₂Cl₂, washed with H₂O and dried over Na₂SO₄. The solvent was removed under reduced pressure and the residue was purified by silica gel column chromatography.

Compound 400b:

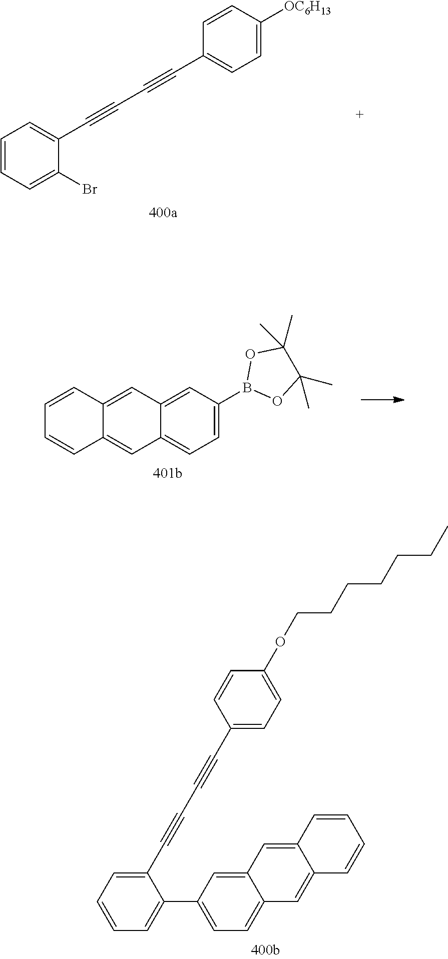

Compound 400b was prepared following general procedure A, using compound 400a (190 mg, 0.500 mmol) and compound 401b (152 mg, 0.500 mmol) as starting material. Purification by flash column chromatography (silica gel, hexane:CH₂Cl₂=6:1, v/v) yielded pure compound 400b as yellow oil (167 mg, 70%). R_{f}=0.3 (hexane:CH₂Cl₂=6:1); FTIR (neat) 3052, 2939, 2869, 2537, 2209, 2142, 1600, 1565, 1509, 1470, 1393, 1286, 1246, 1173, 1026, 907, 893, 734 cm⁻¹; ¹H NMR (400 MHz, CDCl₃) δ 8.49 (m, 2H), 8.22 (m, 1H), 8.13-8.07 (m, 1H), 8.07-7.97 (m, 2H), 7.78 (m, 1H), 7.72 (m, 1H), 7.59-7.54 (m, 1H), 7.53-7.42 (m, 3H), 7.42-7.31 (m, 3H), 6.85-6.71 (m, 2H), 3.92 (t, J=6.6 Hz, 2H), 1.75 (m, 2H), 1.51-1.25 (m, 6H), 0.96-0.85 ppm (m, 3H); ¹³C NMR (100 MHz, CDCl₃) δ 160.0, 144.9, 137.2, 134.6, 134.2, 132.10, 132.07, 131.7, 131.1, 130.0, 129.4, 128.4, 128.34, 128.32, 128.0, 127.4, 127.3, 126.9, 126.1, 125.6, 125.5, 120.9, 114.7, 113.5, 82.5, 80.8, 77.5, 73.0, 68.2, 31.7, 29.2, 25.8, 22.7, 14.2 ppm; HRMS (ESI, positive) m/z calcd for C₃₆H₃₀O [M+Na]⁺ 501.2189, found 501.2189.

Compound 400c:

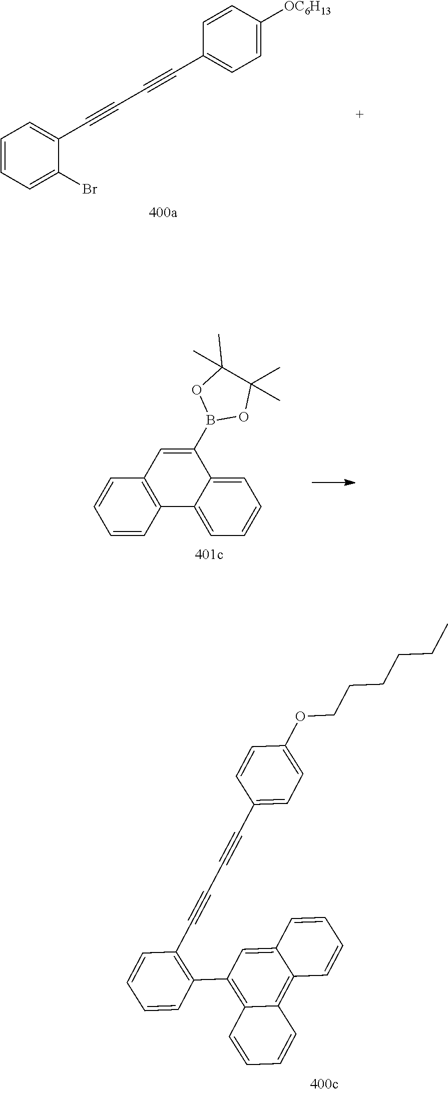

Compound 400c was prepared following general procedure A, using compound 400a (190 mg, 0.500 mmol) and compound 401c (152 mg, 0.500 mmol) as starting materials. Purification by flash column chromatography (silica gel, hexane:CH₂Cl₂=6:1, v/v) yielded pure 400c as yellow oil (194 mg, 81%). R_{f}=0.2 (hexane:CH₂Cl₂=6:1); FTIR (neat) 3061, 2956, 2928, 2856, 2212, 2144, 1602, 1509, 1467, 1288, 1250, 1171, 1023, 831, 767, 746 cm⁻¹; ¹H NMR (400 MHz, CDCl₃) δ 8.64 (m, 2H), 7.82 (d, J=7.9 Hz, 1H), 7.70-7.62 (m, 2H), 7.61-7.46 (m, 4H), 7.46-7.26 (m, 4H), 7.18-7.06 (m, 2H), 6.67-6.52 (m, 2H), 3.75 (m, 2H), 1.69-1.53 (m, 2H), 1.38-1.09 (m, 6H), 0.86-0.70 ppm (m, 3H); ¹³C NMR (100 MHz, CDCl₃) δ 159.9, 143.8, 137.0, 134.0, 133.9, 131.6, 131.3, 131.1, 130.6, 130.4, 129.0, 128.8, 128.2, 127.6, 127.0, 126.85, 126.83, 126.7, 126.6, 123.0, 122.8, 122.7, 114.6, 113.4, 82.3, 80.2, 77.4, 72.9, 68.2, 31.7, 29.2, 25.8, 22.7, 14.1 ppm; HRMS (ESI, positive) m/z calcd for C₃₆H₃₀O [M+H]⁺ 479.2369, found 479.2364.

Compound 400d:

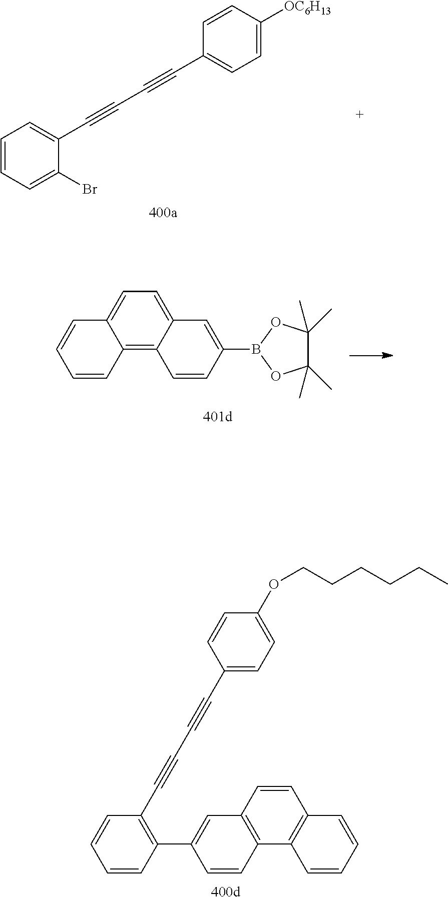

Compound 400d was prepared following general procedure A, using compound 400a (190 mg, 0.500 mmol) and compound 401d (152 mg, 0.500 mmol) as starting materials. Purification by flash column chromatography (silica gel, hexane:CH₂Cl₂=8:1, v/v) yielded pure 400d as yellow oil (160 mg, 67%). R_{f}=0.15 (hexane:CH₂Cl₂=8:1); FTIR (neat) 3054, 2947, 2925, 2542, 2206, 2137, 1601, 1509, 1465, 1288, 1255, 1172, 1027, 829 cm⁻¹; ¹H NMR (400 MHz, CDCl₃) δ 8.78 (d, J=8.6 Hz, 1H), 8.76-8.72 (m, 1H), 8.12 (d, J=1.9 Hz, 1H), 7.96 (dd, J=8.6, 2.0 Hz, 1H), 7.91 (dd, J=7.8, 1.5 Hz, 1H), 7.85-7.76 (m, 2H), 7.74-7.53 (m, 4H), 7.48 (td, J=7.6, 1.4 Hz, 1H), 7.40-7.32 (m, 3H), 6.78 (d, J=8.8 Hz, 2H), 3.93 (t, J=6.6 Hz, 2H), 1.80-1.71 (m, 2H), 1.47-1.30 (m, 6H), 0.95-0.86 ppm (m, 3H); ¹³C NMR (100 MHz, CDCl₃) δ 160.0, 144.7, 138.5, 134.5, 134.2, 132.4, 132.1, 130.3, 130.0, 129.8, 129.4, 129.0, 128.7, 127.9, 127.38, 127.35, 127.3, 126.8, 126.7, 123.0, 122.7, 120.9, 114.7, 113.6, 82.5, 80.7, 77.3, 73.0, 68.2, 31.7, 29.2, 25.8, 22.7, 14.2 ppm; HRMS (ESI, positive) m/z calcd for C₃₆H₃₀O [M+H]⁺ 479.2369, found 479.2353.

Compound 400e:

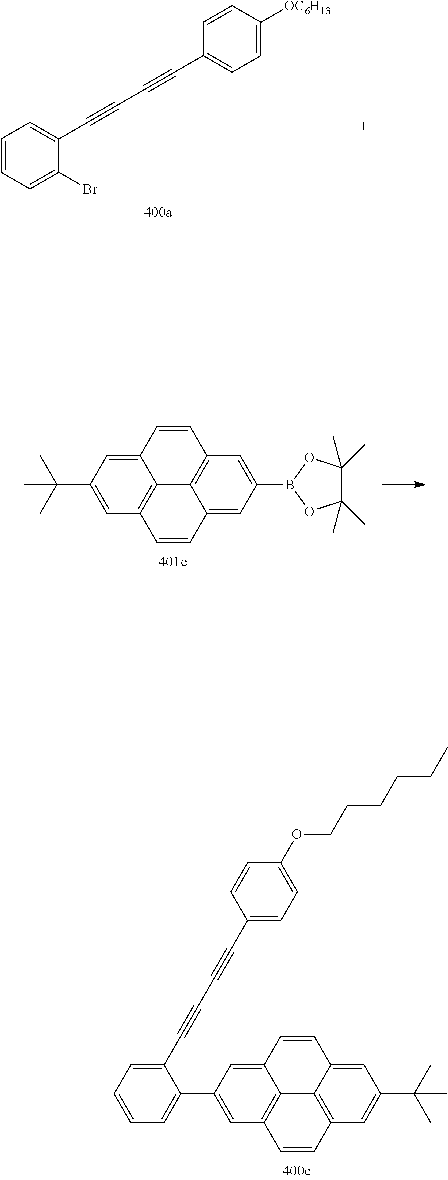

Compound 400e was prepared following general procedure A, using compound 400a (190 mg, 0.500 mmol) and compound 401e (192 mg, 0.500 mmol) as starting materials. Purification by flash column chromatography (silica gel, hexane:CH₂Cl₂=6:1, v/v) yielded pure compound 400e as yellow oil (184 mg, 66%). R_{f}=0.2 (hexane:CH₂Cl₂=5:1); FTIR (neat) 3042, 2952, 2926, 2867, 2539, 2209, 2142, 1924, 1601, 1508, 1467, 1287, 1247, 1225, 1170, 1023, 886, 829, 737 cm⁻¹; ¹H NMR (400 MHz, CDCl₃) δ 8.40 (s, 2H), 8.24 (s, 2H), 8.11 (q, J=9.0 Hz, 4H), 7.76 (d, J=7.7 Hz, 1H), 7.65 (d, J=7.7 Hz, 1H), 7.51 (m, 1H), 7.45-7.36 (m, 1H), 7.37-7.28 (m, 2H), 6.75 (m, 2H), 3.90 (t, J=6.6 Hz, 2H), 1.73 (m, 2H), 1.66 (s, 9H), 1.48-1.26 (m, 6H), 0.95-0.83 ppm (m, 3H); ¹³C NMR (100 MHz, CDCl₃) δ 160.0, 149.3, 145.4, 137.5, 134.4, 134.1, 131.2, 131.0, 130.6, 129.3, 128.0, 127.6, 127.3, 125.7, 124.1, 122.9, 122.4, 121.3, 114.6, 113.6, 82.2, 80.9, 77.1, 72.9, 68.2, 35.4, 32.1, 31.7, 29.2, 25.8, 22.7, 14.2 ppm; HRMS (ESI, positive) m/z calcd for C₄₂H₃₈O [M+H]⁺ 559.2995, found 559.3033.

Compound 400f:

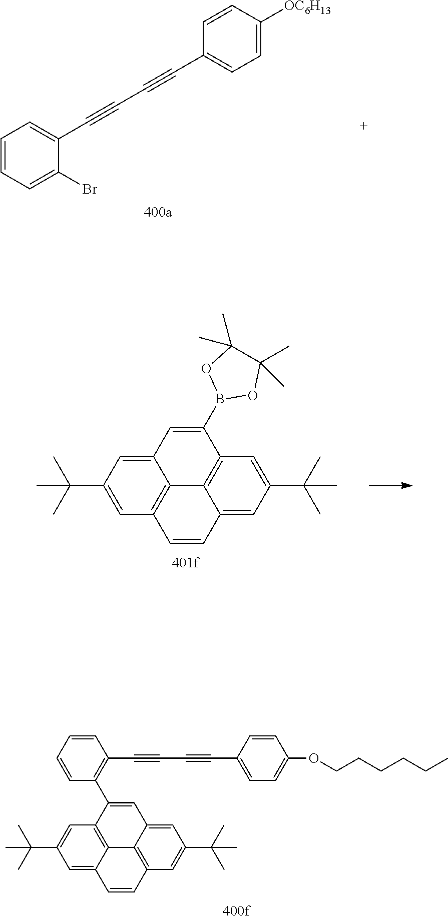

Compound 400f was prepared following general procedure A, using compound 400a (190 mg, 0.500 mmol) and compound 401f (220 mg, 0.500 mmol) as starting materials. Purification by flash column chromatography (silica gel, hexane:CH₂Cl₂=10:1, v/v) yielded pure compound 400f as yellow oil (160 mg, 52%). R_{f}=0.2 (hexane:CH₂Cl₂=10:1); FTIR (neat) 3043, 2953, 2868, 2213, 2144, 1603, 1509, 1468, 1392, 1288, 1249, 1225, 1171, 1025, 886, 830, 761 cm⁻¹; ¹H NMR (400 MHz, CDCl₃) δ 8.35-8.17 (m, 3H), 8.19-7.96 (m, 4H), 7.82 (m, 1H), 7.66-7.42 (m, 3H), 7.25-7.12 (m, 2H), 6.77-6.62 (m, 2H), 3.87 (t, J=6.6 Hz, 2H), 1.80-1.68 (m, 2H), 1.61 (s, 9H), 1.51 (s, 9H), 1.47-1.21 (m, 6H), 1.01-0.83 ppm (m, 3H); ¹³C NMR (100 MHz, CDCl₃) δ 159.9, 148.9, 148.4, 144.2, 137.9, 134.02, 133.96, 131.3, 131.0, 130.9, 130.5, 129.8, 129.0, 128.9, 127.9, 127.6, 127.2, 123.2, 122.9, 122.8, 122.5, 122.3, 121.6, 114.5, 113.5, 82.2, 80.5, 77.5, 72.9, 68.2, 35.4, 35.4, 32.1, 32.0, 31.7, 29.2, 25.8, 22.7, 14.2 ppm; HRMS (ESI, positive) m/z calcd for C₄₆H₄₆O [M+Na]⁺ 637.3446, found 637.3472.

Compound 400g:

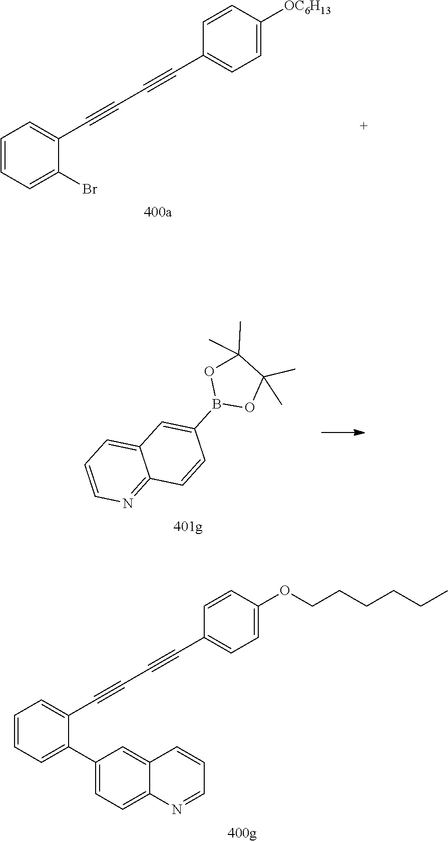

Compound 400g was prepared following general procedure A, using compound 400a (190 mg, 0.500 mmol) and compound 401g (127 mg, 0.500 mmol) as starting material. Purification by flash column chromatography (silica gel, CH₂Cl₂) yielded pure 400g as yellow oil (105 mg, 49%). R_{f}=0.1 (CH₂Cl₂); FTIR (neat) 3062, 2950, 2928, 2856, 2537, 2210, 2142, 1601, 1509, 1475, 1287, 1249, 1171, 1019, 831, 775 cm⁻¹; ¹H NMR (400 MHz, CDCl₃) δ 8.95 (m, 1H), 8.22 (m, 2H), 8.10-7.94 (m, 2H), 7.70 (m, 1H), 7.62-7.28 (m, 6H), 6.87-6.69 (m, 2H), 3.93 (t, J=6.6 Hz, 2H), 1.82-1.69 (m, 2H), 1.53-1.22 (m, 6H), 0.98-0.85 ppm (m, 3H); ¹³C NMR (100 MHz, CDCl₃) δ 160.1, 150.8, 147.9, 144.1, 138.6, 136.5, 134.5, 134.2, 131.2, 130.0, 129.4, 129.3, 128.3, 128.0, 127.7, 121.5, 120.9, 114.7, 113.4, 82.7, 80.4, 77.5, 72.8, 68.3, 31.7, 29.2, 25.8, 22.7, 14.2 ppm; MALDI-TOF m/z calcd for C₃₁H₂₇NO [M+H]⁺ 430.217, found 430.442.

Compound 400g′:

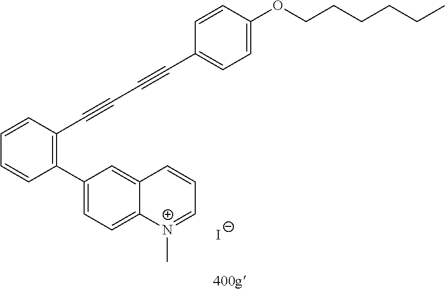 [SMILES: CCCCCCOc1ccc(C#CC#Cc2ccccc2-c2ccc3c(ccc[n+]3C)c2)cc1 ; [I-]]

Compound 400g (43.0 mg, 0.100 mmol) and CH₃I (0.1 mL) were dissolved in toluene (5 ml). The resulting homogeneous solution was refluxed for 24 h. After cooling to room temperature, the residual was purified by flash column chromatography (silica gel, CH₂Cl₂:MeOH=20:1, v/v) yielded pure compound 400g′ as yellow oil (51 mg, 90%). R_{f}=0.1 (CH₂Cl₂:MeOH=20:1); FTIR (neat) 3027, 2954, 2923, 2856, 2210, 2140, 1601, 1510, 1289, 1251, 1172, 1020, 833, 764 cm⁻¹; ¹H NMR (400 MHz, CDCl₃) δ 10.18 (d, J=5.6 Hz, 1H), 9.11 (d, J=7.9 Hz, 1H), 8.56-8.30 (m, 3H), 8.20 (dd, J=8.4, 5.7 Hz, 1H), 7.74-7.62 (m, 1H), 7.55-7.44 (m, 2H), 7.41 (m, 1H), 7.38-7.27 (m, 2H), 6.80-6.70 (m, 2H), 4.91 (s, 3H), 3.89 (t, J=6.6 Hz, 2H), 1.72 (m, 2H), 1.48-1.14 (m, 6H), 0.99-0.75 ppm (m, 3H); ¹³C NMR (100 MHz, CDCl₃) δ 160.3, 150.5, 147.1, 142.3, 140.9, 138.0, 137.5, 134.7, 134.3, 130.1, 129.9, 129.7, 129.0, 123.0, 120.7, 118.6, 114.8, 112.7, 83.7, 79.3, 78.4, 72.4, 68.3, 47.0, 31.6, 29.1, 25.7, 22.7, 14.1 ppm; HRMS (ESI, positive) m/z calcd for C₃₂H₃₀NOI [M+H]⁺ 572.1445, found 572.1456.

Compound 400g″:

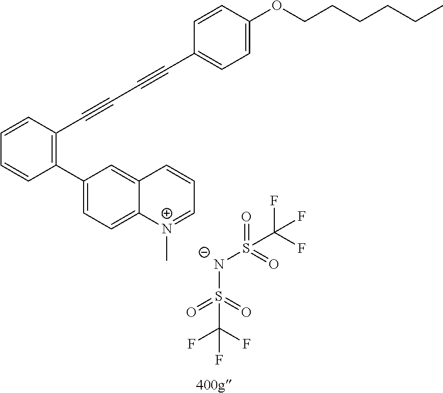 [SMILES: CCCCCCOc1ccc(C#CC#Cc2ccccc2-c2ccc3c(ccc[n+]3C)c2)cc1 ; O=S(=O)([N-]S(=O)(=O)C(F)(F)F)C(F)(F)F]

Compound 400g″ (28.0 mg, 0.0500 mmol) was isolated after treating compound 400g with InCl₃ (1.00 mg, 0.00500 mmol) and AgNTf₂ (2.00 mg, 0.00500 mmol) in toluene at 100° C. for 24 hours. Purification by flash column chromatography (silica gel, CH₂Cl₂:MeOH=20:1, v/v) yielded pure compound 400g″ as yellow oil (7.00 mg, 10%). R_{f}=0.2 (CH₂Cl₂:MeOH=20:1); FTIR (neat) 3096, 2953, 2932, 2852, 2211, 2144, 1602, 1510, 1350, 1251, 1226, 1193, 1137, 1056, 835, 765 cm⁻¹; ¹H NMR (400 MHz, CDCl₃) δ 9.42-9.33 (m, 1H), 9.01 (d, J=8.4 Hz, 1H), 8.52 (m, H), 8.44 (d, J=2.0 Hz, 1H), 8.39-8.32 (m, 1H), 8.07 (dd, J=8.4, 5.8 Hz, 1H), 7.74 (m, 1H), 7.56-7.46 (m, 3H), 7.40-7.32 (m, 2H), 6.83-6.78 (m, 2H), 4.75 (s, 3H), 3.94 (t, J=6.6 Hz, 2H), 1.76 (m, 2H), 1.47-1.41 (m, 2H), 1.33 (m, 4H), 0.92-0.88 ppm (m, 3H); ¹³C NMR (100 MHz, CDCl₃) δ 160.5, 150.1, 147.4, 143.1, 140.8, 138.14, 138.12, 134.8, 134.3, 130.2, 130.00, 129.98, 129.96, 129.3, 122.7, 121.0, 119.9 (q, J=319.6 MHz), 118.0, 114.9, 112.8, 83.8, 79.1, 78.6, 72.2, 68.3, 46.1, 31.7, 29.2, 25.8, 22.7, 14.2 ppm; MALDI-TOF m/z calcd for C₃₄H₃₀F₆N₂O₅S₂ [M-NTf₂]⁺ 444.232, found 444.579.

Compound 116h:

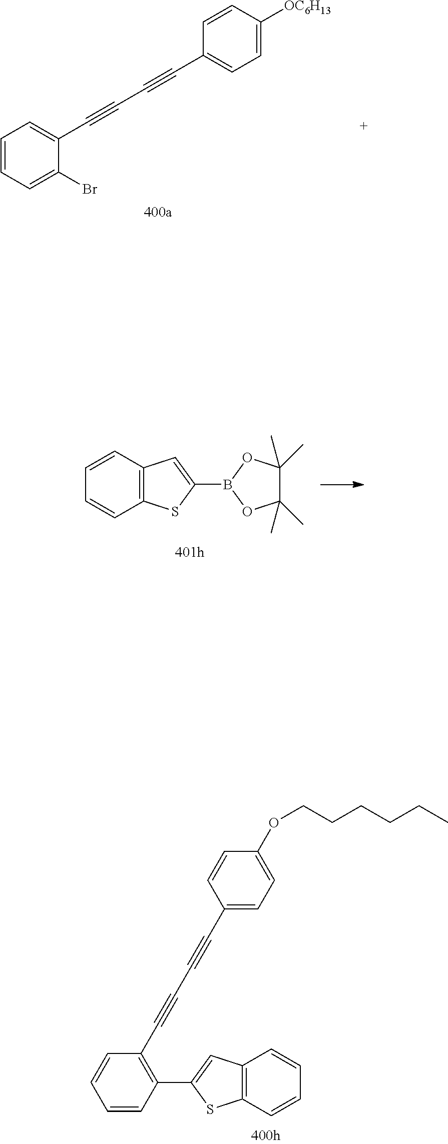

Compound 400h was prepared following general procedure A, using compound 400a (190 mg, 0.500 mmol) and compound 401h (130 mg, 0.500 mmol) as starting materials. Purification by flash column chromatography (silica gel, hexane:CH₂Cl₂=6:1, v/v) yielded pure compound 400h as yellow oil (171 mg, 79%). R_{f}=0.3 (hexane:CH₂Cl₂=6:1); FTIR (neat) 3057, 2953, 2927, 2855, 2210, 2140, 1602, 1509, 1468, 1289, 1250, 1171, 1016, 831, 757 cm⁻¹; ¹H NMR (400 MHz, CDCl₃) δ 7.97-7.78 (m, 3H), 7.66 (m, 2H), 7.52-7.44 (m, 2H), 7.44-7.28 (m, 4H), 6.89-6.80 (m, 2H), 3.96 (t, J=6.6 Hz, 2H), 1.84-1.70 (m, 2H), 1.51-1.40 (m, 2H), 1.39-1.29 (m, 4H), 0.99-0.86 ppm (m, 3H); ¹³C NMR (100 MHz, CDCl₃) δ 160.2, 141.8, 140.4, 140.2, 137.0, 135.2, 134.3, 129.8, 129.4, 127.8, 124.7, 124.5, 124.2, 123.7, 122.2, 120.3, 114.8, 113.4, 83.5, 80.4, 79.1, 73.0, 68.3, 31.7, 29.2, 25.8, 22.7, 14.2 ppm; HRMS (ESI, positive) m/z calcd for C₃₀H₂₆OS [M+H]⁺ 435.1778, found 435.1781.

Compound 400i:

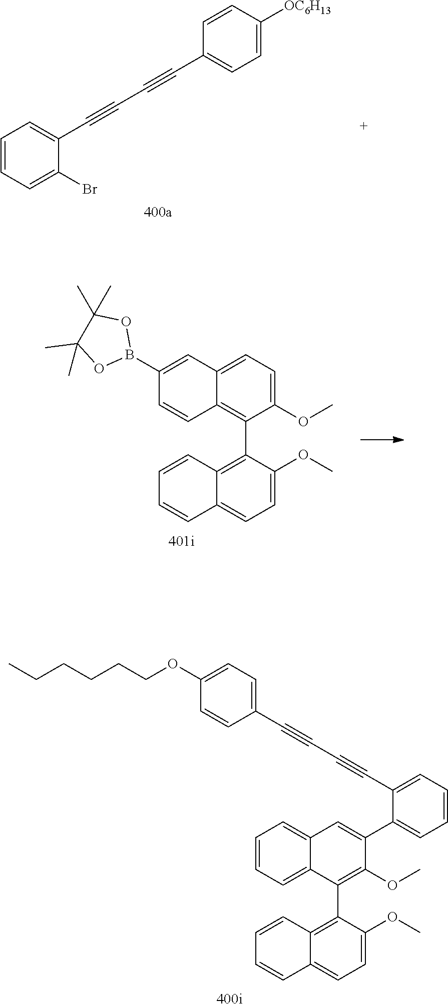

Compound 400i was prepared following general procedure A, using compound 400a (190 mg, 0.500 mmol) and compound 401i (220 mg, 0.500 mmol) as starting material. Purification by flash column chromatography (silica gel, hexane:CH₂Cl₂=1:1, v/v) yielded pure compound 400i as yellow oil (218 mg, 71%). R_{f}=0.2 (hexane:CH₂Cl₂=1:1); FTIR (neat) 3056, 2931, 2857, 2212, 2142, 1601, 1509, 1457, 1404, 1247, 1171, 1086, 1018, 830, 749 cm⁻¹; ¹H NMR (400 MHz, CDCl₃) δ 7.97-7.85 (m, 3H), 7.81 (d, J=7.9 Hz, 1H), 7.64 (d, J=7.4 Hz, 1H), 7.53 (d, J=7.9 Hz, 1H), 7.45-7.15 (m, 10H), 6.77 (d, J=8.7 Hz, 2H), 3.89 (t, J=6.5 Hz, 2H), 3.76 (s, 3H), 3.09 (s, 3H), 1.79-1.67 (m, 2H), 1.48-1.36 (m, 2H), 1.30 (s, 4H), 1.00-0.78 ppm (m, 3H); ¹³C NMR (100 MHz, CDCl₃) δ ¹³C NMR (101 MHz, cdcl₃) δ 159.9, 155.0, 154.3, 142.4, 134.35, 134.33, 134.12, 134.10, 133.6, 130.6(2), 130.4, 129.7, 129.2, 128.8, 128.3, 127.9, 127.4, 127.0, 126.3, 125.65, 125.62, 125.1, 124.9, 123.7, 122.5, 119.5, 114.7, 113.7, 113.5, 82.2, 80.8, 76.8, 73.5, 68.2, 60.7, 56.6, 31.6, 31.6, 29.2, 25.8, 22.7, 14.1 ppm; HRMS (ESI, positive) m/z calcd for C₄₄H₃₈O₃ [M+Na]⁺ 637.2713, found 637.2698.

Compound 400j:

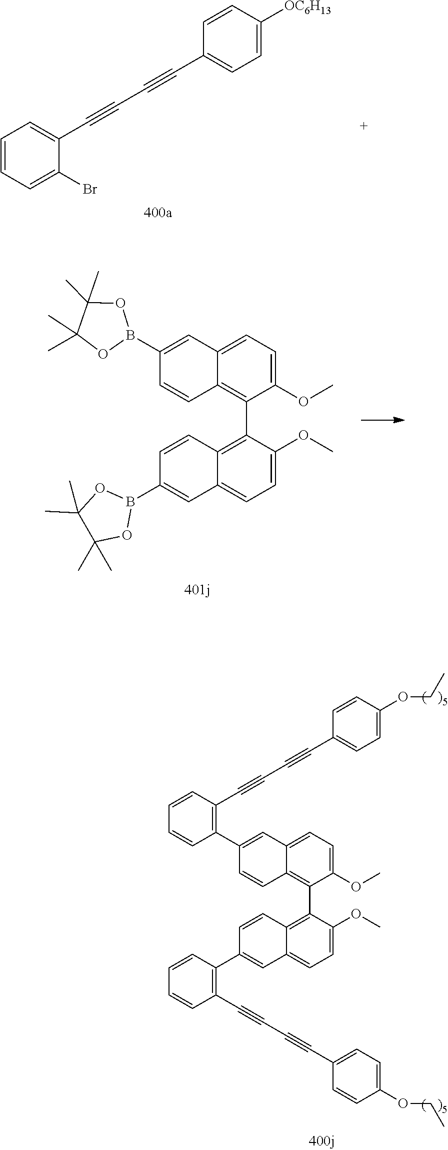

Compound 400j was prepared following general procedure A, using compound 400a (190 mg, 0.500 mmol) and compound 401j (141 mg, 0.250 mmol) as starting materials. Purification by flash column chromatography (silica gel, CH₂Cl₂=2:1, v/v) yielded pure 400j as yellow oil (105 mg, 46%). R_{f}=0.2 (hexane:CH₂Cl₂=2:1); FTIR (neat) 3051, 2929, 2856, 2211, 2140, 1601, 1509, 1466, 1385, 1341, 1248, 1170, 1067, 1045, 830, 757 cm⁻¹; ¹H NMR (400 MHz, CDCl₃) δ 8.13 (d, J=1.9 Hz, 2H), 8.09-8.00 (m, 2H), 7.64 (dd, J=7.7, 1.4 Hz, 2H), 7.55-7.41 (m, 6H), 7.39-7.22 (m, 10H), 6.81-6.67 (m, 4H), 3.89 (t, J=6.6 Hz, 4H), 3.78 (s, 6H), 1.74 (m, 4H), 1.43 (m, 4H), 1.37-1.27 (m, 8H), 0.96-0.83 ppm (m, 6H); ¹³C NMR (100 MHz, CDCl₃) δ 160.0, 155.5, 145.0, 135.2, 134.5, 134.1, 133.5, 130.2, 130.0, 129.3, 129.2, 128.3, 127.9, 126.9, 125.4, 120.6, 119.5, 114.7, 114.5, 113.6, 82.3, 81.0, 77.2, 73.1, 68.2, 57.0, 31.7, 29.2, 25.8, 22.7, 14.2 ppm; MALDI-TOF m/z calcd for C₆₆H₅₈O₄ [M+H]⁺ 915.441, found 915.439.

Compound 400k:

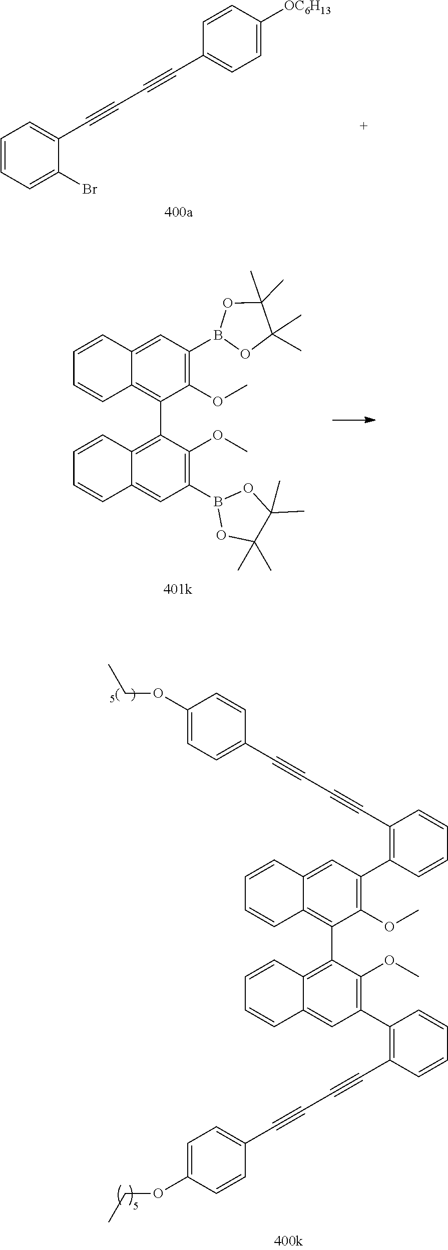

Compound 400k was prepared following general procedure A, using compound 400a (190 mg, 0.500 mmol) and compound 401k (141 mg, 0.250 mmol) as starting material. Purification by flash column chromatography (silica gel, CH₂Cl₂=2:1, v/v) yielded pure compound 400k as yellow oil (121 mg, 53%). R_{f}=0.2 (hexane:CH₂Cl₂=2:1); FTIR (neat) 3052, 2859, 2205, 2142, 1604, 1462, 1337, 1251, 1172, 1049, 835 cm⁻¹; ¹H NMR (400 MHz, CDCl₃) δ 7.96 (s, 2H), 7.94-7.89 (m, 2H), 7.69-7.65 (m, 2H), 7.58-7.54 (m, 2H), 7.47-7.27 (m, 14H), 6.83-6.72 (m, 4H), 3.95 (t, J=6.6 Hz, 4H), 3.22 (s, 6H), 1.81-1.72 (m, 4H), 1.47-1.31 (m, 12H), 0.92-0.88 ppm (m, 6H); ¹³C NMR (100 MHz, CDCl₃) δ 160.0, 154.4, 142.6, 134.4, 134.16, 134.18, 133.6, 131.1, 130.6, 130.5, 128.8, 128.2, 127.4, 126.8, 126.2, 125.0, 122.4, 114.73, 114.70, 113.7, 82.2, 80.7, 76.9, 73.2, 68.3, 61.0, 31.7, 29.3, 25.8, 22.7, 14.2 ppm; HRMS (ESI, positive) m/z calcd for C₆₆H₅₈O₄ [M+H]⁺ 915.441, found 915.341.

Synthesis of Compounds 412, 416, and 420

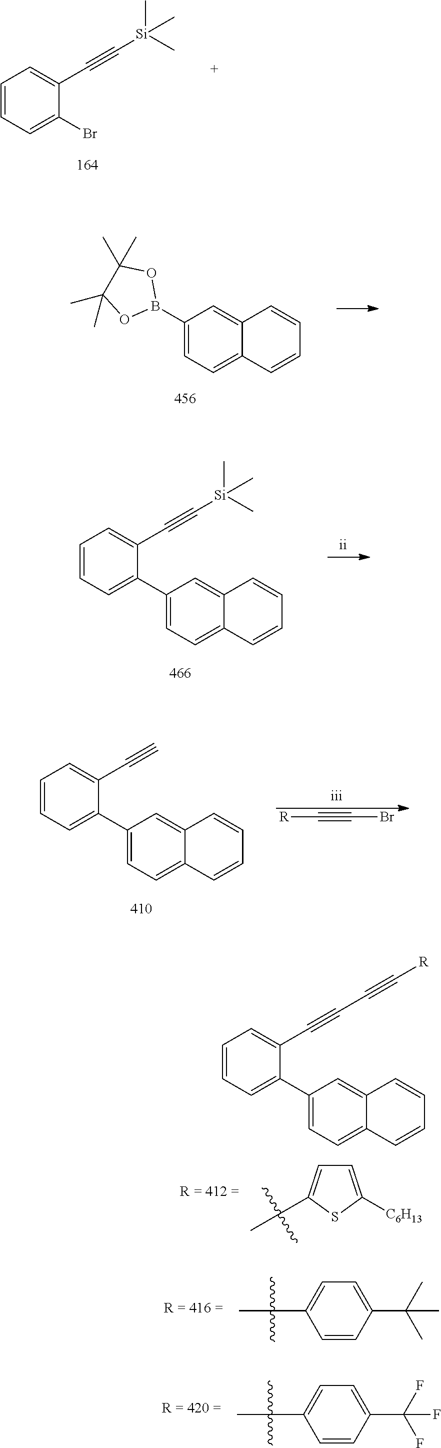

Conditions: i) K₂CO₃, Pd(PPh₃)₄, THF/H₂O, 80° C., 24 h. ii) K₂CO₃, THF/MeOH, r.t., 0.5 h. iii) THF/diisopropylamine, r.t., 12 h.

Compound 466:

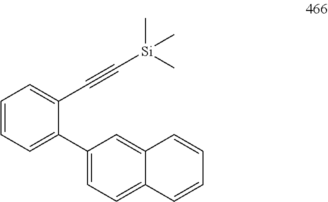 [SMILES: C[Si](C)(C)C#Cc1ccccc1-c1ccc2ccccc2c1]

Compound 466 was prepared as described for compound 400′, using 1-bromo-2-[2-(trimethylsilyl) ethynyl]-benzene (1.27 g, 5.00 mmol) and compound 456 (1.27 g, 5.00 mmol) as starting materials. Purification by flash column chromatography (silica gel, hexane) yielded pure compound 466 as yellow oil (1.09 g, 73%). R_{f}=0.2 (hexane); FTIR (neat) 3054, 2957, 2897, 2155, 1598, 1484, 1464, 1443, 1248, 1210, 1131, 1023, 868, 839, 755 cm⁻¹; ¹H NMR (400 MHz, CDCl₃) δ 8.09 (d, J=1.3 Hz, 1H), 7.91-7.84 (m, 3H), 7.78 (m, 1H), 7.63 (m, 1H), 7.54-7.47 (m, 3H), 7.42 (m, 1H), 7.31 (m, 1H), 0.09 ppm (s, 9H); ¹³C NMR (100 MHz, CDCl₃) δ 144.2, 137.9, 133.6, 133.2, 132.8, 129.8, 128.9, 128.4, 128.3, 127.8, 127.7, 127.3, 127.1, 126.12, 126.11, 121.8, 104.9, 97.9, −0.1 ppm; HRMS (ESI, positive) m/z calcd for C₂₁H₂₀Si [M+H]⁺ 301.1407, found 301.1411.

Compound 410:

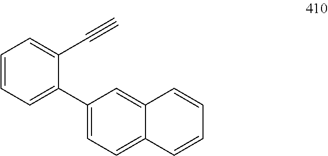 [SMILES: C#Cc1ccccc1-c1ccc2ccccc2c1]

A mixture of compound 466 (900 mg, 3.00 mmol), K₂CO₃ (828 mg, 6.00 mmol), MeOH (30 mL) and THF (30 mL) was stirred at room temperature for 30 min. Then, the reaction mixture was extracted with ethyl acetate and washed with brine. By evaporating the solvent, the title compound 410 was obtained as a colorless solid without further purification (616 mg, 90%). R_{f}=0.2 (hexane); FTIR (neat) 3284, 3054, 2924, 1734, 1593. 1485, 1465, 1442, 1271, 1130, 1023, 898, 857, 820, 757 cm⁻¹; ¹H NMR (400 MHz, CDCl₃) δ 8.08 (d, J=1.8 Hz, 1H), 7.98-7.88 (m, 3H), 7.79 (m, 1H), 7.74-7.68 (m, 1H), 7.57-7.43 (m, 4H), 7.41-7.33 (m, 1H), 3.07 ppm (s, 1H); ¹³C NMR (100 MHz, CDCl₃) δ 144.5, 137.9, 134.0, 133.3, 132.8, 130.0, 129.2, 128.4, 128.3, 127.8, 127.6, 127.5, 127.2, 126.2, 120.8, 83.3, 80.5 ppm; HRMS (ESI, positive) m/z calcd for C₁₈H₁₂ [M+H]⁺ 229.1012, found 229.1011.

Synthesis of Compounds 412, 416 and 420:

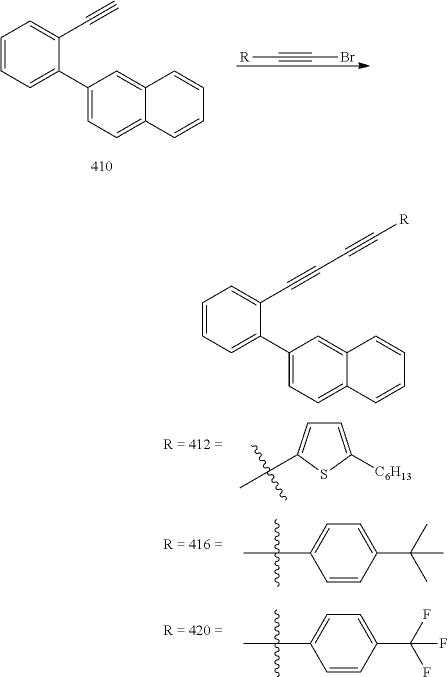

General procedure B: Compound 410 (228 mg, 1.00 mmol), (iPr)₂NH (5 mL) and THF (15 mL) were added into the solution of 2-bromoethynyl (2.00 mmol). After bubbling N₂ for 30 mins, Pd(PPh₃)₂Cl₂ (33.0 mg, 0.0463 mmol) and CuI (17.0 mg, 0.092 mmol) were added. The resulting mixture was stirred at room temperature for 14 h. The ammonium salt was then removed by filtration. The solvent was removed under reduced pressure and the residue was purified by column chromatography.

Compound 412:

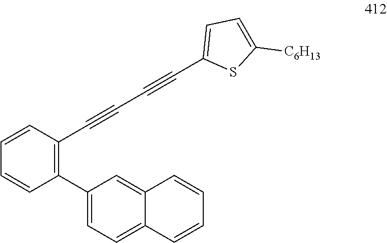

Compound 412 was prepared following general procedure B, using compound 410 (228 mg, 1.00 mmol) and 2-(2-bromoethynyl)-5-hexyl-thiophene (542 mg, 2.00 mmol) as starting material. Purification by flash column chromatography (silica gel, hexane:CH₂Cl₂=8:1, v/v) yielded pure compound 412 as a light yellow solid (213 mg, 51%). R_{f}=0.2 (hexane:CH₂Cl₂=8:1); FTIR (neat) 3056, 2927, 2854, 2199, 2139, 1728, 1486, 1467, 1376, 1270, 1130, 1106, 946, 818 cm⁻¹; ¹H NMR (400 MHz, CDCl₃) δ 8.09 (s, 1H), 8.00-7.90 (m, 3H), 7.82 (m, 1H), 7.73 (m, 1H), 7.59-7.51 (m, 3H), 7.48 (m, 1H), 7.38 (m, 1H), 7.14 (d, J=3.7 Hz, 1H), 6.65 (m, 1H), 2.82-2.74 (m, 2H), 1.72-1.62 (m, 2H), 1.41-1.30 (m, 6H), 0.98-0.91 ppm (m, 3H); ¹³C NMR (100 MHz, CDCl₃) δ 150.1, 144.9, 137.7, 134.6, 134.5, 133.4, 132.9, 130.0, 129.5, 128.5, 128.2, 127.8, 127.4, 127.3, 126.29, 126.27, 124.4, 120.6, 119.3, 83.2, 77.7, 76.9, 75.7, 31.6, 31.5, 30.4, 28.8, 22.7, 14.2 ppm; HRMS (ESI, positive) m/z calcd for C₃₀H₂₆S [M+H]⁺ 419.1828, found 419.1830.

Compound 416:

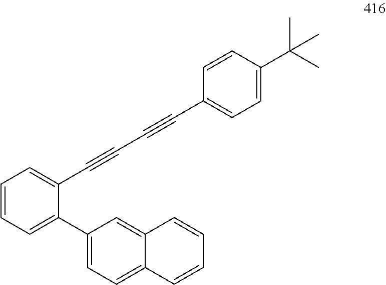 [SMILES: CC(C)(C)c1ccc(C#CC#Cc2ccccc2-c2ccc3ccccc3c2)cc1]

Compound 416 was prepared following general procedure B, using compound 410 (228 mg, 1.00 mmol) and 1-(2-bromoethynyl)-4-tertbutyl-benzene (474 mg, 2.00 mmol) as starting material. Purification by flash column chromatography (silica gel, hexane:CH₂Cl₂=7:1, v/v) yielded pure compound 416 as a light yellow solid (157 mg, 41%). R_{f}=0.3 (hexane:CH₂Cl₂=7:1); FTIR (neat) 3055, 2961, 2903, 2866, 2210, 2146, 1600, 1503, 1486, 1463, 1441, 1362, 1266, 1111, 1016, 893, 834, 757 cm⁻¹; ¹H NMR (400 MHz, CDCl₃) δ 8.10 (m, 1H), 7.99-7.90 (m, 3H), 7.82 (m, 1H), 7.75-7.71 (m, 1H), 7.58-7.51 (m, 3H), 7.48 (m, 1H), 7.43 (m, 2H), 7.40-7.31 (m, 3H), 1.32 ppm (s, 9H); ¹³C NMR (100 MHz, CDCl₃) δ 152.7, 145.0, 137.7, 134.5, 133.4, 132.9, 132.4, 130.1, 129.4, 128.5, 128.2, 127.79, 127.78, 127.5, 127.3, 126.30, 126.27, 125.5, 120.7, 118.8, 82.3, 81.0, 77.1, 73.7, 35.0, 31.2 ppm; HRMS (ESI, positive) m/z calcd for C₃₀H₂₄ [M+K]⁺ 423.1510, found 423.1512.

Compound 420:

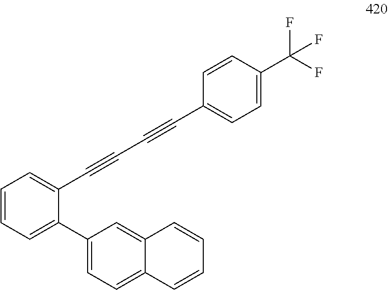 [SMILES: FC(F)(F)c1ccc(C#CC#Cc2ccccc2-c2ccc3ccccc3c2)cc1]

Compound 420 was prepared following general procedure B, using compound 410 (228 mg, 1.00 mmol) and 1-(2-bromoethynyl)-4-trifluoromethyl-benzene (498 mg, 2.00 mmol) as starting material. Purification by flash column chromatography (silica gel, hexane:CH₂Cl₂=20:1, v/v) yielded pure 420 as a light yellow solid (174 mg, 44%). R_{f}=0.1 (hexane); FTIR (neat) 3054, 3023, 2931, 2214, 2140, 1922, 1612, 1485, 1406, 1318, 1166, 1122, 1104, 1065, 1014, 897, 839, 818, 755 cm⁻¹; ¹H NMR (400 MHz, CDCl₃) δ 8.10 (m, 1H), 8.03-7.90 (m, 3H), 7.81 (m, 1H), 7.75 (m, 1H), 7.53 (m, 8H), 7.39 ppm (m, 1H); ¹³C NMR (100 MHz, CDCl₃) δ 145.3, 137.6, 134.6, 133.4, 132.9, 132.7, 130.7 (q, ²J(C,F) 32.7 Hz), 130.1, 129.9, 128.5, 128.3, 127.9, 127.8, 127.38, 127.37, 126.41, 126.37, 125.4 (q, ¹J(C,F) 3.8 Hz), 123.9 (q, ³J(C,F) 270.8 Hz), 120.1, 82.7, 80.3, 76.6, 76.4 ppm; HRMS (ESI, positive) m/z calcd for C₂₇H₁₅F₃ [M+H]⁺ 397.1199, found 397.1207.

Synthesis of Compounds 426 and 430

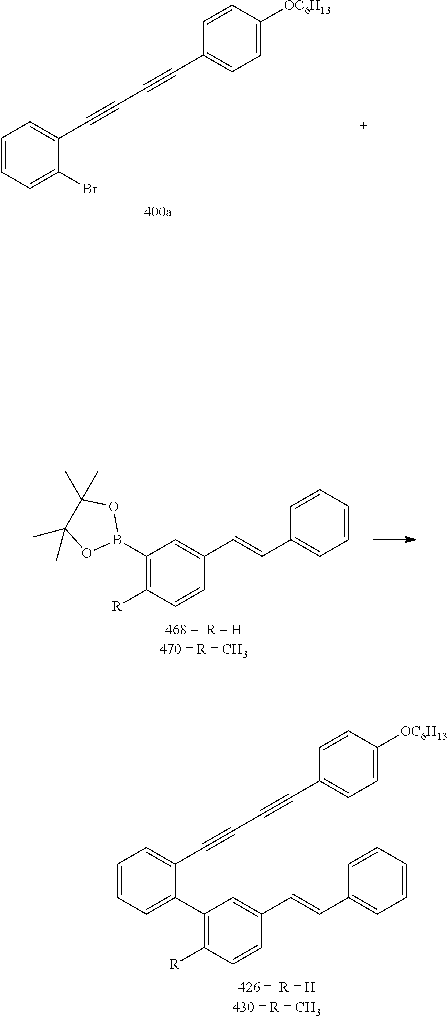

Conditions: i) K₂CO₃, Pd(PPh₃)₄, THF/H₂O, 80° C., 24 h.

Compound 426:

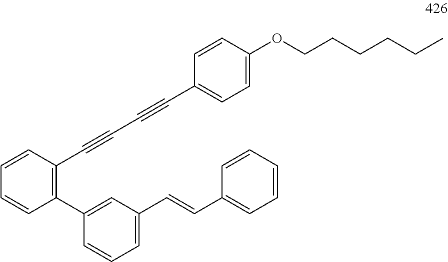 [SMILES: CCCCCCOc1ccc(C#CC#Cc2ccccc2-c2cccc(/C=C/c3ccccc3)c2)cc1]

Compound 426 was prepared following general procedure A, using compound 400a (190 mg, 0.500 mmol) and 468 (153 mg, 0.500 mmol) as starting material. Purification by flash column chromatography (silica gel, hexane:CH₂Cl₂=6:1, v/v) yielded pure 426 as light yellow sticky solid (153 mg, 64%). R_{f}=0.2 (hexane:CH₂Cl₂=6:1); FTIR (neat) 3024, 2927, 2856, 2209, 2142, 1600, 1508, 1467, 1246, 1169, 958, 829 cm⁻¹; ¹H NMR (400 MHz, CDCl₃) δ 7.80-7.91 (m, 1H), 7.63-7.72 (m, 1H), 7.56-7.51 (m, 4H), 7.49-7.40 (m, 3H), 7.40-7.31 (m, 5H), 7.29-7.26 (m, 1H), 7.16-7.24 (m, 2H), 6.85-6.71 (m, 2H), 3.94 (t, J=6.6 Hz, 2H), 1.70-1.84 (m, 2H), 1.50-1.25 (m, 6H), 0.99-0.84 ppm (m, 3H); ¹³C NMR (100 MHz, CDCl₃) δ 160.0, 144.7, 140.5, 137.51, 137.48, 134.4, 134.2, 129.7, 129.4, 129.3, 128.8, 128.74, 128.69, 128.5, 127.7, 127.3, 127.2, 126.7, 126.2, 120.6, 114.7, 113.5, 82.6, 80.7, 77.4, 73.0, 68.2, 31.7, 29.2, 25.8, 22.7, 14.2 ppm; HRMS (ESI, positive) m/z calcd for C36H₃₂0 [M+H]+ 481.2526, found 481.2522.

Compound 430:

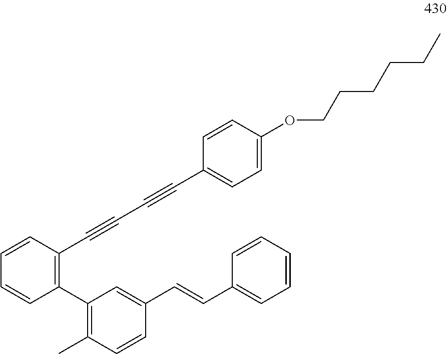 [SMILES: CCCCCCOc1ccc(C#CC#Cc2ccccc2-c2cc(/C=C/c3ccccc3)ccc2C)cc1]

Compound 430 was prepared following general procedure A, using compound 400a (190 mg, 0.500 mmol) and compound 470 (160 mg, 0.500 mmol) as starting material. Purification by flash column chromatography (silica gel, hexane:CH₂Cl₂=10:1, v/v) yielded pure 430 as light yellow sticky solid (173 mg, 70%). R_{f}=0.3 (hexane:CH₂Cl₂=7:1); FTIR (neat) 3022, 2926, 2857, 2211, 2143, 1600, 1508, 1467, 1287, 1246, 1169, 1024, 961 cm⁻¹; ¹H NMR (400 MHz, CDCl₃) δ 7.72-7.61 (m, 1H), 7.57-7.49 (m, 2H), 7.49-7.40 (m, 3H), 7.41-7.20 (m, 8H), 7.19-7.08 (m, 2H), 6.85-6.73 (m, 2H), 3.93 (t, J=6.5 Hz, 2H), 2.24 (s, 3H), 1.80-1.72 (m, 2H), 1.51-1.27 (m, 6H), 1.02-0.86 ppm (m, 3H); ³C NMR (100 MHz, CDCl₃) δ ¹³C NMR (101 MHz, cdcl₃) δ 160.0, 145.2, 140.6, 137.7, 135.9, 134.8, 134.1, 133.6, 130.5, 130.0, 128.9, 128.8, 128.7, 128.2, 128.0, 127.5, 127.3, 126.6, 126.0, 121.9, 114.7, 113.5, 82.4, 80.3, 76.9, 73.0, 68.2, 31.7, 29.2, 25.8, 22.7, 20.0, 14.2 ppm; HRMS (ESI, positive) m/z calcd for C₃₇H₃₄O [M+H]⁺ 495.2682, found 195.2675.

Synthesis of Compounds 434 and 438

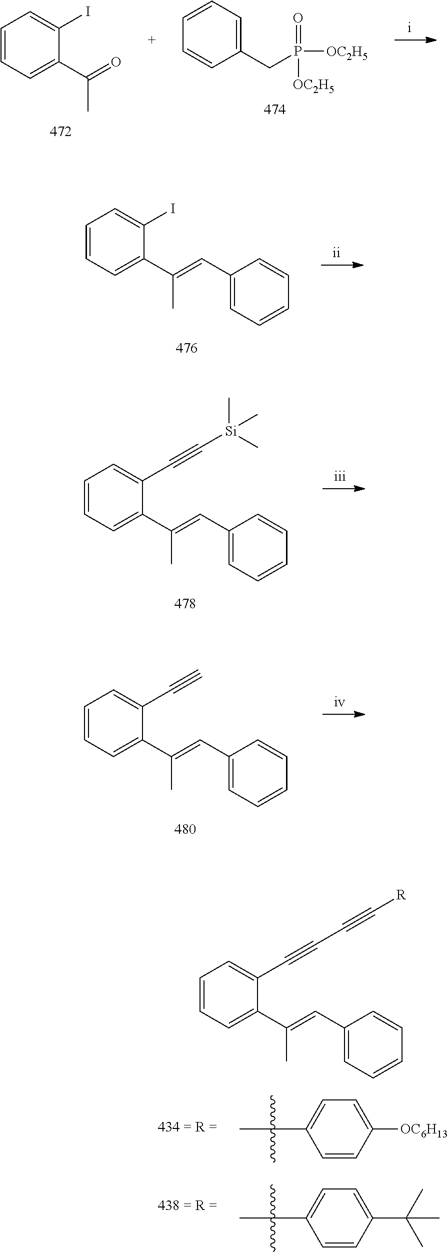

Conditions: i) NaH, THF, 0° C. r.t., 24 h. ii) Pd(PPh₃)₂Cl₂, CuI, THF/Et₃N, r.t., 24 h. iii) K₂CO₃, THF/MeOH, r.t., 0.5 h. iv) THF/diisopropylamine, r.t., 12 h.

Compound 476:

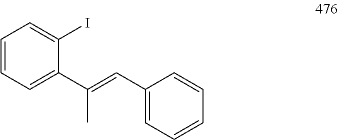 [SMILES: C/C(=C\c1ccccc1)c1ccccc1I]

C₆H₅CH₂P(O)(OC₂H₅)₂ 472 (139 mg, 0.65 mmol) and NaH (24 mg, 1.0 mmol) were placed in a dry flask under N₂. Anhydrous THF (5 mL) was added at 0° C. and the resulting suspension was stirred at rt for 30 min. The solution was cooled at 0° C. and the 2′-iodoacetophenone (123 mg, 0.5 mmol) was added as a solution in dry THF (1 mL). After stirring at r.t. overnight, the reaction was quenched by addition of water (10 mL). The biphasic mixture was extracted with ether (20 mL). The combined organic layers were washed with brine (40 mL), dried over sodium sulfate. The solvent was removed under reduced pressure and the residue was purified by column chromatography (silica gel, hexane) yielded pure compound 476 as white solid (64 mg, 40%). R_{f}=0.3 (hexane); FTIR (neat) 3054, 3021, 2965, 2840, 1598, 1493, 1464, 1427, 1370, 1206, 1011, 914 cm⁻¹; ¹H NMR (400 MHz, CDCl₃) δ 7.92 (d, J=8.0, 1H), 7.35 (td, J=7.5, 1.2 Hz, 1H), 7.17-7.09 (m, 4H), 7.00 (ddd, J=8.0, 7.4, 1.7 Hz, 1H), 6.93-6.87 (m, 2H), 6.55-6.50 (m, 1H), 2.22 ppm (d, J=1.5 Hz, 3H); ¹³C NMR (100 MHz, CDCl₃) δ 147.4, 140.8, 139.6, 136.9, 129.0, 128.9, 128.6, 128.4, 128.1, 128.0, 126.6, 98.0, 26.7 ppm; HRMS (ESI, positive) m/z calcd for C₁₅H₁₃I [M+H]⁺ 321.0135, found 321.0141.

Compound 480:

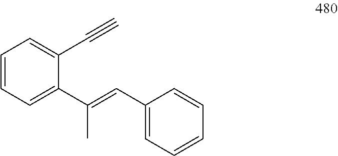 [SMILES: C#Cc1ccccc1/C(C)=C/c1ccccc1]

Compound 476 (320 mg, 1.00 mmol) was dissolved in Et₃N (5 mL) and THF (15 mL). After bubbling N₂ for 30 mins, trimethylsilylacetylene (98.0 mg, 1.00 mmol), Pd(PPh₃)₂Cl₂ (33.0 mg, 0.0463 mmol) and CuI (17.0 mg, 0.092 mmol) were added. The resulting mixture was stirred at room temperature overnight. The ammonium salt was then removed by filtration. The solvent was removed under reduced pressure and the residue was resolved in THF (10 mL) and MeOH (10 mL). K₂CO₃ (276 mg, 2.00 mmol) was added into the above solution. After stirring for 30 min, H₂O(15 mL) was added. The biphasic mixture was extracted with CH₂Cl₂ (60 mL). The combined organic layers were washed with brine (20 mL), dried over sodium sulfate. The solvent was removed under reduced pressure and the residue was purified by column chromatography (silica gel, hexane) yielded pure compound 480 as light yellow oil (179 mg, 82%). R_{f}=0.2 (hexane); FTIR (neat) 3058, 3021, 2965, 2870, 2846, 2106, 1598, 1489, 1477, 1439, 1371, 1031, 1005, 915 cm⁻¹; ¹H NMR (400 MHz, CDCl₃) δ 7.56-7.51 (m, 1H), 7.41-7.28 (m, 1H), 7.27-7.18 (m, 2H), 7.08-7.01 (m, 3H), 6.89-6.83 (m, 2H), 6.55-6.49 (m, 1H), 3.08 (s, 1H), 2.23 ppm (d, J=1.6 Hz, 3H); ¹³C NMR (100 MHz, CDCl₃) δ 146.0, 138.1, 137.4, 133.5, 129.4, 128.6, 128.5, 128.0, 127.9, 126.9, 126.3, 120.9, 82.5, 80.1 ppm; HRMS (ESI, positive) m/z calcd for C₁₇H₁₄ [M+H]⁺ 219.1168, found 219.1183.

Compound 434:

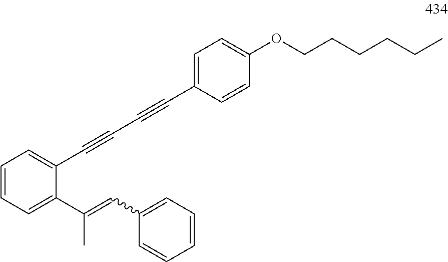 [SMILES: CCCCCCOc1ccc(C#CC#Cc2ccccc2C(C)=Cc2ccccc2)cc1]

Compound 434 was prepared following general procedure B, using compound 480 (109 mg, 0.500 mmol) and 1-(bromoethynyl)-4-(dodecyloxy)-benzene (182 mg, 0.500 mmol) as starting materials. Purification by flash column chromatography (silica gel, hexane:CH₂Cl₂=20:1, v/v) yielded pure compound 434 as light yellow sticky oil (110 mg, 53%). R_{f}=0.2 (hexane:CH₂Cl₂=20:1); FTIR (neat) 3057, 2929, 2857, 2212, 2142, 1601, 1508, 1467, 1440, 1286, 1245, 1170, 1108, 1024, 829 cm⁻¹; ¹H NMR (400 MHz, CDCl₃) δ 7.62-7.55 (m, 1H), 7.50-7.40 (m, 2H), 7.32-7.21 (m, 2H), 7.21-6.99 (m, 4H), 7.01-6.88 (m, 2H), 6.88-6.80 (m, 2H), 6.65-6.53 (m, 1H), 3.97 (t, J=6.6 Hz, 2H), 2.28 (m, 3H), 1.79 (m, 2H), 1.51-1.43 (m, 2H), 1.43-1.27 (m, 4H), 1.02-0.85 ppm (m, 3H); ¹³C NMR (100 MHz, CDCl₃) δ 160.0, 146.6, 137.8, 137.4, 134.2, 133.9, 129.6, 128.7, 128.7, 128.3, 128.0, 127.0, 126.3, 121.0, 114.7, 113.6, 82.4, 80.0, 73.1, 68.2, 31.7, 29.2, 26.7, 25.8, 22.7, 14.2 ppm; HRMS (ESI, positive) m/z calcd for C₃₁H₃₀O [M+H]⁺ 419.2369, found 419.2351.

Compound 438:

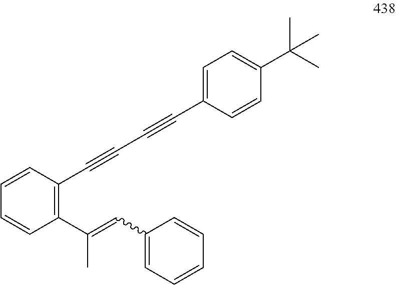 [SMILES: CC(=Cc1ccccc1)c1ccccc1C#CC#Cc1ccc(C(C)(C)C)cc1]

Compound 438 was prepared following general procedure A, using compound 480 (109 mg, 0.500 mmol) and 1-(bromoethynyl)-4-tertbutyl-benzene (118 mg, 0.500 mmol) as starting material. Purification by flash column chromatography (silica gel, hexane, v/v) yielded pure compound 438 as light yellow sticky oil (91 mg, 49%). R_{f}=0.1 (hexane); FTIR (neat) 3058, 2962, 2904, 2867, 2215, 2149, 1598, 1496, 1478, 1462, 1440, 1407, 1363, 1265, 1199, 1107, 1016, 833 cm⁻¹; ¹H NMR (400 MHz, CDCl₃) δ 7.60-7.53 (m, 1H), 7.48-7.41 (m, 2H), 7.37-7.31 (m, 2H), 7.29-7.20 (m, 2H), 7.16-6.97 (m, 4H), 6.95-6.82 (m, 2H), 6.57-6.62 (m, 1H), 2.26 (d, J=1.5 Hz, 1H), 1.31 ppm (s, 9H); ¹³C NMR (100 MHz, CDCl₃) δ 152.7, 146.6, 137.8, 137.4, 134.0, 132.40, 132.38, 129.7, 128.8, 128.7, 128.4, 128.0, 127.0, 126.4, 125.6, 120.9, 82.4, 80.2, 76.9, 73.7, 35.1, 31.3, 26.7 ppm; HRMS (ESI, positive) m/z calcd for C₂₉H₂₆ [M+H]⁺ 375.2107, found 375.2103.

Cascade Benzannulation Reaction of Diynes

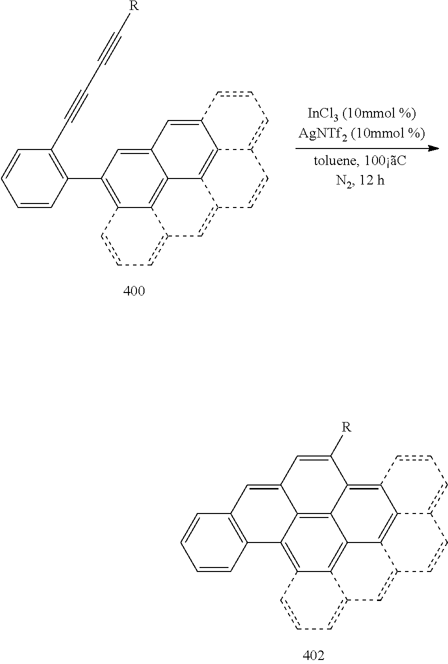

Cascade benzannulation reaction of diynes

General procedure C: In a nitrogen-filled glove box, precursor 400 (0.10 mmol), InCl₃ (2.2 mg, 0.010 mmol), and AgNTf₂ (3.9 mg, 0.010 mmol) were dissolved in toluene (10 mL) in a sealed tube. The resulting mixture was stirred at 100° C. for 12 hour and then cooled down to room temperature. After evaporation of solvent, the residue was purified by silica gel column chromatography to yield corresponding annulated product 402.

Compound 402′:

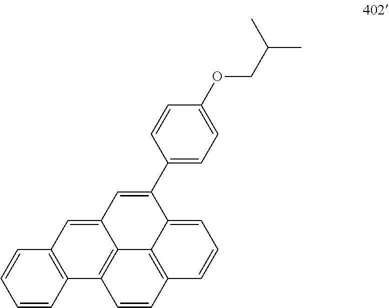 [SMILES: CC(C)COc1ccc(-c2cc3cc4ccccc4c4ccc5cccc2c5c34)cc1]

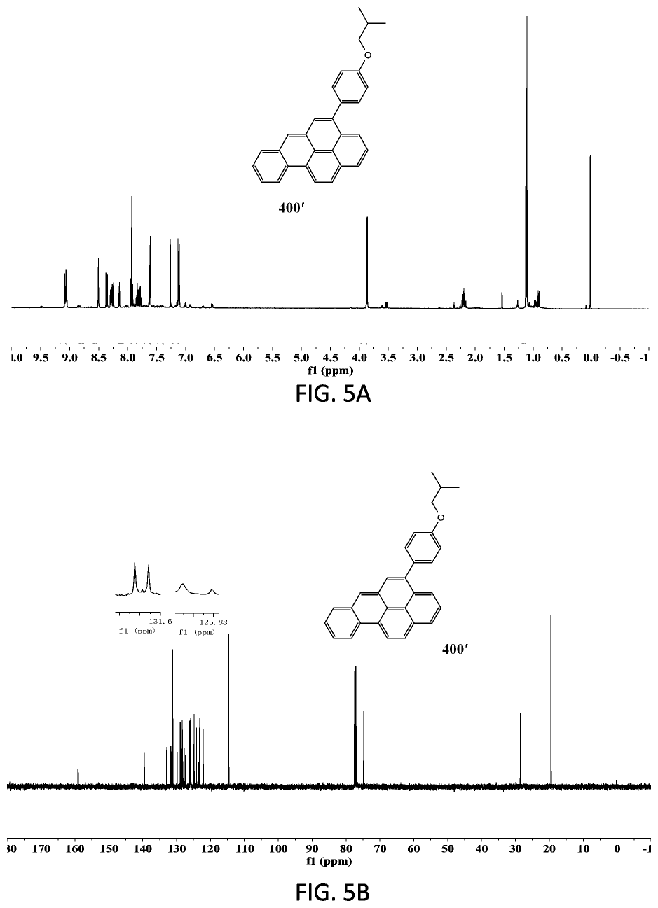

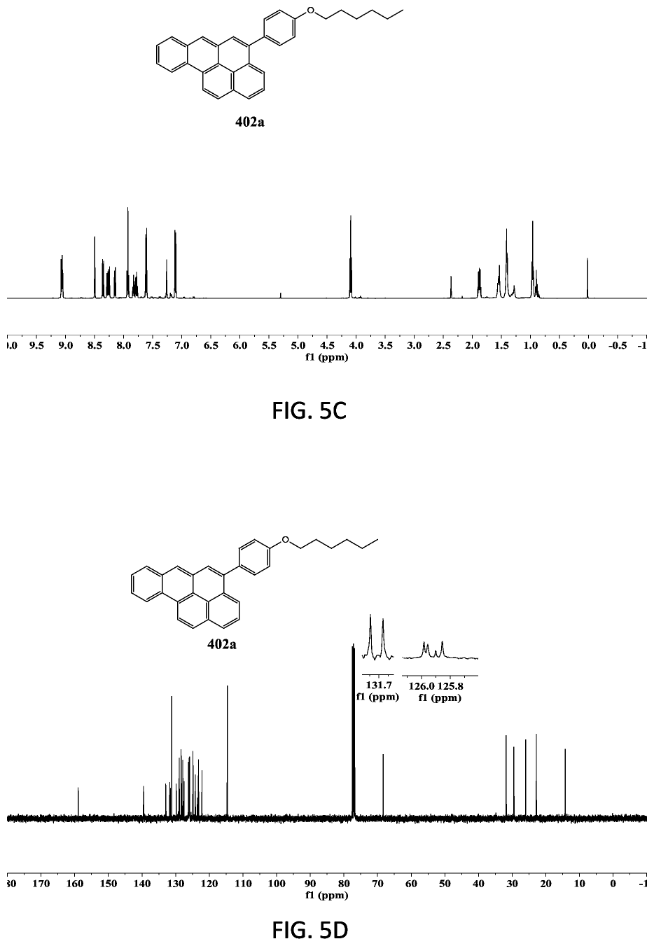

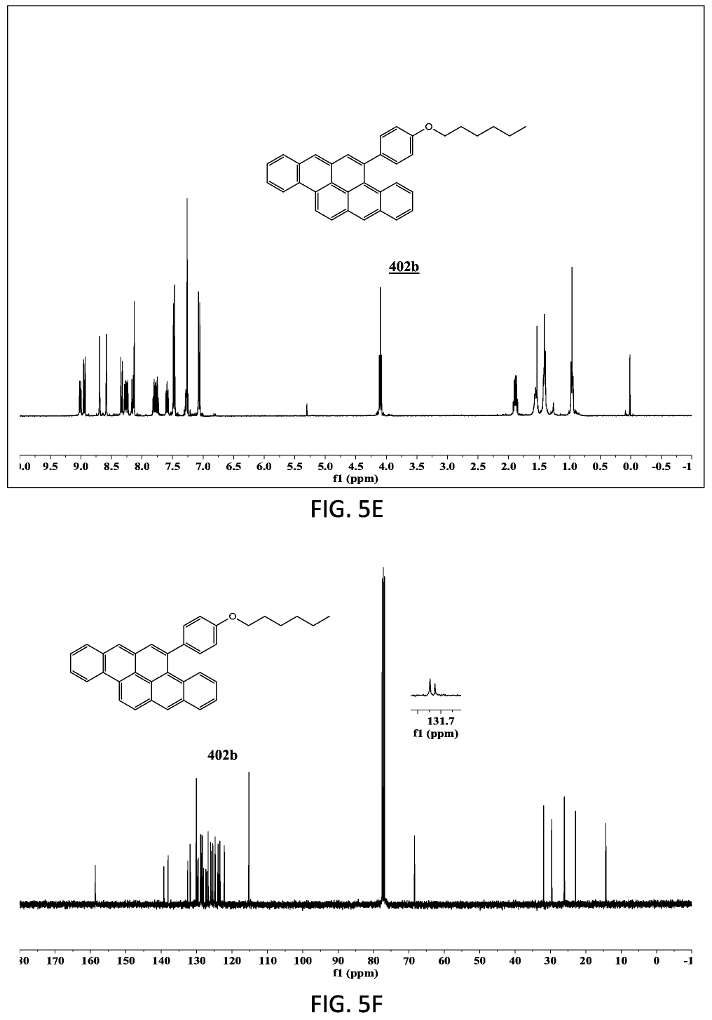

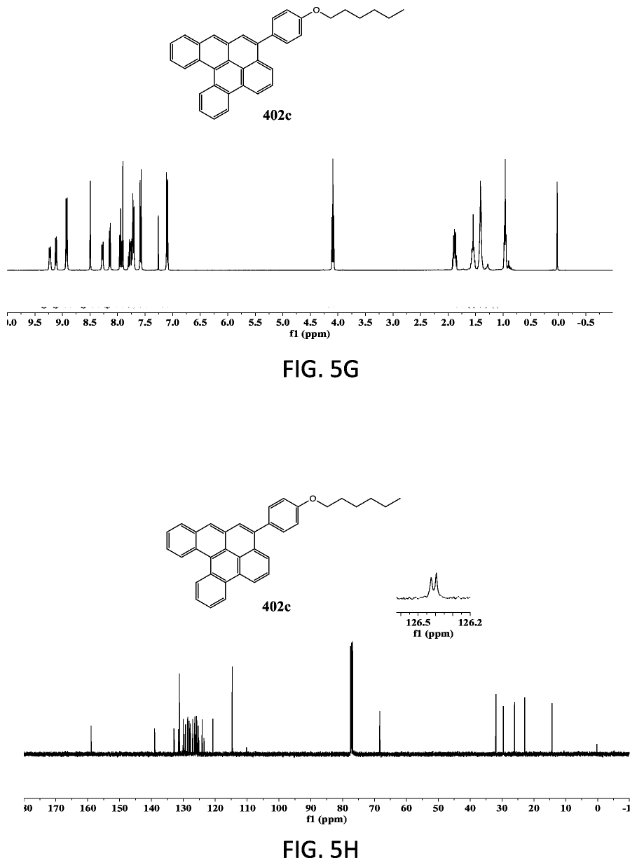

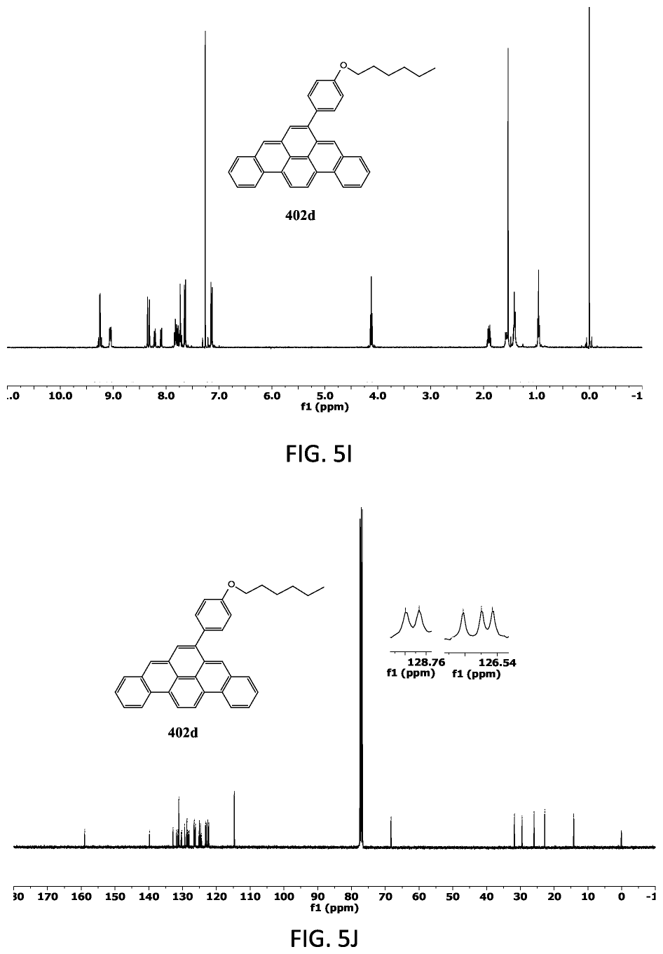

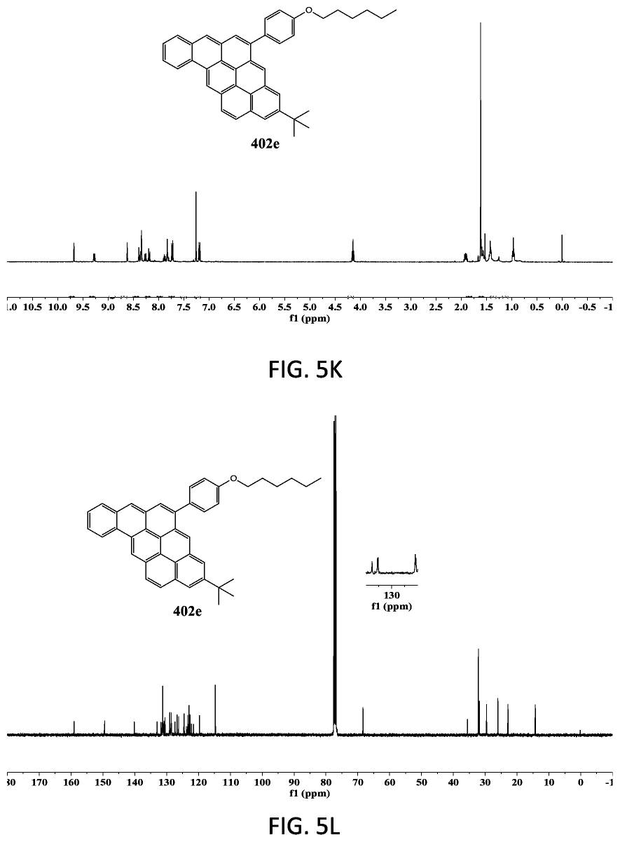

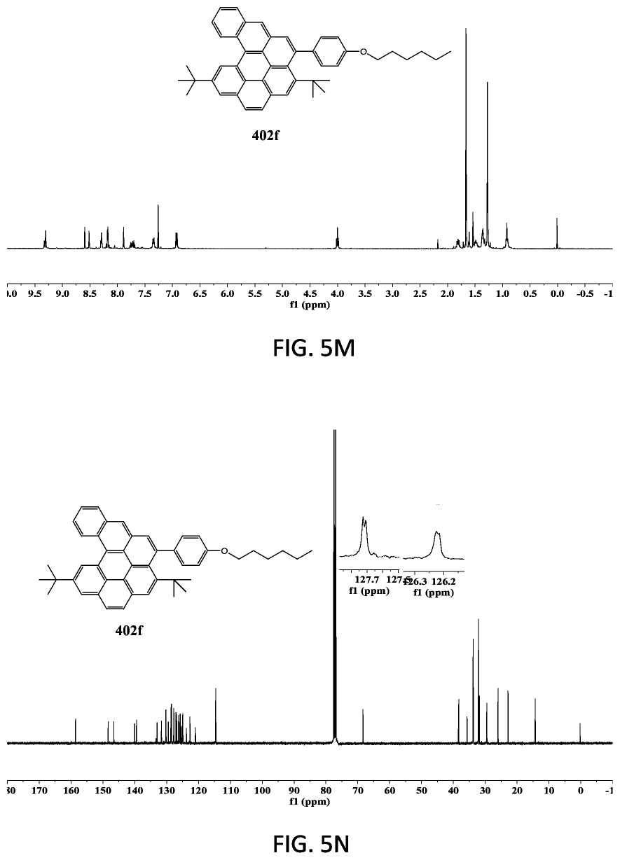

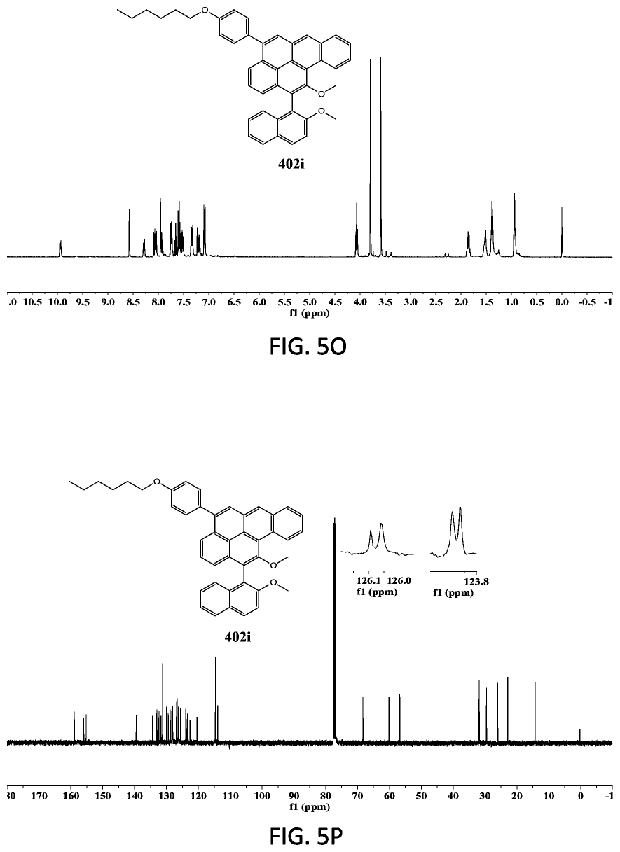

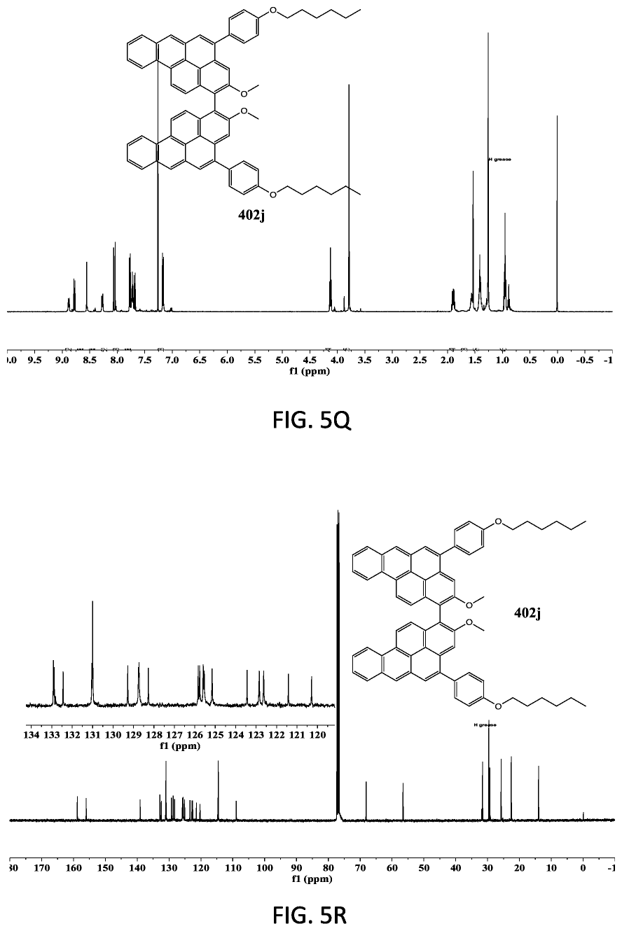

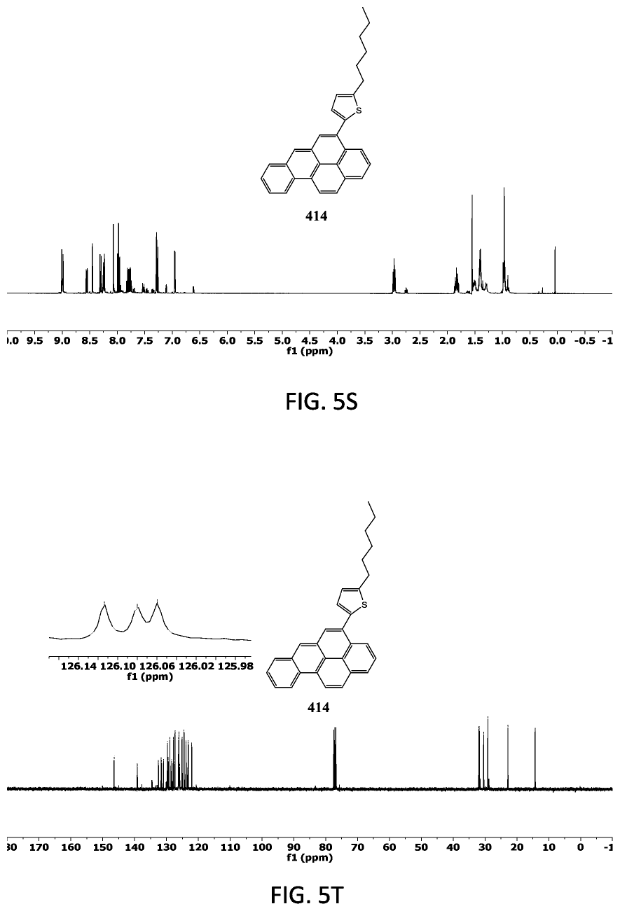

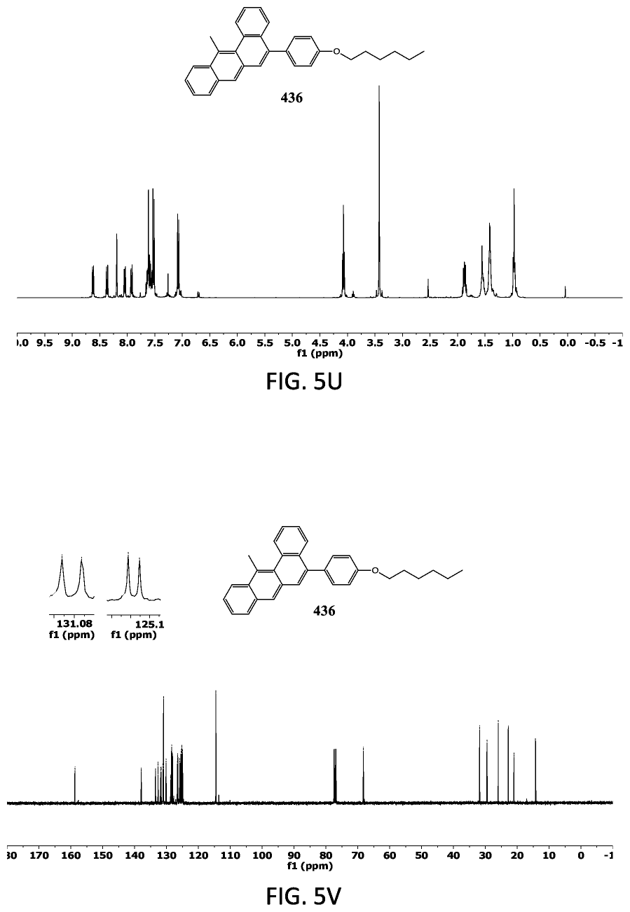

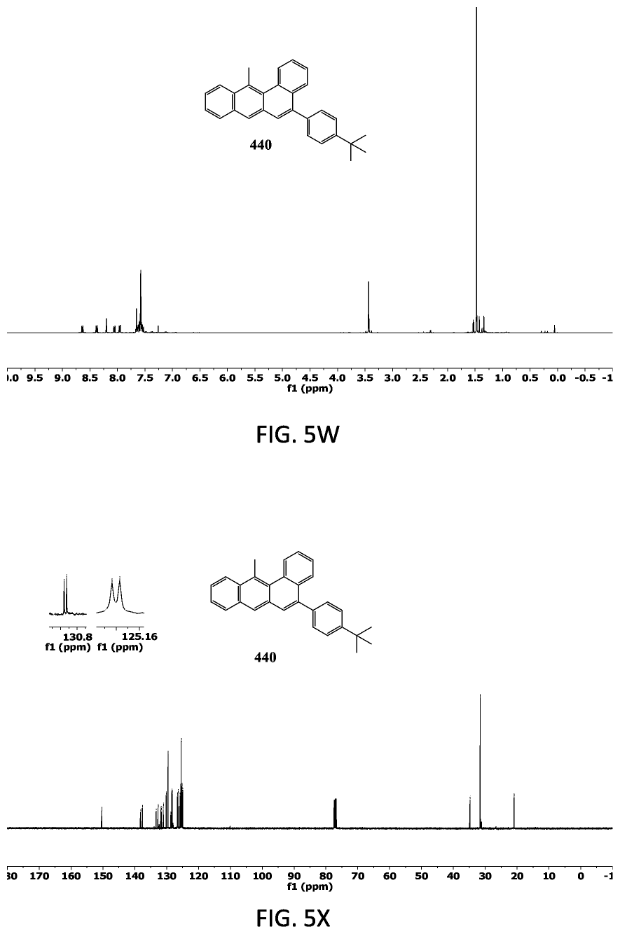

Compound 402′ was prepared following general procedure C, using compound 400′(47.0 mg, 0.100 mmol) as starting material. For example, in a nitrogen-filled glove box, precursor 400′ (0.10 mmol), InCl₃ (2.2 mg, 0.010 mmol), and AgNTf₂ (3.9 mg, 0.010 mmol) were dissolved in toluene (10 mL) in a sealed tube. The resulting mixture was stirred at 100° C. for 12 hour and then cooled down to room temperature. After evaporation of solvent, the residue was purified by silica gel column chromatography to yield corresponding annulated product 402′. Purification by flash column chromatography (silica gel, hexane:CH₂Cl₂=7:1, v/v) yielded pure compound 402′ as yellow solid (37.5 mg, 94%). R_{f}=0.25 (hexane:CH₂Cl₂=7:1); FTIR (neat) 3050, 2957, 2926, 2871, 1608, 1509, 1469, 1394, 1283, 1242, 1174, 1036, 900, 838, 757 cm⁻¹; ¹H NMR (400 MHz, CDCl₃) (FIG. 5A) δ 9.07 (m, 2H), 8.50 (s, 1H), 8.36 (d, J=9.1 Hz, 1H), 8.31-8.23 (m, 2H), 8.15 (d, J=7.7 Hz, 1H), 7.97-7.90 (m, 2H), 7.81 (m, 2H), 7.65-7.59 (m, 2H), 7.14-7.09 (m, 2H), 3.87 (d, J=6.5 Hz, 2H), 2.20 (m, 1H), 1.12 ppm (d, J=6.7 Hz, 6H); ¹³C NMR (100 MHz, CDCl₃) (FIG. 5B) δ 159.1, 139.5, 132.9, 131.73, 131.66, 131.6, 131.1, 129.8, 128.9, 128.4, 128.2, 127.8, 127.4, 126.2, 126.0, 125.94, 125.88, 125.8, 124.8, 124.1, 123.5, 123.2, 122.1, 114.6, 74.7, 28.5, 19.5 ppm; HRMS (ESI, positive) m/z calcd for C₃₀H₂₄O [M+Na]⁺ 423.1719, found 423.1737.

Compound 402a:

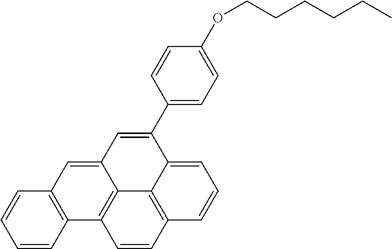 [SMILES: CCCCCCOc1ccc(-c2cc3cc4ccccc4c4ccc5cccc2c5c34)cc1]

Compound 402a was prepared following the general procedure C, using compound 400a (43.0 mg, 0.100 mmol) as starting material. Purification by flash column chromatography (silica gel, hexane:CH₂Cl₂=7:1, v/v) yielded pure 402a as yellow solid (39.0 mg, 91%). R_{f}=0.25 (hexane:CH₂Cl₂=7:1); FTIR (neat) 3053, 2949, 2925, 2855, 2200, 1604, 1508, 1467, 1287, 1245, 1172, 1023, 864, 759 cm⁻¹; ¹H NMR (500 MHz, CDCl₃) (FIG. 5C) δ 9.09-9.03 (m, 2H), 8.50 (s, 1H), 8.35 (d, J=9.1 Hz, 1H), 8.26 (m, 2H), 8.15 (d, J=7.6 Hz, 1H), 7.96-7.91 (m, 2H), 7.83 (m, 1H), 7.78 (m, 1H), 7.61 (d, J=8.6 Hz, 2H), 7.11 (d, J=8.6 Hz, 2H), 4.09 (t, J=6.5 Hz, 2H), 1.93-1.84 (m, 2H), 1.61-1.50 (m, 2H), 1.41 (t, J=5.4 Hz, 4H), 0.97 ppm (m, 3H); ¹³C NMR (100 MHz, CDCl₃) ((FIG. 5D)) δ 159.0, 139.5, 133.0, 131.75, 131.68, 131.6, 131.2, 129.8, 128.9, 128.4, 128.2, 127.9, 127.5, 126.2, 125.98, 125.96, 125.90, 125.86, 124.9, 124.1, 123.5, 123.2, 122.2, 114.6, 68.3, 31.8, 29.5, 26.0, 22.8, 14.2 ppm; HRMS (ESI, positive) m/z calcd for C₃₂H₂₈O [M+H]⁺ 429.2213, found 429.2227.

Compound 402b:

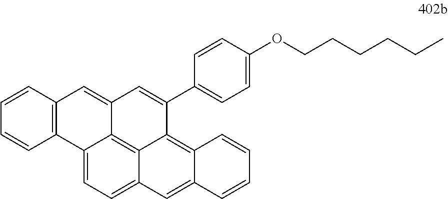 [SMILES: CCCCCCOc1ccc(-c2cc3cc4ccccc4c4ccc5cc6ccccc6c2c5c34)cc1]

Compound 402b was prepared following the general procedure C, using compound 400b (48.0 mg, 0.100 mmol) as starting material. Purification by flash column chromatography (silica gel, hexane:CH₂Cl₂=5:1, v/v) yielded pure compound 402b as yellow solid (42.0 mg, 88%). R_{f}=0.20 (hexane:CH₂Cl₂=4:1); FTIR (neat) 3057, 2953, 2927, 2857, 1647, 1604, 1549, 1510, 1464, 1341, 1275, 1242, 1175, 989, 886, 769 cm⁻¹; ¹H NMR (400 MHz, CDCl₃) (FIG. 5E) δ 9.01 (d, J=8.4 Hz, 1H), 8.94 (d, J=9.4 Hz, 1H), 8.69 (s, 1H), 8.58 (s, 1H), 8.33 (d, J=9.3 Hz, 1H), 8.31-8.20 (m, 2H), 8.15 (d, J=9.0 Hz, 1H), 8.13 (s, 1H), 7.85-7.71 (m, 2H), 7.62-7.55 (m, 1H), 7.47 (d, J=8.7 Hz, 2H), 7.29-7.26 (m, 1H), 7.06 (d, J=8.7 Hz, 2H), 4.10 (t, J=6.6 Hz, 2H), 1.96-1.83 (m, 2H), 1.63-1.48 (m, 2H), 1.48-1.32 (m, 4H), 1.05-0.88 ppm (m, 3H); ¹³C NMR (100 MHz, CDCl₃) (FIG. 5F) δ 158.6, 139.3, 138.1, 132.5, 131.84, 131.78, 130.1, 129.9, 129.6, 128.9, 128.8, 128.7, 128.6, 128.4, 128.0, 127.4, 127.2, 126.8, 126.1, 126.0, 125.8, 125.4, 124.7, 123.9, 123.7, 123.4, 122.2, 115.2, 68.4, 31.8, 29.5, 26.0, 22.8, 14.2 ppm; HRMS (ESI, positive) m/z calcd for C₃₆H₃₀O [M+H]⁺ 479.2369, found 479.2375.

Compound 402c:

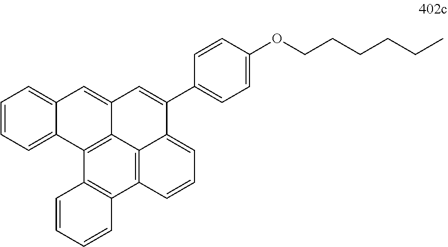 [SMILES: CCCCCCOc1ccc(-c2cc3cc4ccccc4c4c5ccccc5c5cccc2c5c34)cc1]

Compound 402c was prepared following the general procedure C, using compound 400c (48.0 mg, 0.100 mmol) as starting material. Purification by flash column chromatography (silica gel, hexane:CH₂Cl₂=5:1, v/v) yielded pure compound 402c as yellow solid (45.5 mg, 95%). R_{f}=0.20 (hexane:CH₂Cl₂=4:1); FTIR (neat) 3034, 2949, 2925, 2856, 1607, 1510, 1467, 1421, 1406, 1279, 1241, 1174, 1030, 899, 831, 763 cm⁻¹; ¹H NMR (400 MHz, CDCl₃) (FIG. 5G) δ 9.23 (m, 1H), 9.12 (dd, J=8.0, 1.5 Hz, 1H), 8.92 (d, J=7.5 Hz, 2H), 8.49 (s, 1H), 8.27 (m, 1H), 8.14 (dd, J=7.8, 0.9 Hz, 1H), 7.98-7.89 (m, 2H), 7.83-7.65 (m, 4H), 7.58 (d, J=8.6 Hz, 2H), 7.10 (d, J=8.5 Hz, 2H), 4.09 (t, J=6.5 Hz, 2H), 1.88 (m, 2H), 1.60-1.49 (m, 2H), 1.41 (m, 4H), 1.07-0.88 ppm (m, 3H); ¹³C NMR (100 MHz, CDCl₃) (FIG. 5H) δ 158.9, 139.0, 133.0, 132.9, 131.6, 131.5, 131.2, 130.1, 130.0, 129.7, 129.3, 128.6, 128.2, 128.0, 127.8, 127.0, 126.42, 126.40, 126.1, 125.9, 125.7, 125.4, 125.3, 125.1, 124.1, 123.5, 120.7, 114.6, 68.3, 31.8, 29.5, 26.0, 22.8, 14.2 ppm; HRMS (ESI, positive) m/z calcd for C₃₆H₃₀O [M+H]⁺ 479.2369, found 479.2379.

Compound 402d:

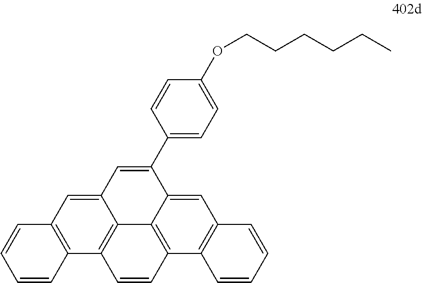 [SMILES: CCCCCCOc1ccc(-c2cc3cc4ccccc4c4ccc5c6ccccc6cc2c5c34)cc1]

Compound 402d was prepared following the general procedure C, using compound 400d (48.0 mg, 0.100 mmol) as starting material. Purification by flash column chromatography (silica gel, hexane:CH₂Cl₂=5:1, v/v) yielded pure compound 402d as yellow solid (41.0 mg, 86%). R_{f}=0.30 (hexane:CH₂Cl₂=4:1); FTIR (neat) 3057, 2928, 2857, 1737, 1607, 1509, 1469, 1283, 1244, 1175, 1025, 899 cm⁻¹; ¹H NMR (400 MHz, CDCl₃) (FIG. 5) δ 9.29-9.21 (m, 2H), 9.05 (dd, J=8.4, 4.3 Hz, 2H), 8.35 (s, 1H), 8.31 (s, 1H), 8.19-8.24 (m, 1H), 8.13-8.06 (m, 1H), 7.86-7.70 (m, 5H), 7.67-7.61 (m, 2H), 7.18-7.10 (m, 2H), 4.12 (t, J=6.6 Hz, 2H), 1.93-1.85 (m, 2H), 1.54-1.66 (m, 2H), 1.47-1.36 (m, 4H), 1.02-0.92 ppm (m, 3H). ¹³C NMR (100 MHz, CDCl₃) (FIG. 5J) δ 159.0, 139.8, 132.9, 131.9, 131.5, 131.1, 130.4, 130.3, 129.4, 128.80, 128.77, 128.7, 128.6, 128.4, 128.1, 126.64, 126.58, 126.55, 126.2, 125.2, 125.1, 124.7, 124.4, 123.2, 123.0, 122.6, 122.3, 114.7, 68.4, 31.8, 29.5, 26.0, 22.8, 14.3 pm. HRMS (ESI, positive) m/z calcd for C₃₆H₃₀O [M+H]⁺ 479.2369, found 479.2368.

Compound 402e:

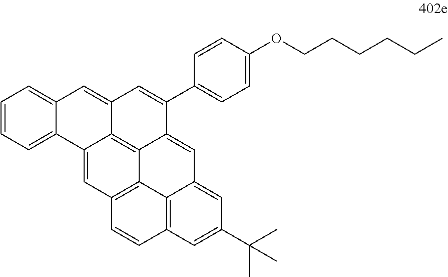 [SMILES: CCCCCCOc1ccc(-c2cc3cc4ccccc4c4cc5ccc6cc(C(C)(C)C)cc7cc2c(c34)c5c67)cc1]

Compound 402e was prepared following the general procedure C, using compound 400e (56.0 mg, 0.100 mmol) as starting material. Purification by flash column chromatography (silica gel, hexane:CH₂Cl₂=5:1, v/v) yielded pure compound 402e as yellow solid (34.0 mg, 61%). R_{f}=0.20 (hexane:CH₂Cl₂=4:1); FTIR (neat) 3033, 2951, 2929, 2859, 1607, 1583, 1510, 1496, 1467, 1390, 1281, 1241, 1174, 1028, 898, 882, 796 cm⁻¹; ¹H NMR (400 MHz, CDCl₃) (FIG. 5K) δ 9.68 (s, 1H), 9.28 (d, J=8.3 Hz, 1H), 8.63 (s, 1H), 8.45-8.31 (m, 4H), 8.26 (d, J=7.9 Hz, 1H), 8.19 (d, J=9.0 Hz, 1H), 7.88 (m, 1H), 7.86-7.79 (m, 2H), 7.79-7.66 (m, 2H), 7.24-7.14 (m, 2H), 4.21-4.09 (m, 2H), 1.92 (m, 2H), 1.62 (m, 11H), 1.49-1.38 (m, 4H), 1.03-0.93 ppm (m, 3H); ¹³C NMR (100 MHz, CDCl₃) (FIG. 5L) δ 159.1, 149.4, 140.2, 133.0, 131.8, 131.4, 131.3, 131.2, 130.8, 130.54, 130.52, 129.10, 129.07, 129.0, 128.6, 128.5, 127.4, 126.7, 126.2, 124.6, 124.5, 123.8, 123.4, 123.0, 122.7, 122.2, 121.5, 119.7, 114.7, 68.4, 35.6, 32.1, 31.8, 29.6, 26.0, 22.8, 14.3 ppm; HRMS (ESI, positive) m/z calcd for C₄₂H₃₈O [M+Na]⁺ 581.2815, found 581.2817.

Compound 402f:

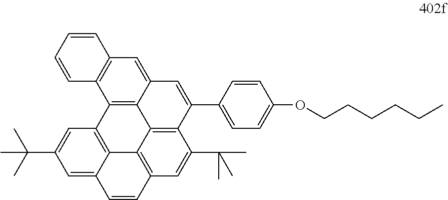 [SMILES: CCCCCCOc1ccc(-c2cc3cc4ccccc4c4c5cc(C(C)(C)C)cc6ccc7cc(C(C)(C)C)c2c(c7c65)c34)cc1]

Compound 402f was prepared following the general procedure C, using compound 400f (61.0 mg, 0.100 mmol) as starting material. Purification by flash column chromatography (silica gel, hexane:CH₂Cl₂=5:1, v/v) yielded pure compound 402f as yellow solid (36.0 mg, 59%). R_{f}=0.20 (hexane:CH₂Cl₂=4:1); FTIR (neat) 3031, 2955, 2929, 2866, 1605, 1510, 1469, 1276, 1244, 1175, 1030, 890, 751 cm⁻¹; ¹H NMR (400 MHz, CDCl₃) (FIG. 5M) δ 9.34-9.27 (m, 2H), 8.59 (s, 1H), 8.51 (s, 1H), 8.30 (d, J=4.7 Hz, 2H), 8.23-8.14 (m, 2H), 7.89 (s, 1H), 7.73 (m, 2H), 7.33 (m, 2H), 6.98-6.88 (m, 2H), 4.00 (t, J=6.6 Hz, 2H), 1.81 (m, 2H), 1.66 (s, 9H), 1.52-1.44 (m, 2H), 1.43-1.34 (m, 4H), 1.27 (s, 9H), 0.96-0.89 ppm (m, 3H); ¹³C NMR (100 MHz, CDCl₃) (FIG. 5N) δ 158.6, 148.3, 146.6, 140.0, 139.4, 133.3, 133.0, 131.6, 130.2, 129.5, 128.6, 128.6, 128.5, 128.4, 127.72, 127.71, 127.0, 126.9, 126.8, 126.222, 126.216, 125.8, 125.7, 125.4, 125.3, 124.9, 123.8, 122.7, 120.9, 114.6, 68.3, 38.2, 35.6, 33.7, 32.0, 31.8, 29.4, 25.9, 22.8, 14.2 ppm; HRMS (ESI, positive) m/z calcd for C₄₆H₄₆O [M+H]⁺ 615.3621, found 615.3622.

Compound 402i:

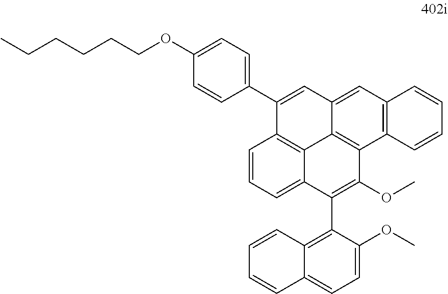 [SMILES: CCCCCCOc1ccc(-c2cc3cc4ccccc4c4c(OC)c(-c5c(OC)ccc6ccccc56)c5cccc2c5c34)cc1]

Compound 402i was prepared following the general procedure C, using compound 400i (61.0 mg, 0.100 mmol) as starting material. Purification by flash column chromatography (silica gel, hexane:CH₂Cl₂=5:1, v/v) yielded pure 402i as yellow solid (56.0 mg, 92%). R_{f}=0.20 (hexane:CH₂Cl₂=4:1); FTIR (neat) 3056, 3030, 2953, 2931, 2857, 1622, 1606, 1593, 1510, 1464, 1269, 1244, 1175, 1148, 1074, 1056, 1020, 833, 810, 763 cm⁻¹; ¹H NMR (400 MHz, CDCl₃) (FIG. 5O) δ 9.99-9.90 (m, 1H), 8.58 (s, 1H), 8.29 (m, 1H), 8.11-8.01 (m, 2H), 7.98-7.89 (m, 2H), 7.78-7.71 (m, 2H), 7.71-7.63 (m, 1H), 7.56 (m, 4H), 7.37-7.30 (m, 2H), 7.26-7.19 (m, 1H), 7.13-7.04 (m, 2H), 4.07 (m, 2H), 3.80 (s, 3H), 3.59 (s, 3H), 1.91-1.80 (m, 2H), 1.52 (m, 2H), 1.44-1.31 (m, 4H), 0.93 ppm (m, 3H); ¹³C NMR (100 MHz, CDCl₃) (FIG. 5P) δ 158.9, 155.9, 155.2, 139.5, 134.4, 133.0, 132.6, 132.3, 131.7, 131.2, 130.0, 129.9, 129.4, 128.8, 128.3, 128.2, 128.1, 128.0, 126.8, 126.7, 126.3, 126.2, 126.09, 126.06, 125.6, 123.90, 123.87, 123.7, 123.4, 122.6, 120.4, 114.6, 113.9, 68.3, 60.1, 56.7, 31.8, 29.5, 26.0, 22.8, 14.2 ppm; HRMS (ESI, positive) m/z calcd for C₄₄H₃₈O₃ [M+H]⁺ 615.2894, found 615.2894.

Compound 402j:

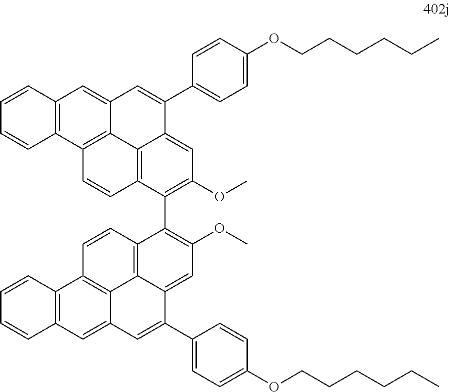 [SMILES: CCCCCCOc1ccc(-c2cc3cc4ccccc4c4ccc5c(-c6c(OC)cc7c(-c8ccc(OCCCCCC)cc8)cc8cc9ccccc9c9ccc6c7c89)c(OC)cc2c5c34)cc1]

Compound 402j was prepared following the general procedure C, using compound 400j (46.0 mg, 0.0500 mmol) as starting material. Purification by flash column chromatography (silica gel, hexane:CH₂Cl₂=2:1, v/v) yielded pure compound 402j as yellow solid (29 mg, 63%). R_{f}=0.40 (hexane:CH₂Cl₂=1:1); FTIR (neat) 2955, 2917, 2849, 1607, 1584, 1554, 1510, 1467, 1264, 1243, 1175, 1097, 1082, 833, 757 cm⁻¹; H NMR (500 MHz, CDCl₃) (FIG. 5Q) δ 8.91-8.85 (m, 2H), 8.78 (d, J=9.5 Hz, 2H), 8.56 (s, 2H), 8.29-8.24 (m, 2H), 8.05 (d, J=14.1 Hz, 4H), 7.80-7.67 (m, 10H), 7.20-7.14 (m, 4H), 4.13 (t, J=6.6 Hz, 4H), 3.79 (s, 6H), 1.94-1.86 (m, 4H), 1.56 (m, 4H), 1.45-1.37 (m, 8H), 0.98-0.92 ppm (m, 6H); ¹³C NMR (100 MHz, CDCl₃) (FIG. 5R) δ 158.8, 156.0, 139.1, 132.92, 132.88, 132.4, 131.04, 131.00, 129.3, 128.8, 128.7, 128.3, 125.84, 125.76, 125.6, 125.5, 125.2, 123.4, 122.9, 122.6, 121.4, 120.3, 114.6, 108.9, 68.2, 56.6, 31.7, 29.4, 25.8, 22.6, 14.1 ppm; MALDI-TOF calcd for C₆₆H₅₈O₄ [M+H]⁺ 915.441, found 915.503.

Compound 400h′:

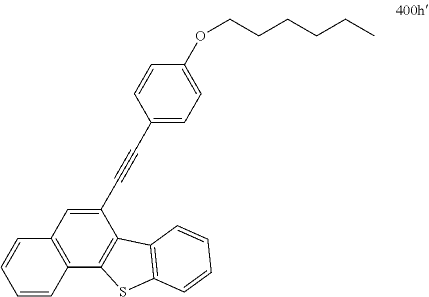 [SMILES: CCCCCCOc1ccc(C#Cc2cc3ccccc3c3sc4ccccc4c23)cc1]

Compound 400h′ was prepared following general procedure C, using compound 400h (43.0 mg, 0.100 mmol) as staring material in DCE with InCl₃ (2.2 mg, 0.0100 mmol). Purification by flash column chromatography (silica gel, hexane:CH₂Cl₂=8:1, v/v) yielded pure compound 400h′ as yellow solid (37.5 mg, 87%). R_{f}=0.4 (hexane:CH₂Cl₂=6:1); FTIR (neat) 3047, 2952, 2923, 2851, 2205, 1605, 1510, 1287, 1250, 1174, 1025, 878, 829, 751 cm⁻¹; ¹H NMR (400 MHz, CDCl₃) δ 9.38-9.24 (m, 1H), 8.20-8.07 (m, 2H), 8.02-7.86 (m, 2H), 7.73-7.44 (m, 6H), 7.03-6.92 (m, 2H), 4.02 (t, J=6.6 Hz, 2H), 1.83 (m, 2H), 1.54-1.43 (m, 2H), 1.42-1.29 (m, 4H), 1.00-0.86 ppm (m, 3H); ¹³C NMR (100 MHz, CDCl₃) δ 159.7, 139.2, 138.3, 136.9, 133.1, 131.3, 131.1, 130.7, 128.5, 127.4, 126.9, 126.2, 124.6, 124.5, 124.4, 122.7, 117.0, 115.2, 114.9, 110.2, 94.5, 87.8, 68.3, 31.7, 29.3, 25.9, 22.8, 14.2 ppm; HRMS (ESI, positive) m/z calcd for C₃₀H₂₆OS [M+H]⁺ 435.1777, found 435.1800.

Compound 414:

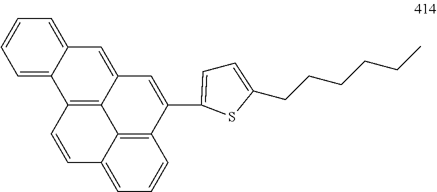 [SMILES: CCCCCCc1ccc(-c2cc3cc4ccccc4c4ccc5cccc2c5c34)s1]

Compound 414 was prepared following general procedure C, using compound 412 (42.0 mg, 0.100 mmol) as starting material. Purification by flash column chromatography (silica gel, hexane:CH₂Cl₂=15:1, v/v) yielded pure compound 414 as yellow solid (34.5 mg, 82%). R_{f}=0.3 (hexane:CH₂Cl₂=15:1); FTIR (neat) 3052, 2952, 2922, 2851, 1619, 1598, 1465, 1413, 1376, 1275, 1221, 1160, 1033, 945, 897 cm⁻¹; ¹H NMR (400 MHz, CDCl₃) (FIG. 5S) δ 9.07-8.94 (m, 2H), 8.55 (dd, J=7.7, 1.1 Hz, 1H), 8.45 (s, 1H), 8.30 (d, J=9.1 Hz, 1H), 8.28-8.22 (m, 2H), 8.07 (s, 1H), 7.94-8.01 (m, 1H), 7.83-7.73 (m, 2H), 7.29 (d, J=3.4 Hz, 1H), 6.95 (dt, J=3.4, 1.0 Hz, 1H), 3.01-2.91 (m, 2H), 1.88-1.79 (m, 2H), 1.53-1.36 (m, 6H), 1.00-0.92 ppm (m, 3H); ¹³C NMR (100 MHz, CDCl₃) (FIG. 5T) δ 146.4, 139.2, 132.5, 131.7, 131.6, 131.0, 129.7, 129.4, 128.9, 128.4, 127.8, 127.4, 127.3, 126.2, 126.11, 126.08, 126.06, 125.9, 125.1, 124.5, 123.9, 123.3, 123.2, 122.1, 31.9, 31.8, 30.4, 29.1, 22.8, 14.3 ppm; HRMS (ESI, positive) m/z calcd for C₃₀H₂₆S [M+H]⁺ 419.1828, found 419.1824.

Compound 424:

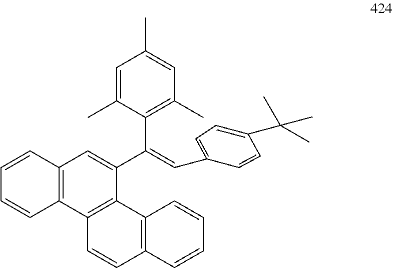 [SMILES: Cc1cc(C)c(/C(=C\c2ccc(C(C)(C)C)cc2)c2cc3ccccc3c3ccc4ccccc4c23)c(C)c1]

Compound 424 was prepared following the general procedure C, using compound 420 (38.0 mg, 0.100 mmol) as a starting material in mesitylene. Purification by flash column chromatography (silica gel, hexane:CH₂Cl₂=16:1, v/v) yielded pure 424 as sticky yellow solid (34.0 mg, 68%). R_{f}=0.20 (hexane:CH₂Cl₂=16:1); FTIR (neat) 3058, 2959, 2917, 2849, 1607, 1507, 1474, 1460, 1360, 1261, 1248, 1149, 1109, 1015, 950, 904, 887, 813, 761 cm⁻¹; ¹H NMR (500 MHz, CDCl₃) δ 9.69 (d, J=8.4 Hz, 1H), 8.70 (d, J=8.4 Hz, 1H), 8.61 (d, J=8.9 Hz, 1H), 7.78 (d, J=7.9 Hz, 1H), 7.73-7.70 (m, 1H), 7.68 (s, 1H), 7.65 (dd, J=8.4, 6.9 Hz, 1H), 7.61-7.58 (m, 1H), 7.55 (dd, J=8.0, 6.9 Hz, 1H), 7.36-7.27 (m, 2H), 7.00 (s, 2H), 6.61 (s, 1H), 6.60-6.50 (m, 2H), 6.48-6.37 (m, 2H), 2.42 (s, 6H), 2.36 (s, 3H), 1.05 ppm (s, 9H); ¹³C NMR (125 MHz, CDCl₃) δ 149.0, 140.8, 139.5, 138.0, 136.7, 136.5, 134.6, 134.5, 132.7, 131.9, 130.7, 130.5, 130.0, 129.8, 129.8, 129.1, 128.8, 128.2, 127.9, 127.8, 127.7, 126.8, 126.5, 125.6, 124.4, 123.6, 123.3, 120.9, 34.3, 31.1, 22.4, 21.1 ppm; HRMS (ESI, positive) m/z calcd for C₃₉H₃₆ [M+H]⁺ 505.2890, found 505.2895.

Compound 436:

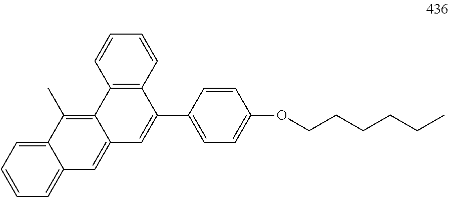 [SMILES: CCCCCCOc1ccc(-c2cc3cc4ccccc4c(C)c3c3ccccc23)cc1]

Compound 436 was prepared following the general procedure C, using compound 434 (42.0 mg, 0.100 mmol) as starting material. Purification by flash column chromatography (silica gel, hexane:CH₂Cl₂=10:1, v/v) yielded pure compound 436 as yellow sticky oil (35.0 mg, 84%). R_{f}=0.20 (hexane:CH₂Cl₂=10:1); FTIR (neat) 3064, 2927, 2857, 1607, 1509, 1495, 1467, 1377, 1240, 1175, 1010, 897 cm⁻¹; ¹H NMR (400 MHz, CDCl₃) (FIG. 5U) δ 8.62 (d, J=8.3 Hz, 1H), 8.37 (d, J=8.6 Hz, 1H), 8.19 (s, 1H), 8.11-7.98 (m, 1H), 7.92 (d, J=8.0 Hz, 1H), 7.81-7.31 (m, 7H), 7.13-7.01 (m, 2H), 4.07 (t, J=6.5 Hz, 2H), 3.42 (s, 3H), 1.94-1.80 (m, 2H), 1.63-1.31 (m, 6H), 1.06-0.89 ppm (m, 3H); ¹³C NMR (100 MHz, CDCl₃) (FIG. 5V) δ 158.8, 138.0, 133.5, 132.7, 132.7, 131.8, 131.7, 131.10, 131.07, 131.0, 130.2, 128.7, 128.4, 128.1, 126.6, 126.5, 125.8, 125.5, 125.21, 125.15, 125.0, 114.5, 68.2, 31.8, 29.5, 26.0, 22.8, 21.0, 14.2 ppm; HRMS (ESI, positive) m/z calcd for C₃₁H₃₀O [M+H]⁺ 419.2369, found 419.2356.

Compound 440:

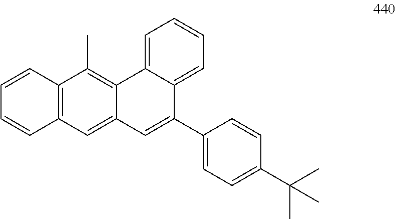 [SMILES: Cc1c2ccccc2cc2cc(-c3ccc(C(C)(C)C)cc3)c3ccccc3c12]

Compound 440 was prepared following the general procedure C, using compound 438 (37.0 mg, 0.100 mmol) as starting material. Purification by flash column chromatography (silica gel, hexane:CH₂Cl₂=20:1, v/v) yielded pure compound 440 as yellow sticky oil (30.0 mg, 82%). R_{f}=0.40 (hexane:CH₂Cl₂=10:1); FTIR (neat) 3066, 2961, 2867, 1509, 1496, 1476, 1449, 1379, 1362, 1268, 1117, 1022, 898 cm⁻¹; H NMR (400 MHz, CDCl₃) (FIG. 5W) δ 8.64 (d, J=8.3 Hz, 1H), 8.38 (d, J=8.7 Hz, 1H), 8.20 (s, 1H), 8.06 (d, J=8.1 Hz, 1H), 7.96 (dd, J=8.0, 1.5 Hz, 1H), 7.70-7.50 (m, 9H), 3.44 (s, 3H), 1.54-1.43 ppm (m, 9H); ¹³C NMR (100 MHz, CDCl₃) (FIG. 5X) δ 150.4, 138.2, 137.6, 133.4, 132.7, 131.8, 131.7, 131.12, 131.06, 130.1, 129.6, 128.7, 128.4, 128.3, 126.7, 126.5, 125.8, 125.5(2), 125.24, 125.22, 125.0, 34.8, 31.6, 21.0 ppm; HRMS (ESI, positive) m/z calcd for C₂₉H₂₆ [M+H]⁺ 375.2107, found 375.2098.

X-Ray Crystallographic Analysis

Crystallographic data for 400′: C₃₀H₂₄O; Mr=400.49; crystal size=0.062×0.049×0.041 mm³; triclinic; space group P-1; a=5.6239(3), b=11.0760(5), c=17.2918(8) Å; α=77.4740(10°), β=88.9730(10°), γ=83.4970(10°); V=1044.68(9) Å³; Z=2, ρ_{calcd}=1.273 Mg/m³; μ=0.075 mm⁻¹; λ=0.71073 Å; T=100(2) K; 2θₘₐₓ=50.00°; reflections measured 22510, independent 5038 [R(int)=0.0405]; R₁=0.0451, wR₂=0.1140 (I>2σ(I)); residual electron density=0.373 and −0.246 eÅ⁻³.

Crystallographic data for 402c: C₃₆H₃₀O; Mr=478.60; crystal size=0.123×0.051×0.019 mm³; triclinic; space group P-1; a=9.3672(18), b=10.255(2), c=13.408(3) Å; α=100.699(4°), β=93.795(4°), γ=103.928(4°); V=1220.1(4) Å³; Z=2, ρ_{calcd}=1.303 Mg/m³; μ=0.076 mm⁻¹; λ=0.71073 Å; T=100(2) K; 2θₘₐₓ=50.00°; reflections measured 21655, independent 4469 [R(int)=0.0807]; R₁=0.0541, wR₂=0.1209 (I>2σ(I)); residual electron density=0.397 and −0.215 eÅ⁻³.

Crystallographic data for 402d: C₇₂H₆₀O₂; Mr=957.20; crystal size=0.162×0.065×0.046 mm³; triclinic; space group P-1; a=9.5302(7), b=15.6426(12), c=18.1126(14) Å; α=105.9010(10°), β=104.9150(10°), γ=97.0170(10°); V=2454.8(3) Å³; Z=2, ρ_{calcd}=1.295 Mg/m³; μ=0.076 mm⁻¹; λ=0.71073 Å; T=100(2) K; 2θₘₐₓ=50.00°; reflections measured 41633, independent 8646 [R(int)=0.0587]; R₁=0.0456, wR₂=0.1023 (I>2σ(I)); residual electron density=0.201 and −0.237 eÅ⁻³.

Crystallographic data for 402e: C₄₂H₃₈O; Mr=558.72; crystal size=0.081×0.038×0.034 mm³; triclinic; space group P-1; a=10.2031(8), b=11.2183(9), c=14.4925(12) Å; α=99.686(2°), β=100.203(2°), γ=105.446(2°); V=1532.5(2) Å³; Z=2, ρ_{calcd}=1.211 Mg/m³; μ=0.070 mm⁻¹; λ=0.71073 Å; T=100(2) K; 2θₘₐₓ=50.00°; reflections measured 26678, independent 5415 [R(int)=0.0965]; R₁=0.0549, wR₂=0.1055 (I>2σ(I)); residual electron density=0.181 and −0.242 eÅ⁻³.

Crystallographic data for 438: C₂₉H₂₆; Mr=374.50; crystal size=0.136×0.072×0.053 mm³; monoclinic; space group P2₁/c; a=13.695(3), b=11.301(2), c=13.800(3) Å; α=90°, β=107.150(3°), γ=90°; V=2040.9(6) Å³; Z=4, ρ_{calcd}=1.219 Mg/m³; μ=0.069 mm⁻¹; λ=0.71073 A; T=100(2) K; 2θₘₐₓ=50.00°; reflections measured 26142, independent 2672 [R(int)=0.1159]; R₁=0.0481, wR₂=0.1121 (I>2σ(I)); residual electron density=0.279 and −0.197 eÅ⁻³.

Crystallographic data for 400h′: C₃₀H₂₆OS; Mr=434.57; crystal size=0.135×0.048×0.043 mm³; orthorhombic; space group Pbca; a=13.5759(5), b=12.7385(5), c=25.7233(10) Å; α=90°, β=90°, γ=90°; V=4448.5(3) Å³; Z=8, ρ_{calcd}=1.298 Mg/m³; μ=0.166 mm⁻¹; λ=0.71073 A; T=100(2) K; 2θₘₐₓ=50.00°; reflections measured 88009, independent 5107 [R(int)=0.0855]; R₁=0.0439, wR₂=0.1076 (I>2σ(I)); residual electron density=0.338 and −0.380 eÅ⁻³.

Crystallographic data for 424: C₃₉H₃₆; Mr=504.68; crystal size=0.125×0.037×0.033 mm³; orthorhombic; space group Pbca; a=18.263(9), b=9.068(4), c=35.036(17) Å; α=90°, β=90°, γ=90°; V=5802(5) Å³; Z=8, ρ_{calcd}=1.155 Mg/m³; μ=0.065 mm⁻¹; λ=0.71073 A; T=100(2) K; 2θₘₐₓ=50.00°; reflections measured 92787, independent 5027 [R(int)=0.1486]; R₁=0.0484, wR₂=0.1086 (I>2σ(I)); residual electron density=0.163 and −0.208 eÅ⁻³.

Additional compound embodiments and method of making the same are described below:

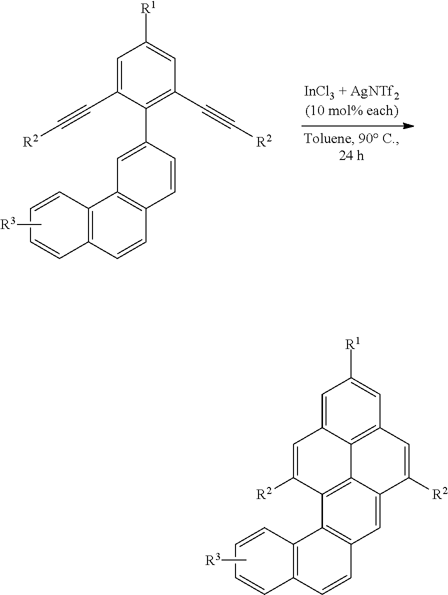

A flame-dried round-bottom flask was charged with a magnetic stirring bar, the corresponding precursors (1 equivalent)—obtained from combining a diyene pinacol borane starting material as described herein and 3-bromophenanthrene (or a substituted derivative thereof)—and anhydrous toluene (25 mL). 10 mol % each of InCl₃ and silver bistriflimide were added to the solution inside the glovebox after degassing the mixture via bubbling nitrogen for 30 min, then the resulting mixture was heated at 100° C. under a N₂ atmosphere. After reaction completion, the solvent was removed under reduced pressure and the residue was purified by column chromatography.

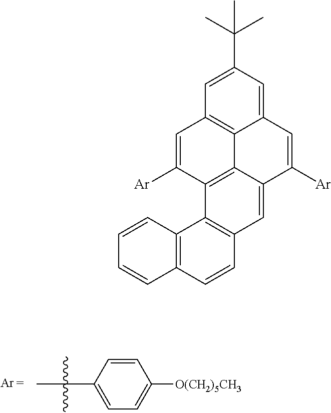

Rf=0.3 (hexane:DCM 2:1). FTIR (neat) 2953.07, 2929.20, 1605.23, 1490.28, 1095.26, 826.77 cm-1. ¹H NMR (400 MHz, chloroform-d) δ 8.61 (s, 1H), 8.36 (d, J=15.8 Hz, 2H), 8.27 (s, 1H), 8.15 (d, J=8.1 Hz, 1H), 8.01 (s, 1H), 7.95 (d, J=8.7 Hz, 1H), 7.84 (d, J=8.7 Hz, 1H), 7.72 (m, J=17.9, 7.6 Hz, 4H), 7.30 (m, J=7.5 Hz, 1H), 7.16 (d, J=7.0 Hz, 3H), 7.05 (m, J=7.6 Hz, 1H), 6.95-5.66 (m, 3H), 4.13 (t, J=6.5 Hz, 2H), 3.83 (q, J=6.2, 5.6 Hz, 2H), 1.91 (m, J=7.5 Hz, 2H), 1.73-1.69 (m, 2H), 1.64 (s, 9H), 1.52-1.30 (m, 12H), 1.02-0.92 (m, 6H). ¹³C NMR (101 MHz, cdcl₃) δ 158.9, 158.9, 157.8, 149.8, 139.4, 139.4, 136.7, 133.4, 131.3, 131.3, 131.3, 131.2, 131.2, 131.1, 131.0, 130.6, 130.5, 130.4, 130.3, 129.6, 129.5, 129.5, 129.2, 129.2, 127.9, 127.5, 126.8, 126.7, 126.1, 125.6, 125.6, 125.1, 125.1, 125.0, 124.3, 124.3, 124.0, 122.9, 122.8, 122.2, 122.1, 121.9, 114.6, 114.6, 68.3, 68.3, 68.1, 35.4, 32.0, 32.0, 31.8, 31.8, 31.7, 31.7, 29.5, 29.5, 29.2, 29.2, 26.0, 26.0, 25.8, 25.7, 22.8, 22.8, 22.8, 22.7, 14.3, 14.2. Mass calculated for [C52H54O2]+710.2124, found 610.2124.

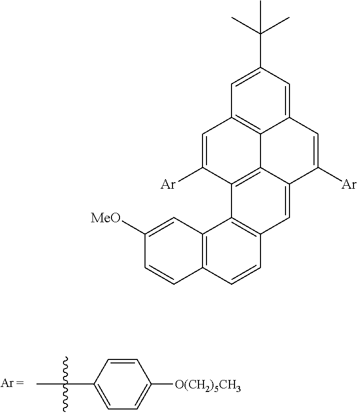

Rf=0.33 (hexane:DCM 1:1). FTIR (neat) 2953.07, 1608.66, 12043.31, 1174.56, 830.41 cm-1. ¹H NMR (400 MHz, chloroform-d) δ 8.53 (s, 1H), 8.32 (d, J=8.3 Hz, 2H), 8.22 (s, 1H), 7.96 (s, 1H), 7.82-7.76 (m, 2H), 7.67 (m, J=8.5, 3.1 Hz, 3H), 7.57 (d, J=2.5 Hz, 1H), 7.18-7.12 (m, 2H), 6.94 (m, 1H), 6.57 (s, 4H), 4.16-4.10 (m, 2H), 3.88-3.75 (m, 5H), 1.90 (p, J=6.6 Hz, 2H), 1.74-1.67 (m, 2H), 1.62 (s, 9H), 1.49-1.28 (m, 12H), 0.99-0.87 (m, 6H). ¹³C NMR (101 MHz, cdcl₃) δ 159.0, 157.9, 156.6, 149.8, 139.5, 139.0, 136.5, 133.5, 131.4, 131.2, 131.1, 131.1, 130.8, 130.3, 129.6, 129.2, 127.9, 127.8, 127.1, 126.5, 126.4, 125.0, 124.8, 124.5, 124.0, 122.9, 122.1, 121.9, 116.0, 114.7, 112.8, 68.4, 68.2, 55.2, 35.4, 32.1, 31.8, 31.8, 29.6, 29.3, 26.0, 25.8, 22.8, 22.8, 14.2, 14.2. APPI HRMS calcd for [C53H56O3]+ 640.4229, found 640.4225.

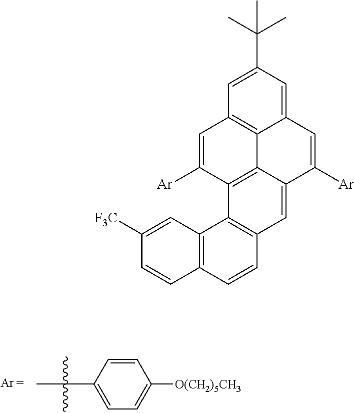

Rf=0.3 (hexane:EtOAc 1:20). FTIR (neat) 2952.85, 1607.74, 1508.96, 1242.41, 1119.53, 831.16 cm-1. ¹H NMR (400 MHz, Chloroform-d) δ 8.60 (s, 1H), 8.45-8.35 (m, 3H), 8.28 (s, 1H), 8.08-8.00 (m, 2H), 7.84 (m, J=8.6, 3.4 Hz, 2H), 7.68 (d, J=6.7 Hz, 2H), 7.46 (d, J=8.4 Hz, 1H), 7.16 (d, J=7.3 Hz, 2H), 6.12 (s, 4H), 4.13 (t, J=6.5 Hz, 2H), 3.86 (m, J=6.2 Hz, 2H), 1.96-1.87 (m, 2H), 1.71 (t, J=7.2 Hz, 2H), 1.65 (d, J=1.3 Hz, 9H), 1.56-1.28 (m, 12H), 1.02-0.90 (m, 6H). ¹³C NMR (101 MHz, cdcl₃) δ 159.0, 158.0, 150.3, 139.3, 139.0, 135.8, 133.2, 132.9, 131.3, 131.3, 131.1, 131.0, 130.5, 130.2, 129.7, 129.4, 129.3, 129.2, 129.12, 129.0, 128.7, 128.5, 128.4, 128.1, 128.0, 128.0, 127.8, 127.2, 126.9, 126.6, 126.2, 126.0, 125.9, 125.6, 125.3, 125.2, 124.8, 124.0, 123.3, 122.6, 121.7, 121.2, 114.7, 68.3, 68.2, 35.5, 32.0, 31.8, 31.7, 29.5, 29.2, 26.0, 25.8, 22.8, 22.8, 14.2, 14.2. APPI HRMS calcd for [C53H53F3O2]+ 778.3998, Found 778.3998.

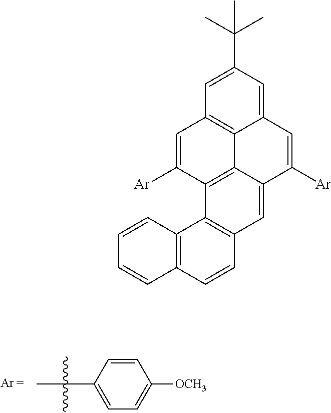

Rf=0.3 (hexane:DCM 1:1). FTIR (neat) 2959.96, 1607.97, 1463.45, 1175.78, 1037.18, 832.33 cm-1. ¹H NMR (400 MHz, chloroform-d) δ 8.57 (s, 1H), 8.36 (d, J=1.9 Hz, 1H), 8.32 (s, 1H), 8.25 (d, J=1.8 Hz, 1H), 8.13 (m, J=8.3, 1.3, 0.7 Hz, 1H), 7.99 (s, 1H), 7.94 (d, J=8.7 Hz, 1H), 7.86-7.80 (m, 1H), 7.76-7.67 (m, 3H), 7.29 (m, J=8.0, 7.0, 1.2 Hz, 1H), 7.19-7.15 (m, 2H), 7.04 (m, J=8.3, 7.0, 1.4 Hz, 1H), 6.90-6.15 (m, 4H), 3.98 (s, 2H), 3.69 (s, 2H), 1.64 (s, 9H). ¹³C NMR (400 MHz, cdcl₃) δ 159.4, 158.3, 149.9, 139.4, 139.3, 136.9, 133.7, 131.4, 131.3, 131.2, 131.1, 130.6, 130.4, 129.7, 129.5, 129.2, 128.0, 127.5, 126.9, 126.7, 126.2, 125.7, 125.1, 125.0, 124.4, 124.0, 122.9, 122.2, 121.9, 114.1, 55.6, 55.4, 35.4, 32.0. APPI HRMS calcd for [C42H34O2]+570.2559, found 570.2567.

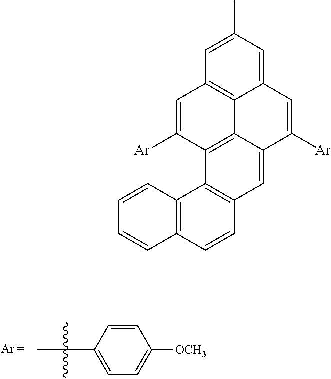

Rf=0.32 (hexane:DCM 1:1). FTIR (neat) 2926.20, 1739.00, 1620.04, 1506.24, 1218.38, 1168.89, 827.98 cm-1. ¹H NMR (400 MHz, chloroform-d) δ 8.56 (s, 1H), 8.23 (s, 1H), 8.12 (s, 2H), 8.00 (s, 1H), 7.96-7.90 (m, 2H), 7.83 (d, J=8.7 Hz, 1H), 7.71 (m, J=16.4, 7.5 Hz, 3H), 7.31-7.26 (m, 1H), 7.16 (d, J=8.8 Hz, 2H), 7.03 (m, J=8.3, 7.0, 1.4 Hz, 1H), 6.85-6.12 (m, 4H), 3.98 (s, 2H), 3.68 (s, 2H), 2.84 (s, 3H). ¹³C NMR (400 MHz, cdcl₃) δ 159.4, 158.3, 139.5, 139.4, 136.9, 136.5, 133.7, 131.4, 131.4, 131.4, 131.3, 130.3, 130.1, 129.7, 129.4, 129.2, 127.5, 126.8, 126.3, 126.2, 125.8, 125.7, 125.2, 124.9, 124.4, 124.1, 121.9, 114.2, 55.6, 55.4, 22.2. APPI HRMS calcd for [C39H28O2]+ 528.2089, found 528.2114.

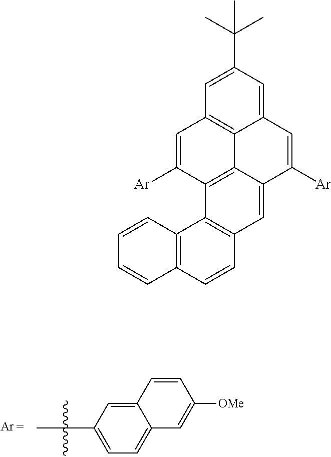

Rf=0.26 (hexane:DCM 1:1). FTIR(neat) 3026.27, 2919.55, 1604.32, 1495.09, 1246.86, 1029.81 cm-1 ¹H NMR (500 MHz, Chloroform-d) δ 8.62 (s, 1H), 8.48 (s, 1H), 8.42 (d, J=1.8 Hz, 1H), 8.30 (d, J=1.8 Hz, 1H), 8.22-8.18 (m, 2H), 8.11 (s, 1H), 7.99 (d, J=8.4 Hz, 1H), 7.93-7.85 (m, 4H), 7.81 (d, J=8.7 Hz, 1H), 7.63 (d, J=7.9 Hz, 2H), 7.34 (d, J=2.6 Hz, 1H), 7.29 (m, J=8.8, 2.5 Hz, 1H), 7.06 (m, J=7.1 Hz, 2H), 6.95 (d, J=13.1 Hz, 2H), 6.83 (s, 2H), 4.03 (s, 3H), 3.88 (s, 3H), 1.65 (s, 9H). ¹³C NMR (126 MHz, cdcl₃) δ 158.2, 157.5, 150.0, 139.8, 139.8, 136.7, 134.2, 133.2, 131.4, 131.3, 131.1, 130.4, 129.9, 129.6, 129.4, 129.3, 129.2, 129.2, 128.8, 128.1, 127.6, 127.6, 126.9, 126.8, 126.1, 125.7, 125.3, 125.0, 124.2, 124.08, 123.2, 122.5, 122.1, 119.4, 118.6, 106.0, 105.6, 55.6, 55.3, 35.5, 32.1. APPI HRMS calculated for [C50H38O2]+670.2872, found 670.2874.

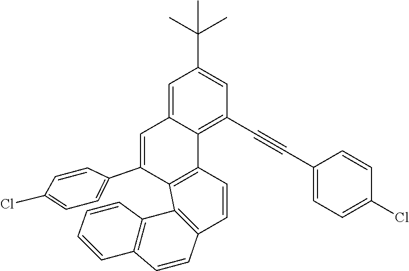 [SMILES: CC(C)(C)c1cc(C#Cc2ccc(Cl)cc2)c2c(c1)cc(-c1ccc(Cl)cc1)c1c2ccc2ccc3ccccc3c21]

Rf=0.37 (hexane:DCM 4:1). ¹H NMR (400 MHz, chloroform-d) δ 10.07 (d, J=8.7 Hz, 1H), 8.08 (s, 1H), 8.00 (d, J=10.5 Hz, 2H), 7.90 (d, J=8.4 Hz, 2H), 7.84 (m, J=9.5, 5.7 Hz, 3H), 7.71 (d, J=7.9 Hz, 1H), 7.61 (d, J=8.3 Hz, 2H), 7.40 (d, J=8.1 Hz, 2H), 7.26 (d, J=14.6 Hz, 2H), 7.00 (m, J=7.6 Hz, 4H), 1.53 (s, 9H). ¹³C NMR (101 MHz, cdcl₃) δ 149.1, 142.4, 138.8, 134.6, 133.3, 133.2, 132.8, 132.8, 132.2, 131.6, 131.4, 130.5, 130.4, 130.1, 129.7, 129.0, 128.9, 128.3, 128.1, 127.5, 127.01, 127.0, 126.8, 125.8, 125.6, 125.6, 125.3, 125.2, 124.8, 124.0, 122.3, 119.0, 93.4, 92.9, 34.9, 31.4. APPI HRMS mass calculated for [C40H28Cl2]+ 578.1568, found 578.1574.

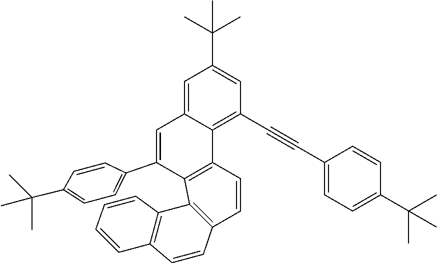 [SMILES: CC(C)(C)c1ccc(C#Cc2cc(C(C)(C)C)cc3cc(-c4ccc(C(C)(C)C)cc4)c4c(ccc5ccc6ccccc6c54)c23)cc1]

Rf=0.31 (hexane:EtOAc 20:1). IR(neat) 3026.23, 2919.20, 1604.23, 1495.00, 1459.43, 1080.95, 1029.77 cm-1. ¹H NMR (400 MHz, chloroform-d) δ 10.22 (d, J=8.4 Hz, 1H), 8.10 (s, 1H), 8.02 (d, J=8.7 Hz, 2H), 7.93 (s, 1H), 7.91-7.80 (m, 4H), 7.72-7.61 (m, 3H), 7.48 (m, J=8.3, 1.8 Hz, 2H), 7.21-7.12 (m, 1H), 7.11-6.78 (m, 4H), 1.55 (s, 9H), 1.39 (s, 9H), 1.19 (s, 9H). ¹³C NMR (101 MHz, cdcl₃) δ 151.8, 149.3, 148.8, 140.8, 139.8, 133.0, 132.9, 132.3, 131.3, 131.3, 130.6, 130.1, 129.9, 128.8, 127.8, 127.7, 127.4, 127.2, 126.3, 125.7, 125.5, 125.3, 125.1, 125.1, 124.4, 121.0, 119.5, 94.2, 92.0, 35.0, 34.8, 34.4, 31.5, 31.4, 31.3. APPI HRMS calcd for [C48H46]+ 622.36, found 622.3601.

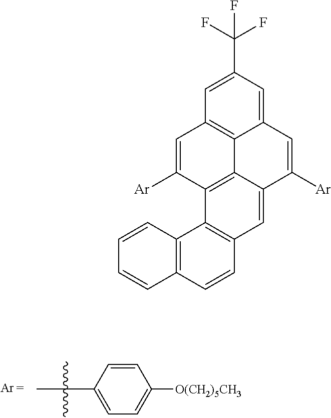

Rf=0.07 (hexane:DCM 1:4). ¹H NMR (400 MHz, chloroform-d) δ 8.65 (s, 1H), 8.52 (s, 1H), 8.38-8.29 (m, 2H), 8.10 (d, J=7.8 Hz, 1H), 8.00 (d, J=4.6 Hz, 1H), 7.94 (m, J=8.9, 3.3 Hz, 1H), 7.87 (m, J=8.7, 2.1 Hz, 1H), 7.75 (d, J=7.8 Hz, 1H), 7.65 (m, J=6.6 Hz, 2H), 7.31 (t, J=6.9 Hz, 1H), 7.26 (s, 1H), 7.18-7.13 (m, 2H), 7.06 (m, J=8.4, 7.1, 1.4 Hz, 1H), 6.55 (s, 3H), 4.13 (m, J=6.6 Hz, 2H), 3.82 (m, J=6.4 Hz, 2H), 1.95-1.86 (m, 2H), 1.70 (m, J=6.8 Hz, 2H), 1.56 (m, J=8.0 Hz, 2H), 1.46-1.38 (m, 6H), 1.37-1.30 (m, 4H), 1.00-0.90 (m, 6H). ¹³C NMR (101 MHz, cdcl₃) δ 159.3, 158.2, 158.1, 141.0, 140.7, 136.0, 133.0, 131.5, 131.5, 131.5, 131.3, 131.2, 131.0, 130.0, 129.7, 129.7, 129.0, 128.5, 128.4, 128.2, 127.9, 127.2, 126.6, 126.3, 126.3, 126.1, 126.1, 125.9, 125.6, 125.2, 124.9, 124.7, 123.6, 121.3, 121.3, 120.8, 120.7, 114.8, 68.4, 68.2, 31.8, 31.7, 29.5, 29.2, 26.0, 25.8, 22.8, 22.8, 14.2, 14.2. APPI HRMS calculated for [C49H45F3O2]+ 722.3372, found 722.3372.

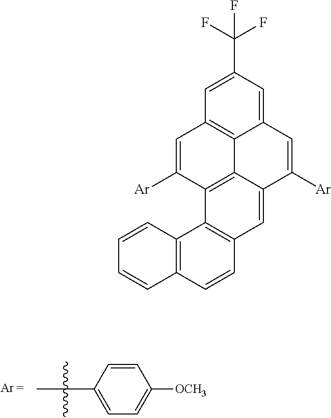

Rf=0.38 (hexane:DCM 1:1). IR(neat) 3026.27, 2919.55, 1604.32, 1495.09, 1246.86, 1029.81, 895.32 cm-1. ¹H NMR (500 MHz, chloroform-d) δ 8.63 (s, 1H), 8.52 (s, 1H), 8.35 (d, J=1.7 Hz, 1H), 8.33 (s, 1H), 8.08 (d, J=8.2 Hz, 1H), 7.98 (s, 1H), 7.94 (d, J=8.6 Hz, 1H), 7.87 (d, J=8.6 Hz, 1H), 7.75 (d, J=6.6 Hz, 1H), 7.65 (d, J=7.9 Hz, 2H), 7.31 (m, J=8.0, 6.9, 1.2 Hz, 1H), 7.16 (d, J=8.8 Hz, 3H), 7.06 (m, J=8.3, 7.0, 1.3 Hz, 1H), 6.92-6.16 (m, 3H), 3.98 (s, 3H), 3.69 (s, 3H). ¹³C NMR (126 MHz, cdcl₃) δ 159.7, 158.6, 140.9, 140.6, 136.2, 132.9, 131.5, 131.4, 131.1, 131.0, 130.0, 129.8, 129.6, 129.0, 128.5, 128.4, 128.2, 127.2, 126.6, 126.3, 126.1, 126.1, 125.9, 125.5, 125.1, 124.9, 124.7, 123.9, 120.8, 114.26, 55.6, 55.4. APPI HRMS calcd for [C39H25F3O2]+ 582.1807, found 582.1807.

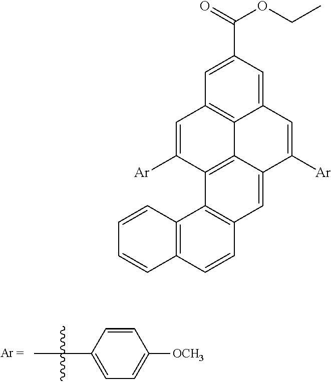

Rf=0.38 (hexane:DCM 3:2). FTIR(neat) 2917.23, 2848.98, 1714.61, 1608.06, 1245.65, 1221.56, 1210.14, 1176.83, 1034.46, 833.67 cm-1. ¹H NMR (400 MHz, chloroform-d) δ 8.98 (s, 1H), 8.82 (s, 1H), 8.62 (s, 1H), 8.37 (s, 1H), 8.11 (d, J=8.2 Hz, 1H), 8.04 (s, 1H), 7.95 (d, J=8.7 Hz, 1H), 7.87 (d, J=8.7 Hz, 1H), 7.74 (d, J=7.9 Hz, 1H), 7.69 (d, J=8.1 Hz, 2H), 7.31 (m, J=7.4 Hz, 1H), 7.18 (d, J=8.3 Hz, 2H), 7.06 (m, J=7.4 Hz, 1H), 6.55 (s, 4H), 4.58 (q, J=7.1 Hz, 2H), 3.99 (s, 3H), 3.69 (s, 3H), 1.54 (s, 3H). ¹³C NMR (101 MHz, cdcl₃) δ 149.1, 142.4, 138.8, 134.6, 133.3, 133.2, 132.8, 132.8, 132.2, 131.6, 131.4, 130.5, 130.4, 130.1, 129.7, 129.0, 128.9, 128.3, 128.1, 127.5, 127.1, 127.0, 126.8, 125.8, 125.6, 125.6, 125.3, 125.2, 124.8, 124.0, 122.3, 119.0, 93.4, 92.9, 34.9, 31.4. APPI HRMS calcd for [C41H30O4]+ 586.2144, found 586.2158.

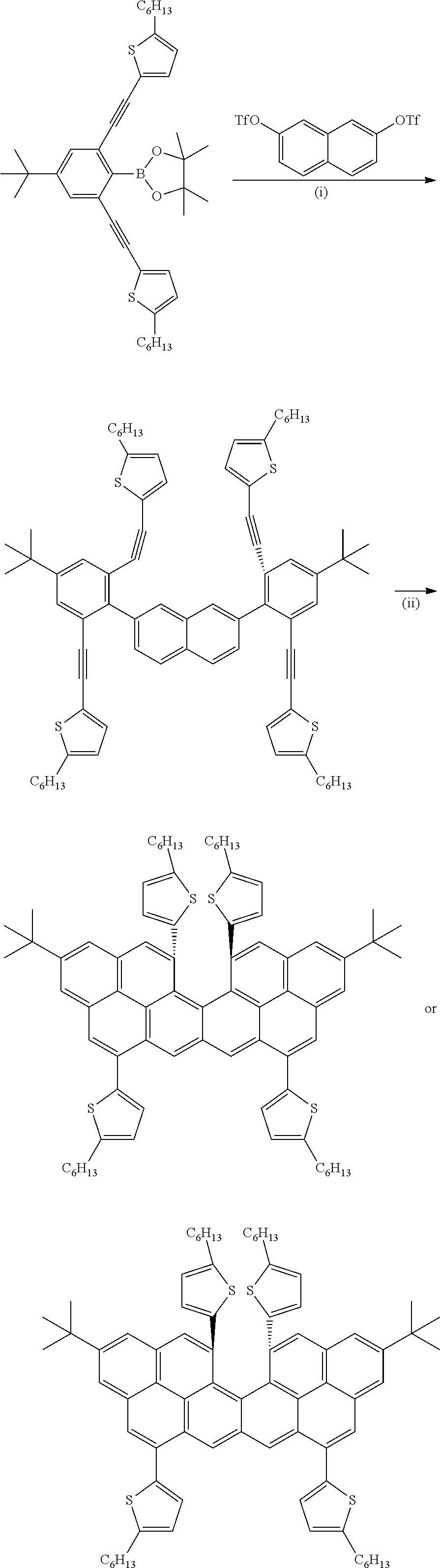

i) Pd(PPh₃)₄, K₂CO₃, THF/H₂O, 80° C., 24 h; ii) TfOH, CH₂Cl₂, −40° C. Both M and P enantiomers can be obtained. Rf=0.30 (hexane:CH2Cl2=4:1); FTIR (neat) 2954, 2931, 2858, 1712, 1604, 1508, 1464 cm-1; 1H NMR (400 MHz, CDCl3) δ 9.17 (s, 2H), 8.13 (s, 2H), 8.09 (s, 2H), 8.06 (s, 2H), 7.85 (s, 2H), 7.45 (d, J=3.4 Hz, 2H), 7.02 (d, J=3.5 Hz, 2H), 6.18 (d, J=3.5 Hz, 2H), 5.98 (d, J=3.6 Hz, 2H), 3.01 (t, J=7.7 Hz, 4H), 2.40-2.24 (m, 4H), 1.92-1.80 (m, 4H), 1.58 (s, 18H), 1.55-1.48 (m, 8H), 1.43-1.38 (m, 8H), 1.28-1.21 (m, 4H), 1.17-10.8 (m, 8H), 0.97-0.92 ppm (m, 6H), 0.91-0.84 ppm (m, 6H); 13C NMR (100 MHz, CDCl3) δ 149.5, 146.3, 144.9, 142.9, 139.9, 132.8, 132.3, 131.5, 130.8, 129.3, 129.2, 129.1, 128.1, 127.4, 126.9, 126.4, 124.7, 124.1, 123.6, 122.83, 122.81, 122.3, 121.4, 119.6, 35.3, 32.0, 31.9, 31.8, 31.6, 31.5, 30.5, 29.8, 29.2, 28.7, 22.80, 22.75, 14.12, 14.11 ppm; HRMS (APPI, positive) m/z calcd for C78H88S4 [M]+ 1152.5763, found 1152.5777.

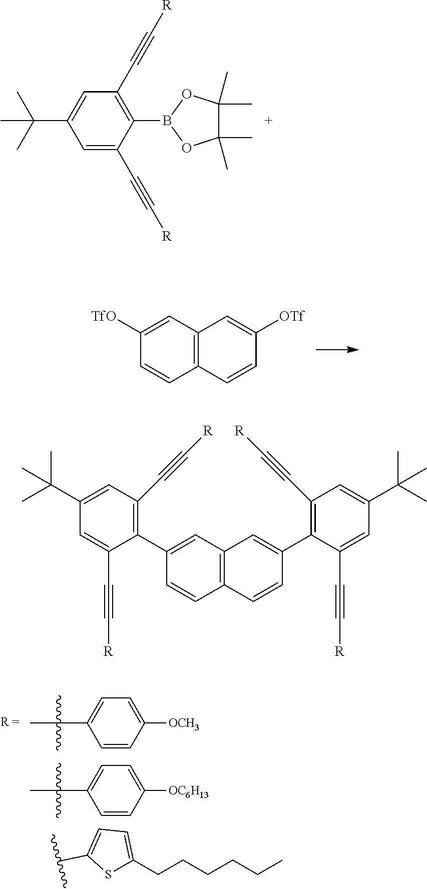

In a 25 mL sealed tube, bis(trifluoromethanesulfonyloxy)naphthalene (150 mg, 0.35 mmol), 2,6-diynylphenyl borate (400 mg, 0.77 mmol) and K₂CO₃ (242 mg, 1.8 mmol) were dissolved in THF (16 mL) and water (4 mL) solution. Pd(PPh₃)₄ (20 mg, 0.02 mmol) was added to the solution before degassing the mixture via bubbling nitrogen for 30 min. The resulting mixture was stirred under a N₂ atmosphere at 80° C. for 24 h. After the reaction was complete, the mixture was diluted with CH₂C2, washed with H₂O and dried over Na₂SO₄. The solvent was removed under reduced pressure and the residue was purified by column chromatography.

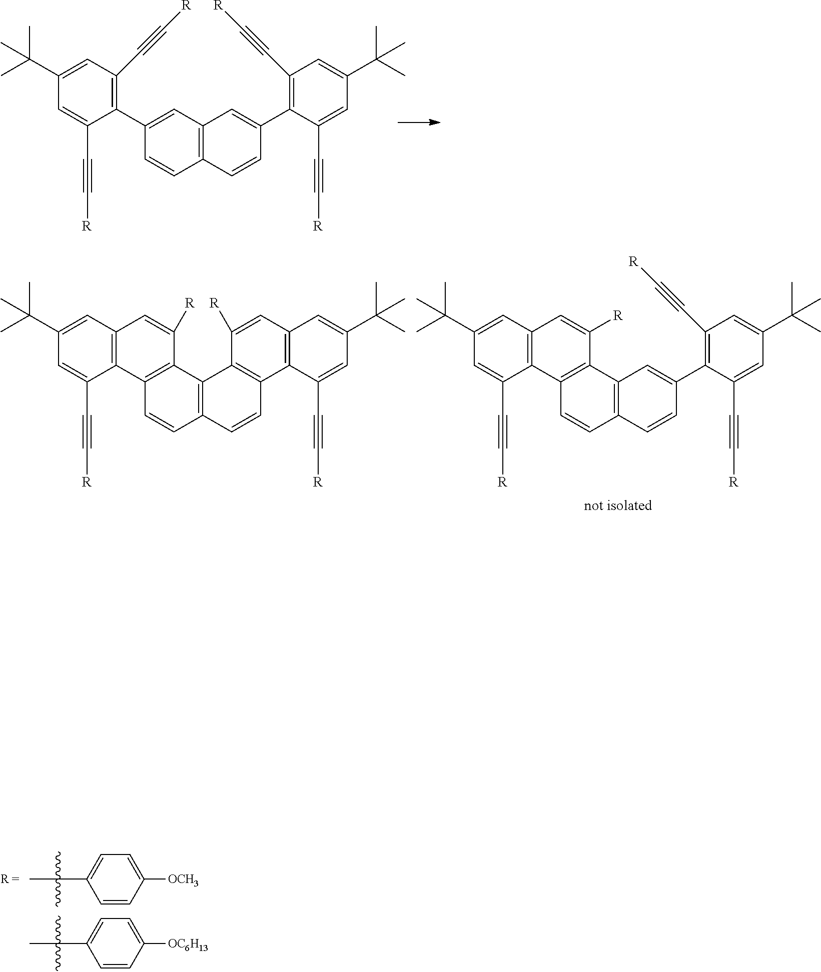

In a flame dried flask under a nitrogen atmosphere, compound 3 (123 mg 0.13 mmol) was dissolved in anhydrous CH₂Cl₂ (20 mL) and cooled to 0° C. TFA (749 mg, 6.57 mmol) was added dropwise by the help of syringe to the solution. After stirring for an hour at 0° C., the reaction was quenched with saturated NaHCO₃ solution (5 mL). The solution was then washed with H₂O (2×30 mL) and dried over anhydrous Na₂SO₄. After removing the solvent under reduced pressure, the residue was purified by flash column chromatography.

Characterization data for certain compounds is provided below.

Rf=0.20 (hexane:CH2Cl2=1:1); FTIR (neat) 2956, 2834, 2206, 1605, 1510, 1464, 1440 cm-1; 1H NMR (400 MHz, CDCl3) δ 10.17 (d, J=8.7 Hz, 2H), 8.04 (d, J=8.8 Hz, 2H), 7.96 (d, J=2.2 Hz, 2H), 7.78-7.69 (m, 4H), 7.64 (d, J=2.2, 2H), 7.22 (s, 2H), 7.06-6.98 (m, 4H), 6.88-6.57 (br, 4H), 6.35-6.14 (br, 4H), 3.90 (s, 6H), 3.26 (s, 6H), 1.48 ppm (s, 18H); 13C NMR (100 MHz, CDCl3) δ 159.9, 158.0, 148.0, 137.8, 133.2, 133.08, 133.05, 132.1, 130.7, 130.5, 130.30, 130.27, 127.4, 126.6, 125.4, 125.1, 124.7, 123.7, 119.0, 116.3, 114.4, 93.7, 91.8, 55.5, 55.4, 34.7, 31.5 ppm; HRMS (ESI, positive) m/z calcd for C66H57O4 [M+Na]+ 935.4071, found 935.4087.

Rf=0.30 (hexane:CH2Cl2=2:1); FTIR (neat) 3726, 3703, 3625, 3600, 2954, 2930, 2869, 2343, 1605, 1510, 1468 cm-1; 1H NMR (400 MHz, CDCl3) δ 10.20 (d, J=8.8 Hz, 2H), 8.03 (d, J=8.8 Hz, 2H), 7.94 (d, J=2.2 Hz, 2H), 7.74-7.69 (m, 4H), 7.62 (d, J=2.1 Hz, 2H), 7.21 (s, 2H), 7.04-6.97 (m, 4H), 6.85-6.55 (br, 4H), 6.35-6.10 (br, 4H) 4.05 (t, J=6.6 Hz, 4H), 3.50 (dt, J=9.2, 6.7 Hz, 2H), 3.29 (dt, J=9.2, 6.6 Hz, 2H), 1.88-1.80 (m, 4H), 1.52-1.45 (m 24H), 1.43-1.32 (s, 10H), 1.13-1.23 (m, 12H), 0.96-0.86 ppm (m, 12H); 13C NMR (100 MHz, CDCl3) δ 159.5, 157.5, 147.9, 138.0, 133.3, 133.1, 133.0, 132.1, 130.7, 130.32, 130.31, 127.7, 126.5, 125.5, 124.7, 123.8, 119.0, 116.0, 114.9, 93.8, 91.8, 68.3, 68.0, 34.7, 31.8, 31.5, 29.4, 29.3, 25.9, 25.8, 22.78, 22.76, 14.2 ppm; HRMS (APPI, positive) m/z calcd for C66H56O4 [M]+ 912.7303, found 912.7299.

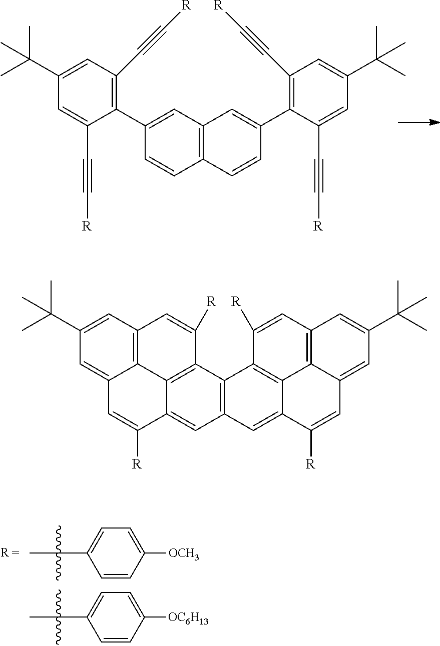

In a 200 mL flame-dried flask, the starting material (100 mg, 0.11 mmol) was dissolved in 100 mL of anhydrous CH2C12 and cooled to 0° C. TFA (624 mg, 5 mmol) was added dropwise by the help of syringe to the solution. After stirring for 2 hours at 0° C., the reaction cooled to −40° C. A cold CH₂C12 solution (0.4 mL) of triflic acid (165 mg, 1.1 mmol) was slowly added into the reaction mixture at −40° C., and the color of the solution changed to dark blue slowly. After stirring for 30 min at −40° C., the reaction was quenched with saturated NaHCO₃ solution (6-7 mL). The solution was then washed with H₂O (2×30 mL) after it is warmed to room temperature and dried over anhydrous Na₂SO₄. After removing the solvent under reduced pressure, the residue for product 7 was purified by flash column chromatography. Characterization data for certain compounds is provided below.

Rf=0.20 (hexane:CH2Cl2=1:1); FTIR (neat) 2951, 2832, 1606, 1508, 1462, 1439 cm-1; 1H NMR (400 MHz, CDCl₃) δ 8.66 (s, 2H), 8.12 (d, J=1.8 Hz, 2H), 8.05 (d, J=1.7 Hz, 2H), 7.88 (s, 2H), 7.79-7.74 (m, 4H), 7.69 (s, 2H), 7.19-7.16 (m, 4H), 6.73 (br, 4H), 6.21 (br, 4H), 3.99 (s, 6H), 3.30 (s, 6H), 1.59 ppm (s, 18H); 13C NMR (100 MHz, CDCl3) δ 159.3, 158.0, 149.4, 139.2, 137.9, 133.8, 133.5, 131.5, 131.4, 130.0, 130.2, 129.8, 128.6, 128.0, 127.2, 125.9, 124.5, 123.6, 122.4, 121.8, 121.5, 120.1, 114.2, 112.6, 55.6, 55.4, 35.3, 32.0 ppm; HRMS (APPI, positive) m/z calcd for C66H56O4 [M]+912.4173, found 912.4203.

Rf=0.20 (hexane:CH2Cl2=2:1); FTIR (neat) 2954, 2927, 2857, 2360, 2341, 1608, 1509, 1466 cm-1; 1H NMR (400 MHz, CDCl3) δ 8.66 (s, 2H), 8.10 (s, 2H), 8.03 (s, 2H), 7.87 (s, 2H), 7.82-7.72 (m, 4H), 7.70 (s, 2H), 7.20-7.12 (m, 4H), 6.90-6.55 (br, 4H), 6.40-6.05 (br, 4H), 4.14 (t, J=6.5 Hz, 4H), 3.52 (dt, J=9.0, 6.7 Hz, 2H), 3.30 (dt, J=9.1, 6.6 Hz, 2H), 1.98-1.84 (m, 4H), 1.75-1.50 (m, 24H), 1.72-1.47 (m, 10H), 1.31-1.08 (m, 12H), 1.04-0.92 (m, 6H), 0.91-0.81 ppm (m, 6H); 13C NMR (100 MHz, CDCl3) δ 158.9, 157.6, 149.3, 139.3, 138.1, 133.6, 133.5, 131.6, 131.4, 131.1, 130.1, 129.8, 128.6, 127.9, 127.4, 125.9, 124.6, 123.7, 122.4, 121.7, 121.5, 120.2, 114.7, 113.1, 68.3, 68.1, 35.3, 32.0, 31.9, 31.7, 29.6, 29.3, 26.0, 25.8, 22.8, 22.7, 14.3, 14.2 ppm; HRMS (APPI, positive) m/z calcd for C86H96O4 [M]+ 1192.7303, found 1192.7318.

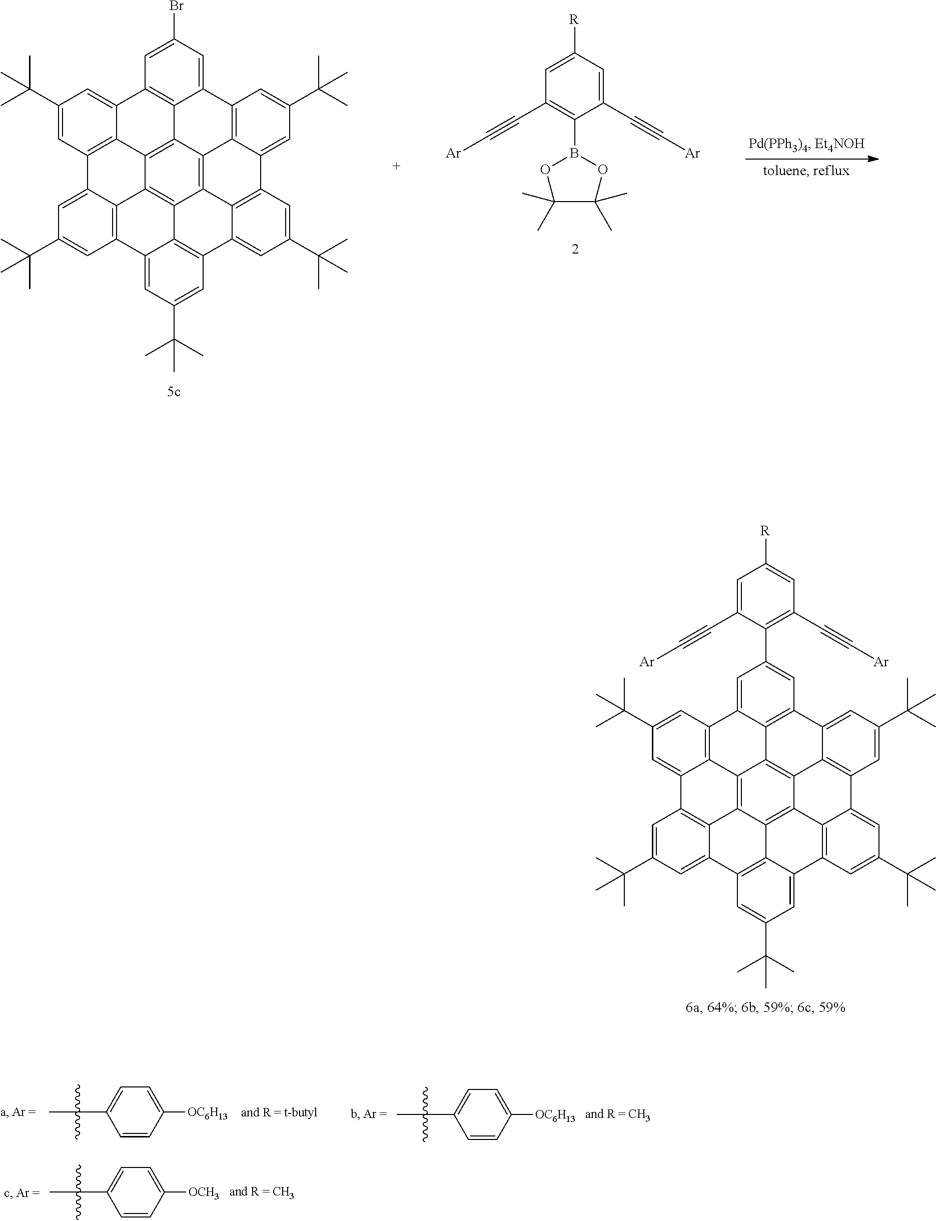

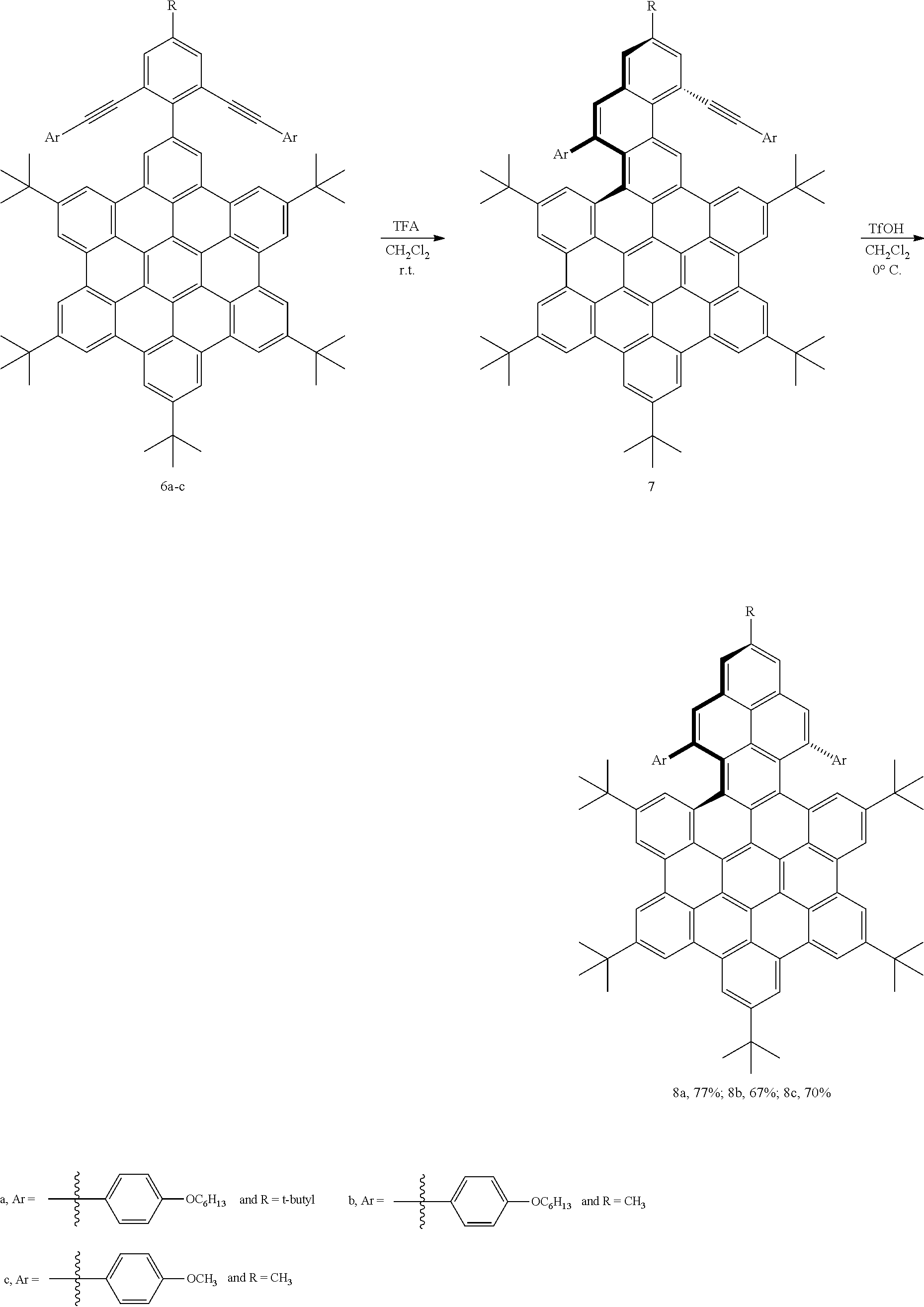

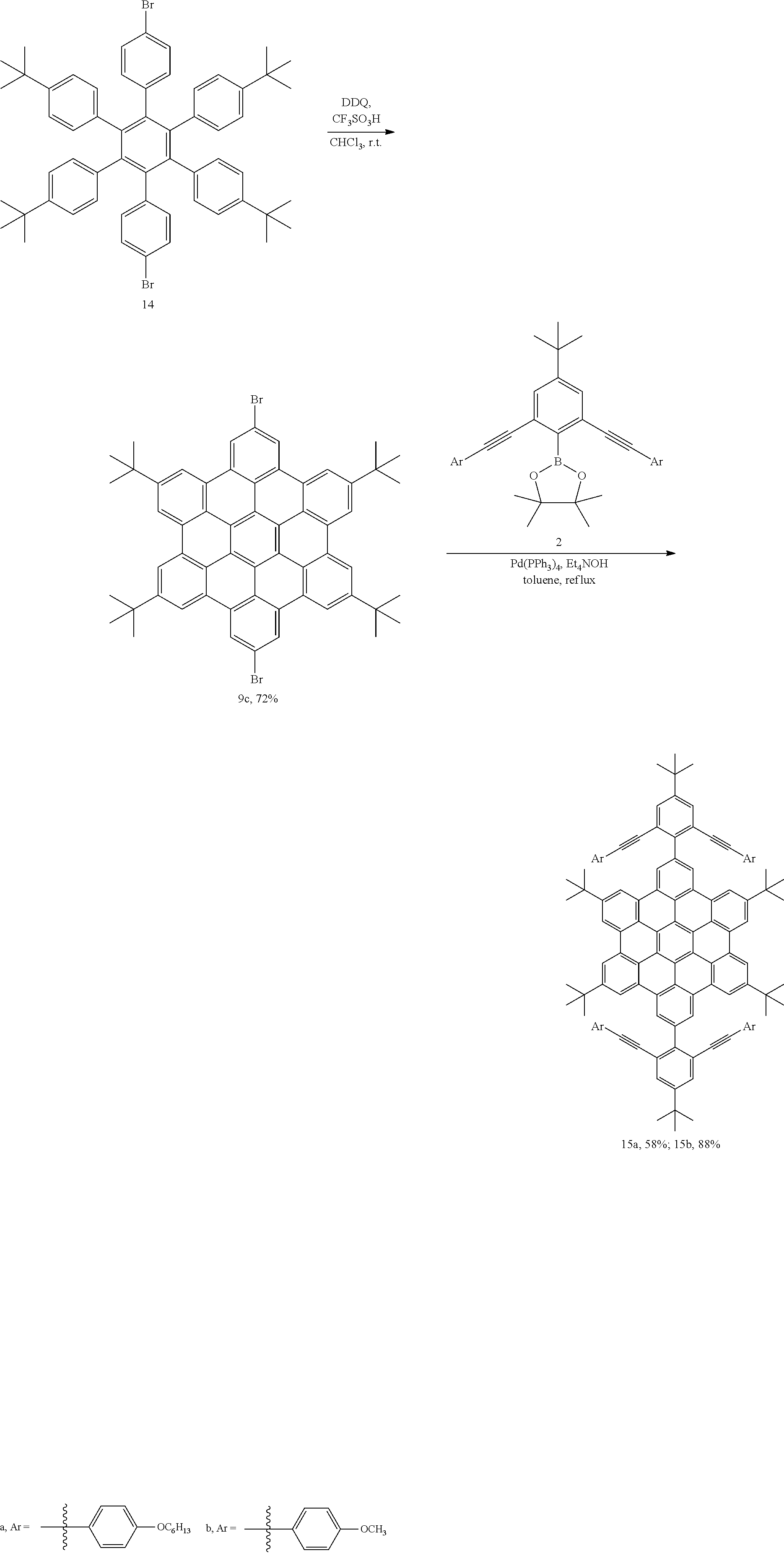

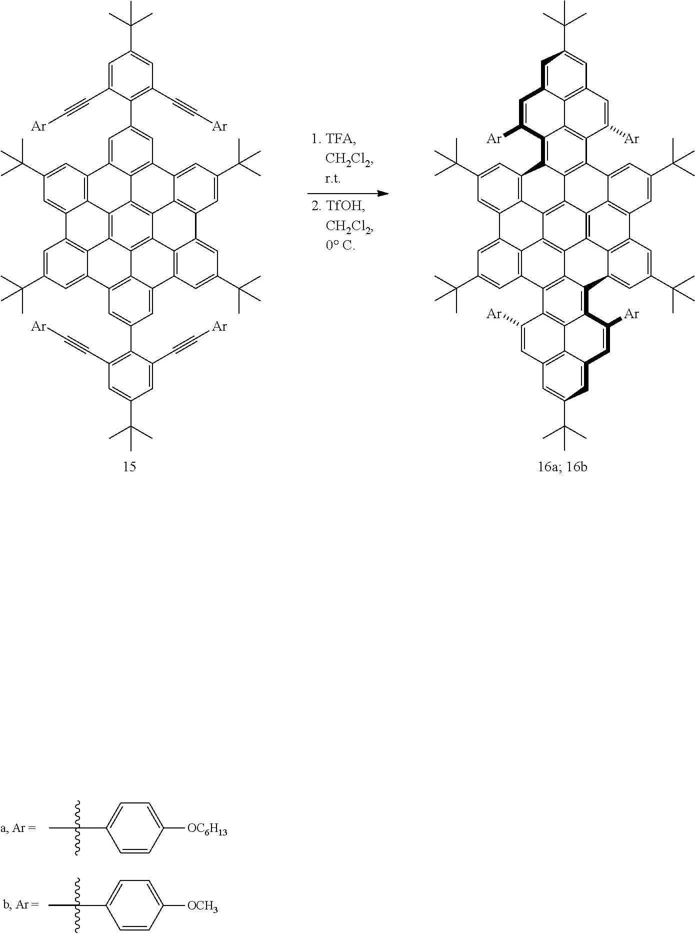

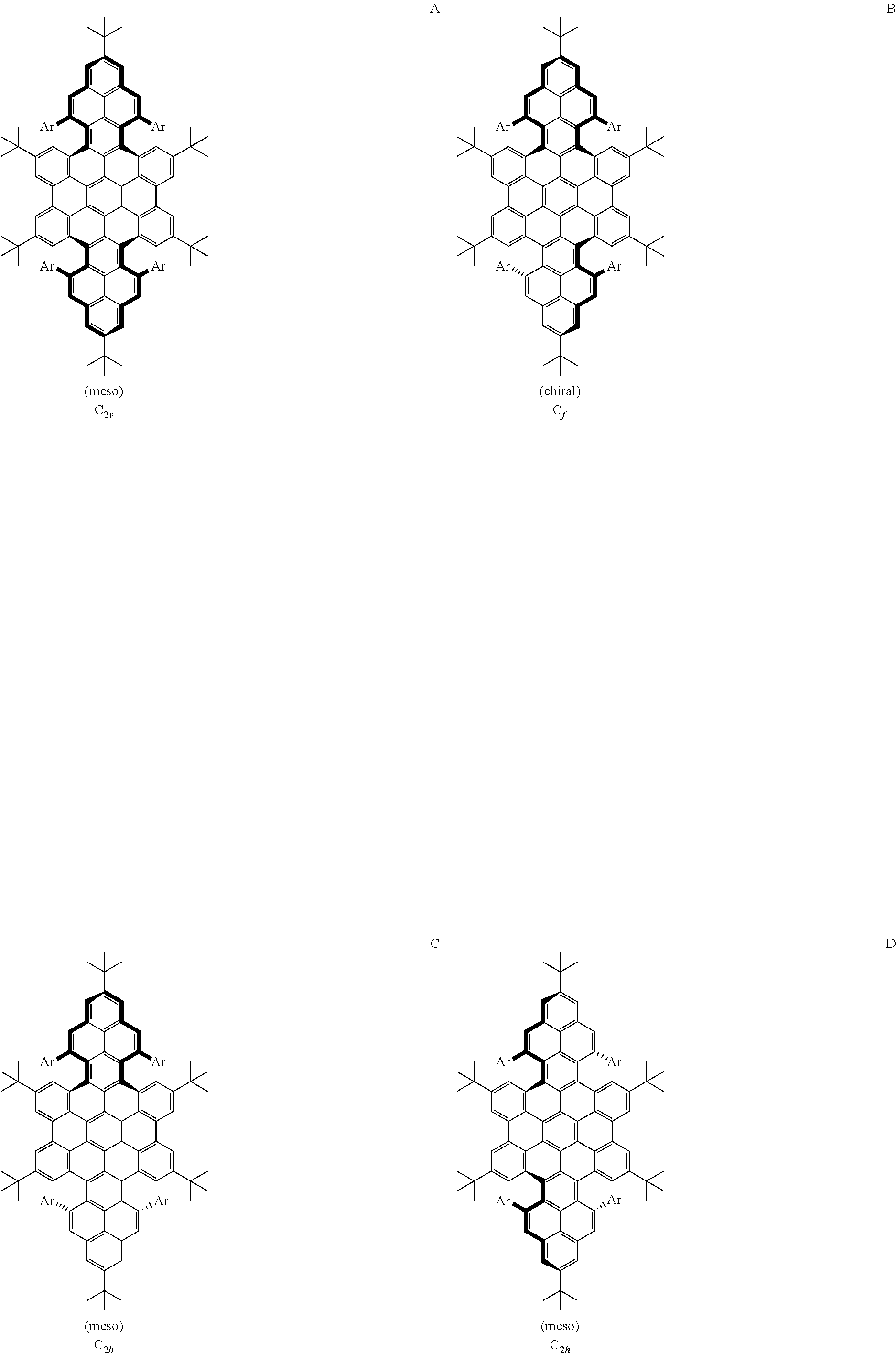

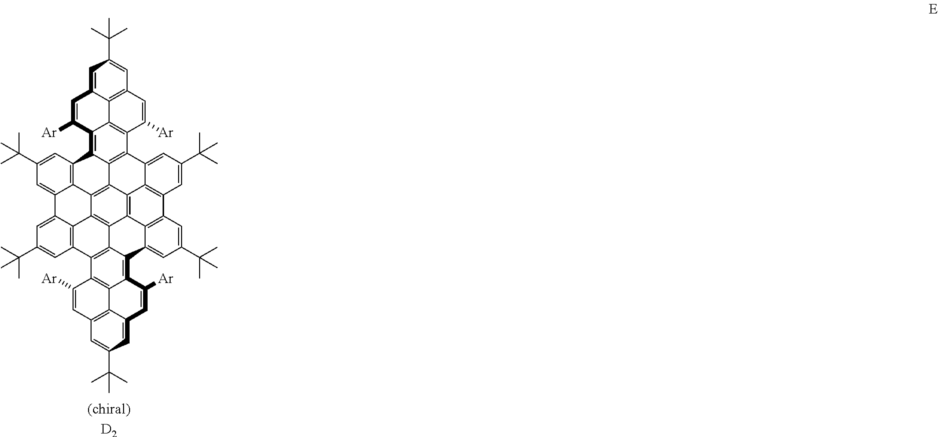

Depending upon the modes of four-fold alkyne cyclization, compound 16 above can possibly exist as five isomers: A, B, C, D and E (illustrated above). Isomers B and E are chiral (no plane of symmetry) whereas A, C and D have plane of symmetry and hence are achiral (meso compounds). Any molecule that lacks both inversion center (i) and mirror plane (o) is chiral. Isomers B and E belong to point groups C₁ and D₂ respectively. Similarly, achiral isomers A, C and D belong to point groups C₂ᵥ, C₂ₕ and C₂ₕ respectively.

In view of the many possible embodiments to which the principles of the present disclosure may be applied, it should be recognized that the illustrated embodiments are only preferred examples and should not be taken as limiting. Rather, the scope of the disclosure is defined by the following claims. We therefore claim as our invention all that comes within the scope and spirit of these claims.

1. A compound, having a structure satisfying a formula 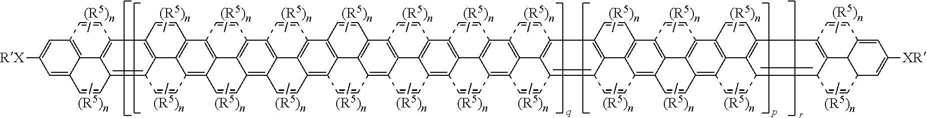
wherein
X is a heteroatom selected from O, S, or N(R″)₂ wherein R″ is hydrogen, aliphatic, haloaliphatic, haloheteroaliphatic, heteroaliphatic, aromatic or any combination thereof;
each R′ independently is selected from hydrogen, aliphatic, or —C(O)R²⁰, wherein R²⁰ is hydrogen, aliphatic, haloaliphatic, haloheteroaliphatic, heteroaliphatic, aromatic or any combination thereof;
each R⁵, if present, independently is aliphatic, heteroaliphatic, aliphatic-aromatic, heteroaliphatic-aromatic, aromatic, or any combination thereof;
each n independently is 0, 1, or 2;
q is an integer selected from 0 to 1000;
p is an integer selected from 0 to 1000; and
r is an integer selected from 1 to 1000, provided that when p is 0, r is the same integer as q.

2. The compound of claim 1, having a structure satisfying a formula 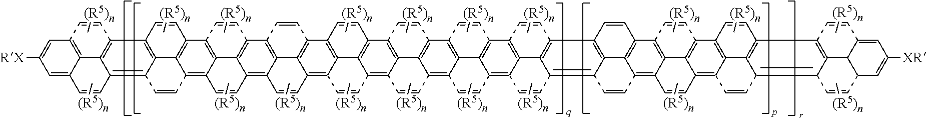
wherein
X is a heteroatom selected from O, S, or N(R″)₂ wherein R″ is hydrogen, aliphatic, haloaliphatic, haloheteroaliphatic, heteroaliphatic, aromatic or any combination thereof;
each R′ independently is selected from hydrogen, aliphatic, or —C(O)R²⁰, wherein R²⁰ is hydrogen, aliphatic, haloaliphatic, haloheteroaliphatic, heteroaliphatic, aromatic or any combination thereof; and
each R⁵, if present, independently is aliphatic, heteroaliphatic, aliphatic-aromatic, heteroaliphatic-aromatic, aromatic, or any combination thereof;
each n independently is 0, 1, or 2;
q is an integer selected from 0 to 1000;
p is an integer selected from 0 to 1000; and
r is an integer selected from 1 to 1000, provided that when p is 0, r is the same integer as q.

3. The compound of claim 2, wherein the compound has a structure satisfying one or more of the formulas below

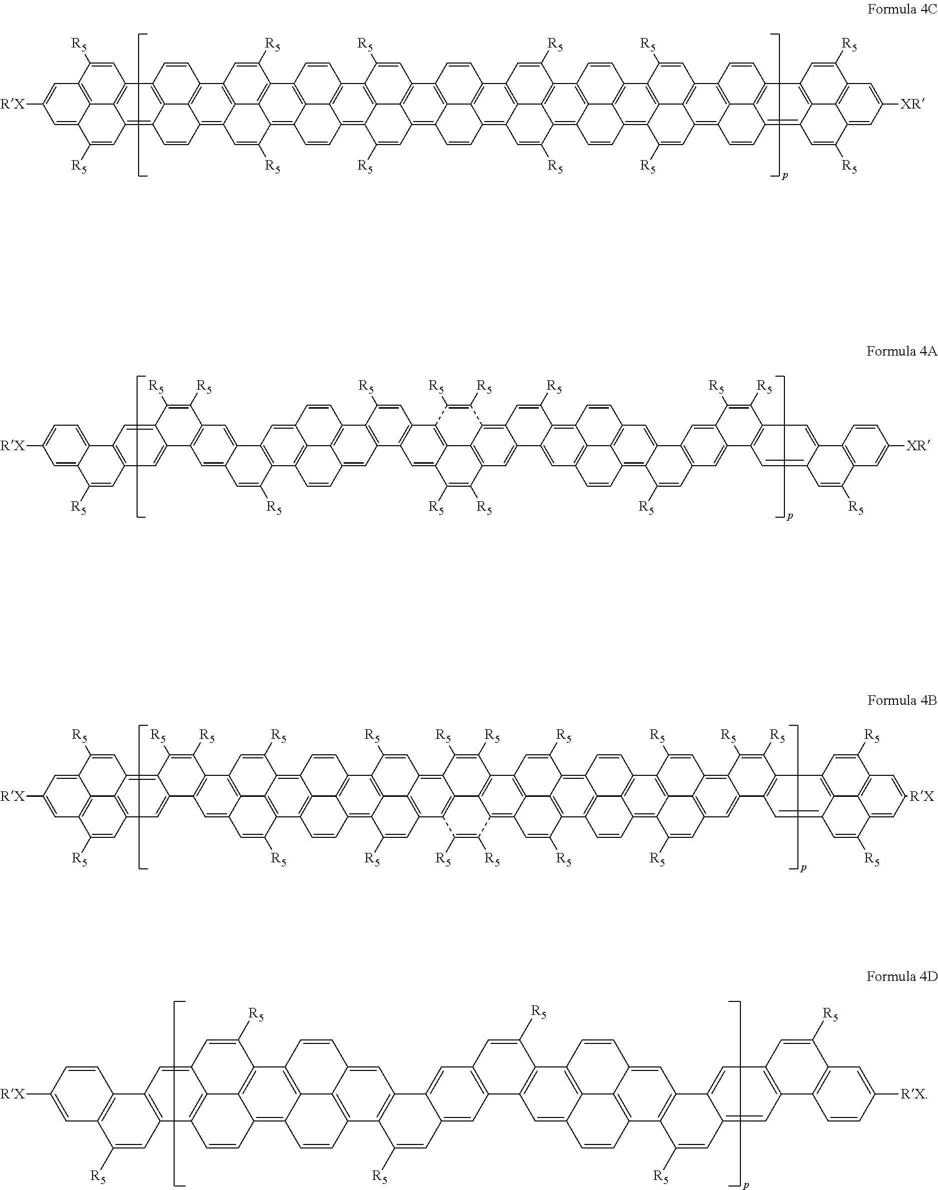

4. The compound of claim 1, wherein the compound has a structure satisfying one or more of the formulas below 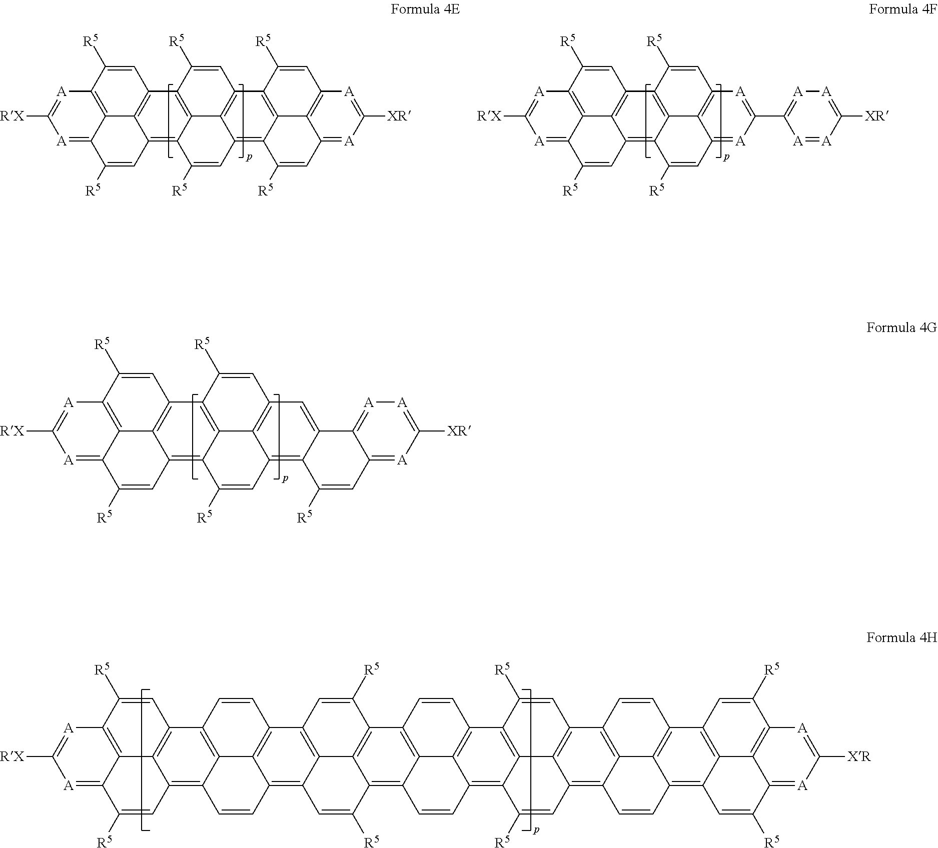
wherein each A independently is CH or nitrogen.

5. The compound of claim 1, wherein X, together with R′ is —OH, —OCH₃, —OC(O)CH₃, —SH, —SCH₃, —SC(O)CH₃, —NH₂, —NHCH₃, or —NHC(O)CH₃, and/or wherein each R⁵ independently is an alkyl group, alkoxy group, an alkylene oxide group, or an aromatic group.

6. (canceled)

7. The compound of claim 1, wherein the compound is selected from

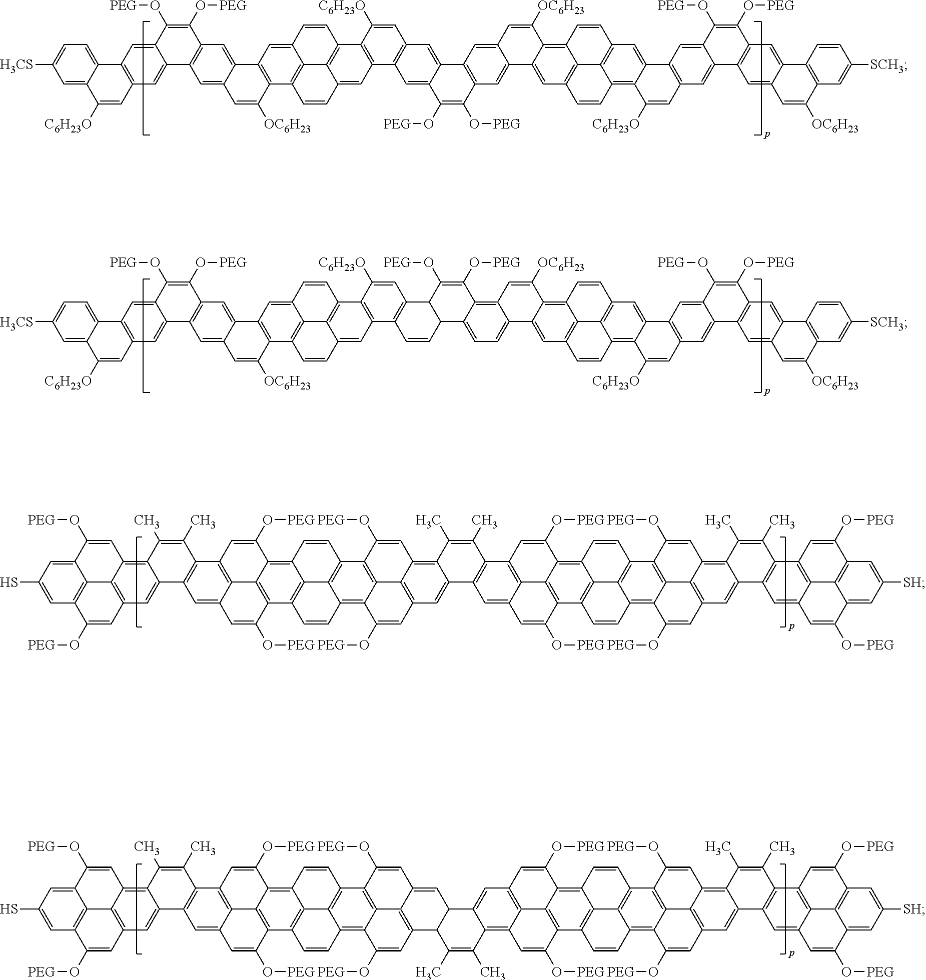

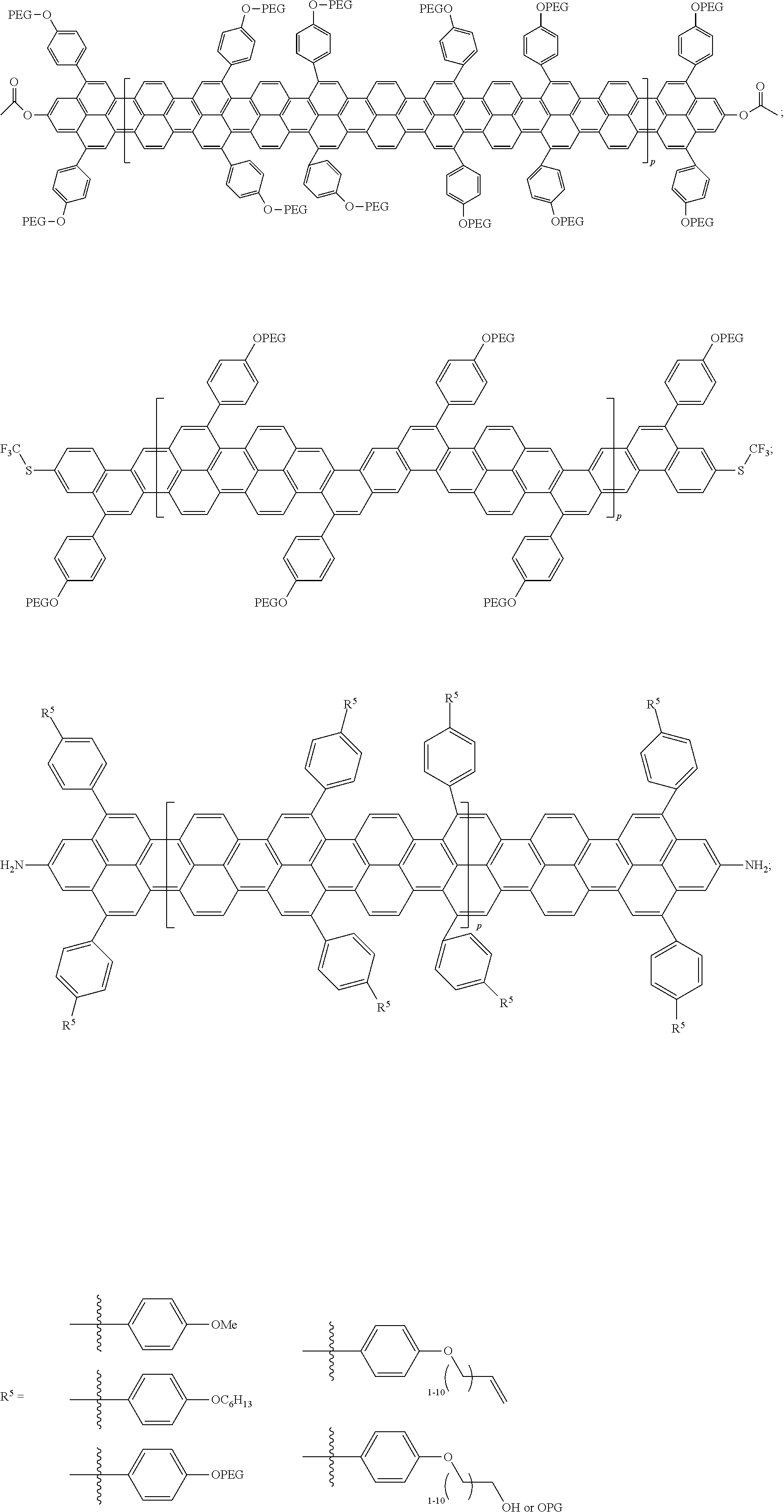

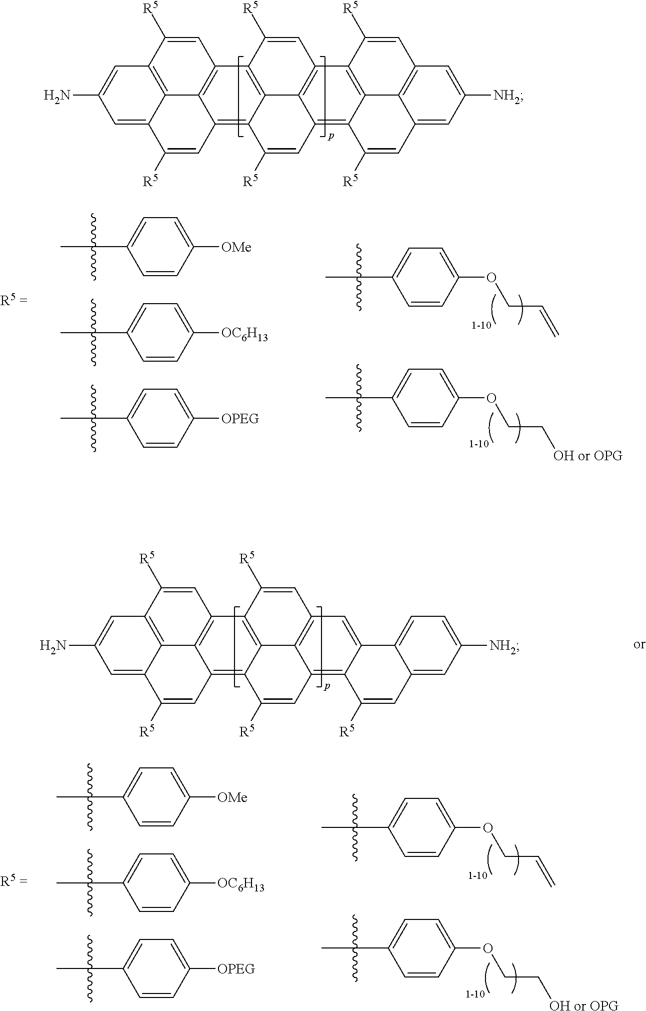

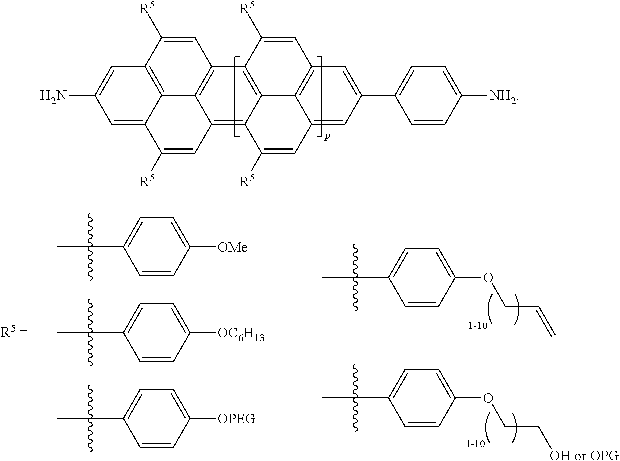

8. (canceled)

9. A compound, having a structure satisfying Formula 1 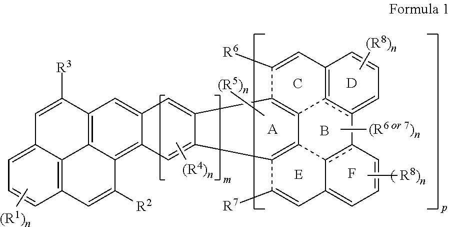
wherein
each R¹ if present, independently is aliphatic, haloaliphatic, haloheteroaliphatic, heteroaliphatic, aromatic, aliphatic-aromatic, heteroaliphatic-aromatic, or any combination thereof;
each R⁴ if present, independently is aliphatic, haloaliphatic, haloheteroaliphatic, heteroaliphatic, aromatic, aliphatic-aromatic, heteroaliphatic-aromatic, or any combination thereof;
each R⁵ if present, independently is aliphatic, haloaliphatic, haloheteroaliphatic, heteroaliphatic, aromatic, aliphatic-aromatic, heteroaliphatic-aromatic, or any combination thereof;
each R⁸ if present, independently is aliphatic, haloaliphatic, haloheteroaliphatic, heteroaliphatic, aromatic, aliphatic-aromatic, heteroaliphatic-aromatic, or any combination thereof;
each R² independently is hydrogen, aliphatic, haloaliphatic, haloheteroaliphatic, heteroaliphatic, aromatic, aliphatic-aromatic, heteroaliphatic-aromatic, or any combination thereof;
each R³ independently is hydrogen, aliphatic, haloaliphatic, haloheteroaliphatic, heteroaliphatic, aromatic, aliphatic-aromatic, heteroaliphatic-aromatic, or any combination thereof;
each R⁶ independently is hydrogen, aliphatic, haloaliphatic, haloheteroaliphatic, heteroaliphatic, aromatic, aliphatic-aromatic, heteroaliphatic-aromatic, or any combination thereof;
each R⁷ independently is hydrogen, aliphatic, haloaliphatic, haloheteroaliphatic, heteroaliphatic, aromatic, aliphatic-aromatic, heteroaliphatic-aromatic, or any combination thereof;
each n independently is an integer selected from 0 to 4;
p is 0 or 1, and when p is 0, none of the rings A, B, C, D, or E are present;
m is an integer selected from 0 to 10; and
ring A, when present, is phenyl such that none of the dashed bonds illustrated in Formula 1 are present, or ring A joins together with (i) rings B, C, and D to form a pyrene group; or (ii) rings B, E, and F to form a pyrene group; or (iii) ring E to form a naphthyl group.

10. The compound of claim 9, having a structure satisfying any one or more of Formulas 1A-1E:

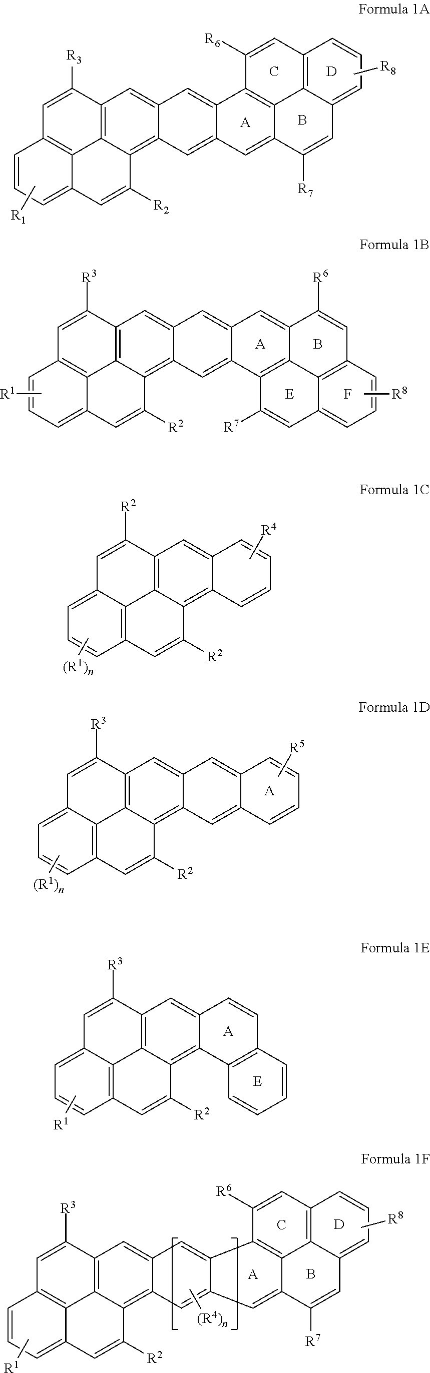

11. The compound of claim 9, wherein the compound is selected from:

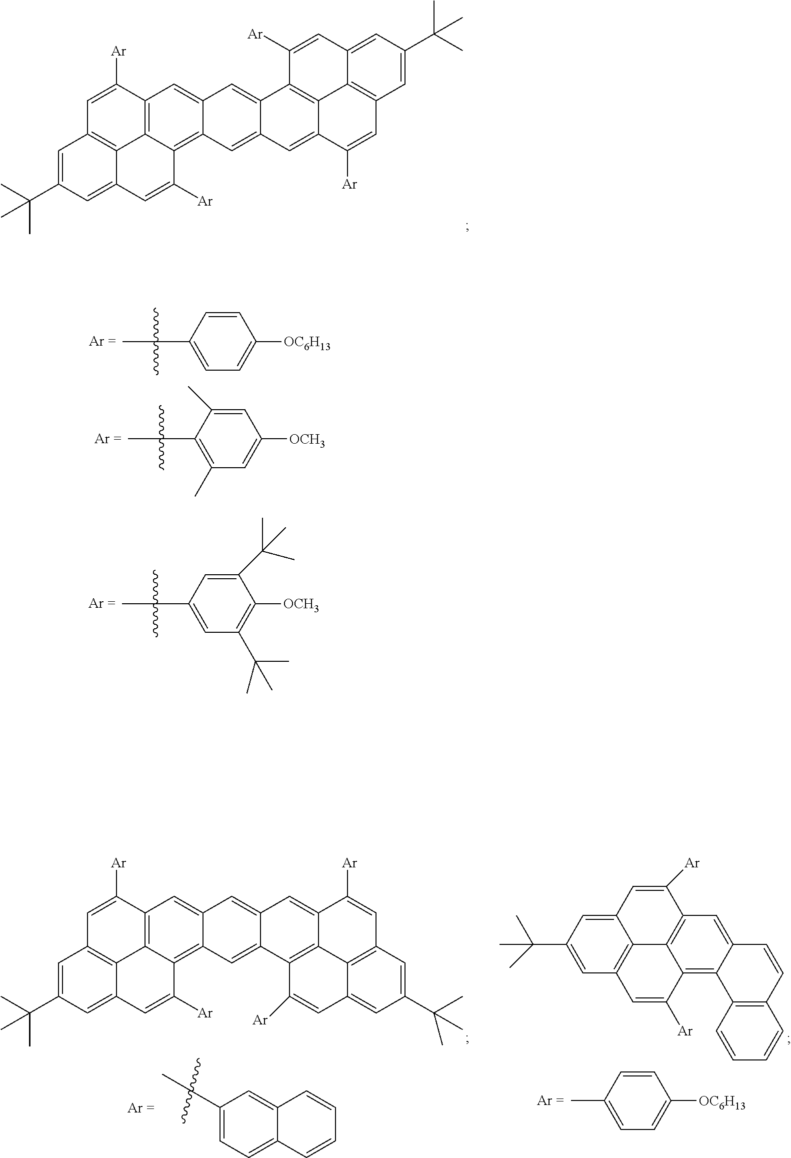

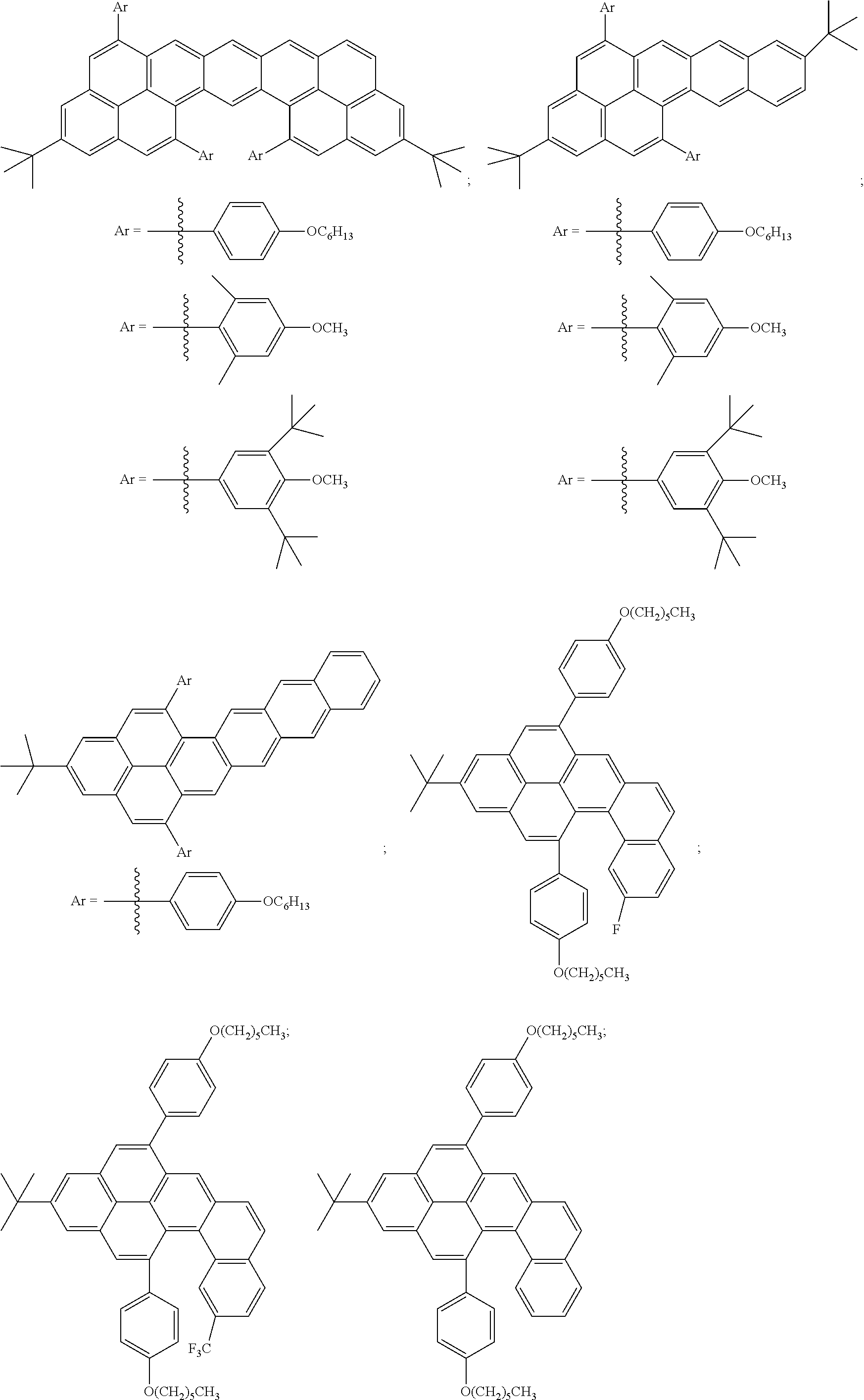

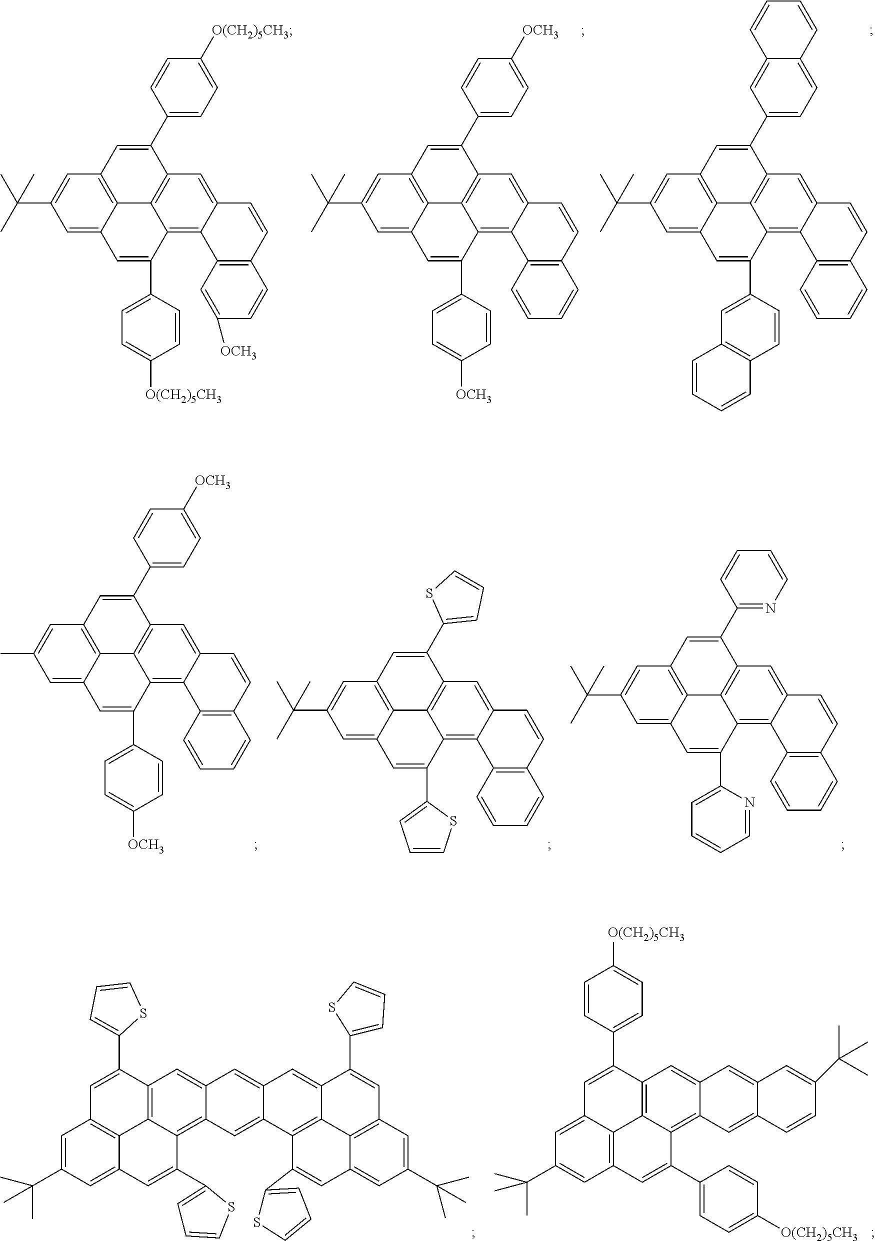

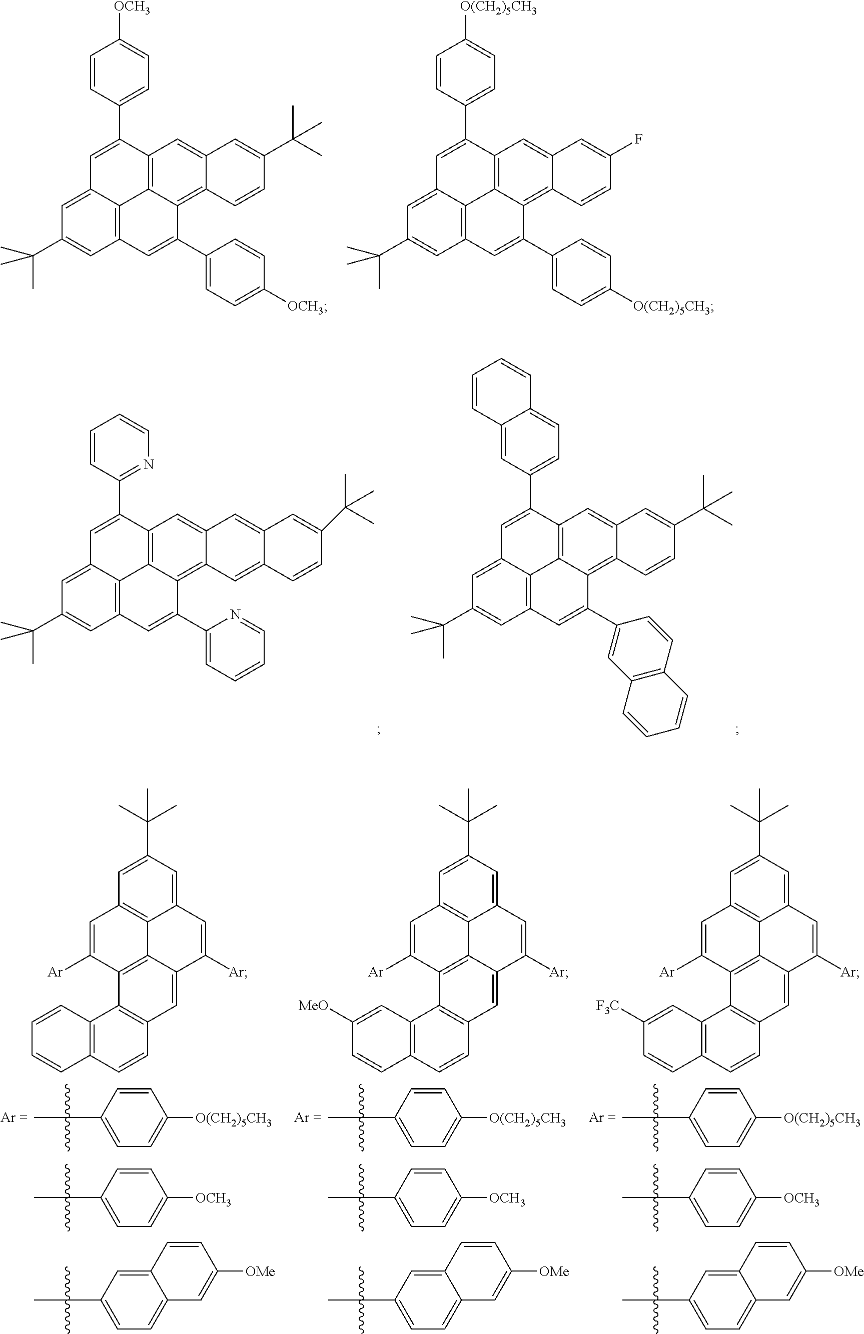

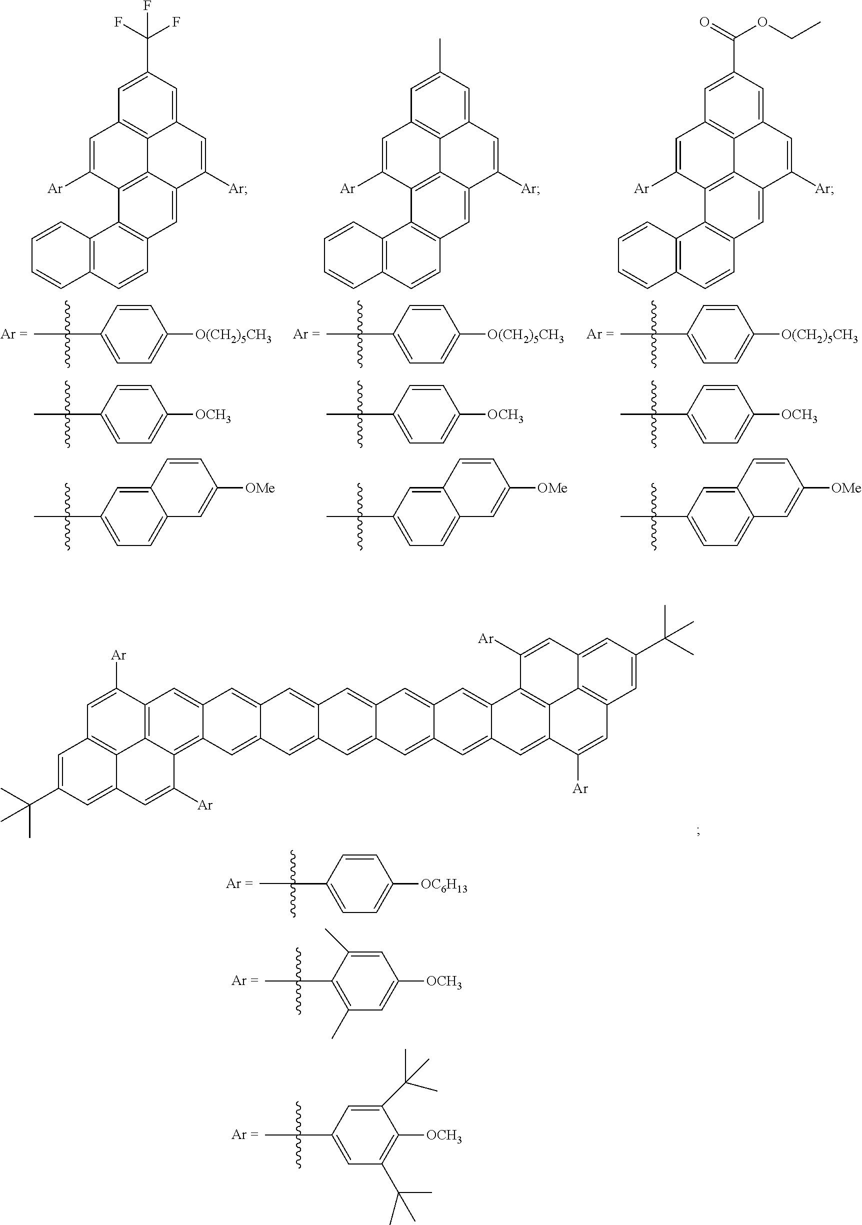

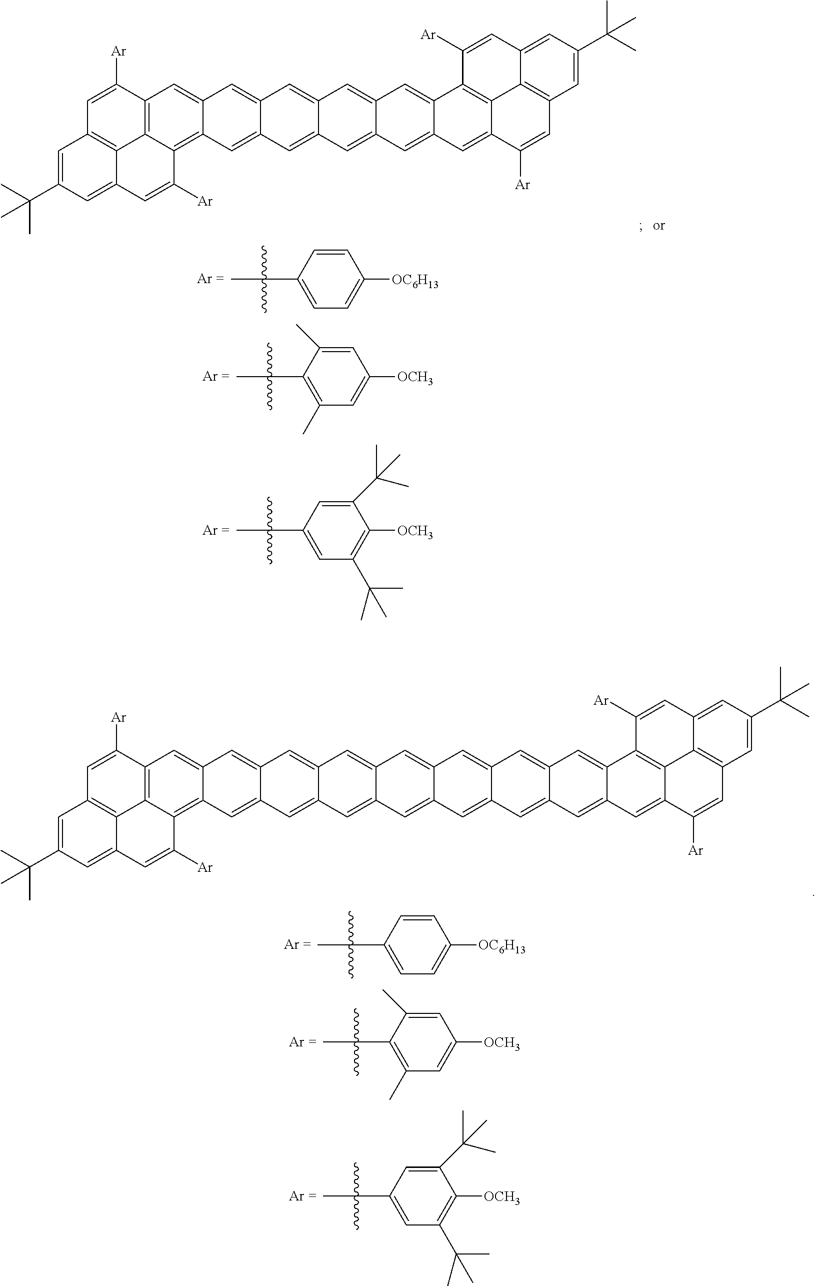

12. A compound, having a structure satisfying Formula 2 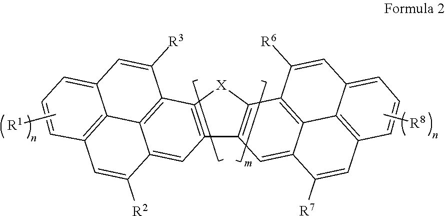
wherein
X is selected from O, S, C=O, C=S, SO₂, SO, C(R′)₂, N(R′), or N⁺(R′)₂ wherein each R′ independently is hydrogen, aliphatic, haloaliphatic, haloheteroaliphatic, heteroaliphatic, aromatic or any combination thereof;
each R¹ if present, independently is selected from aliphatic, haloaliphatic, haloheteroaliphatic, heteroaliphatic, aromatic, aliphatic-aromatic, heteroaliphatic-aromatic, or any combination thereof;
each R⁸ if present independently is selected from aliphatic, haloaliphatic, haloheteroaliphatic, heteroaliphatic, aromatic, aliphatic-aromatic, heteroaliphatic-aromatic, or any combination thereof;
each R² independently is hydrogen, aliphatic, haloaliphatic, haloheteroaliphatic, heteroaliphatic, aromatic, aliphatic-aromatic, heteroaliphatic-aromatic, or any combination thereof;
each R³ independently is hydrogen, aliphatic, haloaliphatic, haloheteroaliphatic, heteroaliphatic, aromatic, aliphatic-aromatic, heteroaliphatic-aromatic, or any combination thereof;
each R⁶ independently is hydrogen, aliphatic, haloaliphatic, haloheteroaliphatic, heteroaliphatic, aromatic, aliphatic-aromatic, heteroaliphatic-aromatic, or any combination thereof;
each R⁷ independently is hydrogen, aliphatic, haloaliphatic, haloheteroaliphatic, heteroaliphatic, aromatic, aliphatic-aromatic, heteroaliphatic-aromatic, or any combination thereof;
m is 0 or 1; and
each n independently is an integer selected from 0 to 3.

13. The compound of claim 12, wherein the compound has a structure satisfying one or more of Formulas 2A-2F

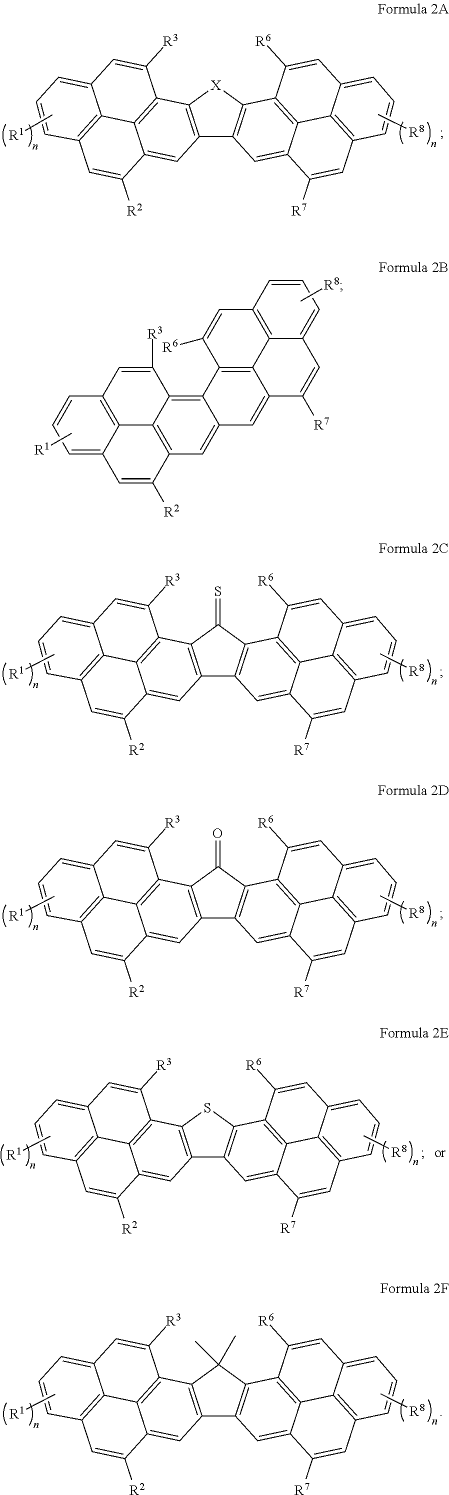

14. (canceled)

15. The compound of claim 12, wherein the compound is selected from

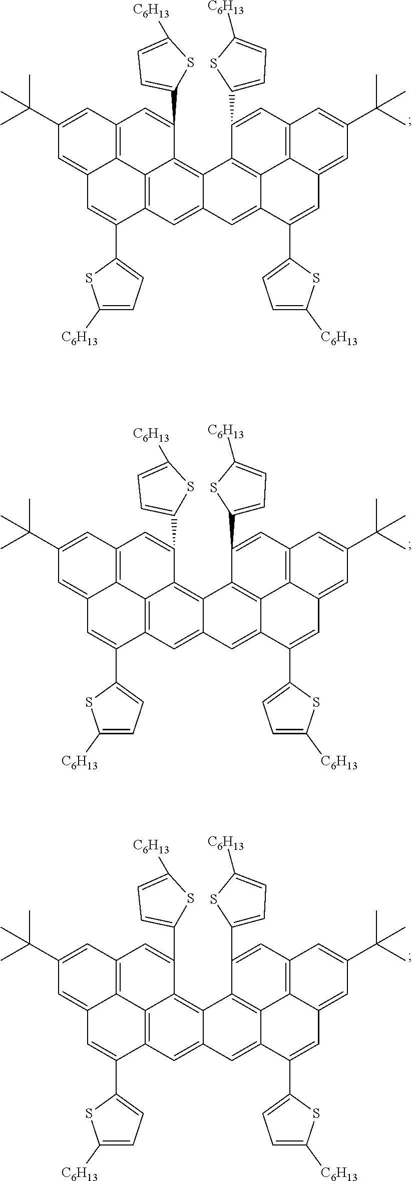

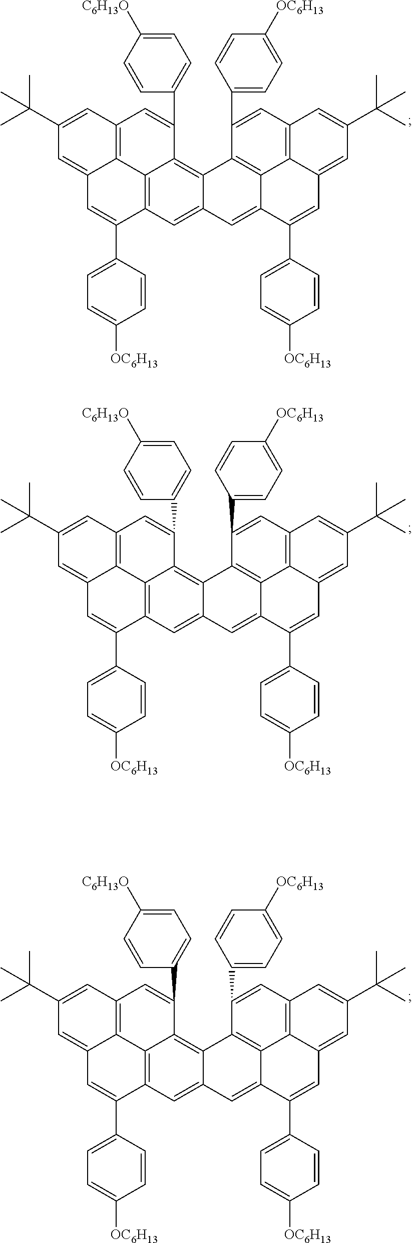

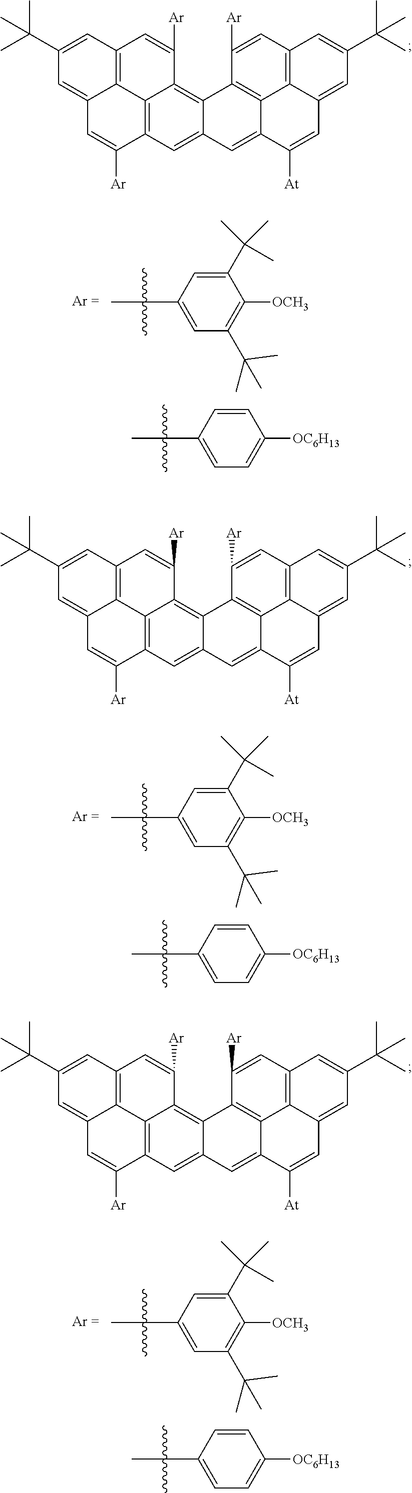

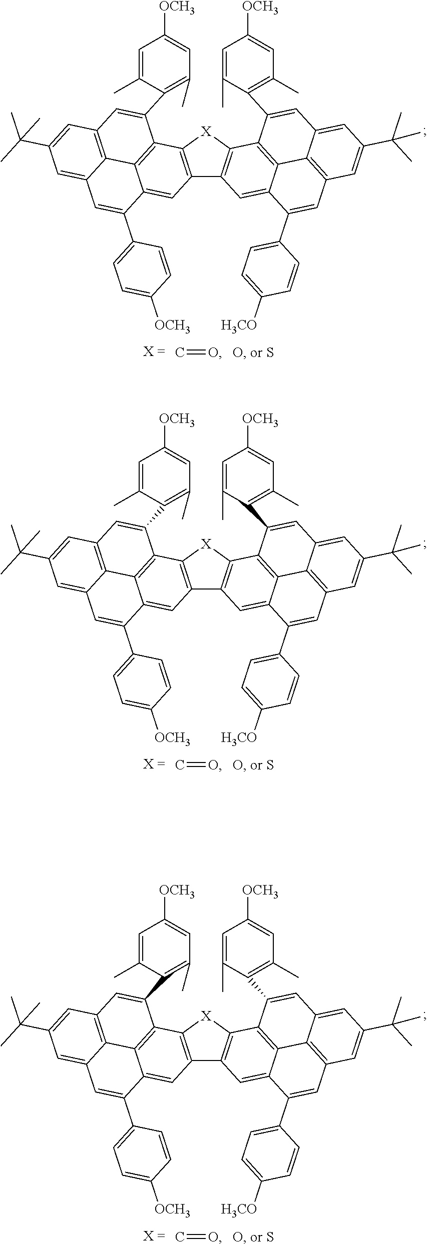

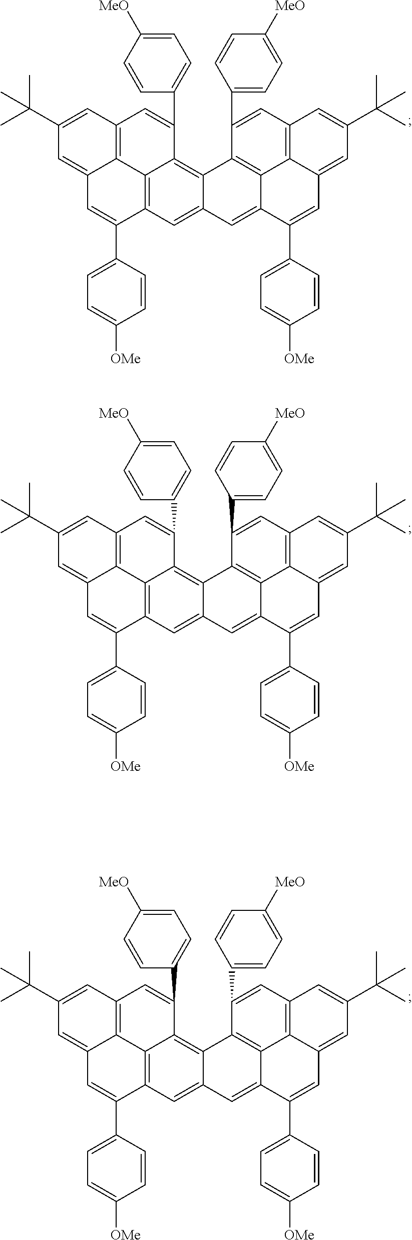

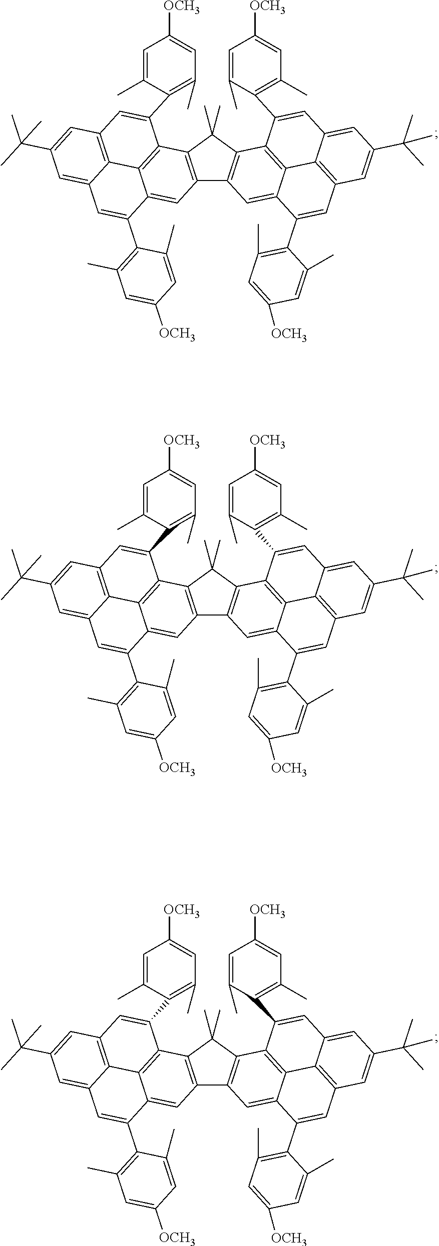

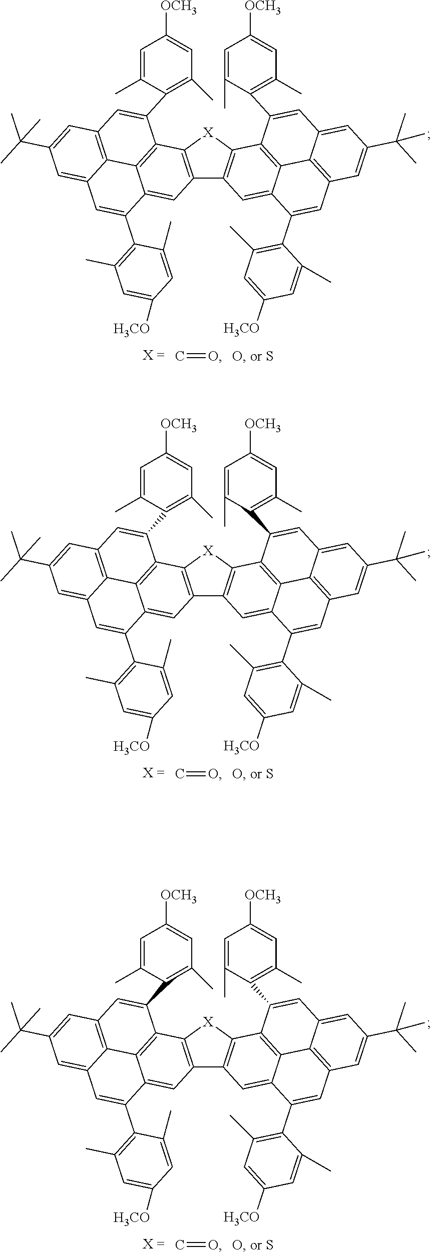

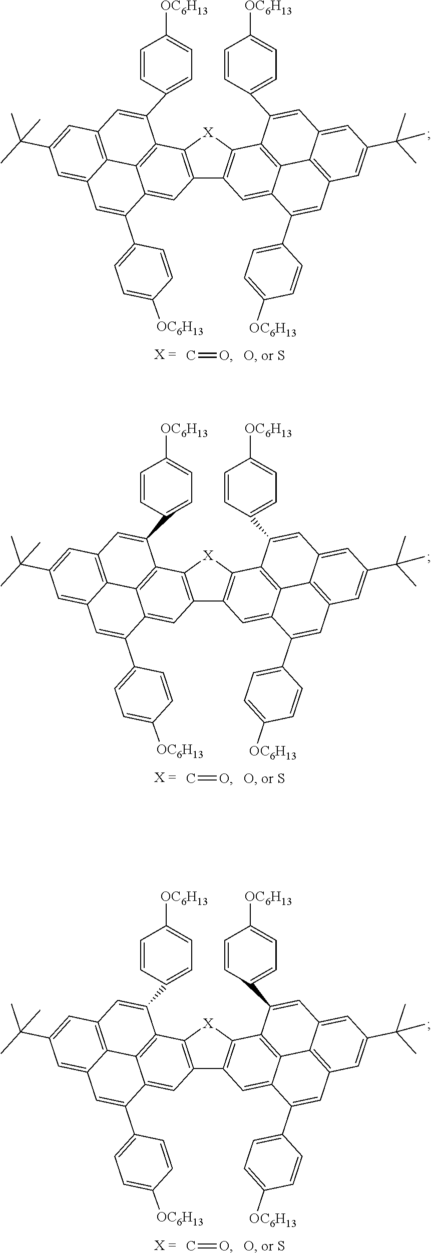

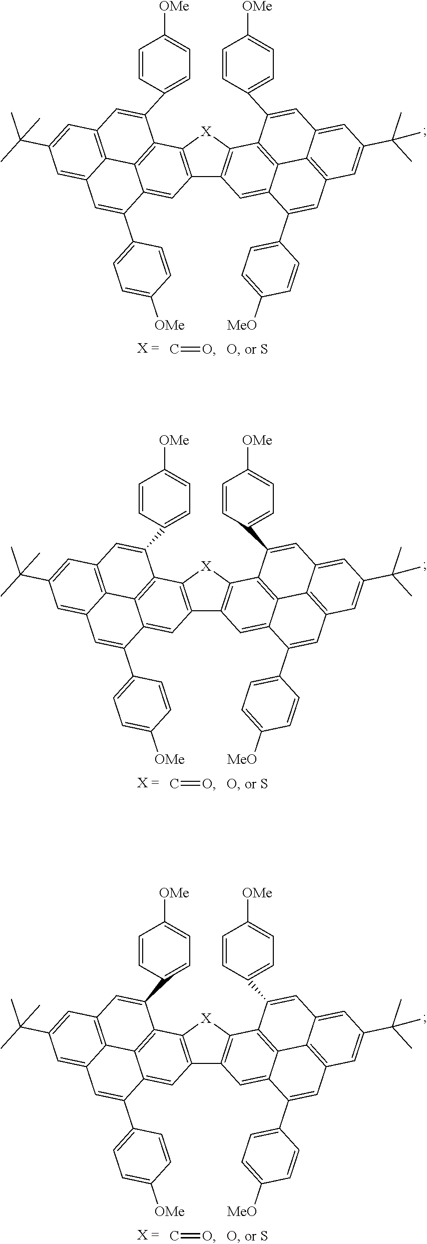

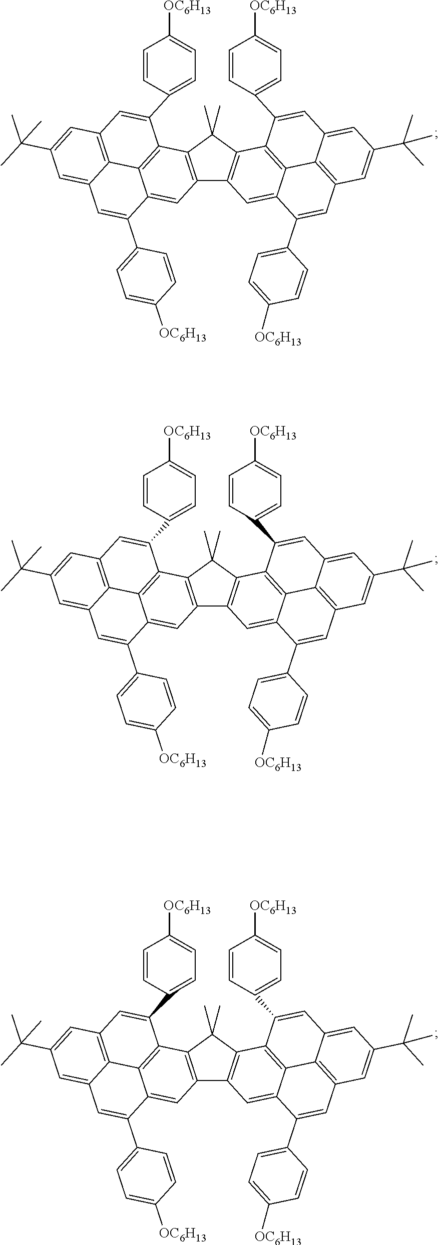

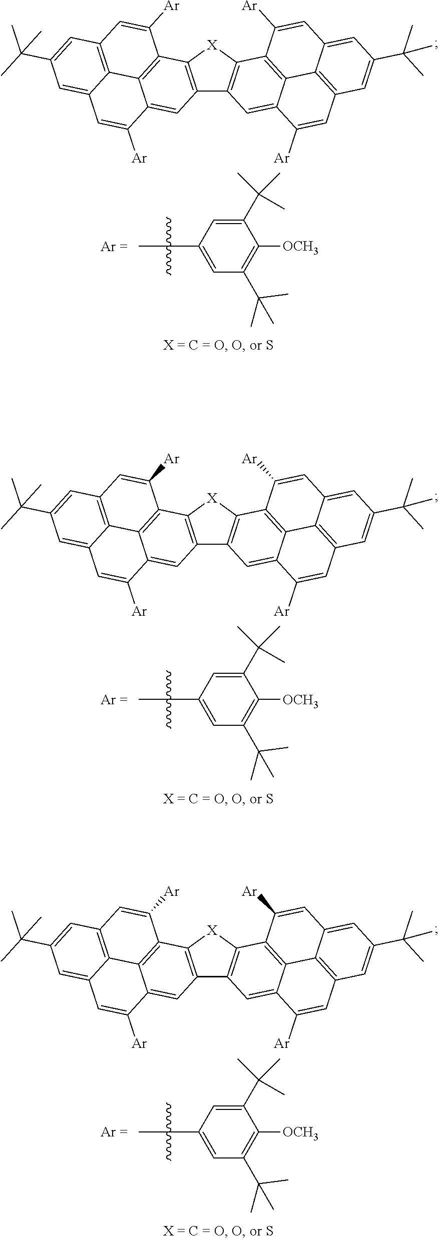

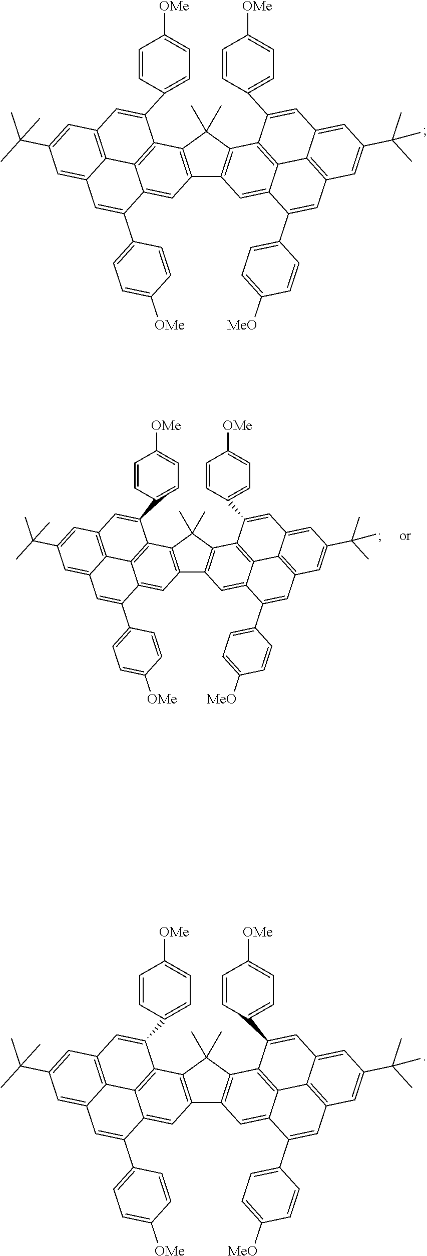

16. A compound, having a structure satisfying Formula 3 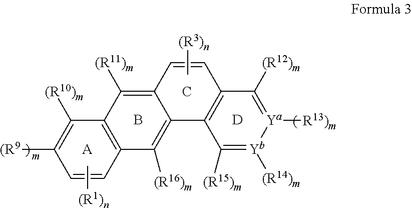
wherein
Y^{a} is carbon, CH (when R⁷ is not present, as in when m is 0), or nitrogen;
Y^{b} is carbon, CH (when R⁸ is not present, as in when m is 0), or nitrogen;
each of R¹, R³, R⁹, R¹⁰, R¹¹, R¹², R¹³, R¹⁴, R¹⁵, and R¹⁶, in each independent instance, independently is selected from aliphatic, haloaliphatic, haloheteroaliphatic, heteroaliphatic, aliphatic-aromatic, heteroaliphatic-aromatic, or aromatic; or any one or more of (i)-(iv) apply, wherein
(i) R¹ and R⁹ join together to form an aryl group or a heteroaryl group, or any combination thereof;
(ii) R⁹ and R¹⁰ join together to form an aryl group or a heteroaryl group, or any combination thereof;
(iii) R¹⁰ and R¹¹ join together to form an aryl group or a heteroaryl group, or any combination thereof;
(iv) R¹² and R¹³ join together to form an aryl group or a heteroaryl group, or any combination thereof;
(v) R¹², R¹³, and R¹⁴ join together to form an aromatic group having a structure 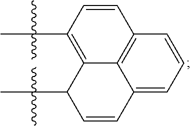 [SMILES: *c1ccc2cccc3c2c1C(*)C=C3]
or an aromatic group having a structure 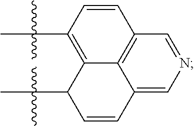 [SMILES: *c1ccc2cncc3c2c1C(*)C=C3]
(vi) R¹³ and R¹⁴ can join together to form an aryl group or a heteroaryl group, or any combination thereof; and/or
(vii) R¹⁵ and R¹⁶ can join together to form an aryl group or a heteroaryl group, or any combination thereof;
each n independently is an integer selected from 0, 1 or 2; and
each m independently is 0 or 1.

17. The compound of claim 16, wherein the compound has a structure satisfying one or more of Formulas 3A-3R 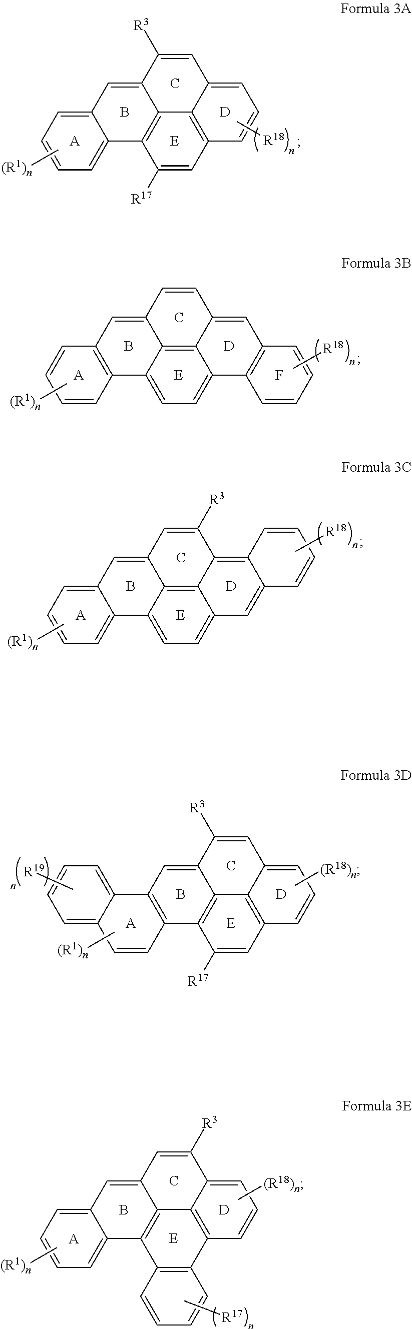 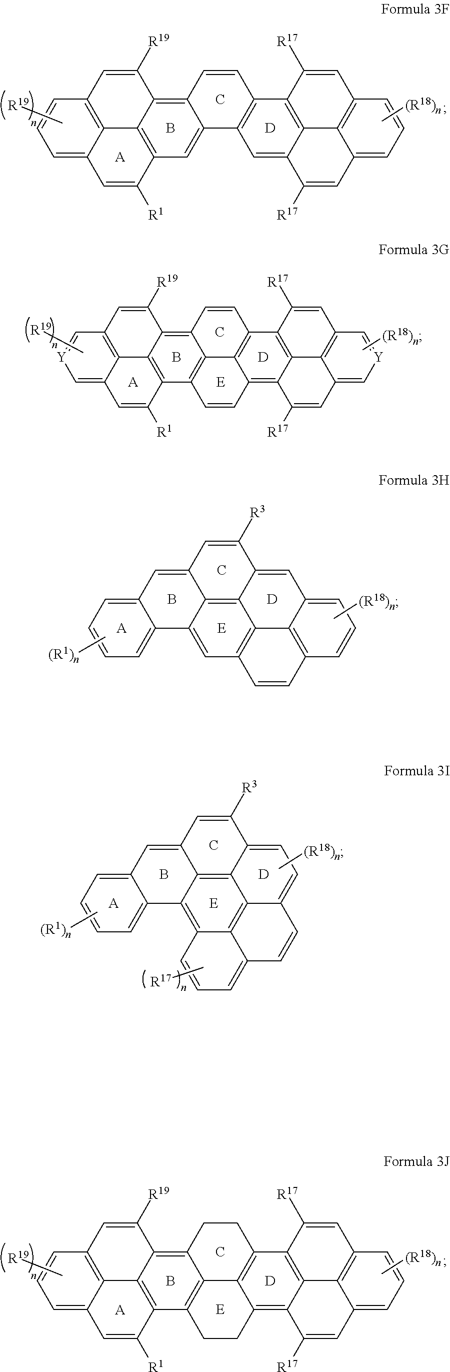 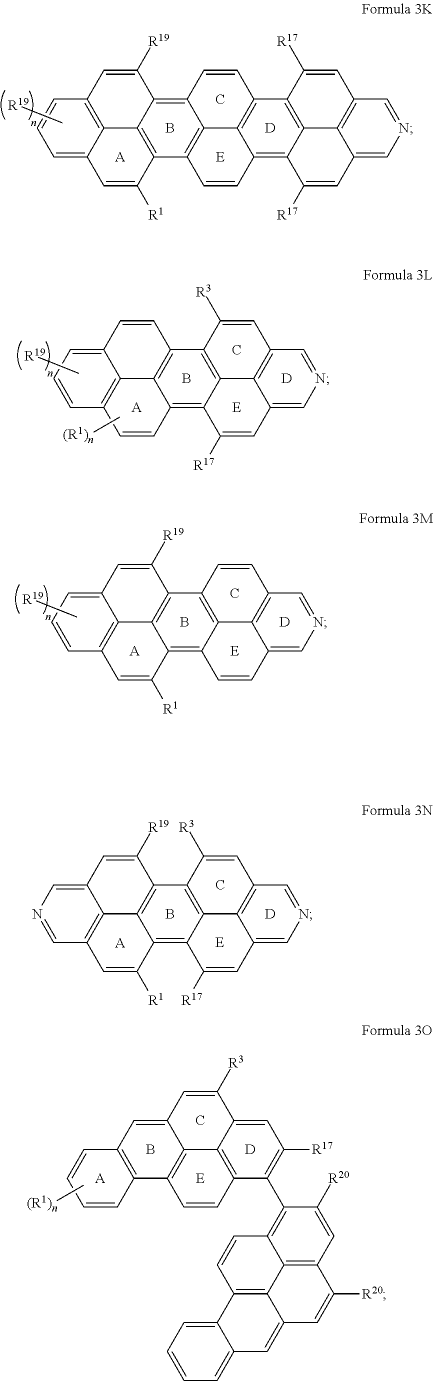 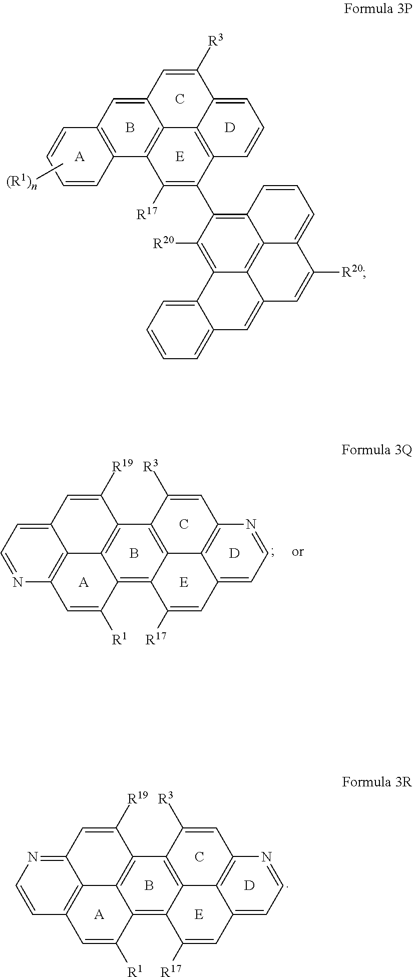
wherein
each R¹⁷ independently are selected from aliphatic, haloaliphatic, haloheteroaliphatic, heteroaliphatic, aliphatic-aromatic, heteroaliphatic-aromatic, or aromatic;
each R¹⁸ independently are selected from aliphatic, haloaliphatic, haloheteroaliphatic, heteroaliphatic, aliphatic-aromatic, heteroaliphatic-aromatic, or aromatic;
each R¹⁹ independently are selected from aliphatic, haloaliphatic, haloheteroaliphatic, heteroaliphatic, aliphatic-aromatic, heteroaliphatic-aromatic, or aromatic;
each R²⁰ independently are selected from aliphatic, haloaliphatic, haloheteroaliphatic, heteroaliphatic, aliphatic-aromatic, heteroaliphatic-aromatic, or aromatic; and
each Y independently is CH or nitrogen.

18. (canceled)

19. The compound of claim 16, wherein the compound is selected from

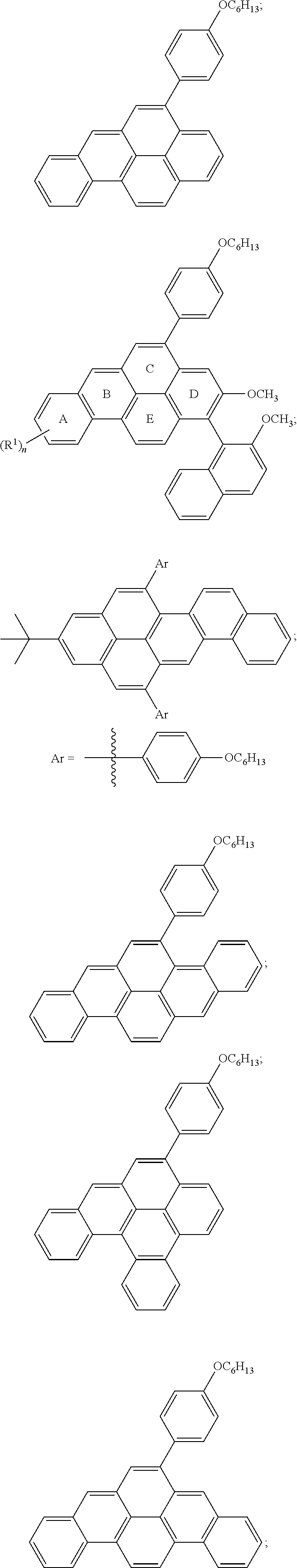

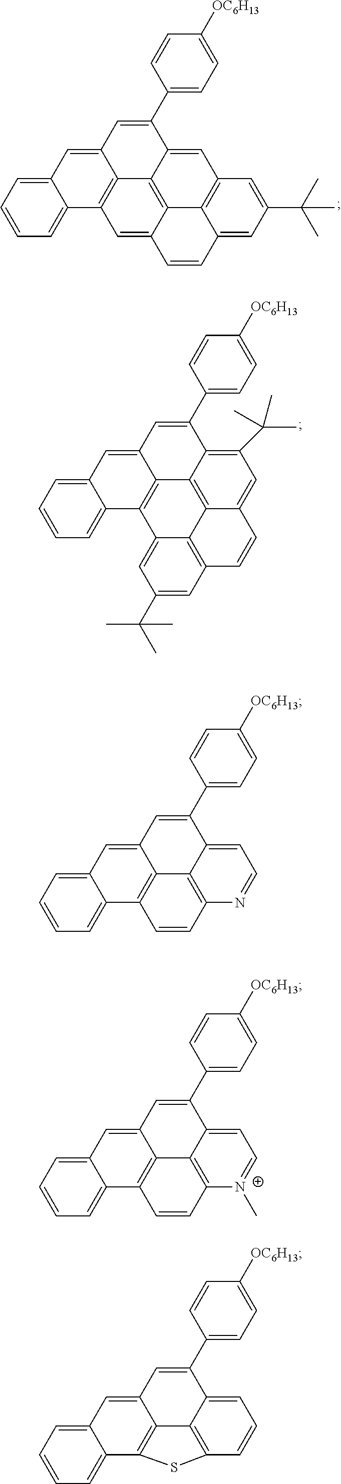

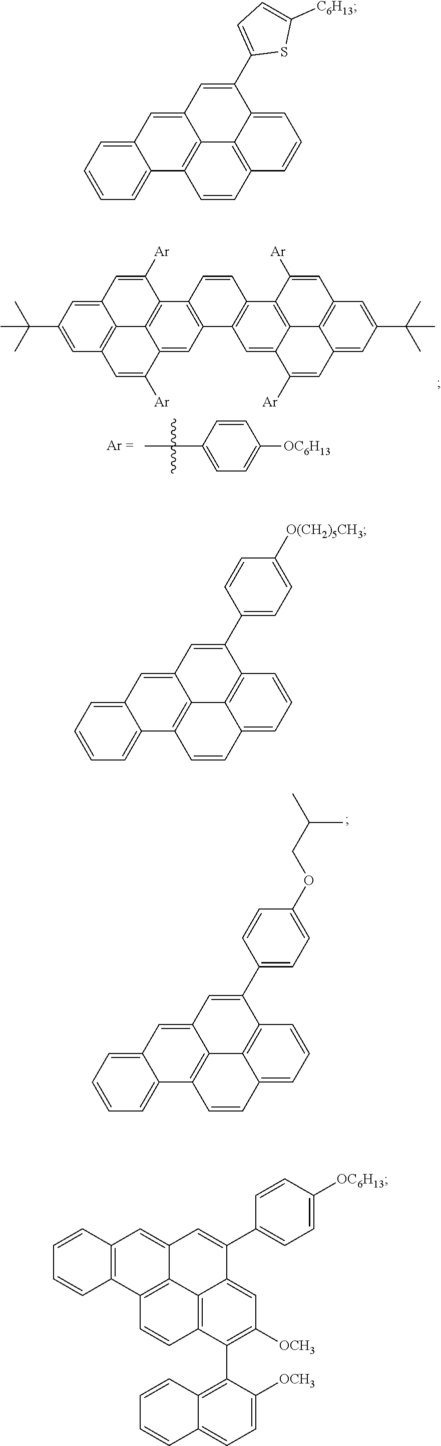

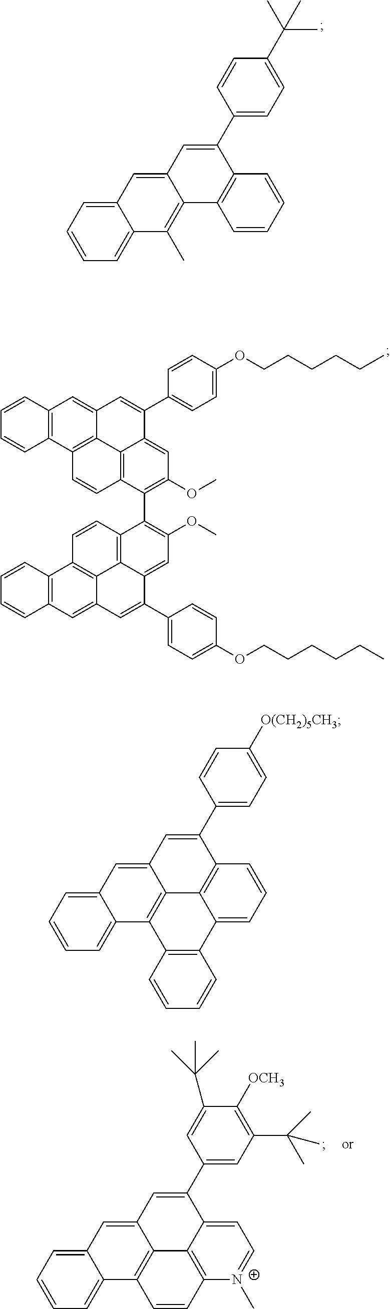

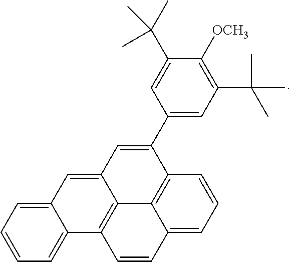 [SMILES: COc1c(C(C)(C)C)cc(-c2cc3cc4ccccc4c4ccc5cccc2c5c34)cc1C(C)(C)C]

20. A compound, having a structure satisfying Formula 5 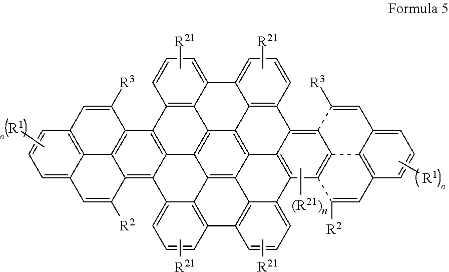
wherein
each R¹ independently is selected from halogen, aliphatic, haloaliphatic, haloheteroaliphatic, heteroaliphatic, aromatic, aliphatic-aromatic, heteroaliphatic-aromatic or any combination thereof;
each R² independently is selected from halogen, aliphatic, haloaliphatic, haloheteroaliphatic, heteroaliphatic, aromatic, aliphatic-aromatic, heteroaliphatic-aromatic or any combination thereof;
each R³ independently is selected from halogen, aliphatic, haloaliphatic, haloheteroaliphatic, heteroaliphatic, aromatic, aliphatic-aromatic, heteroaliphatic-aromatic or any combination thereof;
each R²¹ independently is aliphatic, heteroaliphatic, aliphatic-aromatic, heteroaliphatic-aromatic, aromatic, or any combination thereof; and
each n independently is an integer selected from 0 to 3.

21. The compound of claim 20, wherein the compound has a structure satisfying one or more of Formulas 5A, 5B, or 5C 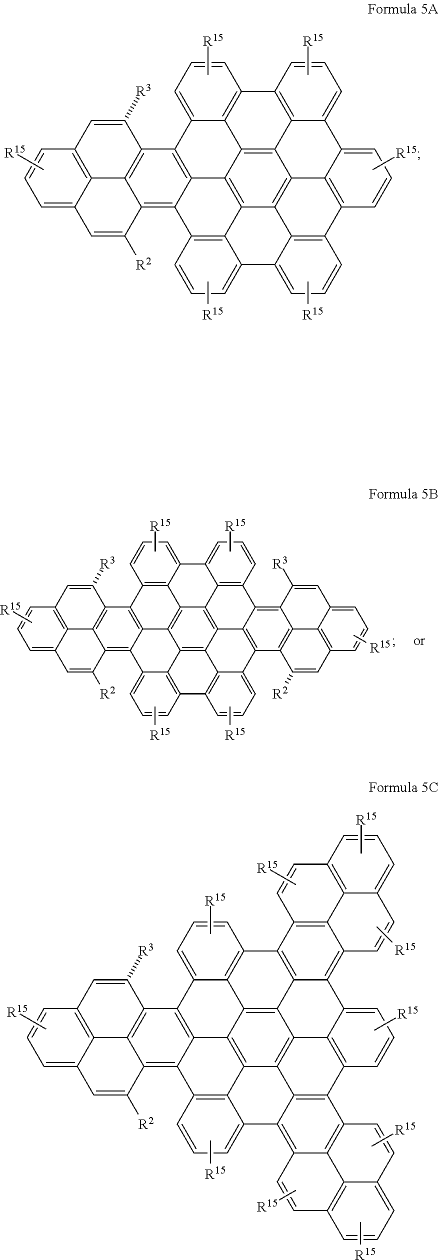
wherein each R¹⁵ independently is selected from alkyl, alkenyl, alkynyl, heteroalkyl, heteroalkenyl, heterolkynyl, haloalkyl, haloalkenyl, haloalkynyl, haloheteroalkyl, haloheteroalkenyl, haloheteroalkynyl, aryl, heteroaryl, alkyl-aryl/alkeny-aryl/alkynyl-aryl, alkyl-heteroaryl/alkenyl-heteroaryl/alkynyl-heteroaryl, heteroalkyl-aryl/heteroalkenyl-aryl/heteroalkynyl-aryl, heteroalkyl-heteroaryl/heteroalkenyl-heteroaryl/heteroalkynyl-heteroaryl or any combination thereof.

22. The compound of claim 20, wherein the compound is selected from

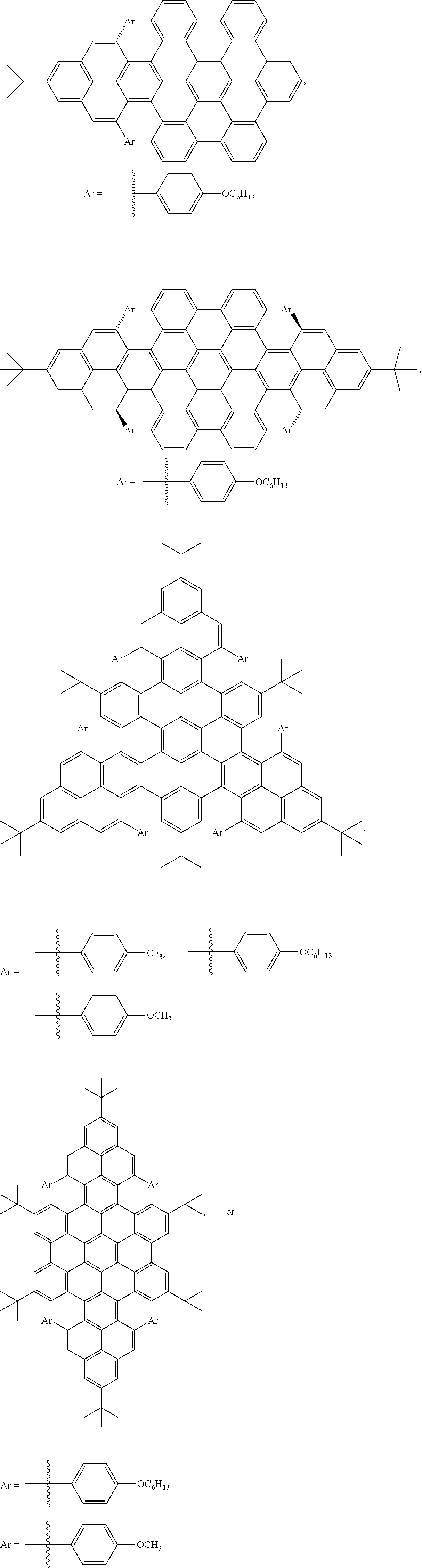

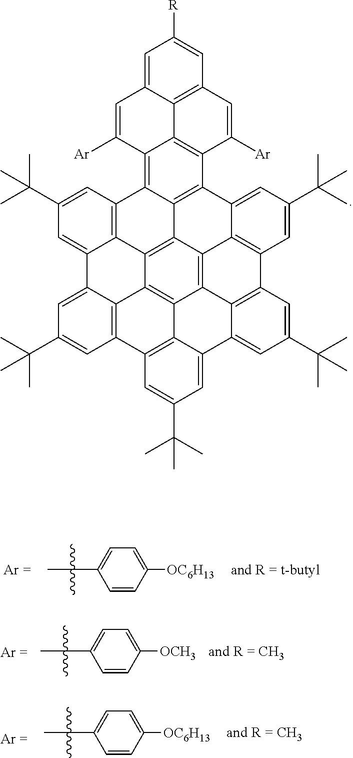

23. A method for making the compound of claim 1, comprising: exposing a compound comprising a first aromatic group functionalized with (i) one or more alkyne moieties and (ii) a second aromatic group to a catalyst effective to promote forming an intramolecular bond between the one or more alkyne moieties of the first aromatic group and a carbon atom of the second aromatic group.

24. The method of claim 23, wherein the method further comprises pre-forming the compound by using a transition metal to couple a starting material comprising the first aromatic group and further comprising a boronic acid or a boronic ester with a starting material comprising the second aromatic group and further comprising a halogen atom.

25. The method of claim 23, wherein the compound further comprises a third aromatic group functionalized with one or more alkyne moieties.

26. The method of claim 23, wherein the catalyst is effective to promote forming an intramolecular bond between the one or more alkyne moieties of the third aromatic group and a carbon atom of the second aromatic group.

27. The method of claim 23, wherein the catalyst is a Brønsted acid or a Lewis acid selected from HCO₂CF₃, HOSO₂CH₃, HOSO₂CF₃, InCl₃, PtCl₂, AuCl₃, or AuCl(PPh₃).

28. (canceled)

29. A device, comprising a compound according to claim 1, wherein the device is an electronic device selected from an organic transistor, a light-emitting device, a sensor device, or an organic photovoltaic device; or a device for detecting biological compounds.
